SEQUENCE LISTING (110 sequences)

SEQ ID NO: 1 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 2 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 3 - protein, 5 aa, synthetic construct
  REGION 1..5: note = FAP (212) CDR-H1
DYNMD

SEQ ID NO: 4 - protein, 17 aa, synthetic construct
  REGION 1..17: note = FAP (212) CDR-H2
DIYPNTGGTIYNQKFKG

SEQ ID NO: 5 - protein, 10 aa, synthetic construct
  REGION 1..10: note = FAP (212) CDR-H3
FRGIHYAMDY

SEQ ID NO: 6 - protein, 15 aa, synthetic construct
  REGION 1..15: note = FAP (212) CDR-L1
RASESVDNYGLSFIN

SEQ ID NO: 7 - protein, 7 aa, synthetic construct
  REGION 1..7: note = FAP (212) CDR-L2
GTSNRGS

SEQ ID NO: 8 - protein, 9 aa, synthetic construct
  REGION 1..9: note = FAP (212) CDR-L3
QQSNEVPYT

SEQ ID NO: 9 - protein, 119 aa, synthetic construct
  REGION 1..119: note = FAP (212) VH
EVLLQQSGPELVKPGASVKIACKASGYTLTDYNMDWVRQSHGKSLEWIGDIYPNTGGTIYNQKFKGKATLTIDKSSSTAYMDLRSLTSEDTAVYYCTRFRGIHYAMDYWGQGTSVTVSS

SEQ ID NO: 10 - protein, 111 aa, synthetic construct
  REGION 1..111: note = FAP (212) VL
DIVLTQSPVSLAVSLGQRATISCRASESVDNYGLSFINWFQQKPGQPPKLLIYGTSNRGSGVPARFSGSGSGTDFSLNIHPMEEDDTAMYFCQQSNEVPYTFGGGTNLEIK

SEQ ID NO: 11 - protein, 17 aa, synthetic construct
  REGION 1..17: note = FAP (VH1G3a) CDR-H2
DIYPNTGGTIYAQKFQG

SEQ ID NO: 12 - protein, 17 aa, synthetic construct
  REGION 1..17: note = FAP (VH2G3a) CDR-H2
DIYPNTGGTIYADSVKG

SEQ ID NO: 13 - protein, 15 aa, synthetic construct
  REGION 1..15: note = FAP (VL1G3a) CDR-L1
RASESVDNYGLSFLA

SEQ ID NO: 14 - protein, 15 aa, synthetic construct
  REGION 1..15: note = FAP (VL2G3a) CDR-L1
RASESIDNYGLSFLN

SEQ ID NO: 15 - protein, 119 aa, synthetic construct
  REGION 1..119: note = FAP (VH1G1a)
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSS

SEQ ID NO: 16 - protein, 119 aa, synthetic construct
  REGION 1..119: note = FAP (VH1G2a)
QVQLVQSGAEVKKPGASVKVSCKASGYTLTDYNMDWVRQAPGKSLEWIGDIYPNTGGTIYNQKFKGRVTMTIDKSTSTVYMELSSLRSEDTAVYYCTRFRGIHYAMDYWGQGTTVTVSS

SEQ ID NO: 17 - protein, 119 aa, synthetic construct
  REGION 1..119: note = FAP (VH1G3a)
QVQLVQSGAEVKKPGASVKVSCKASGYTLTDYNMDWVRQAPGQGLEWIGDIYPNTGGTIYAQKFQGRVTMTIDTSTSTVYMELSSLRSEDTAVYYCTRFRGIHYAMDYWGQGTTVTVSS

SEQ ID NO: 18 - protein, 119 aa, synthetic construct
  REGION 1..119: note = FAP (VH2G1a)
EVQLLESGGGLVQPGGSLRLSCAASGYTLTDYNMDWVRQAPGKGLEWVGDIYPNTGGTIYNQKFKGRFTISRDNSKNTLYLQMNSLRAEDTAVYYCTRFRGIHYAMDYWGQGTTVTVSS

SEQ ID NO: 19 - protein, 119 aa, synthetic construct
  REGION 1..119: note = FAP (VH2G2a)
EVQLLESGGGLVQPGGSLRLSCAASGYTLTDYNMDWVRQAPGKGLEWVGDIYPNTGGTIYNQKFKGRFTISIDKSKNTAYLQMNSLRAEDTAVYYCTRFRGIHYAMDYWGQGTTVTVSS

SEQ ID NO: 20 - protein, 119 aa, synthetic construct
  REGION 1..119: note = FAP (VH2G3a)
EVQLLESGGGLVQPGGSLRLSCAASGYTLTDYNMDWVRQAPGKGLEWVGDIYPNTGGTIYADSVKGRFTISRDNSKNTLYLQMNSLRAEDTAVYYCTRFRGIHYAMDYWGQGTTVTVSS

SEQ ID NO: 21 - protein, 111 aa, synthetic construct
  REGION 1..111: note = FAP (VL1G1a)
EIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIK

SEQ ID NO: 22 - protein, 111 aa, synthetic construct
  REGION 1..111: note = FAP (VL1G2a)
EIVLTQSPATLSLSPGERATLSCRASESVDNYGLSFINWFQQKPGQPPRLLIYGTSNRGSGIPARFSGSGSGTDFTLTISSLEPEDFAVYFCQQSNEVPYTFGGGTKVEIK

SEQ ID NO: 23 - protein, 111 aa, synthetic construct
  REGION 1..111: note = FAP (VL1G3a)
EIVLTQSPATLSLSPGERATLSCRASESVDNYGLSFLAWFQQKPGQAPRLLIYGTSNRGSGIPARFSGSGSGTDFTLTISSLEPEDFAVYFCQQSNEVPYTFGGGTKVEIK

SEQ ID NO: 24 - protein, 111 aa, synthetic construct
  REGION 1..111: note = FAP (VL2G1a)
DIQMTQSPSSLSASVGDRVTITCRASESVDNYGLSFINWFQQKPGKAPKLLIYGTSNRGSGVPSRFSGSGSGTDFTLTISSLQPEDFATYFCQQSNEVPYTFGGGTKVEIK

SEQ ID NO: 25 - protein, 111 aa, synthetic construct
  REGION 1..111: note = FAP (VL2G2a)
DIQMTQSPSSLSASVGDRVTITCRASESVDNYGLSFINWFQQKPGQPPKLLIYGTSNRGSGVPSRFSGSGSGTDFTLTISSLQPEDFAMYFCQQSNEVPYTFGGGTKVEIK

SEQ ID NO: 26 - protein, 111 aa, synthetic construct
  REGION 1..111: note = FAP (VL2G3a)
DIQMTQSPSSLSASVGDRVTITCRASESIDNYGLSFLNWFQQKPGKAPKLLIYGTSNRGSGVPSRFSGSGSGTDFTLTISSLQPEDFATYFCQQSNEVPYTFGGGTKVEIK

SEQ ID NO: 27 - protein, 5 aa, synthetic construct
  REGION 1..5: note = hu CD40 CDR-H1
GYYIH

SEQ ID NO: 28 - protein, 17 aa, synthetic construct
  REGION 1..17: note = hu CD40 CDR-H2
RVIPNAGGTSYNQKFKG

SEQ ID NO: 29 - protein, 5 aa, synthetic construct
  REGION 1..5: note = hu CD40 CDR-H3
EGIYW

SEQ ID NO: 30 - protein, 16 aa, synthetic construct
  REGION 1..16: note = hu CD40 CDR-L1
RSSQSLVHSNGNTFLH

SEQ ID NO: 31 - protein, 7 aa, synthetic construct
  REGION 1..7: note = hu CD40 CDR-L2
TVSNRFS

SEQ ID NO: 32 - protein, 9 aa, synthetic construct
  REGION 1..9: note = hu CD40 CDR-L3
SQTTHVPWT

SEQ ID NO: 33 - protein, 114 aa, synthetic construct [sequence omitted]

SEQ ID NO: 34 - protein, 112 aa, synthetic construct [sequence omitted]

SEQ ID NO: 35 - protein, 114 aa, synthetic construct
  REGION 1..114: note = CD40 (S2C6) VH
EVQLQQSGPDLVKPGASVKISCKASGYSFTGYYIHWVKQSHGKSLEWIGRVIPNNGGTSYNQKFKGKAILTVDKSSSTAYMELRSLTSEDSAVYYCAREGIYWWGHGTTLTVSS

SEQ ID NO: 36 - protein, 112 aa, synthetic construct
  REGION 1..112: note = CD40 (S2C6) VL
DVVVTQTPLSLPVSLGAQASISCRSSQSLVHSNGNTFLHWYLQKPGQSPKLLIYTVSNRFSGVPDRFSGSGSGTDFTLKISRVEAEDLGVYFCSQTTHVPWTFGGGTKLEIQ

SEQ ID NO: 37 - protein, 114 aa, synthetic construct
  REGION 1..114: note = VH1a (CD40)
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSS

SEQ ID NO: 38 - protein, 114 aa, synthetic construct
  REGION 1..114: note = VH1b (CD40)
QVQLVQSGAEVKKPGASVKVSCKASGYSFTGYYIHWVRQAPGKSLEWMGRVIPNAGGTSYNQKFKGRVTLTVDKSISTAYMELSRLRSDDTAVYYCAREGIYWWGQGTTVTVSS

SEQ ID NO: 39 - protein, 114 aa, synthetic construct
  REGION 1..114: note = VH1c (CD40)
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGHGTTVTVSS

SEQ ID NO: 40 - protein, 114 aa, synthetic construct
  REGION 1..114: note = VH1d (CD40)
QVQLVQSGAEVKKPGASVKVSCKASGYSFTGYYIHWVRQAPGQSLEWMGRVIPNAGGTSYNQKFKGRVTLSVDKSISTAYMELSRLRSDDTAVYYCAREGIYWWGQGTTVTVSS

SEQ ID NO: 41 - protein, 112 aa, synthetic construct
  REGION 1..112: note = VL1a (CD40)
DIVMTQTPLSLSVTPGQPASISCRSSQSLVHSNGNTFLHWYLQKPGQSPQLLIYTVSNRFSGVPDRFSGSGSGTDFTLKISRVEAEDVGVYFCSQTTHVPWTFGGGTKVEIK

SEQ ID NO: 42 - protein, 112 aa, synthetic construct
  REGION 1..112: note = VL1b (CD40)
DIVVTQTPLSLSVTPGQPASISCRSSQSLVHSNGNTFLHWYLQKPGQSPQLLIYTVSNRFSGVPDRFSGSGSGTDFTLKISRVEAEDVGVYFCSQTTHVPWTFGGGTKVEIK

SEQ ID NO: 43 - protein, 112 aa, synthetic construct
  REGION 1..112: note = VL1c (CD40)
DVVVTQTPLSLSVTPGQPASISCRSSQSLVHSNGNTFLHWYLQKPGQSPQLLIYTVSNRFSGVPDRFSGSGSGTDFTLKISRVEAEDVGVYFCSQTTHVPWTFGGGTKVEIK

SEQ ID NO: 44 - protein, 112 aa, synthetic construct
  REGION 1..112: note = VL1d (CD40)
DVVVTQTPLSLSVTPGQPASISCRSSQSLVHSNGNTFLHWYLQKPGQSPQLLIYTVSNRFSGVPDRFSGSGSGTDFTLKISRVEAEDVGVYFCSQTTHVPWTFGGGTKLEIK

SEQ ID NO: 45 - protein, 114 aa, synthetic construct
  REGION 1..114: note = VH2a (CD40)
EVQLLESGGGLVQPGGSLRLSCAASGYSFTGYYIHWVRQAPGKGLEWVGRVIPNAGGTSYNQKFKGRFTISVDNSKNTAYLQMNSLRAEDTAVYYCAREGIYWWGQGTTVTVSS

SEQ ID NO: 46 - protein, 114 aa, synthetic construct
  REGION 1..114: note = VH2b (CD40)
EVQLLESGGGLVQPGGSLRLSCAASGYSFTGYYIHWVRQAPGKSLEWVGRVIPNAGGTSYNQKFKGRFTISVDNSKNTAYLQMNSLRAEDTAVYYCAREGIYWWGQGTTVTVSS

SEQ ID NO: 47 - protein, 114 aa, synthetic construct
  REGION 1..114: note = VH2c (CD40)
EVQLLESGGGLVQPGGSLRLSCAASGYSFTGYYIHWVRQAPGKGLEWVGRVIPNAGGTSYNQKFKGRFTISVDNSKNTAYLQMNSLRAEDTAVYYCAREGIYWWGHGTTVTVSS

SEQ ID NO: 48 - protein, 114 aa, synthetic construct
  REGION 1..114: note = VH2d (CD40)
EVQLLESGGGLVQPGGSLRLSCAASGYSFTGYYIHWVRQAPGKGLEWVGRVIPNAGGTSYGDSVKGRFTISVDNSKNTAYLQMNSLRAEDTAVYYCAREGIYWWGQGTTVTVSS

SEQ ID NO: 49 - protein, 114 aa, synthetic construct
  REGION 1..114: note = VH2ab (CD40)
EVQLLESGGGLVQPGGSLRLSCAASGYSFTGYYMHWVRQAPGKGLEWVGRVIPNAGGTSYNQKFKGRFTISVDNSKNTAYLQMNSLRAEDTAVYYCAREGIYWWGQGTTVTVSS

SEQ ID NO: 50 - protein, 114 aa, synthetic construct
  REGION 1..114: note = VH2ac (CD40)
EVQLLESGGGLVQPGGSLRLSCAASGYSFTGYYIHWVRQAPGKGLEWVGRVIPNAGGTSYNQKVKGRFTISVDNSKNTAYLQMNSLRAEDTAVYYCAREGIYWWGQGTTVTVSS

SEQ ID NO: 51 - protein, 112 aa, synthetic construct
  REGION 1..112: note = VL2a (CD40)
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGGGTKVEIK

SEQ ID NO: 52 - protein, 112 aa, synthetic construct
  REGION 1..112: note = VL2b (CD40)
DIQMTQSPSSLSASVGDRVTITCRSSQSLVHSNGNTFLHWYQQKPGQSPKLLIYTVSNRFSGVPSRFSGSGSGTDFTLTISSLQPEDFATYFCSQTTHVPWTFGGGTKVEIK

SEQ ID NO: 53 - protein, 112 aa, synthetic construct
  REGION 1..112: note = VL2ab (CD40)
DIQMTQSPSSLSASVGDRVTITCRASQSLVHSNGNTFLHWYQQKPGKAPKLLIYTVSNRFSGVPSRFSGSGSGTDFTLTISSLQPEDFATYFCSQTTHVPWTFGGGTKVEIK

SEQ ID NO: 54 - protein, 112 aa, synthetic construct
  REGION 1..112: note = VL2ac (CD40)
DIQMTQSPSSLSASVGDRVTITCRSSQSIVHSNGNTFLHWYQQKPGKAPKLLIYTVSNRFSGVPSRFSGSGSGTDFTLTISSLQPEDFATYFCSQTTHVPWTFGGGTKVEIK

SEQ ID NO: 55 - protein, 443 aa, synthetic construct
  REGION 1..443: note = P1AE0400 Heavy chain
EVQLLESGGGLVQPGGSLRLSCAASGYSFTGYYIHWVRQAPGKGLEWVGRVIPNAGGTSYNQKFKGRFTISVDNSKNTAYLQMNSLRAEDTAVYYCAREGIYWWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 219 aa, synthetic construct
  REGION 1..219: note = P1AE0400 light chain
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 443 aa, synthetic construct
  REGION 1..443: note = P1AE0403 heavy chain
EVQLLESGGGLVQPGGSLRLSCAASGYSFTGYYIHWVRQAPGKGLEWVGRVIPNAGGTSYGDSVKGRFTISVDNSKNTAYLQMNSLRAEDTAVYYCAREGIYWWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 219 aa, synthetic construct; identical to SEQ ID NO: 56
  REGION 1..219: note = P1AE0403 light chain

SEQ ID NO: 59 - protein, 443 aa, synthetic construct
  REGION 1..443: note = P1AE0817 heavy chain
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 219 aa, synthetic construct
  REGION 1..219: note = P1AE0817 light chain
DIVMTQTPLSLSVTPGQPASISCRSSQSLVHSNGNTFLHWYLQKPGQSPQLLIYTVSNRFSGVPDRFSGSGSGTDFTLKISRVEAEDVGVYFCSQTTHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 226 aa, synthetic construct
  REGION 1..226: note = (P1AE1689) light chain cross VH-Ckappa
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 219 aa, synthetic construct
  REGION 1..219: note = VL1a (CD40) light chain (charged)
DIVMTQTPLSLSVTPGQPASISCRSSQSLVHSNGNTFLHWYLQKPGQSPQLLIYTVSNRFSGVPDRFSGSGSGTDFTLKISRVEAEDVGVYFCSQTTHVPWTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 679 aa, synthetic construct
  REGION 1..679: note = VH1a (CD40) (VHCH1 charged) Fc knob_PGLALA_(P1AE1689) (VL-CH1)
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 64 - protein, 443 aa, synthetic construct
  REGION 1..443: note = VH1a (CD40) (VHCH1 charged) Fc hole_PGLALA
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 65 - protein, 216 aa, synthetic construct
  REGION 1..216: note = (P1AE1689) light chain cross VL-CH1
EIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 66 - protein, 219 aa, synthetic construct; identical to SEQ ID NO: 60
  REGION 1..219: note = VL1a (CD40) light chain

SEQ ID NO: 67 - protein, 689 aa, synthetic construct
  REGION 1..689: note = VH1a (CD40) (VHCH1) Fc knob_PGLALA_(P1AE1689) (VH-Ckappa)
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSQVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 443 aa, synthetic construct
  REGION 1..443: note = VH1a (CD40) (VHCH1) Fc hole_PGLALA
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 69 - protein, 907 aa, synthetic construct
  REGION 1..907: note = VH1a (CD40) (VHCH1 charged_VH1a (CD40) (VHCH1 charged)-Fc knob_PGLALA_(P1AE1689) (VL-CH1)
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 70 - protein, 671 aa, synthetic construct
  REGION 1..671: note = VH1a (CD40) (VHCH1 charged)_VH1a (CD40) (VHCH1 charged)-Fc hole_PGLALA
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 71 - protein, 213 aa, synthetic construct
  REGION 1..213: note = 4B9 light chain cross VL-CH1
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 72 - protein, 687 aa, synthetic construct
  REGION 1..687: note = VH1a (CD40) (VHCH1) Fc knob_PGLALA_4B9 (VH-Ckappa)
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 915 aa, synthetic construct
  REGION 1..915: note = VH1a (CD40) (VHCH1) _VH1a (CD40) (VHCH1)-Fc knob_PGLALA_(4B9) (VH-Ckappa)
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 671 aa, synthetic construct
  REGION 1..671: note = VH1a (CD40) (VHCH1)_VH1a (CD40) (VHCH1)-Fc hole_PGLALA
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 75 - protein, 223 aa, synthetic construct
  REGION 1..223: note = 28H1 light chain cross VH-Ckappa
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 676 aa, synthetic construct
  REGION 1..676: note = VH1a (CD40) (VHCH1 charged) Fc knob_PGLALA_28H1 (VL-CH1)
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 77 - protein, 904 aa, synthetic construct
  REGION 1..904: note = VH1a (CD40) (VHCH1 charged) _VH1a (CD40) (VHCH1 charged) Fc knob_PGLALA_28H1 (VL-CH1)
QVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQVQLVQSGAEVKKPGASVKVSCKASGYSFTGYYIHWVRQAPGQSLEWMGRVIPNAGGTSYNQKFKGRVTLTVDKSISTAYMELSRLRSDDTAVYYCAREGIYWWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 78 - protein, 748 aa, synthetic construct
  REGION 1..748: note = hu FAP ectodomain+poly-lys-tag+his6-tag
RPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSDGKKKKKKGHHHHHH

SEQ ID NO: 79 - protein, 761 aa, Mus musculus
MKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 80 - protein, 749 aa, synthetic construct
  REGION 1..749: note = Murine FAP ectodomain+poly-lys-tag+his6-tag
RPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGILSGRSQNHLYTHMTHFLKQCFSLSDGKKKKKKGHHHHHH

SEQ ID NO: 81 - protein, 748 aa, synthetic construct
  REGION 1..748: note = Cynomolgus FAP ectodomain+poly-lys-tag+his6-tag
RPPRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPFVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEDYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSDGKKKKKKGHHHHHH

SEQ ID NO: 82 - protein, 5 aa, synthetic construct
  REGION 1..5: note = Peptide linker G4S
GGGGS

SEQ ID NO: 83 - protein, 10 aa, synthetic construct
  REGION 1..10: note = Peptide linker (G4S)2
GGGGSGGGGS

SEQ ID NO: 84 - protein, 10 aa, synthetic construct
  REGION 1..10: note = Peptide linker (SG4)2
SGGGGSGGGG

SEQ ID NO: 85 - protein, 14 aa, synthetic construct
  REGION 1..14: note = Peptide linker G4(SG4)2
GGGGSGGGGSGGGG

SEQ ID NO: 86 - protein, 10 aa, synthetic construct
  REGION 1..10: note = Peptide linker
GSPGSSSSGS

SEQ ID NO: 87 - protein, 15 aa, synthetic construct
  REGION 1..15: note = Peptide linker (G4S)3
GGGGSGGGGSGGGGS

SEQ ID NO: 88 - protein, 20 aa, synthetic construct
  REGION 1..20: note = Peptide linker (G4S)4
GGGGSGGGGSGGGGSGGGGS

SEQ ID NO: 89 - protein, 8 aa, synthetic construct
  REGION 1..8: note = Peptide linker
GSGSGSGS

SEQ ID NO: 90 - protein, 8 aa, synthetic construct
  REGION 1..8: note = Peptide linker
GSGSGNGS

SEQ ID NO: 91 - protein, 8 aa, synthetic construct
  REGION 1..8: note = Peptide linker
GGSGSGSG

SEQ ID NO: 92 - protein, 6 aa, synthetic construct
  REGION 1..6: note = Peptide linker
GGSGSG

SEQ ID NO: 93 - protein, 4 aa, synthetic construct
  REGION 1..4: note = Peptide linker
GGSG

SEQ ID NO: 94 - protein, 8 aa, synthetic construct
  REGION 1..8: note = Peptide linker
GGSGNGSG

SEQ ID NO: 95 - protein, 8 aa, synthetic construct
  REGION 1..8: note = Peptide linker
GGNGSGSG

SEQ ID NO: 96 - protein, 6 aa, synthetic construct
  REGION 1..6: note = Peptide linker
GGNGSG

SEQ ID NO: 97 - protein, 227 aa, synthetic construct
  REGION 1..227: note = Fc knob chain
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 227 aa, synthetic construct
  REGION 1..227: note = Fc hole chain
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 99 - protein, 20 aa, Homo sapiens
YYYYYGMDVWGQGTTVTVSS

SEQ ID NO: 100 - protein, 12 aa, Homo sapiens
LTFGGGTKVEIK

SEQ ID NO: 101 - protein, 20 aa, Homo sapiens
YYYYYGMDVWGQGTTVTVSS

SEQ ID NO: 102 - protein, 12 aa, Homo sapiens
LTFGGGTKVEIK

SEQ ID NO: 103 - protein, 20 aa, Homo sapiens
YYYYYGMDVWGQGTTVTVSS

SEQ ID NO: 104 - protein, 12 aa, Homo sapiens
LTFGGGTKVEIK

SEQ ID NO: 105 - protein, 219 aa, synthetic construct
  REGION 1..219: note = huCD40 light chain (charged)
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 676 aa, synthetic construct
  REGION 1..676: note = huCD40 (VHCH1 charged) Fc PGLALA FAP (VL-CH1)
EVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 107 - protein, 118 aa, synthetic construct [sequence omitted]

SEQ ID NO: 108 - protein, 107 aa, synthetic construct [sequence omitted]

SEQ ID NO: 109 - protein, 121 aa, synthetic construct
  REGION 1..121: note = VH (PD-L1)
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 110 - protein, 108 aa, synthetic construct
  REGION 1..108: note = VL (PD-L1)
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

(19) United States
(12) Patent Application Publication
(10) Pub. No.: US 2025/0320305 A1
(43) Pub. Date: Oct. 16, 2025

(54) BISPECIFIC ANTIGEN BINDING MOLECULES COMPRISING ANTI-FAP CLONE 212

(71) Applicant: Hoffmann-La Roche Inc., Little Falls, NJ (US)

(72) Inventors: Peter BRUENKER, Hittnau (CH); Harald DUERR, Starnberg (DE); Christian KLEIN, Bonstetten (CH); Pablo UMANA, Wollerau (CH); Alexander BUJOTZEK, Munchen (DE); Joerg ZIELONKA, Bulach (CH); Christine TRUMPFHELLER, Zurich (CH); Moritz RAPP, Zurich (CH); Marine LE CLECH, Zurich (CH)

(73) Assignee: Hoffmann-La Roche Inc., Little Falls, NJ (US)

(21) Appl. No.: 19/089,910

(22) Filed: Mar. 25, 2025

Related U.S. Application Data
(63) Continuation of application No. 17/557,647, filed on Dec. 21, 2021, now Pat. No. 12,275,794.
Application No. 17/557,647 is a division of application No. 16/588,780, filed on Sep. 30, 2019, now Pat. No. 11,242,396.

(30) Foreign Application Priority Data
Oct. 1, 2018 (EP) 18197866.9

(51) Int. Cl. C07K 16/28; A61K 39/00; A61K 39/395; A61P 35/00; C07K 1/16; C07K 16/40

(52) U.S. Cl. CPC C07K 16/2878; A61K 39/3955; A61P 35/00; C07K 1/16; C07K 16/40; A61K 2039/505; C07K 2317/24; C07K 2317/31; C07K 2317/565; C07K 2317/94

(57) ABSTRACT

The invention relates to novel bispecific antigen binding molecules, comprising (a) at least one antigen binding domain capable of specific binding to Fibroblast Activation Protein (FAP) comprising FAP clone 212 or variants thereof, and (b) at least one antigen binding domain capable of specific binding to CD40, and to methods of producing these molecules and to methods of using the same.

### CROSS-REFERENCE TO RELATED APPLICATIONS

This application is a continuation of U.S. patent application Ser. No. 17/557,647, filed Dec. 21, 2021, which is a divisional of U.S. patent application Ser. No. 16/588,780 filed Sep. 30, 2019, now U.S. Pat. No. 11,242,396, issued Feb. 8, 2022, which claims priority to European Patent Application No. 18197866.9, filed Oct. 1, 2018, each of which are incorporated herein by reference in their entirety.

### SEQUENCE LISTING

The instant application contains a Sequence Listing which has been submitted electronically in XML format and is hereby incorporated by reference in its entirety. Said XML copy, created on March 24, 205, is named P35043-US-2.xml and is 155,211 bytes in size.

### FIELD OF THE INVENTION

The invention relates to new bispecific antigen binding molecules, comprising at least one antigen binding domain capable of specific binding to Fibroblast Activation Protein (FAP), at least one antigen binding domain capable of specific binding to CD40, and a Fc domain composed of a first and a second subunit capable of stable association. The invention further relates to a new anti-FAP clone 212. Further aspects of the invention are methods of producing these molecules and methods of using the same.

### BACKGROUND

Multiple molecular signals are required during the generation of a potent adaptive immune response. Signal one involves the binding of a T-cell antigen receptor (TCR) to its cognate antigen presented on the surface of antigen-presenting cells (APCs). Signal two consists of the engagement of costimulatory receptors with their respective ligands between T cells and APCs. One of the best studied and most important costimulatory effectors is the tumor necrosis factor receptor (TNFR) family member CD40 and its ligand CD40L (Elgueta R. et al., Immunol Rev. 2009; 229 (1): 152-72). Several members of the TNFR family including CD40 function after initial T cell activation to sustain APC and T cell responses and thus have pivotal roles in the organization and function of the immune system (Watts T. H. (2005) Annu. Rev. Immunol. 23, 23-68). The combination of different costimulatory TNFR family members allows a sequential and transient regulation of APC and T cell activation and survival resulting in increased immune responses while maintaining tight control of APC and T cell function. Depending on the disease condition, stimulation via costimulatory TNF family members can exacerbate or ameliorate diseases. Activation or blockade of TNFR family costimulators shows promise for several therapeutic applications in multiple fields including cancer, infectious disease, transplantation, and autoimmunity.

Among several costimulatory molecules, the TNFR family member CD40 plays a key role in triggering immune responses by inducing maturation, survival, antigen presentation, cytokine production, and expression of costimulatory molecules of APCs, which then drive antigen-specific T cell responses and NK cell activation by proinflammatory cytokines. CD40 regulates immune responses against infections, tumors and self-antigens and its expression has been demonstrated on the surface of APCs such as B cells, dendritic cells (DCs), monocytes, and macrophages as well as platelets, and cells of non-hematopoietic origin such as myofibroblasts, fibroblasts, epithelial, and endothelial cells (Elgueta R. et al., Immunol Rev. 2009; 229 (1): 152-72). The CD40 ligand CD40L is expressed on activated CD4⁺ helper T cells, platelets, monocytic cells, natural killer cell, mast cells, and basophils (Carbone E. et. al., J Exp Med. 1997; 185 (12): 2053-2060, or Elgueta R. et al., Immunol Rev. 2009; 229 (1): 152-72). Expression of CD40 and CD40L is strongly upregulated in response to various immune stimulatory signals and CD40-CD40L interaction between APCs and CD4⁺ T cells contributes to increased APC activation and antigen-specific CD8⁺ T cell responses (Bevan M J., Nat Rev Immunol. 2014; 4 (8): 595-602). Similar immune stimulatory results were observed by using CD40 agonistic antibodies (Vonderheide R H and Glennie M J., Clin Cancer Res. 2013; 19 (5): 1035-43).

Engagement of the type I transmembrane receptor CD40 by its natural ligand CD40L, a type II transmembrane protein or by agonistic antibodies promotes CD40 clustering and induces the recruitment of adapter proteins to the cytoplasmic receptor domain. The recruitment of these adapter proteins known as TNF receptor-associated factors (TRAFs) leads to synergistic activation of mitogen-activated protein kinases (MAPKs), phosphoinositide 3-kinase (PI3K) as well as canonical and non-canonical nuclear factor κB (NFκB) signaling pathways (Elgueta R. et al., Immunol Rev. 2009; 229 (1): 152-72). In turn, this results in APC maturation and activation, which then maximizes antigen-specific T cell responses. Recent studies have shown two different modes of action of agonistic CD40 antibodies in harnessing anti-tumor immunity. Beside its indirect mode of action by mediated tumor cell killing through the activation of the adaptive immune system, agonistic CD40 antibodies can induce direct tumor cell killing through inducing apoptosis of CD40-expressing solid tumor cells (Eliopoulos A G. et al., Mol Cell Biol. 2000; 20 (15): 5503-15). The direct CD40 antibody-mediated killing of tumor cells can provide a source of tumor antigens that can be processed and presented by APC simultaneously activated by CD40 engagement via anti-CD40 antibodies which then can induce tumor antigen-specific T cells, a postulated mechanism known as endogenous vaccination. Given that CD40 engagement can mount in an efficient anti-cancer immune response, agonistic CD40 antibodies have been used successfully in a variety of preclinical tumor models, both as a single-agent and in combination with chemotherapy (Vonderheide R H and Glennie M J., Clin Cancer Res. 2013; 19 (5): 1035-43).

To date, six CD40 mAb are under investigation in clinical trials: Chi Lob 7/4 (CD40 agonistic IgG1 chimeric mAb; Cancer Research UK; Chowdhury F. et al., Cancer Immunol Res. 2013; 2:229-40), ADC1013 (fully human, CD40 agonistic IgG1 antibody; Alligator Bioscience and Johnson & Johnson; Mangsbo S M. et al., Clin Cancer Res. 2015 Mar. 1; 21 (5): 1115-26), APX-005 (fully humanized, CD40 agonistic IgG1 mAb; Apexigen; Bjorck P. et al. J Immunother Cancer. 2015; 3 (Suppl 2): P198), SEA-CD40 (CD40 agonistic IgG1 chimeric mAb; Seattle Genetics; Gardai S J. et al. AACR 106th Annual Meeting 2015; April 18-22, abstract 2472), as well as RO7009789 (fully human, CD40 super agonistic IgG2 mAb) are investigated in clinical phase I studies, and dacetuzumab (CD40 partial agonistic IgG1 chimeric mAb; Seattle Genetics; Khubchandani S. et al., Curr Opin Investig Drugs. 2009; 10,579-87) is investigated in a clinical phase II study. Eligible patients for these studies have solid tumors, classical Hodgkin lymphoma (HL), diffuse large B-cell lymphoma (DLBCL), or indolent lymphoma (including follicular lymphoma). Diverse activities ranging from Fc-dependent cytotoxicity of CD40⁺ tumor cells via complement mediated cytotoxicity (CMC) or antibody dependent cellular cytoxicity (ADCC) to APC activation to induce anti-tumor T cell responses as well as macrophage activation to deplete tumor and tumor stroma have been shown for these CD40 agonistic antibodies. So far there is no conclusive explanation for this observed heterogeneity. However, recent studies indicate that this mode of action diversity can be explained, at least in part, by differences of the anti-CD40 antibodies in epitope specificity, isotype or Fc: FcγR interaction. For example, it appears that CD40 agonistic antibodies in vivo require crosslinking CD40, bound by its Fab fragment on the target cell, to a Fcγ receptor, bound by its Fc fragment on a cell other than the target cell as has been described for agonistic antibodies specific to other apoptosis-inducing or immunomodulatory members of the TNFR-superfamily (Dahan R., Cancer Cell. 2016 Jun. 13; 29 (6): 820-31; Li F. and Ravetch J. V. Science, 2011; 333, 1030-1034; Teng M. W. et al., J. Immunol. 2009; 183, 1911-1920). The proposed mechanism includes Fcγ receptor mediated clustering of CD40 transmembrane molecules on target cells and subsequent heightened CD40 signaling to achieve potent in vivo efficacy.

The clinical development of agonistic CD40 antibodies has provided promising initial results. In a first clinical trial CP-870,893 has shown clinical efficacy in patients with advanced cancer. Four out of 29 patients with advanced cancer showed partial responses after receiving a single intravenous infusion of CP-870,893 (Vonderheide R H., J Clin Oncol. 2007 Mar. 1; 25 (7): 876-83). One out of these four patients treated with 9 subsequent doses of CP-870,893 over one and a half years remained in complete remission for more than 5 years. However, the most common side effects of CP-870,893 are cytokine release syndromes and thromboembolic events, so that with the dose schedules and routes of administration used the combined data of the phase 1 clinical studies with more than 140 patients only indicates a limited clinical efficacy and a local administration of the antibody was suggested (Vonderheide R H, Glennie M, Clin Cancer Res. 2013, 19 (5), 1035-1043). The lack of single agent responses occurs in part due to severe on target/off tumor effects caused by broad CD40 expression, which results in dose limiting toxicity (e.g. cytokine release syndrome). The development of an agonistic CD40 antibody that specifically activates APCs when CD40 is cross-linked by a tumor-specific target could reduce side effects and decrease dose limitations, offering new therapeutic options with the potential to generate an efficient long lasting anti-cancer immunity.

The available pre-clinical and clinical data clearly demonstrate that there is a high clinical need for effective agonists of CD40 that are able to induce and enhance effective endogenous immune responses to cancer. However, almost never are the effects limited to a single type of cells or acting via a single mechanism and studies designed to elucidate inter- and intracellular signaling mechanisms have revealed increasing levels of complexity. Known CD40 antibodies can only be administered in relatively low doses due to dose-limiting toxicities such as cytokine release syndrome and thrombocyte/endothelial cell activation, resulting in an insufficient activation of the pathway on target APCs and a narrow therapeutic index. Thus, there is a need of “targeted” agonists that preferably act on a single type of cells.

The invention relates to new bispecific antigen binding molecules capable of specific binding to CD40 and Fibroblast Activation Protein (FAP) and thus combine a moiety capable of binding to FAP with a moiety capable of agonistic binding to CD40, wherein the activation of APCs through CD40 is provided by cross-linking through FAP expressed on tumor stroma cells and potentially also through FAP intermediately expressed in secondary lymphoid tissues. In contrast to bispecific antigen binding molecules capable of specific binding to CD40 and to immune checkpoint receptors on activated T cells, such as CTLA-4 or PD-1, targeting to a tumor target such as FAP enables CD40-mediated APC activation mainly in the tumor stroma and tumor-draining lymph nodes where fibroblasts express increased levels of FAP compared to other tissues. The antigen binding molecules of this invention may thus be able to trigger the CD40 receptor not only effectively, but also very selectively at the desired site while overcoming the need for FcγR cross-linking thereby reducing side effects. The new bispecific antigen binding molecules are further characterized by comprising a new FAP antigen binding domain that does not lose its excellent binding properties when it is fused to the C-terminus of the Fc domain.

### SUMMARY OF THE INVENTION

The present invention relates to bispecific antigen binding molecules combining at least one antigen binding domain capable of specific binding to the costimulatory TNF receptor family member CD40, with at least one antigen binding domain targeting Fibroblast Activation Protein (FAP) comprising a new murine anti-human FAP clone 212 and humanized variants thereof. These bispecific antigen binding molecules are advantageous as they will preferably activate costimulatory CD40 receptors at tumor-associated site where FAP is expressed because they are able to bind to FAP with high affinity.

In one aspect, the invention provides a bispecific antigen binding molecule, comprising
(a) at least one antigen binding domain capable of specific binding to CD40, and
(b) at least one antigen binding domain capable of specific binding to Fibroblast Activation Protein (FAP) comprising a heavy chain variable region (VHFAP) comprising (i) CDR-H1 comprising the amino acid sequence of SEQ ID NO:3, (ii) CDR-H2 comprising the amino acid sequence selected from the group consisting of SEQ ID NO:4, SEQ ID NO:11 and SEQ ID NO:12, and (iii) CDR-H3 comprising the amino acid sequence of SEQ ID NO:5, and a light chain variable region (VLFAP) comprising (iv) CDR-L1 comprising the amino acid sequence selected from the group consisting of SEQ ID NO: 6, SEQ ID NO:13 and SEQ ID NO:14, (v) CDR-L2 comprising the amino acid sequence of SEQ ID NO:7, and (vi) CDR-L3 comprising the amino acid sequence of SEQ ID NO: 8.

In a particular aspect, the bispecific antigen binding molecule comprises (a) at least one antigen binding domain capable of specific binding to CD40, (b) at least one antigen binding domain capable of specific binding to FAP comprising a heavy chain variable region (VHFAP) comprising (i) CDR-H1 comprising the amino acid sequence of SEQ ID NO: 3, (ii) CDR-H2 comprising the amino acid sequence selected from the group consisting of SEQ ID NO:4, SEQ ID NO:11 and SEQ ID NO:12, and (iii) CDR-H3 comprising the amino acid sequence of SEQ ID NO:5, and a light chain variable region (VLFAP) comprising (iv) CDR-L1 comprising the amino acid sequence selected from the group consisting of SEQ ID NO:6, SEQ ID NO:13 and SEQ ID NO:14, (v) CDR-L2 comprising the amino acid sequence of SEQ ID NO:7, and (vi) CDR-L3 comprising the amino acid sequence of SEQ ID NO:8, and (c) a Fc domain composed of a first and a second subunit capable of stable association. More particularly, the Fc domain composed of a first and a second subunit capable of stable association comprises mutations that reduce effector function.

In one aspect, the at least one antigen binding domain capable of specific binding to FAP comprises a heavy chain variable region (V_{H}FAP) comprising (i) CDR-H1 comprising the amino acid sequence of SEQ ID NO:3, (ii) CDR-H2 comprising the amino acid sequence selected from the group consisting of SEQ ID NO:4, and (iii) CDR-H3 comprising the amino acid sequence of SEQ ID NO:5, and a light chain variable region (V_{L}FAP) comprising (iv) CDR-L1 comprising the amino acid sequence selected from the group consisting of SEQ ID NO:6, (v) CDR-L2 comprising the amino acid sequence of SEQ ID NO:7, and (vi) CDR-L3 comprising the amino acid sequence of SEQ ID NO:8. In one aspect, the antigen binding domain capable of specific binding to FAP comprises a heavy chain variable region (V_{H}FAP) comprising an amino acid sequence that is at least about 90% identical to the amino acid sequence of SEQ ID NO:9, and a light chain variable region (V_{L}FAP) comprising an amino acid sequence that is at least about 90% identical to the amino acid sequence of SEQ ID NO: 10. In a particular aspect, the antigen binding domain capable of specific binding to FAP comprises a heavy chain variable region (V_{H}FAP) comprising the amino acid sequence of SEQ ID NO:9, and a light chain variable region (V_{L}FAP) comprising the amino acid sequence of SEQ ID NO:10.

In another aspect, the antigen binding domain capable of specific binding to FAP comprises a heavy chain variable region (V_{H}FAP) comprising an amino acid sequence selected from the group consisting of SEQ ID NO:15, SEQ ID NO:16, SEQ ID NO:17, SEQ ID NO:18. SEQ ID NO:19 and SEQ ID NO:20, and a light chain variable region (V_{L}FAP) comprising an amino acid sequence selected from the group consisting of SEQ ID NO: 21, SEQ ID NO:22, SEQ ID NO:23, SEQ ID NO:24, SEQ ID NO:25 and SEQ ID NO: 26. In particular, the antigen binding domain capable of specific binding to FAP comprises (a) a heavy chain variable region (V_{H}FAP) comprising the amino acid sequence of SEQ ID NO:15 and a light chain variable region (V_{L}FAP) comprising the amino acid sequence of SEQ ID NO:21, (b) a heavy chain variable region (V_{H}FAP) comprising the amino acid sequence of SEQ ID NO:16 and a light chain variable region (V_{L}FAP) comprising the amino acid sequence of SEQ ID NO:21, (c) a heavy chain variable region (V_{H}FAP) comprising the amino acid sequence of SEQ ID NO:16 and a light chain variable region (V_{L}FAP) comprising the amino acid sequence of SEQ ID NO:22, or (d) a heavy chain variable region (V_{H}FAP) comprising the amino acid sequence of SEQ ID NO:19 and a light chain variable region (V_{L}FAP) comprising the amino acid sequence of SEQ ID NO: 25. Particularly, the antigen binding domain capable of specific binding to FAP comprises (a) a heavy chain variable region (V_{H}FAP) comprising the amino acid sequence of SEQ ID NO:15 and a light chain variable region (V_{L}FAP) comprising the amino acid sequence of SEQ ID NO:21.

In one aspect, the antigen binding domain capable of specific binding to CD40 binds to a polypeptide comprising, or consisting of, the amino acid sequence of SEQ ID NO:1.

In a further aspect, provided is a bispecific antigen binding molecule, wherein the at least one antigen binding domain capable of specific binding to CD40 comprises a heavy chain variable region (V_{H}CD40) comprising (i) CDR-H1 comprising the amino acid sequence of SEQ ID NO:27, (ii) CDR-H2 comprising the amino acid sequence of SEQ ID NO: 28, and (iii) CDR-H3 comprising the amino acid sequence of SEQ ID NO:29, and a light chain variable region (V_{L}CD40) comprising (iv) CDR-L1 comprising the amino acid sequence of SEQ ID NO:30, (v) CDR-L2 comprising the amino acid sequence of SEQ ID NO: 31, and (vi) CDR-L3 comprising the amino acid sequence of SEQ ID NO:32.

In one aspect, provided is a bispecific antigen binding molecule as defined herein before, wherein the at least one antigen binding domain capable of specific binding to CD40 comprises
(i) a heavy chain variable region (V_{H}CD40) comprising an amino acid sequence selected from the group consisting of SEQ ID NO:37, SEQ ID NO:38, SEQ ID NO:39 and SEQ ID NO:40, and
(ii) a light chain variable region (V_{L}CD40) comprising the amino acid sequence selected from the group consisting of SEQ ID NO:41, SEQ ID NO:42, SEQ ID NO:43, and SEQ ID NO:44.

In another aspect, provided is a bispecific antigen binding molecule as defined herein before, wherein the at least one antigen binding domain capable of specific binding to CD40 comprises (i) a heavy chain variable region (V_{H}CD40) comprising an amino acid sequence selected from the group consisting of SEQ ID NO:45, SEQ ID NO:46, SEQ ID NO: 47, SEQ ID NO:48, SEQ ID NO:49 and SEQ ID NO:50, and
(ii) a light chain variable region (V_{L}CD40) comprising the amino acid sequence selected from the group consisting of SEQ ID NO:51, SEQ ID NO:52, SEQ ID NO:53, and SEQ ID NO:54.

Furthermore, provided is a bispecific antigen binding molecule as defined herein before, wherein the at least one antigen binding domain capable of specific binding to CD40 comprises
(a) a VH comprising the amino acid sequence of SEQ ID NO:37 and a VL comprising the amino acid sequence of SEQ ID NO:41, or
(b) a VH comprising the amino acid sequence of SEQ ID NO:37 and a VL comprising the amino acid sequence of SEQ ID NO:42, or
(c) a VH comprising the amino acid sequence of SEQ ID NO:37 and a VL comprising the amino acid sequence of SEQ ID NO:43, or
(d) a VH comprising the amino acid sequence of SEQ ID NO:37 and a VL comprising the amino acid sequence of SEQ ID NO:44, or
(e) a VH comprising the amino acid sequence of SEQ ID NO:38 and a VL comprising the amino acid sequence of SEQ ID NO:41, or
(f) a VH comprising the amino acid sequence of SEQ ID NO:38 and a VL comprising the amino acid sequence of SEQ ID NO:42, or
(g) a VH comprising the amino acid sequence of SEQ ID NO:38 and a VL comprising the amino acid sequence of SEQ ID NO:43, or
(h) a VH comprising the amino acid sequence of SEQ ID NO:38 and a VL comprising the amino acid sequence of SEQ ID NO:44, or
(i) a VH comprising the amino acid sequence of SEQ ID NO:39 and a VL comprising the amino acid sequence of SEQ ID NO:41, or
(j) a VH comprising the amino acid sequence of SEQ ID NO:39 and a VL comprising the amino acid sequence of SEQ ID NO:42, or
(k) a VH comprising the amino acid sequence of SEQ ID NO:39 and a VL comprising the amino acid sequence of SEQ ID NO:43, or
(l) a VH comprising the amino acid sequence of SEQ ID NO:39 and a VL comprising the amino acid sequence of SEQ ID NO:44, or
(m) a VH comprising the amino acid sequence of SEQ ID NO:40 and a VL comprising the amino acid sequence of SEQ ID NO:41, or
(n) a VH comprising the amino acid sequence of SEQ ID NO:40 and a VL comprising the amino acid sequence of SEQ ID NO:42, or
(o) a VH comprising the amino acid sequence of SEQ ID NO:40 and a VL comprising the amino acid sequence of SEQ ID NO:43, or
(p) a VH comprising the amino acid sequence of SEQ ID NO:40 and a VL comprising the amino acid sequence of SEQ ID NO:44.

In a particular aspect, a bispecific antigen binding molecule is provided, wherein the at least one antigen binding domain capable of specific binding to CD40 comprises a VH comprising the amino acid sequence of SEQ ID NO:37 and a VL comprising the amino acid sequence of SEQ ID NO:41.

In a further aspect, provided is a bispecific antigen binding molecule as defined herein before, wherein the at least one antigen binding domain capable of specific binding to CD40 comprises
(a) a VH comprising the amino acid sequence of SEQ ID NO:45 and a VL comprising the amino acid sequence of SEQ ID NO:51, or
(b) a VH comprising the amino acid sequence of SEQ ID NO:46 and a VL comprising the amino acid sequence of SEQ ID NO:51, or
(c) a VH comprising the amino acid sequence of SEQ ID NO:47 and a VL comprising the amino acid sequence of SEQ ID NO:51, or
(d) a VH comprising the amino acid sequence of SEQ ID NO:48 and a VL comprising the amino acid sequence of SEQ ID NO:51, or
(e) a VH comprising the amino acid sequence of SEQ ID NO:45 and a VL comprising the amino acid sequence of SEQ ID NO:52, or
(f) a VH comprising the amino acid sequence of SEQ ID NO:46 and a VL comprising the amino acid sequence of SEQ ID NO:52, or
(g) a VH comprising the amino acid sequence of SEQ ID NO:47 and a VL comprising the amino acid sequence of SEQ ID NO:52, or
(h) a VH comprising the amino acid sequence of SEQ ID NO:48 and a VL comprising the amino acid sequence of SEQ ID NO:52, or
(i) a VH comprising the amino acid sequence of SEQ ID NO:49 and a VL comprising the amino acid sequence of SEQ ID NO:53, or
(j) a VH comprising the amino acid sequence of SEQ ID NO:50 and a VL comprising the amino acid sequence of SEQ ID NO:53, or
(k) a VH comprising the amino acid sequence of SEQ ID NO:49 and a VL comprising the amino acid sequence of SEQ ID NO:54, or
(l) a VH comprising the amino acid sequence of SEQ ID NO:50 and a VL comprising the amino acid sequence of SEQ ID NO:54.

In a further particular aspect, a bispecific antigen binding molecule is provided, wherein the at least one antigen binding domain capable of specific binding to CD40 comprises a VH comprising the amino acid sequence of SEQ ID NO:45 and a VL comprising the amino acid sequence of SEQ ID NO:51 or wherein the antigen binding domain capable of specific binding to CD40 comprises a VH comprising the amino acid sequence of SEQ ID NO:48 and a VL comprising the amino acid sequence of SEQ ID NO: 51.

More particularly, provided is a bispecific antigen binding molecule, comprising
(i) at least one antigen binding domain capable of specific binding to CD40, comprising a heavy chain variable region (V_{H}CD40) comprising the amino acid sequence of SEQ ID NO:37 and a light chain variable region (V_{L}CD40) comprising the amino acid sequence of SEQ ID NO:41, and
(ii) at least one antigen binding domain capable of specific binding to FAP, comprising a heavy chain variable region (V_{H}FAP) comprising an amino acid sequence of SEQ ID NO:15 and a light chain variable region (V_{L}FAP) comprising an amino acid sequence of SEQ ID NO:21.

In one aspect, the bispecific antigen binding molecule is a humanized or a chimeric antibody. In a further aspect, the bispecific antigen binding molecule comprises an IgG Fc region, particularly an IgG1 Fc region or an IgG4 Fc region. In particular, the Fc region comprises one or more amino acid substitution that reduces the binding affinity of the antibody to an Fc receptor and/or effector function. In a particular aspect, provided is a bispecific antigen binding molecule, wherein the Fc region is of human IgG1 subclass with the amino acid mutations L234A, L235A and P329G (numbering according to Kabat EU index).

In another aspect, provided is a bispecific antigen binding molecule as defined herein before, wherein the first subunit of the Fc region comprises knobs and the second subunit of the Fc region comprises holes according to the knobs into holes method. In particular, provided is a bispecific antigen binding molecule, wherein (i) the first subunit of the Fc region comprises the amino acid substitutions S354C and T366W (numbering according to Kabat EU index) and the second subunit of the Fc region comprises the amino acid substitutions Y349C, T366S and Y407V (numbering according to Kabat EU index), or (ii) the first subunit of the Fc region comprises the amino acid substitutions K392D and K409D (numbering according to Kabat EU index) and the second subunit of the Fc region comprises the amino acid substitutions E356K and D399K (numbering according to Kabat EU index). More particularly, provided is a bispecific antigen binding molecule, wherein the first subunit of the Fc region comprises the amino acid substitutions S354C and T366W (numbering according to Kabat EU index) and the second subunit of the Fc region comprises the amino acid substitutions Y349C, T366S and Y407V (numbering according to Kabat EU index).

In a further aspect, provided is a bispecific antigen binding molecule, wherein the bispecific antigen binding molecule comprises
(a) at least two Fab fragments capable of specific binding to CD40 connected to a Fc region, and
(b) one antigen binding domain capable of specific binding to FAP connected to the C-terminus of the Fc region.

Thus, provided is a bispecific antigen binding molecule that provides bivalent binding towards CD40 and monovalent binding towards FAP.

In another aspect, provided is a bispecific antigen binding molecule, wherein the bispecific antigen binding molecule comprises
(a) at least two Fab fragments capable of specific binding to CD40 fused to a Fc region, and
(b) one antigen binding domain capable of specific binding to FAP fused to the C-terminus of the Fc region.

In a particular aspect, the antigen binding domain capable of specific binding to FAP connected to the C-terminus of the Fc region is a cross-fab fragment. Thus, provided is a bispecific antigen binding molecule, wherein the bispecific antigen binding molecule comprises
(a) at least two Fab fragments capable of specific binding to CD40 fused to a Fc region, and
(b) a cross-fab fragment capable of specific binding to FAP fused to the C-terminus of the Fc region.

In a further aspect, the bispecific antigen binding molecule comprises four Fab fragments capable of specific binding to CD40. Thus, provided is a bispecific antigen binding molecule that provides tetravalent binding towards CD40 and monovalent binding towards FAP.

In one aspect, provided is a bispecific antigen binding molecule comprising
(a) two light chains and two heavy chains of an antibody comprising two Fab fragments capable of specific binding to CD40, and a Fc region, and
(b) a VH and a VL of an antigen binding domain capable specific binding to FAP, wherein the VH is fused to the C-terminus of one of the two heavy chains of (a), and wherein the VL is fused to the C-terminus of the other of the two heavy chains of (a).

In another aspect, provided is a bispecific antigen binding molecule comprising
(a) two light chains and two heavy chains of an antibody comprising two Fab fragments capable of specific binding to CD40, and a Fc region, and
(b) a cross-fab fragment capable specific binding to FAP, wherein the VH-Ckappa chain is fused to the C-terminus of one of the two heavy chains of (a).

In yet another aspect, provided is a bispecific antigen binding molecule comprising
(a) two light chains and two heavy chains of an antibody comprising two Fab fragments capable of specific binding to CD40, and a Fc region, and
(b) a cross-fab fragment capable specific binding to FAP, wherein the VL-CH1 chain is fused to the C-terminus of one of the two heavy chains of (a).

Furthermore, provided is a bispecific antigen binding molecule comprising
(a) two light chains and two heavy chains of an antibody comprising two Fab fragments capable of specific binding to CD40, and a Fc region, and
(b) two Fab fragments capable of specific binding to FAP, wherein one of the Fab fragments is connected to the C-terminus of one of the two heavy chains of (a), and the other of the Fab fragments is connected to the C-terminus of the other of the two heavy chains of (a).

In another aspect, provided is a bispecific antigen binding molecule, wherein the bispecific antigen binding molecule comprises
(a) two heavy chains, each heavy chain comprising a VH and CH1 domain of a Fab fragment capable of specific binding to CD40, and a Fc region subunit,
(b) two light chains, each light chain comprising a VL and CL domain of a Fab fragment capable of specific binding to CD40, and
(c) one Fab fragment capable of specific binding to FAP, wherein the Fab fragments is connected to the C-terminus of one of the two heavy chains of (a).

In another aspect, the Fab fragment capable of specific binding to FAP is a cross-Fab fragment comprising a VL-CH1 chain and a VH-Ckappa chain, and wherein the VH-Ckappa chain or the VL-CH1 chain is connected to the C-terminus of one of the two heavy chains of (a).

In one aspect, provided is a bispecific antigen binding molecule, wherein the bispecific antigen binding molecule comprises four Fab fragments capable of specific binding to CD40. In a particular aspect, provided is a bispecific antigen binding molecule, wherein each of the two heavy chains of (a) as defined herein before comprises two VH-CH1 chains of a Fab fragment capable of specific binding to CD40 that are connected to each other, optionally by a peptide linker.

Thus, in one aspect, the invention provides a bispecific antigen binding molecule comprising
(a) two heavy chains, each heavy chain comprising two VH-CH1 chains of a Fab fragment capable of specific binding to CD40 that are connected to each other, optionally by a peptide linker, and a Fc region subunit,
(b) four light chains, each light chain comprising a VL and Ckappa domain of a Fab fragment capable of specific binding to CD40, and
(c) a cross-Fab fragment capable of specific binding to FAP comprising a VL-CH1 chain and a VH-Ckappa chain, wherein the VH-Ckappa chain or the VL-CH1 chain is connected to the C-terminus of one of the two heavy chains of (a). optionally by a peptide linker.

In another aspect, provided is a bispecific antigen binding molecule comprising
(a) two heavy chains, each heavy chain comprising two VH-CH1 chains of a Fab fragment capable of specific binding to CD40 that are connected to each other, optionally by a peptide linker, and a Fc region subunit,
(b) four light chains, each light chain comprising a VL and CL domain of a Fab fragment capable of specific binding to CD40, and
(c) a cross-fab fragment capable specific binding to FAP, wherein the VH-CL chain of said cross-fab fragment is connected to the C-terminus of one of the two heavy chains of (a).

In yet another aspect, provided is a bispecific antigen binding molecule comprising
(a) two heavy chains, each heavy chain comprising two VH-CH1 chains of a Fab fragment capable of specific binding to CD40 that are connected to each other, optionally by a peptide linker, and a Fc region subunit,
(b) four light chains, each light chain comprising a VL and CL domain of a Fab fragment capable of specific binding to CD40, and
(c) a cross-fab fragment capable specific binding to FAP, wherein the VL-CH1 chain of said cross-fab fragment is connected to the C-terminus of one of the two heavy chains of (a).

In another aspect, provided is an antibody that specifically binds to FAP, wherein said antibody comprises a heavy chain variable region (V_{H}FAP) comprising (i) CDR-H1 comprising the amino acid sequence of SEQ ID NO:3, (ii) CDR-H2 comprising the amino acid sequence selected from the group consisting of SEQ ID NO:4, SEQ ID NO:11 and SEQ ID NO:12, and (iii) CDR-H3 comprising the amino acid sequence of SEQ ID NO:5, and a light chain variable region (V_{L}FAP) comprising (iv) CDR-L1 comprising the amino acid sequence selected from the group consisting of SEQ ID NO:6, SEQ ID NO:13 and SEQ ID NO:14, (v) CDR-L2 comprising the amino acid sequence of SEQ ID NO:7, and (vi) CDR-L3 comprising the amino acid sequence of SEQ ID NO:8. In a particular aspect, provided is an antibody that specifically binds to FAP, wherein said antibody comprises a heavy chain variable region (V_{H}FAP) comprising (i) CDR-H1 comprising the amino acid sequence of SEQ ID NO:3, (ii) CDR-H2 comprising the amino acid sequence selected from the group consisting of SEQ ID NO:4, and (iii) CDR-H3 comprising the amino acid sequence of SEQ ID NO:5, and a light chain variable region (V_{L}FAP) comprising (iv) CDR-L1 comprising the amino acid sequence selected from the group consisting of SEQ ID NO: 6, (v) CDR-L2 comprising the amino acid sequence of SEQ ID NO:7, and (vi) CDR-L3 comprising the amino acid sequence of SEQ ID NO:8.

In a further aspect, provided is an antibody comprising (a) a heavy chain variable region (V_{H}FAP) comprising the amino acid sequence of SEQ ID NO:15 and a light chain variable region (V_{L}FAP) comprising the amino acid sequence of SEQ ID NO:21, (b) a heavy chain variable region (V_{H}FAP) comprising the amino acid sequence of SEQ ID NO: 16 and a light chain variable region (V_{L}FAP) comprising the amino acid sequence of SEQ ID NO:21, (c) a heavy chain variable region (V_{H}FAP) comprising the amino acid sequence of SEQ ID NO: 16 and a light chain variable region (V_{L}FAP) comprising the amino acid sequence of SEQ ID NO:22, or (d) a heavy chain variable region (V_{H}FAP) comprising the amino acid sequence of SEQ ID NO:19 and a light chain variable region (V_{L}FAP) comprising the amino acid sequence of SEQ ID NO:25.

According to another aspect of the invention, there is provided isolated nucleic acid encoding a bispecific antigen binding molecule as described herein before. Also provided is isolated nucleic acid encoding an antibody as described herein before. The invention further provides a vector, particularly an expression vector, comprising the isolated nucleic acid of the invention and a host cell comprising the isolated nucleic acid or the expression vector of the invention. In some aspects the host cell is a eukaryotic cell, particularly a mammalian cell. In another aspect, provided is a method of producing a bispecific antigen binding molecule or an antibody as described herein before, comprising culturing the host cell as described above under conditions suitable for the expression of the bispecific antigen binding molecule or the antibody, and isolating the bispecific antigen binding molecule or the antibody. The invention also encompasses the bispecific antigen binding molecule that specifically binds to CD40 and to FAP or the antibody that specifically binds to FAP produced by the method of the invention.

The invention further provides a pharmaceutical composition comprising a bispecific antigen binding molecule as described herein before or the antibody as described herein before and a pharmaceutically acceptable carrier. In one aspect, the pharmaceutical composition comprises an additional therapeutic agent.

Also encompassed by the invention is the bispecific antigen binding molecule or the antibody as described herein before, or the pharmaceutical composition comprising the bispecific antigen binding molecule, for use as a medicament.

In one aspect, provided is a bispecific antigen binding molecule as described herein before or the pharmaceutical composition of the invention, for use
(i) in inducing immune stimulation by CD40 expressing antigen-presenting cells (APCs),
(ii) in stimulating tumor-specific T cell response,
(iii) in causing apoptosis of tumor cells,
(iv) in the treatment of cancer,
(v) in delaying progression of cancer,
(vi) in prolonging the survival of a patient suffering from cancer,
(vii) in the treatment of infections.

In a specific aspect, provided is the bispecific antigen binding molecule as described herein before or the pharmaceutical composition of the invention, for use in the treatment of cancer. In another specific aspect, the invention provides the bispecific antigen binding molecule as described herein before for use in the treatment of cancer, wherein the bispecific antigen binding molecule is administered in combination with a chemotherapeutic agent, radiation and/or other agents for use in cancer immunotherapy. In one aspect, the bispecific antigen binding molecule as described herein is for use in the treatment of cancer, wherein the bispecific antigen binding molecule is for administration in combination with an agent blocking PD-L1/PD-1 interaction. In another aspect, provided is the bispecific antigen binding molecule as described herein before or the pharmaceutical composition of the invention, for use in up-regulating or prolonging cytotoxic T cell activity. In a further aspect, provided is an antibody as described herein before, for use in the treatment of cancer.

In a further aspect, the invention provides a method of inhibiting the growth of tumor cells in an individual comprising administering to the individual an effective amount of the bispecific antigen binding molecule as described herein before, or the pharmaceutical composition of the invention, to inhibit the growth of the tumor cells. In another aspect, the invention provides a method of treating or delaying cancer in an individual comprising administering to the individual an effective amount of the bispecific antigen binding molecule as described herein before, or the pharmaceutical composition of the invention.

Also provided is the use of the bispecific antigen binding molecule as described herein before for the manufacture of a medicament for the treatment of a disease in an individual in need thereof, in particular for the manufacture of a medicament for the treatment of cancer, as well as a method of treating a disease in an individual, comprising administering to said individual a therapeutically effective amount of a composition comprising the bispecific antigen binding molecule of the invention in a pharmaceutically acceptable form. In a specific aspect, the disease is cancer. In any of the above aspects the individual is a mammal, particularly a human.

### BRIEF DESCRIPTION OF THE DRAWINGS

FIG. 1A, FIG. 1B, FIG. 1C, FIG. 1D, FIG. 1E, and FIG. 1F show schematic representations of the bispecific antigen binding molecules which specifically bind to human CD40 and to FAP. FIG. 1A shows a schematic representation of a bispecific FAP-CD40 antibody in a 2+1 format consisting of two CD40 binding moieties combined with one FAP (212) binding moiety as crossover fab fragment, wherein the VL-CH1 chain is fused at the C-terminus of the Fc knob chain (bivalent for CD40 and monovalent for FAP). FIG. 1B shows a schematic representation of a bispecific FAP-CD40 antibody in a 2+1 format consisting of two CD40 binding moieties combined with one FAP (4B9) binding moiety as crossover fab fragment, wherein the VH-Ckappa chain is fused at the C-terminus of the Fc knob chain (bivalent for CD40 and monovalent for FAP). FIG. 1C shows a schematic representation of a bispecific FAP-CD40 antibody in a 2+1 format consisting of two CD40 binding moieties combined with one FAP (28H1) binding moiety as crossover fab fragment, wherein the VL-CH1 chain is fused at the C-terminus of the Fc knob chain (bivalent for CD40 and monovalent for FAP). FIG. 1D shows a schematic representation of a bispecific FAP-CD40 antibody in a 4+1 format consisting of four CD40 binding moieties combined with one FAP (212) binding moiety as crossover fab fragment, wherein the VL-CH1 chain is fused at the C-terminus of the Fc knob chain (tetravalent for CD40 and monovalent for FAP). FIG. 1E shows a schematic representation of a bispecific FAP-CD40 antibody in a 4+1 format consisting of four CD40 binding moieties combined with one FAP (4B9) binding moiety as crossover fab fragment, wherein the VL-CH1 chain is fused at the C-terminus of the Fc knob chain (tetravalent for CD40 and monovalent for FAP). FIG. 1F shows a schematic representation of a bispecific FAP-CD40 antibody in a 4+1 format consisting of four CD40 binding moieties combined with one FAP (28H1) binding moiety as crossover fab fragment, wherein the VL-CH1 chain is fused at the C-terminus of the Fc knob chain (tetravalent for CD40 and monovalent for FAP). The black point symbolizes knob-into-hole mutations. Alternatively, in all molecules the VH-Ckappa chain of the crossfab may be fused at the C-terminus of the Fc knob chain.

FIG. 2A and FIG. 2B show the cellular binding of immunization derived FAP clones to human FAP expressed on transfected HEK cells in competition to FAP clones 4B9 and 28H1. FIG. 2A shows that all tested hybridoma-derived murine clones (named 209, 210, 211, 212, 213, 214, 215, 216, 217 and 218) did not compete for binding with anti FAP antibody 4B9 and FIG. 2B shows that the same clones did not compete for binding with anti-FAP antibody 28H1. MFI was measured by flow cytometry. The x-axis shows the concentration of the FAP antibody.

FIG. 3A, FIG. 3B, and FIG. 3C show schematic representations of antibody constructs that were made to determine if the binding properties of the anti-FAP clones are not lost when they are C-terminally fused to an Fc domain. FIG. 3A shows a construct comprising a Fc knob chain and a Fc hole chain wherein the VH domain is fused to the C-terminus of the Fc knob chain and the VL domain is fused to the C-terminus of the Fc hole chain (C-term VH/VL fusion). FIG. 3B shows a construct comprising a Fc knob chain and a Fc hole chain wherein the whole Fab is fused with its VH domain to the C-terminus of the Fc knob chain (C-term Fab fusion). FIG. 3C shows the setup for the epitope binning which was performed using a surface plasmon resonance (SPR) based assay on a Biacore T200 instrument (see Example 1.9).

FIG. 4 shows the binding of human tetravalent or bivalent anti-CD40 antibodies in a FAP (212), FAP (4B9) or FAP (28H1)-targeted monovalent format to human FAP-positive NIH/3T3 cells. The transgenic modified mouse embryonic fibroblast NIH/3T3-hFAP cell line expresses high levels of human fibroblast activation protein (huFAP). All depicted anti-CD40 antigen binding molecules with a FAP binding moiety efficiently bind to NIH/3T3-hFAP cells but vary in their binding strength (EC₅₀ values as well as signal strength) to NIH/3T3-hFAP cells. The FAP-CD40 constructs with a C-terminal FAP (212) or FAP (4B9) binding domain bind stronger than the FAP-CD40 constructs with a C-terminal FAP (28H1) binding domain. Shown is the binding as median of fluorescence intensity (MFI) of phycoerythrin (PE)-labeled anti-human IgG Fcγ-specific goat IgG F(ab′)₂ fragment which is used as secondary detection antibody. The MFI was measured by flow cytometry and the baseline was corrected by subtracting the MFI of the blank control. The x-axis shows the concentration of antibody constructs.

FIG. 5 shows the binding of human tetravalent or bivalent anti-CD40 antibodies in a FAP (212) or FAP (4B9)-targeted monovalent format to primary human B cells with high surface expression levels of human CD40. All depicted constructs bind to CD40 but vary in their binding strength (EC₅₀ values as well as signal strength) to CD40-positive B cells. Bivalent anti-CD40 antibodies show higher EC₅₀ levels and reach higher binding plateaus compared to tetravalent anti-CD40 antibodies, irrespective of their FAP binding moiety. Binding of anti-CD40 antibodies to cell surface proteins was detected with an anti-human IgG Fcγ-specific goat IgG F(ab)₂ fragment conjugated to phycoerythrin (PE) using FACS analysis. The MFI was measured by flow cytometry and baseline corrected by subtracting the MFI of the blank control. The x-axis shows the concentration of antibody constructs.

FIG. 6A and FIG. 6B show the in vitro activation of human Daudi cells by monovalent FAP (212), FAP (4B9) or FAP (28H1)-targeted human anti-CD40 constructs in the presence of FAP-coated (FIG. 6A) or uncoated Dynabeads® (FIG. 6B) after 2 days incubation. With FAP-coated beads all depicted bispecific antibodies monovalent for FAP induced an increase of the B cell activation marker expression CD70. The B cell activation marker upregulation by bispecific FAP-CD40 antibodies in a 2+1 format with a FAP (212) or FAP (4B9) binding domain was higher compared to the upregulation induced by the bispecific FAP-CD40 antibody in a 2+1 format with a FAP (28H1) binding domain, the bispecific FAP-CD40 antibodies in a 4+1 format with a FAP (212), FAP (4B9) or FAP (28H1) binding domain or the FAP-independent positive control antibody. In the absence of FAP (uncoated beads) no increase of CD70 was observed with the depicted FAP-targeted bispecific antibodies bivalent for CD40, while tetravalent CD40 binding molecules induced an upregulation of CD70, but to a lesser extent than in the presence of FAP. Shown is the percentage of CD70-positive vital Daudi cells after 2 days incubation with the indicated titrated antibodies. The x-axis shows the concentration of antibody constructs.

FIG. 7A and FIG. 7B show the in vitro activation of human B cells by monovalent FAP (212), FAP (4B9) or FAP (28H1)-targeted human anti-CD40 constructs in the presence of FAP-coated (FIG. 7A) or uncoated Dynabeads® (FIG. 7B) after 2 days incubation. With FAP-coated beads all depicted bispecific antibodies monovalent for FAP induced an increase of the B cell activation marker expression CD86. Compared to the FAP-independent upregulation of CD86 induced by the cross-linked anti-CD40 antibody (P1AD4470), the CD86 upregulation induced by FAP-dependent bispecific antigen binding molecules was similar or slightly lower. At lower antibody concentrations the B cell activation marker upregulation by bispecific FAP-CD40 antibodies in a 2+1 format with a FAP (212) or FAP (4B9) binding domain was lower compared to the upregulation induced by the bispecific FAP-CD40 antibody in a 2+1 format with a FAP (28H1) binding domain, the bispecific FAP-CD40 antibodies in a 4+1 format with a FAP (212), FAP (4B9) or FAP (28H1) binding domain or the FAP-independent positive control antibody. In the absence of FAP (uncoated beads) no increase of CD86 expression was observed with the bispecific antigen binding molecules, while the positive control antibody induced an upregulation of CD86. Shown is the percentage of CD86-positive vital B cells after 2 days incubation with the indicated titrated antibodies. The x-axis shows the concentration of antibody constructs.

FIG. 8A and FIG. 8B show the T cell priming of OVA-pulsed DCs activated by FAP-targeted anti-CD40 binding molecules in the presence (FIG. 8A) or absence (FIG. 8B) of FAP. DCs isolated from huCD40 transgenic mice, treated with DEC205-OVA conjugate and stimulated with FAP-dependent bispecific anti-CD40 antibodies as well as FAP-coated beads induced a strong proliferation of antigen-specific T cells. In contrast, in the absence of FAP (uncoated beads) no T cell proliferation was induced by DCs stimulated with FAP-targeted anti-CD40 antibodies. The T cell proliferation induced by DCs stimulated with the human bispecific antigen binding molecules with two or four CD40 and one FAP (212) or FAP (4B9) binding moieties was comparable. DCs pulsed with high amounts of SIINFEKL instead of DEC205-OVA conjugate induced a strong T cell proliferation. Shown is the percentage of proliferating (CFSE-low) vital CFSE-labeled murine CD3⁺CD8⁺ OT-1 T cells co-cultured with huCD40 tg DCs pre-incubated with the indicated titrated antibodies in the presence of OVA (FIG. 8A and FIG. 8B). The x-axis shows the concentration of antibody constructs.

FIG. 9 shows the effect of simultaneous binding of the bispecific FAP×CD40 antibodies in the two cell line bridging assay as described in Example 5.3. Shown is the optical density (OD) against the concentration of the bispecific antibodies in different formats.

In FIG. 10 the potential of FAP×CD40 antibodies in different formats to activate the CD40 receptor (independent from FAP) is shown. In the reporter cell line assay the signal transduction results in the induction of NF□B-dependent production of secreted embryonic alkaline phosphatase (SEAP) and the activity of SEAP is measured. The mean of the optical density (OD) of n=6 and the 3×STDEV is plotted against the molecules.

FIG. 11 shows the study design of an in vivo mouse study to evaluation the safety, pharmacokinetic and pharmacodynamic profile of FAP-targeted anti-CD40 binding molecules in comparison to a non-targeted anti-CD40 antibody in a subcutaneous MC38-FAP tumor model.

FIG. 12 shows the tumor growth upon the 1st and 2nd (re-challenge) MC38-FAP tumor cell injection of mice treated with the indicated single or combination antibody therapies. Arrows indicate the days of therapy injection and re-challenge. The y-axis represents the tumor volume in mm³ and the x-axis the days upon the 1st MC38-FAP tumor cell injection. A complete tumor regression of MC38-FAP tumors upon FAP-CD40 antibody treatment, independently of the format and the co-injection of anti-PD-L1 antibody was observed. In contrast, in mice treated with anti-PD-L1 antibody alone, anti-CD40 antibody alone or anti-CD40 in combination with anti-PD-L1 antibody, tumor growth was comparable to mice treated with a vehicle control. Moreover, upon re-challenge, no MC38-FAP tumor grew in the groups previously treated with FAP-CD40 antibody whereas in naïve mice 100% of the tumors grew.

FIG. 13A is a statistical table comparing all treatment groups at day 28 (6 days upon therapy) and FIG. 13B is a statistical table comparing all treatment from day 31 on (9 days upon therapy).

FIG. 14 shows the pharmacokinetic profile of a 4+1 and 2+1 FAP-targeted anti-CD40 binding molecule as well as a non-targeted parental anti-CD40 antibody in mice injected with a FAP-expressing murine colon adenocarcinoma tumor cell line (MC38-FAP). The y-axis represents the dose normalized concentration in the serum and the x-axis the time upon antibody injection. The highest clearance rate was observed for the non-targeted anti-CD40 antibody. The clearance rate of the FAP-targeted anti-CD40 binding molecule in a 4+1 format was lower compared to the non-target anti-CD40 molecule and the lowest clearance rate was observed for the FAP-targeted anti-CD40 binding molecule in a 2+1 format.

FIG. 15A, FIG. 15B, FIG. 15C, FIG. 15D, FIG. 16A, FIG. 16B, FIG. 16C, FIG. 16D, and FIG. 17A, FIG. 17B, FIG. 17C, FIG. 17D show the DC, T cell and B cell activation in draining lymph nodes (LN, FIG. 15A, FIG. 16A and FIG. 17A), non-draining lymph nodes (FIG. 15B, FIG. 16B and FIG. 17B), spleen (FIG. 15C, FIG. 16C and FIG. 17C) and tumor (FIG. 15D, FIG. 16D and FIG. 17D) of mice injected with a FAP-expressing murine colon adenocarcinoma tumor cell line (MC38-FAP) and treated with either non-targeted anti-CD40 (P1AE2301), anti-CD40-FAP 4+1 (P1AE2024), anti-CD40-FAP 2+1 (P1AE2302) or vehicle alone. DC and T cell activation in the tumor four days after therapy injection (FIG. 15D and FIG. 16D) was significantly increased in animals treated with anti-CD40-FAP 2+1 (P1AE2302) compared to vehicle-treated animals. In all other analyzed tissues (draining LN, non-draining LN and spleen) non-targeted anti-CD40, anti-CD40-FAP 4+1 and anti-CD40-FAP 2+1 induced a significant DC and T cell activation compared to vehicle group. In contrast, only the non-targeted anti-CD40 mediated a significant B cell activation in all analyzed tissues compared to the vehicle control group (FIG. 17A, FIG. 17B, FIG. 17C, and FIG. 17D, *p<0.05, ** p<0.01, *** p<0.001, **** p<0.0001, unpaired, two-tailed Student's test).

FIG. 18 shows the body weight of mice injected with a FAP-expressing murine colon adenocarcinoma tumor cell line (MC38-FAP) and treated with either non-targeted anti-CD40 (P1AE2301), anti-CD40-FAP 4+1 (P1AE2024), anti-CD40-FAP 2+1 (P1AE2302) or vehicle with or without co-injection of anti-PD-L1 antibody (P1AE0099). The y-axis shows the body weight in percent of the body weight prior to treatment and the x-axis shows the days after therapy injection. Only in mice treated with the non-targeted anti-CD40 antibody alone or in combination with anti-PD-L1 a clear body weight reduction was observed.

### DETAILED DESCRIPTION OF THE INVENTION

### Definitions

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

As used herein, the term “antigen binding molecule” refers in its broadest sense to a molecule that specifically binds an antigenic determinant. Examples of antigen binding molecules are antibodies, antibody fragments and scaffold antigen binding proteins.

As used herein, the term “antigen binding domain capable of specific binding to a target cell antigen” or “moiety capable of specific binding to a target cell antigen” refers to a polypeptide molecule that specifically binds to an antigenic determinant. In one aspect, the antigen binding domain is able to activate signaling through its target cell antigen. In a particular aspect, the antigen binding domain is able to direct the entity to which it is attached (e.g. the CD40 agonist) to a target site, for example to a specific type of tumor cell or tumor stroma bearing the antigenic determinant. Antigen binding domains capable of specific binding to a target cell antigen include antibodies and fragments thereof as further defined herein. In addition, antigen binding domains capable of specific binding to a target cell antigen include scaffold antigen binding proteins as further defined herein, e.g. binding domains which are based on designed repeat proteins or designed repeat domains (see e.g. WO 2002/020565).

In relation to an antibody or fragment thereof, the term “antigen binding domain capable of specific binding to a target cell antigen” refers to the part of the molecule that comprises the area which specifically binds to and is complementary to part or all of an antigen. An antigen binding domain capable of specific antigen binding may be provided, for example, by one or more antibody variable domains (also called antibody variable regions). Particularly, an antigen binding domain capable of specific antigen binding comprises an antibody light chain variable region (VL) and an antibody heavy chain variable region (VH). In another aspect, the “antigen binding domain capable of specific binding to a target cell antigen” can also be a Fab fragment or a cross-Fab fragment.

The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

The term “monoclonal antibody” as used herein refers to an antibody obtained from a population of substantially homogeneous antibodies, i.e., the individual antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

The term “monospecific” antibody as used herein denotes an antibody that has one or more binding sites each of which bind to the same epitope of the same antigen. The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells. A bispecific antigen binding molecule as described herein can also form part of a multispecific antibody.

The term “valent” as used within the current application denotes the presence of a specified number of binding sites specific for one distinct antigenic determinant in an antigen binding molecule that are specific for one distinct antigenic determinant. As such, the terms “bivalent”, “tetravalent”, and “hexavalent” denote the presence of two binding sites, four binding sites, and six binding sites specific for a certain antigenic determinant, respectively, in an antigen binding molecule. In particular aspects of the invention, the bispecific antigen binding molecules according to the invention can be monovalent for a certain antigenic determinant, meaning that they have only one binding site for said antigenic determinant or they can be bivalent or tetravalent for a certain antigenic determinant, meaning that they have two binding sites or four binding sites, respectively, for said antigenic determinant.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called a (IgA), δ (IgD), ε (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies, triabodies, tetrabodies, cross-Fab fragments; linear antibodies; single-chain antibody molecules (e.g. scFv); and single domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′) 2 fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteines from the antibody hinge region. Fab′-SH are Fab′ fragments wherein the cysteine residue(s) of the constant domains bear a free thiol group. Pepsin treatment yields an F(ab′)₂ fragment that has two antigen-combining sites (two Fab fragments) and a part of the Fc region. According to the present invention, the term “Fab fragment” also includes “cross-Fab fragments” or “crossover Fab fragments” as defined below.

The term “cross-Fab fragment” or “xFab fragment” or “crossover Fab fragment” refers to a Fab fragment, wherein either the variable regions or the constant regions of the heavy and light chain are exchanged. Two different chain compositions of a crossover Fab molecule are possible and comprised in the bispecific antibodies of the invention: On the one hand, the variable regions of the Fab heavy and light chain are exchanged, i.e. the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab_{(VLVH)}. On the other hand, when the constant regions of the Fab heavy and light chain are exchanged, the crossover Fab molecule comprises a peptide chain composed of the heavy chain variable region (VH) and the light chain constant region (CL), and a peptide chain composed of the light chain variable region (VL) and the heavy chain constant region (CH1). This crossover Fab molecule is also referred to as CrossFab_{(CLCH1)}.

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g. position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen-binding site which binds specifically to an antigen and wherein said linker is a polypeptide of at least 30 amino acids. In addition, these x-scFab molecules might be further stabilized by generation of interchain disulfide bonds via insertion of cysteine residues (e.g. position 44 in the variable heavy chain and position 100 in the variable light chain according to Kabat numbering).

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

“Scaffold antigen binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13:695-701 (2008). In one aspect of the invention, a scaffold antigen binding protein is selected from the group consisting of CTLA-4 (Evibody), Lipocalins (Anticalin), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), V_{NAR} fragments, a fibronectin (AdNectin), a C-type lectin domain (Tetranectin); a variable domain of a new antigen receptor beta-lactamase (VNAR fragments), a human gamma-crystallin or ubiquitin (Affilin molecules); a kunitz type domain of human protease inhibitors, microbodies such as the proteins from the knottin family, peptide aptamers and fibronectin (adnectin). CTLA-4 (Cytotoxic T Lymphocyte-associated Antigen 4) is a CD28-family receptor expressed on mainly CD4⁺ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies (e.g. U.S. Pat. No. 7,166,697B1). Evibodies are around the same size as the isolated variable region of an antibody (e.g. a domain antibody). For further details see Journal of Immunological Methods 248 (1-2), 31-45 (2001). Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size, and are derived from lipocalins. For further details see Biochim Biophys Acta 1482:337-350 (2000), U.S. Pat. No. 7,250,297B1 and US20070224633. An affibody is a scaffold derived from Protein A of Staphylococcus aureus which can be engineered to bind to antigen. The domain consists of a three-helical bundle of approximately 58 amino acids. Libraries have been generated by randomization of surface residues. For further details see Protein Eng. Des. Sel. 2004, 17, 455-462 and EP 1641818A1. Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see Nature Biotechnology 23 (12), 1556-1561 (2005) and Expert Opinion on Investigational Drugs 16 (6), 909-917 (June 2007). A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999). Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100 (4), 1700-1705 (2003) and J. Mol. Biol. 369, 1015-1028 (2007) and US20040132028A1. A single-domain antibody is an antibody fragment consisting of a single monomeric variable antibody domain. The first single domains were derived from the variable domain of the antibody heavy chain from camelids (nanobodies or V_{H}H fragments). Furthermore, the term single-domain antibody includes an autonomous human heavy chain variable domain (aVH) or V_{NAR} fragments derived from sharks. Fibronectin is a scaffold which can be engineered to bind to antigen. Adnectins consists of a backbone of the natural amino acid sequence of the 10th domain of the 15 repeating units of human fibronectin type III (FN3). Three loops at one end of the .beta.-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1. Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005). Microbodies are derived from naturally occurring microproteins of 25-50 amino acids in length which contain 3-4 cysteine bridges—examples of microproteins include KalataBI and conotoxin and knottins. The microproteins have a loop which can be engineered to include up to 25 amino acids without affecting the overall fold of the microprotein. For further details of engineered knottin domains, see WO2008098796.

An “antigen binding molecule that binds to the same epitope” as a reference molecule refers to an antigen binding molecule that blocks binding of the reference molecule to its antigen in a competition assay by 50% or more, and conversely, the reference molecule blocks binding of the antigen binding molecule to its antigen in a competition assay by 50% or more. An “antigen binding molecule that does not bind to the same epitope” as a reference molecule refers to an antigen binding molecule that does not block binding of the reference molecule to its antigen in a competition assay by 50% or more, and conversely, the reference molecule does not block binding of the antigen binding molecule to its antigen in a competition assay by 50% or more.

The term “antigen binding domain” or “antigen-binding site” refers to the part of an antigen binding molecule that comprises the area which specifically binds to and is complementary to part or all of an antigen. Where an antigen is large, an antigen binding molecule may only bind to a particular part of the antigen, which part is termed an epitope. An antigen binding domain may be provided by, for example, one or more variable domains (also called variable regions). Preferably, an antigen binding domain comprises an antibody light chain variable region (VL) and an antibody heavy chain variable region (VH).

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, an molecule that binds to the antigen has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g. from 10⁻⁹ M to 10⁻¹³ M).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (Kd), which is the ratio of dissociation and association rate constants (koff and kon, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

A “target cell antigen” as used herein refers to an antigenic determinant presented on the surface of a target cell, in particular a target cell in a tumor such as a cancer cell or a cell of the tumor stroma. Thus, the target cell antigen is a tumor-associated antigen. In particular, the tumor target cell antigen is Fibroblast Activation Protein (FAP).

The term “Fibroblast activation protein (FAP)”, also known as Prolyl endopeptidase FAP or Seprase (EC 3.4.21), refers to any native FAP from any vertebrate source, including mammals such as primates (e.g. humans) non-human primates (e.g. cynomolgus monkeys) and rodents (e.g. mice and rats), unless otherwise indicated. The term encompasses “full-length,” unprocessed FAP as well as any form of FAP that results from processing in the cell. The term also encompasses naturally occurring variants of FAP, e.g., splice variants or allelic variants. In one embodiment, the antigen binding molecule of the invention is capable of specific binding to human, mouse and/or cynomolgus FAP. The amino acid sequence of human FAP is shown in UniProt (www.uniprot.org) accession no. Q12884 (version 149, SEQ ID NO:2), or NCBI (www.ncbi.nlm.nih.gov/) RefSeq NP_004451.2. The extracellular domain (ECD) of human FAP extends from amino acid position 26 to 760. The amino acid sequence of a His-tagged human FAP ECD is shown in SEQ ID NOs 78. The amino acid sequence of mouse FAP is shown in UniProt accession no. P97321 (version 126, SEQ ID NO:79), or NCBI RefSeq NP_032012.1. The extracellular domain (ECD) of mouse FAP extends from amino acid position 26 to 761. SEQ ID NO:80 shows the amino acid of a His-tagged mouse FAP ECD. SEQ ID NO:81 shows the amino acid of a His-tagged cynomolgus FAP ECD. Preferably, an anti-FAP binding molecule of the invention binds to the extracellular domain of FAP.

The term “variable region” or “variable domain” refers to the domain of an antibody heavy or light chain that is involved in binding the antigen binding molecule to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1 (CDR-L1)-FR2-CDR-H2 (CDR-L2)-FR3-CDR-H3 (CDR-L3)-FR4.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG₁, IgG₂, IgG₃, IgG₄, IgA₁, and IgA₂. The heavy chain constant domains that correspond to the different classes of immunoglobulins are called α, δ, ε, γ, and μ respectively.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

The term “Fc domain” or “Fc region” herein is used to define a C-terminal region of an antibody heavy chain that contains at least a portion of the constant region. The term includes native sequence Fc regions and variant Fc regions. An IgG Fc region comprises an IgG CH2 and an IgG CH3 domain. The “CH2 domain” of a human IgG Fc region usually extends from an amino acid residue at about position 231 to an amino acid residue at about position 340. In one embodiment, a carbohydrate chain is attached to the CH2 domain. The CH2 domain herein may be a native sequence CH2 domain or variant CH2 domain. The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical antibody heavy chains as herein described. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The “knob-into-hole” technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)).

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “effector function” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

Fc receptor binding dependent effector functions can be mediated by the interaction of the Fc-region of an antibody with Fc receptors (FcRs), which are specialized cell surface receptors on hematopoietic cells. Fc receptors belong to the immunoglobulin superfamily, and have been shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC) (see e.g. Van de Winkel, J. G. and Anderson, C. L., J. Leukoc. Biol. 49 (1991) 511-524). FcRs are defined by their specificity for immunoglobulin isotypes: Fc receptors for IgG antibodies are referred to as FcγR. Fc receptor binding is described e.g. in Ravetch, J. V. and Kinet, J. P., Annu. Rev. Immunol. 9 (1991) 457-492, Capel, P. J., et al., Immunomethods 4 (1994) 25-34; de Haas, M., et al., J. Lab. Clin. Med. 126 (1995) 330-341; and Gessner, J. E., et al., Ann. Hematol. 76 (1998) 231-248.

Cross-linking of receptors for the Fc-region of IgG antibodies (FcγR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are:
Fc□RI (CD64) binds monomeric IgG with high affinity and is expressed on macrophages, monocytes, neutrophils and eosinophils. Modification in the Fc-region IgG at least at one of the amino acid residues E233-G236, P238, D265, N297, A327 and P329 (numbering according to EU index of Kabat) reduce binding to FcγRI. IgG2 residues at positions 233-236, substituted into IgG1 and IgG4, reduced binding to FcγRI by 103-fold and eliminated the human monocyte response to antibody-sensitized red blood cells (Armour, K. L., et al., Eur. J. Immunol. 29 (1999) 2613-2624).
Fc□RII (CD32) binds complexed IgG with medium to low affinity and is widely expressed. This receptor can be divided into two sub-types, Fc□RIIA and Fc□RIIB. Fc□RIIA is found on many cells involved in killing (e.g. macrophages, monocytes, neutrophils) and seems able to activate the killing process. FcγRIIB seems to play a role in inhibitory processes and is found on B cells, macrophages and on mast cells and eosinophils. On B-cells it seems to function to suppress further immunoglobulin production and isotype switching to, for example, the IgE class. On macrophages, FcγRIIB acts to inhibit phagocytosis as mediated through FcγRIIA. On eosinophils and mast cells the B-form may help to suppress activation of these cells through IgE binding to its separate receptor. Reduced binding for FcγRIIA is found e.g. for antibodies comprising an IgG Fc-region with mutations at least at one of the amino acid residues E233-G236, P238, D265, N297, A327, P329, D270, Q295, A327, R292, and K414 (numbering according to EU index of Kabat).
Fc□RIII (CD16) binds IgG with medium to low affinity and exists as two types. Fc□RIIIA is found on NK cells, macrophages, eosinophils and some monocytes and T cells and mediates ADCC. FcγRIIIB is highly expressed on neutrophils. Reduced binding to Fc□RIIIA is found e.g. for antibodies comprising an IgG Fc-region with mutation at least at one of the amino acid residues E233-G236, P238, D265, N297, A327, P329, D270, Q295, A327, S239, E269, E293, Y296, V303, A327, K338 and D376 (numbering according to EU index of Kabat).

Mapping of the binding sites on human IgG1 for Fc receptors, the above mentioned mutation sites and methods for measuring binding to FcγRI and FcγRIIA are described in Shields, R. L., et al. J. Biol. Chem. 276 (2001) 6591-6604.

The term “ADCC” or “antibody-dependent cellular cytotoxicity” is a function mediated by Fc receptor binding and refers to lysis of target cells by an antibody as reported herein in the presence of effector cells. The capacity of the antibody to induce the initial steps mediating ADCC is investigated by measuring their binding to Fcγ receptors expressing cells, such as cells, recombinantly expressing FcγRI and/or FcγRIIA or NK cells (expressing essentially FcγRIIIA). In particular, binding to FcγR on NK cells is measured.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89). A particular activating Fc receptor is human FcγRIIIa (see UniProt accession no. P08637, version 141).

The term “CD40”, as used herein, refers to any native CD40 from any vertebrate source, including mammals such as primates (e.g. humans) and rodents (e.g., mice and rats), unless otherwise indicated. The term encompasses “full-length,” unprocessed CD40 as well as any form of CD40 that results from processing in the cell. The term also encompasses naturally occurring variants of CD40, e.g., splice variants or allelic variants. The amino acid sequence of an exemplary human CD40 is shown in SEQ ID NO:1 (Uniprot P25942, version 200) and the amino acid sequence of an exemplary mouse CD40 is shown in SEQ ID NO: 146 (Uniprot P27512, version 160). The CD40 antigen is a 50 kDa cell surface glycoprotein which belongs to the Tumor Necrosis Factor Receptor (TNF-R) family. (Stamenkovic et al. (1989), EMBO J. 8:1403-10). CD40 is expressed in many normal and tumor cell types, including B lymphocytes, dendritic cells, monocytes, macrophages, thymus epithelium, endothelial cells, fibroblasts, and smooth muscle cells. CD40 is expressed in all B-lymphomas and in 70% of all solid tumors and is up-regulated in antigen presenting cells (APCs) by maturation signals, such as IFN-gamma and GM-CSF. CD40 activation also induces differentiation of monocytes into functional dendritic cells (DCs) and enhances cytolytic activity of NK cells through APC-CD40 induced cytokines. Thus CD40 plays an essential role in the initiation and enhancement of immune responses by inducing maturation of APCs, secretion of helper cytokines, upregulation of costimulatory molecules, and enhancement of effector functions.

The term “CD40 agonist” as used herein includes any moiety that agonizes the CD40/CD40L interaction. CD40 as used in this context refers preferably to human CD40, thus the CD40 agonist is preferably an agonist of human CD40. Typically, the moiety will be an agonistic CD40 antibody or antibody fragment.

The terms “anti-CD40 antibody”, “anti-CD40”, “CD40 antibody and “an antibody that specifically binds to CD40” refer to an antibody that is capable of binding CD40 with sufficient affinity such that the antibody is useful as a diagnostic and/or therapeutic agent in targeting CD40. In one aspect, the extent of binding of an anti-CD40 antibody to an unrelated, non-CD40 protein is less than about 10% of the binding of the antibody to CD40 as measured, e.g., by a radioimmunoassay (RIA) or flow cytometry (FACS). In certain embodiments, an antibody that binds to CD40 has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁶M or less, e.g. from 10⁻⁶⁸ M to 10⁻¹³ M, e.g., from 10⁻⁸ M to 10⁻¹⁰ M).

The term “peptide linker” refers to a peptide comprising one or more amino acids, typically about 2 to 20 amino acids. Peptide linkers are known in the art or are described herein. Suitable, non-immunogenic linker peptides are, for example, (G₄S)ₙ, (SG₄)ₙ or G₄(SG₄)ₙ peptide linkers, wherein “n” is generally a number between 1 and 10, typically between 2 and 4, in particular 2, i.e. the peptides selected from the group consisting of GGGGS (SEQ ID NO:82), GGGGSGGGGS (SEQ ID NO:83), SGGGGSGGGG (SEQ ID NO: 84) and GGGGSGGGGSGGGG (SEQ ID NO:85), but also include the sequences GSPGSSSSGS (SEQ ID NO:86), (G4S)₃ (SEQ ID NO:87), (G4S)₄ (SEQ ID NO:88), GSGSGSGS (SEQ ID NO:89), GSGSGNGS (SEQ ID NO:90), GGSGSGSG (SEQ ID NO: 91), GGSGSG (SEQ ID NO:92), GGSG (SEQ ID NO:93), GGSGNGSG (SEQ ID NO: 94), GGNGSGSG (SEQ ID NO:95) and GGNGSG (SEQ ID NO:96). Peptide linkers of particular interest are (G4S) (SEQ ID NO:82), (G4S)₂ or GGGGSGGGGS (SEQ ID NO: 83), (G4S)₃ (SEQ ID NO:87) and (G4S)₄ (SEQ ID NO:88).

The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

By “fused” or “connected” is meant that the components (e.g. a heavy chain of an antibody and a Fab fragment) are linked by peptide bonds, either directly or via one or more peptide linkers.

“Percent (%) amino acid sequence identity” with respect to a reference polypeptide (protein) sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN. SAWI or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In certain embodiments, amino acid sequence variants of the bispecific antigen binding molecules provided herein are contemplated. For example, it may be desirable to improve the binding affinity and/or other biological properties of the TNF ligand trimer-containing antigen binding molecules. Amino acid sequence variants of the TNF ligand trimer-containing antigen binding molecules may be prepared by introducing appropriate modifications into the nucleotide sequence encoding the molecules, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Sites of interest for substitutional mutagenesis include the HVRs and Framework (FRs). Conservative substitutions are provided in Table B under the heading “Preferred Substitutions” and further described below in reference to amino acid side chain classes (1) to (6). Amino acid substitutions may be introduced into the molecule of interest and the products screened for a desired activity, e.g., retained/improved antigen binding, decreased immunogenicity, or improved ADCC or CDC.

TABLE A

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; | Ile |
| | Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “amino acid sequence variants” includes substantial variants wherein there are amino acid substitutions in one or more hypervariable region residues of a parent antigen binding molecule (e.g. a humanized or human antibody). Generally, the resulting variant(s) selected for further study will have modifications (e.g., improvements) in certain biological properties (e.g., increased affinity, reduced immunogenicity) relative to the parent antigen binding molecule and/or will have substantially retained certain biological properties of the parent antigen binding molecule. An exemplary substitutional variant is an affinity matured antibody, which may be conveniently generated, e.g., using phage display-based affinity maturation techniques such as those described herein. Briefly, one or more HVR residues are mutated and the variant antigen binding molecules displayed on phage and screened for a particular biological activity (e.g. binding affinity). In certain embodiments, substitutions, insertions, or deletions may occur within one or more HVRs so long as such alterations do not substantially reduce the ability of the antigen binding molecule to bind antigen. For example, conservative alterations (e.g., conservative substitutions as provided herein) that do not substantially reduce binding affinity may be made in HVRs. A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antigen binding molecule complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include bispecific antigen binding molecules of the invention with an N-terminal methionyl residue. Other insertional variants of the molecule include the fusion to the N- or C-terminus to a polypeptide which increases the serum half-life of the bispecific antigen binding molecules.

In certain embodiments, the bispecific antigen binding molecules provided herein are altered to increase or decrease the extent to which the antibody is glycosylated. Glycosylation variants of the molecules may be conveniently obtained by altering the amino acid sequence such that one or more glycosylation sites is created or removed. Where the TNF ligand trimer-containing antigen binding molecule comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in TNF family ligand trimer-containing antigen binding molecule may be made in order to create variants with certain improved properties. In one aspect, variants of bispecific antigen binding molecules or antibodies of the invention are provided having a carbohydrate structure that lacks fucose attached (directly or indirectly) to an Fc region. Such fucosylation variants may have improved ADCC function, see e.g. US Patent Publication Nos. US 2003/0157108 (Presta, L.) or US 2004/0093621 (Kyowa Hakko Kogyo Co., Ltd). In another aspect, variants of the bispecific antigen binding molecules or antibodies of the invention are provided with bisected oligosaccharides, e.g., in which a biantennary oligosaccharide attached to the Fc region is bisected by GlcNAc. Such variants may have reduced fucosylation and/or improved ADCC function, see for example WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such antibody variants may have improved CDC function and are described, e.g., in WO 1997/30087 (Patel et al.); WO 1998/58964 (Raju, S.); and WO 1999/22764 (Raju, S.).

In certain aspects, it may be desirable to create cysteine engineered variants of the bispecific antigen binding molecules of the invention, e.g., “thioMAbs,” in which one or more residues of the molecule are substituted with cysteine residues. In particular aspects, the substituted residues occur at accessible sites of the molecule. By substituting those residues with cysteine, reactive thiol groups are thereby positioned at accessible sites of the antibody and may be used to conjugate the antibody to other moieties, such as drug moieties or linker-drug moieties, to create an immunoconjugate. In certain aspects, any one or more of the following residues may be substituted with cysteine: V205 (Kabat numbering) of the light chain; A118 (EU numbering) of the heavy chain; and S400 (EU numbering) of the heavy chain Fc region. Cysteine engineered antigen binding molecules may be generated as described, e.g., in U.S. Pat. No. 7,521,541.

The term “nucleic acid” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al, Nature Medicine 2017, published online 12 Jun. 2017, doi: 10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding a bispecific antigen binding molecule or antibody” refers to one or more nucleic acid molecules encoding the heavy and light chains (or fragments thereof) of the bispecific antigen binding molecule or antibody, including such nucleic acid molecule(s) in a single vector or separate vectors, and such nucleic acid molecule(s) present at one or more locations in a host cell.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NSO cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

The term “cancer” as used herein refers to proliferative diseases, such as lymphomas, lymphocytic leukemias, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers, or a combination of one or more of the above cancers.

The term “chemotherapeutic agent” as used herein refers to a chemical compound useful in the treatment of cancer. In one aspect, the chemotherapeutic agent is an antimetabolite. In one aspect, the antimetabolite is selected from the group consisting of Aminopterin, Methotrexate, Pemetrexed, Raltitrexed, Cladribine, Clofarabine, Fludarabine, Mercaptopurine, Pentostatin, Thioguanine, Capecitabine, Cytarabine, Fluorouracil, Floxuridine, and Gemcitabine. In one particular aspect, the antimetabolite is capecitabine or gemcitabine. In another aspect, the antimetabolite is fluorouracil. In one aspect, the chemotherapeutic agent is an agent that affects microtubule formation. In one aspect, the agent that affects microtubule formation is selected from the group consisting of: paclitaxel, docetaxel, vincristine, vinblastine, vindesine, vinorelbin, taxotere, etoposide, and teniposide. In another aspect, the chemotherapeutic agent is an alkylating agent such as cyclophosphamide. In one aspect, the chemotherapeutic agent is a cytotoxic antibiotic such as a topoisomerase II inhibitor. In one aspect, the topoisomerase II inhibitor is doxorubicin.

### Bispecific Antibodies of the Invention

The invention provides novel bispecific antigen binding molecules comprising a new anti-FAP antibody (clone 212). The bispecific antigen binding molecules comprising this new anti-FAP antibody possess particularly advantageous properties such as producibility, stability, binding affinity, biological activity, targeting efficiency, reduced internalization, superior pharmacokinetic (PK) properties, reduced toxicity, an extended dosage range that can be given to a patient and thereby a possibly enhanced efficacy.

### Exemplary Bispecific Antigen Binding Molecules

In one aspect, the invention provides bispecific antigen binding molecules that are characterized by targeted agonistic binding to CD40. In particular, the bispecific antigen binding molecule is a CD40 agonist that is targeted against FAP. In another particular aspect, the bispecific antigen binding molecules of the invention comprise a Fc region composed of a first and a second subunit capable of stable association which comprises mutations that reduce effector function. The use of a Fc region comprising mutations that reduce or abolish effector function will prevent unspecific agonism by crosslinking via Fc receptors and will prevent ADCC of CD40⁺ cells. The bispecific antigen binding molecules as described herein possess the advantage over conventional antibodies capable of specific binding to CD40 in that they selectively induce immune response at the target cells, which are typically cancer cells or tumor stroma.

The bispecific antigen binding molecules are thus characterized by FAP-targeted agonistic binding to CD40. In the presence of FAP-expressing cells the bispecific antigen binding molecules are able to activate antigen presenting cells (APCs), to activate human B cells (Example 5.1.2), human Daudi cells (Example 5.1.1) and human monocyte-derived dendritic cells (moDCs).

In one aspect, provided is a bispecific antigen binding molecule comprising (a) at least one antigen binding domain capable of specific binding to CD40, and (b) at least one antigen binding domain capable of specific binding to Fibroblast Activation Protein (FAP) comprising a heavy chain variable region (V_{H}FAP) comprising (i) CDR-H1 comprising the amino acid sequence of SEQ ID NO:3, (ii) CDR-H2 comprising the amino acid sequence selected from the group consisting of SEQ ID NO:4, SEQ ID NO:11 and SEQ ID NO:12, and (iii) CDR-H3 comprising the amino acid sequence of SEQ ID NO:5, and a light chain variable region (V_{L}FAP) comprising (iv) CDR-L1 comprising the amino acid sequence selected from the group consisting of SEQ ID NO: 6, SEQ ID NO:13 and SEQ ID NO:14, (v) CDR-L2 comprising the amino acid sequence of SEQ ID NO:7, and (vi) CDR-L3 comprising the amino acid sequence of SEQ ID NO: 8.

In a particular aspect, provided is a bispecific antigen binding molecules, comprising a) at least one antigen binding domain capable of specific binding to CD40, and (b) at least one antigen binding domain capable of specific binding to Fibroblast Activation Protein (FAP) comprising a heavy chain variable region (V_{H}FAP) comprising (i) CDR-H1 comprising the amino acid sequence of SEQ ID NO:3, (ii) CDR-H2 comprising the amino acid sequence selected from the group consisting of SEQ ID NO:4, and (iii) CDR-H3 comprising the amino acid sequence of SEQ ID NO:5, and a light chain variable region (V_{L}FAP) comprising (iv) CDR-L1 comprising the amino acid sequence selected from the group consisting of SEQ ID NO:6, (v) CDR-L2 comprising the amino acid sequence of SEQ ID NO:7, and (vi) CDR-L3 comprising the amino acid sequence of SEQ ID NO:8.

In one aspect, provided is a bispecific antigen binding molecule, wherein the antigen binding domain capable of specific binding to FAP comprises a heavy chain variable region (V_{H}FAP) comprising an amino acid sequence that is at least about 90% identical to the amino acid sequence of SEQ ID NO:9, and a light chain variable region (V_{L}FAP) comprising an amino acid sequence that is at least about 90% identical to the amino acid sequence of SEQ ID NO:10.

In another aspect, provided is a bispecific antigen binding molecule, wherein the antigen binding domain capable of specific binding to FAP comprises a heavy chain variable region (V_{H}FAP) comprising an amino acid sequence selected from the group consisting of SEQ ID NO:15, SEQ ID NO:16, SEQ ID NO:17, SEQ ID NO: 18. SEQ ID NO:19 and SEQ ID NO:20, and a light chain variable region (V_{L}FAP) comprising an amino acid sequence selected from the group consisting of SEQ ID NO:21, SEQ ID NO:22, SEQ ID NO:23, SEQ ID NO: 24, SEQ ID NO:25 and SEQ ID NO:26.

In one aspect, the bispecific antigen binding molecule comprises
(a) a heavy chain variable region (V_{H}FAP) comprising the amino acid sequence of SEQ ID NO:15 and a light chain variable region (V_{L}FAP) comprising the amino acid sequence of SEQ ID NO:21,
(b) a heavy chain variable region (V_{H}FAP) comprising the amino acid sequence of SEQ ID NO:16 and a light chain variable region (V_{L}FAP) comprising the amino acid sequence of SEQ ID NO:21,
(c) a heavy chain variable region (V_{H}FAP) comprising the amino acid sequence of SEQ ID NO:16 and a light chain variable region (V_{L}FAP) comprising the amino acid sequence of SEQ ID NO:22, or
(d) a heavy chain variable region (V_{H}FAP) comprising the amino acid sequence of SEQ ID NO:19 and a light chain variable region (V_{L}FAP) comprising the amino acid sequence of SEQ ID NO:25.

In a particular aspect, the bispecific antigen binding molecule comprises a heavy chain variable region (V_{H}FAP) comprising the amino acid sequence of SEQ ID NO:15 and a light chain variable region (V_{L}FAP) comprising the amino acid sequence of SEQ ID NO: 21.

In another aspect, provided is a bispecific antigen binding molecule, wherein the antigen binding domain capable of specific binding to CD40 comprises a heavy chain variable region (V_{H}CD40) comprising (i) CDR-H1 comprising the amino acid sequence of SEQ ID NO:27, (ii) CDR-H2 comprising the amino acid sequence of SEQ ID NO:28, and (iii) CDR-H3 comprising the amino acid sequence of SEQ ID NO:29, and a light chain variable region (V_{L}CD40) comprising (iv) CDR-L1 comprising the amino acid sequence of SEQ ID NO:30, (v) CDR-L2 comprising the amino acid sequence of SEQ ID NO:31, and (vi) CDR-L3 comprising the amino acid sequence of SEQ ID NO:32.

In a further aspect, provided is a bispecific antigen binding molecule, wherein the antigen binding domain capable of specific binding to CD40 comprises
(i) a heavy chain variable region (V_{H}CD40) comprising an amino acid sequence selected from the group consisting of SEQ ID NO:37, SEQ ID NO:38, SEQ ID NO:39 and SEQ ID NO:40, and
(ii) a light chain variable region (V_{L}CD40) comprising the amino acid sequence selected from the group consisting of SEQ ID NO:41, SEQ ID NO:42, SEQ ID NO:43, and SEQ ID NO:44.

In one aspect, provided is a bispecific antigen binding molecule, wherein the antigen binding domain capable of specific binding to CD40 comprises
(a) a VH comprising the amino acid sequence of SEQ ID NO:37 and a VL comprising the amino acid sequence of SEQ ID NO:41, or
(b) a VH comprising the amino acid sequence of SEQ ID NO:37 and a VL comprising the amino acid sequence of SEQ ID NO:42, or
(c) a VH comprising the amino acid sequence of SEQ ID NO:37 and a VL comprising the amino acid sequence of SEQ ID NO:43, or
(d) a VH comprising the amino acid sequence of SEQ ID NO:37 and a VL comprising the amino acid sequence of SEQ ID NO:44, or
(e) a VH comprising the amino acid sequence of SEQ ID NO:38 and a VL comprising the amino acid sequence of SEQ ID NO:41, or
(f) a VH comprising the amino acid sequence of SEQ ID NO:38 and a VL comprising the amino acid sequence of SEQ ID NO:42, or
(g) a VH comprising the amino acid sequence of SEQ ID NO:38 and a VL comprising the amino acid sequence of SEQ ID NO:43, or
(h) a VH comprising the amino acid sequence of SEQ ID NO:38 and a VL comprising the amino acid sequence of SEQ ID NO:44, or
(i) a VH comprising the amino acid sequence of SEQ ID NO:39 and a VL comprising the amino acid sequence of SEQ ID NO:41, or
(j) a VH comprising the amino acid sequence of SEQ ID NO:39 and a VL comprising the amino acid sequence of SEQ ID NO:42, or
(k) a VH comprising the amino acid sequence of SEQ ID NO:39 and a VL comprising the amino acid sequence of SEQ ID NO:43, or
(l) a VH comprising the amino acid sequence of SEQ ID NO:39 and a VL comprising the amino acid sequence of SEQ ID NO:44, or
(m) a VH comprising the amino acid sequence of SEQ ID NO:40 and a VL comprising the amino acid sequence of SEQ ID NO:41, or
(n) a VH comprising the amino acid sequence of SEQ ID NO:40 and a VL comprising the amino acid sequence of SEQ ID NO:42, or
(o) a VH comprising the amino acid sequence of SEQ ID NO:40 and a VL comprising the amino acid sequence of SEQ ID NO:43, or
(p) a VH comprising the amino acid sequence of SEQ ID NO:40 and a VL comprising the amino acid sequence of SEQ ID NO:44.

In a particular aspect, the bispecific antigen binding molecule comprises an antigen binding domain capable of specific binding to CD40 comprises a VH comprising the amino acid sequence of SEQ ID NO:37 and a VL comprising the amino acid sequence of SEQ ID NO:41.

In a further aspect, provided is a bispecific antigen binding molecule, wherein the antigen binding domain capable of specific binding to CD40 comprises
(i) a heavy chain variable region (V_{H}CD40) comprising an amino acid sequence selected from the group consisting of SEQ ID NO:45, SEQ ID NO:46, SEQ ID NO:47, SEQ ID NO:48, SEQ ID NO:49 and SEQ ID NO:50, and
(ii) a light chain variable region (V_{L}CD40) comprising the amino acid sequence selected from the group consisting of SEQ ID NO:51, SEQ ID NO:52, SEQ ID NO:53, and SEQ ID NO:54.

In one aspect, provided is a bispecific antigen binding molecule, wherein the antigen binding domain capable of specific binding to CD40 comprises
(a) a VH comprising the amino acid sequence of SEQ ID NO:45 and a VL comprising the amino acid sequence of SEQ ID NO:51, or
(b) a VH comprising the amino acid sequence of SEQ ID NO:46 and a VL comprising the amino acid sequence of SEQ ID NO:51, or
(c) a VH comprising the amino acid sequence of SEQ ID NO:47 and a VL comprising the amino acid sequence of SEQ ID NO:51, or
(d) a VH comprising the amino acid sequence of SEQ ID NO:48 and a VL comprising the amino acid sequence of SEQ ID NO:51, or
(e) a VH comprising the amino acid sequence of SEQ ID NO:45 and a VL comprising the amino acid sequence of SEQ ID NO:52, or
(f) a VH comprising the amino acid sequence of SEQ ID NO:46 and a VL comprising the amino acid sequence of SEQ ID NO:52, or
(g) a VH comprising the amino acid sequence of SEQ ID NO:47 and a VL comprising the amino acid sequence of SEQ ID NO:52, or
(h) a VH comprising the amino acid sequence of SEQ ID NO:48 and a VL comprising the amino acid sequence of SEQ ID NO:52, or
(i) a VH comprising the amino acid sequence of SEQ ID NO:49 and a VL comprising the amino acid sequence of SEQ ID NO:53, or
(j) a VH comprising the amino acid sequence of SEQ ID NO:50 and a VL comprising the amino acid sequence of SEQ ID NO:53, or
(k) a VH comprising the amino acid sequence of SEQ ID NO:49 and a VL comprising the amino acid sequence of SEQ ID NO:54, or
(l) a VH comprising the amino acid sequence of SEQ ID NO:50 and a VL comprising the amino acid sequence of SEQ ID NO:54.

In a particular aspect, provided is a bispecific antigen binding molecule which comprises (i) at least one antigen binding domain capable of specific binding to CD40, comprising a heavy chain variable region (V_{H}CD40) comprising the amino acid sequence of SEQ ID NO:37 and a light chain variable region (V_{L}CD40) comprising the amino acid sequence of SEQ ID NO:41, and (ii) at least one antigen binding domain capable of specific binding to FAP, comprising a heavy chain variable region (V_{H}FAP) comprising an amino acid sequence of SEQ ID NO:15 and a light chain variable region (V_{L}FAP) comprising an amino acid sequence of SEQ ID NO:21.

### Bispecific Antigen Binding Molecules Binding to CD40 and FAP

In another aspect, provided is a bispecific antigen binding molecule, comprising
(i) at least one antigen binding domain capable of specific binding to CD40, comprising a heavy chain variable region (V_{H}CD40) comprising an amino acid sequence selected from the group consisting of SEQ ID NO:37, SEQ ID NO:38, SEQ ID NO:39 and SEQ ID NO:40, and a light chain variable region (V_{L}CD40) comprising the amino acid sequence selected from the group consisting of SEQ ID NO:41, SEQ ID NO:42, SEQ ID NO: 43, and SEQ ID NO:44, and
(ii) at least one antigen binding domain capable of specific binding to FAP, comprising a heavy chain variable region (V_{H}FAP) comprising an amino acid sequence selected from the group consisting of SEQ ID NO:15, SEQ ID NO:16, SEQ ID NO:17, SEQ ID NO:18. SEQ ID NO:19 and SEQ ID NO:20, and a light chain variable region (V_{L}FAP) comprising an amino acid sequence selected from the group consisting of SEQ ID NO: 21, SEQ ID NO:22, SEQ ID NO:23, SEQ ID NO:24, SEQ ID NO:25 and SEQ ID NO: 26.

In a further aspect, provided is a bispecific antigen binding molecule, comprising
(i) at least one antigen binding domain capable of specific binding to CD40, comprising a heavy chain variable region (V_{H}CD40) comprising an amino acid sequence selected from the group consisting of SEQ ID NO:45, SEQ ID NO:46, SEQ ID NO:47, SEQ ID NO:48, SEQ ID NO:49 and SEQ ID NO:50, and a light chain variable region (V_{L}CD40) comprising the amino acid sequence selected from the group consisting of SEQ ID NO: 51, SEQ ID NO:52, SEQ ID NO:53, and SEQ ID NO:54, and
(ii) at least one antigen binding domain capable of specific binding to FAP, comprising a heavy chain variable region (V_{H}FAP) comprising an amino acid sequence selected from the group consisting of SEQ ID NO:15, SEQ ID NO:16, SEQ ID NO:17, SEQ ID NO:18. SEQ ID NO:19 and SEQ ID NO:20, and a light chain variable region (V_{L}FAP) comprising an amino acid sequence selected from the group consisting of SEQ ID NO: 21, SEQ ID NO:22, SEQ ID NO:23, SEQ ID NO:24, SEQ ID NO:25 and SEQ ID NO: 26.

In a particular aspect, provided is a bispecific antigen binding molecule, comprising (i) at least one antigen binding domain capable of specific binding to CD40, comprising a heavy chain variable region (V_{H}CD40) comprising an amino acid sequence of SEQ ID NO:37 and a light chain variable region (V_{L}CD40) comprising an amino acid sequence of SEQ ID NO:41, and
(ii) at least one antigen binding domain capable of specific binding to FAP, comprising a heavy chain variable region (V_{H}FAP) comprising an amino acid sequence selected from the group consisting of SEQ ID NO:15, SEQ ID NO:16, SEQ ID NO:17, SEQ ID NO:18. SEQ ID NO:19 and SEQ ID NO:20, and a light chain variable region (V_{L}FAP) comprising an amino acid sequence selected from the group consisting of SEQ ID NO: 21, SEQ ID NO:22, SEQ ID NO:23, SEQ ID NO:24, SEQ ID NO:25 and SEQ ID NO: 26.

In another particular aspect, provided is a bispecific antigen binding molecule, comprising
(i) at least one antigen binding domain capable of specific binding to CD40, comprising a heavy chain variable region (V_{H}CD40) comprising an amino acid of SEQ ID NO: 45 or SEQ ID NO:48 and a light chain variable region (V_{L}CD40) comprising an amino acid sequence of SEQ ID NO:51, and
(ii) at least one antigen binding domain capable of specific binding to FAP, comprising a heavy chain variable region (V_{H}FAP) comprising an amino acid sequence selected from the group consisting of SEQ ID NO:15, SEQ ID NO:16, SEQ ID NO:17, SEQ ID NO:18. SEQ ID NO:19 and SEQ ID NO:20, and a light chain variable region (V_{L}FAP) comprising an amino acid sequence selected from the group consisting of SEQ ID NO: 21, SEQ ID NO:22, SEQ ID NO:23, SEQ ID NO:24, SEQ ID NO:25 and SEQ ID NO: 26.

In a further aspect, provided is a bispecific antigen binding molecule, wherein
(i) the antigen binding domain capable of specific binding to CD40 comprises a heavy chain variable region (V_{H}CD40) comprising an amino acid sequence selected from the group consisting of SEQ ID NO:37, SEQ ID NO:38, SEQ ID NO:39 and SEQ ID NO:40, and a light chain variable region (V_{L}CD40) comprising the amino acid sequence selected from the group consisting of SEQ ID NO:41, SEQ ID NO:42, SEQ ID NO:43, and SEQ ID NO: 44, and
(ii) the antigen binding domain capable of specific binding to FAP comprises a heavy chain variable region VH comprising an amino acid sequence of SEQ ID NO:15 and a light chain variable region comprising an amino acid sequence of SEQ ID NO:21.

Furthermore, provided is a bispecific antigen binding molecule, wherein
(i) the antigen binding domain capable of specific binding to CD40 comprises a heavy chain variable region (V_{H}CD40) comprising an amino acid sequence selected from the group consisting of SEQ ID NO:45, SEQ ID NO:46, SEQ ID NO:47, SEQ ID NO:48, SEQ ID NO:49 and SEQ ID NO:50, and a light chain variable region (V_{L}CD40) comprising the amino acid sequence selected from the group consisting of SEQ ID NO: 51, SEQ ID NO:52, SEQ ID NO:53, and SEQ ID NO:54, and
(ii) the antigen binding domain capable of specific binding to FAP comprises a heavy chain variable region VH comprising an amino acid sequence of SEQ ID NO:15 and a light chain variable region comprising an amino acid sequence of SEQ ID NO:21.

In a particular aspect, provided is a bispecific antigen binding molecule which comprises (i) at least one antigen binding domain capable of specific binding to CD40, comprising a heavy chain variable region (V_{H}CD40) comprising the amino acid sequence of SEQ ID NO:37 and a light chain variable region (V_{L}CD40) comprising the amino acid sequence of SEQ ID NO:41, and (ii) at least one antigen binding domain capable of specific binding to FAP, comprising a heavy chain variable region (V_{H}FAP) comprising an amino acid sequence of SEQ ID NO:15 and a light chain variable region (V_{L}FAP) comprising an amino acid sequence of SEQ ID NO:21.

### Bispecific, Monovalent Antigen Binding Molecules (1+1 Format)

In one aspect, the invention relates to bispecific antigen binding molecules comprising
(a) one antigen binding domain capable of specific binding to CD40,
(b) one antigen binding domain capable of specific binding to Fibroblast Activation Protein (FAP) comprising a heavy chain variable region (V_{H}FAP) comprising
(i) CDR-H1 comprising the amino acid sequence of SEQ ID NO:3, (ii) CDR-H2 comprising the amino acid sequence selected from the group consisting of SEQ ID NO:4, SEQ ID NO:11 and SEQ ID NO:12, and (iii) CDR-H3 comprising the amino acid sequence of SEQ ID NO:5, and a light chain variable region (V_{L}FAP) comprising (iv) CDR-L1 comprising the amino acid sequence selected from the group consisting of SEQ ID NO: 6, SEQ ID NO:13 and SEQ ID NO:14, (v) CDR-L2 comprising the amino acid sequence of SEQ ID NO:7, and (vi) CDR-L3 comprising the amino acid sequence of SEQ ID NO: 8 and (c) a Fc domain composed of a first and a second subunit capable of stable association.

In one particular aspect, provided is a bispecific antigen binding molecule, wherein said molecule comprises (a) a first Fab fragment capable of specific binding to CD40, (b) a second Fab fragment capable of specific binding to FAP comprising a heavy chain variable region (V_{H}FAP) comprising (i) CDR-H1 comprising the amino acid sequence of SEQ ID NO: 3, (ii) CDR-H2 comprising the amino acid sequence selected from the group consisting of SEQ ID NO:4, SEQ ID NO:11 and SEQ ID NO:12, and (iii) CDR-H3 comprising the amino acid sequence of SEQ ID NO:5, and a light chain variable region (V_{L}FAP) comprising (iv) CDR-L1 comprising the amino acid sequence selected from the group consisting of SEQ ID NO:6, SEQ ID NO:13 and SEQ ID NO:14, (v) CDR-L2 comprising the amino acid sequence of SEQ ID NO:7, and (vi) CDR-L3 comprising the amino acid sequence of SEQ ID NO:8, and (c) a Fc domain composed of a first and a second subunit capable of stable association.

In one particular aspect, the invention provides a bispecific antigen binding molecule comprising (a) a first heavy chain comprising the amino acid sequence of SEQ ID NO:63, a second heavy chain comprising the amino acid sequence of SEQ ID NO:98, a first light chain comprising the amino acid sequence of SEQ ID NO:61, a second light chain comprising the amino acid sequence of SEQ ID NO:62.

### Bispecific Antigen Binding Molecules Bivalent for Binding to CD40 and Monovalent for Binding to the Target Cell Antigen (2+1 Format)

In another aspect, the invention provides a bispecific antigen binding molecule comprising
(a) two antigen binding domains capable of specific binding to CD40,
(b) one antigen binding domain capable of specific binding to FAP comprising a heavy chain variable region (V_{H}FAP) comprising (i) CDR-H1 comprising the amino acid sequence of SEQ ID NO:3, (ii) CDR-H2 comprising the amino acid sequence selected from the group consisting of SEQ ID NO:4, SEQ ID NO:11 and SEQ ID NO:12, and (iii) CDR-H3 comprising the amino acid sequence of SEQ ID NO:5, and a light chain variable region (V_{L}FAP) comprising (iv) CDR-L1 comprising the amino acid sequence selected from the group consisting of SEQ ID NO:6, SEQ ID NO:13 and SEQ ID NO:14, (v) CDR-L2 comprising the amino acid sequence of SEQ ID NO:7, and (vi) CDR-L3 comprising the amino acid sequence of SEQ ID NO:8, and
(c) a Fc domain composed of a first and a second subunit capable of stable association.

Thus, provided is a bispecific antigen binding molecule, wherein the bispecific antigen binding molecule binds bivalently to CD40 and monovalently to FAP.

In one aspect, the bispecific antigen binding molecule comprises
(a) two light chains and two heavy chains of an antibody comprising two Fab fragments capable of specific binding to CD40 and the Fc domain, and
(b) a VH and VL domain capable of specific binding to a target cell antigen, wherein the VH domain and the VL domain are each connected via a peptide linker to one of the C-termini of the two heavy chains.

In a further aspect, provided is a bispecific antigen binding molecule, comprising
(a) two heavy chains, each heavy chain comprising a VH and CH1 domain of a Fab fragment capable of specific binding to CD40 and a Fc region subunit,
(b) two light chains, each light chain comprising a VL and CL domain of a Fab fragment capable of specific binding to CD40, and
(c) a cross-fab fragment capable of specific binding to FAP comprising a VL-CH1 chain and a VH-CL chain, wherein the VH-CL chain is connected to the C-terminus of one of the two heavy chains of (a).

In one aspect, the VH-CL (VH-Ckappa) chain is connected to the C-terminus of the Fc knob heavy chain.

In another aspect, provided is a bispecific antigen binding molecule, comprising
(a) two heavy chains, each heavy chain comprising a VH and CH1 domain of a Fab fragment capable of specific binding to CD40 and a Fc region subunit,
(b) two light chains, each light chain comprising a VL and CL domain of a Fab fragment capable of specific binding to CD40, and
(c) a cross-fab fragment capable of specific binding to FAP comprising a VL-CH1 chain and a VH-CL chain, wherein the VL-CH1 chain is connected to the C-terminus of one of the two heavy chains of (a).

In one aspect, the VL-CH1 chain is connected to the C-terminus of the Fc knob heavy chain.

In one particular aspect, the invention provides a bispecific antigen binding molecule comprising
(a) two light chains, each comprising the amino acid sequence of SEQ ID NO:62, one light chain comprising the amino acid sequence of SEQ ID NO:61, a first heavy chain comprising the amino acid sequence of SEQ ID NO:63, and a second heavy chain comprising the amino acid sequence of SEQ ID NO:64, or
(b) two light chains, each comprising the amino acid sequence of SEQ ID NO:66, one light chain comprising the amino acid sequence of SEQ ID NO:65, a first heavy chain comprising the amino acid sequence of SEQ ID NO:67, and a second heavy chain comprising the amino acid sequence of SEQ ID NO:68.

In one particular aspect, the invention provides a bispecific antigen binding molecule comprising
(a) two light chains, each comprising the amino acid sequence of SEQ ID NO:62, one light chain comprising the amino acid sequence of SEQ ID NO:61, a first heavy chain comprising the amino acid sequence of SEQ ID NO:63, and a second heavy chain comprising the amino acid sequence of SEQ ID NO:64.

### Bispecific Antigen Binding Molecules in Head-to-Tail Format (2+1)

In another aspect, provided is a bispecific antigen binding molecule, comprising
(a) a heavy chain comprising the VH and CH1 domain of a Fab fragment capable of specific binding to CD40 and a Fc region subunit,
(b) a heavy chain comprising a VH and CH1 domain of a Fab fragment capable of specific binding to CD40, a VL and CH1 domain of a Fab fragment capable of specific binding to FAP and a Fc region subunit,
(c) two light chains, each light chain comprising a VL and CL domain of a Fab fragment capable of specific binding to CD40, and
(d) a light chain comprising a VH and CL domain of a Fab fragment capable of specific binding to FAP.

### Bispecific Antigen Binding Molecules Bivalent for Binding to CD40 and Bivalent for Binding to the Target Cell Antigen (2+2 Format)

In another aspect, the invention provides a bispecific antigen binding molecule comprising
(a) two antigen binding domains capable of specific binding to CD40,
(b) two antigen binding domains capable of specific binding to FAP comprising (i) CDR-H1 comprising the amino acid sequence of SEQ ID NO:3, (ii) CDR-H2 comprising the amino acid sequence selected from the group consisting of SEQ ID NO:4, SEQ ID NO: 11 and SEQ ID NO:12, and (iii) CDR-H3 comprising the amino acid sequence of SEQ ID NO: 5, and a light chain variable region (V_{L}FAP) comprising (iv) CDR-L1 comprising the amino acid sequence selected from the group consisting of SEQ ID NO:6, SEQ ID NO: 13 and SEQ ID NO:14, (v) CDR-L2 comprising the amino acid sequence of SEQ ID NO: 7, and (vi) CDR-L3 comprising the amino acid sequence of SEQ ID NO:8, and
(c) a Fc domain composed of a first and a second subunit capable of stable association.

Thus, provided is a bispecific antigen binding molecule, wherein the bispecific antigen binding molecule binds bivalently to CD40 and bivalently to FAP.

In one aspect, provided is a bispecific antigen binding molecule, wherein the bispecific antigen binding molecule comprises
(a) two heavy chains, each heavy chain comprising a VH and CH1 domain of a Fab fragment capable of specific binding to CD40, and a Fc region subunit,
(b) two light chains, each light chain comprising a VL and CL domain of a Fab fragment capable of specific binding to CD40, and
(c) two Fab fragments capable of specific binding to FAP, wherein one of the Fab fragments is connected to the C-terminus of one of the two heavy chains of (a), and the other of the Fab fragments is connected to the C-terminus of the other of the two heavy chains of (a).

### Bispecific Antigen Binding Molecules Tetravalent for Binding to CD40 and Monovalent for Binding to the Target Cell Antigen (4+1 Format)

In another aspect, the invention provides a bispecific antigen binding molecule comprising
(a) four antigen binding domains capable of specific binding to CD40,
(b) one antigen binding domain capable of specific binding to FAP comprising (i) CDR-H1 comprising the amino acid sequence of SEQ ID NO:3, (ii) CDR-H2 comprising the amino acid sequence selected from the group consisting of SEQ ID NO:4, SEQ ID NO: 11 and SEQ ID NO:12, and (iii) CDR-H3 comprising the amino acid sequence of SEQ ID NO: 5, and a light chain variable region (V_{L}FAP) comprising (iv) CDR-L1 comprising the amino acid sequence selected from the group consisting of SEQ ID NO:6, SEQ ID NO: 13 and SEQ ID NO:14, (v) CDR-L2 comprising the amino acid sequence of SEQ ID NO: 7, and (vi) CDR-L3 comprising the amino acid sequence of SEQ ID NO:8, and
(c) a Fc domain composed of a first and a second subunit capable of stable association.

Thus, provided is a bispecific antigen binding molecule, wherein the bispecific antigen binding molecule binds tetravalently to CD40 and monovalently to FAP.

In one aspect, provided is a bispecific antigen binding molecule, wherein the four antigen binding domains capable of specific binding to CD40 are Fab fragments and each two thereof are fused to each other at the heavy chain, optionally via a peptide linker. In a particular aspect, the peptide linker comprises the amino acid sequence of SEQ ID NO:83. More particularly, the antigen binding molecule comprises two heavy chains comprising each a VHCH1-peptide linker-VHCH1 fragment. In a particular aspect, the peptide linker has the amino acid sequence of SEQ ID NO:83.

In a particular aspect, the bispecific antigen binding molecule comprises
(a) four light chains, each light chain comprising a VL and CL domain of a Fab fragment capable of specific binding to CD40,
(b) two heavy chains, wherein each of the heavy chain comprises a VH and CH1 domain of a Fab fragment capable of specific binding to CD40 fused to a VH and CH1 domain of a second Fab fragment capable of specific binding to CD40, and a Fc region subunit, and
(c) a cross-fab fragment capable of specific binding to FAP, wherein the VH domain is connected via a peptide linker to the C-terminus of one of the heavy chains.

In another aspect, the bispecific antigen binding molecule comprises
(a) four light chains, each light chain comprising a VL and CL domain of a Fab fragment capable of specific binding to CD40,
(b) two heavy chains, wherein each of the heavy chain comprises a VH and CH1 domain of a Fab fragment capable of specific binding to CD40 fused to a VH and CH1 domain of a second Fab fragment capable of specific binding to CD40, and a Fc region subunit, and
(c) a cross-fab fragment capable of specific binding to FAP, wherein the VL domain is connected via a peptide linker to the C-terminus of one of the heavy chains.

In a particular aspect, the peptide linker comprises an amino acid sequence selected from SEQ ID NO:82, SEQ ID NO:83, SEQ ID NO:87 and SEQ ID NO:88. More particularly, the peptide linker comprises the SEQ ID NO:83.

In a particular aspect, the invention provides a bispecific antigen binding molecule comprising
(a) four light chains, each comprising the amino acid sequence of SEQ ID NO:62, one light chain comprising the amino acid sequence of SEQ ID NO:61, a first heavy chain comprising the amino acid sequence of SEQ ID NO:69, and a second heavy chain comprising the amino acid sequence of SEQ ID NO:70.

### Bispecific Antigen Binding Molecules Tetravalent for Binding to CD40 and Bivalent for Binding to the Target Cell Antigen (4+2 Format)

In another aspect, the invention provides a bispecific antigen binding molecule comprising
(a) four antigen binding domains capable of specific binding to CD40,
(b) two antigen binding domains capable of specific binding to FAP comprising
(i) CDR-H1 comprising the amino acid sequence of SEQ ID NO:3, (ii) CDR-H2 comprising the amino acid sequence selected from the group consisting of SEQ ID NO:4, SEQ ID NO:11 and SEQ ID NO:12, and (iii) CDR-H3 comprising the amino acid sequence of SEQ ID NO:5, and a light chain variable region (V_{L}FAP) comprising (iv) CDR-L1 comprising the amino acid sequence selected from the group consisting of SEQ ID NO: 6, SEQ ID NO:13 and SEQ ID NO:14, (v) CDR-L2 comprising the amino acid sequence of SEQ ID NO:7, and (vi) CDR-L3 comprising the amino acid sequence of SEQ ID NO: 8, and
(c) a Fc domain composed of a first and a second subunit capable of stable association:

Thus, provided is a bispecific antigen binding molecule, wherein the bispecific antigen binding molecule binds tetravalently to CD40 and bivalently to FAP.

In one aspect, provided is a bispecific antigen binding molecule, wherein the four antigen binding domains capable of specific binding to CD40 are Fab fragments and each two thereof are fused to each other, optionally via a peptide linker. In a particular aspect, the peptide linker comprises the amino acid sequence of SEQ ID NO:83. More particularly, the antigen binding molecule comprises two heavy chains comprising each a VHCH1-peptide linker-VHCH1 fragment. In a particular aspect, the peptide linker has the amino acid sequence of SEQ ID NO:83.

In another aspect, a bispecific antigen binding molecule is provided, wherein the antigen binding domains capable of specific binding to a target cell antigen are Fab fragments and wherein the first Fab fragment is connected via a peptide linker to the C-terminus of the first subunit of the Fc domain and the second Fab fragment is connected via a peptide linker to the C-terminus of the second subunit of the Fc domain. In one aspect, the two Fab fragments capable of specific binding to the target cell antigen are crossover Fab fragments each comprising a VL-CH1 chain and a VH-CL chain, and wherein the VL-CH1 chain is connected to the C-terminus of one of the two heavy chains.

Fc Domain Modifications Reducing Fc Receptor Binding and/or Effector Function

The bispecific antigen binding molecules of the invention further comprise a Fc domain composed of a first and a second subunit capable of stable association.

In certain aspects, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

The Fc domain confers favorable pharmacokinetic properties to the bispecific antibodies of the invention, including a long serum half-life which contributes to good accumulation in the target tissue and a favorable tissue-blood distribution ratio. At the same time it may, however, lead to undesirable targeting of the bispecific antibodies of the invention to cells expressing Fc receptors rather than to the preferred antigen-bearing cells. Accordingly, in particular embodiments the Fc domain of the bispecific antibodies of the invention exhibits reduced binding affinity to an Fc receptor and/or reduced effector function, as compared to a native IgG Fc domain, in particular an IgG1 Fc domain or an IgG4 Fc domain. More particularly, the Fc domain is an IgG1 Fc domain.

In one such aspect the Fc domain (or the bispecific antigen binding molecule of the invention comprising said Fc domain) exhibits less than 50%, preferably less than 20%, more preferably less than 10% and most preferably less than 5% of the binding affinity to an Fc receptor, as compared to a native IgG1 Fc domain (or the bispecific antigen binding molecule of the invention comprising a native IgG1 Fc domain), and/or less than 50%, preferably less than 20%, more preferably less than 10% and most preferably less than 5% of the effector function, as compared to a native IgG1 Fc domain (or the bispecific antigen binding molecule of the invention comprising a native IgG1 Fc domain). In one aspect, the Fc domain (or the bispecific antigen binding molecule of the invention comprising said Fc domain) does not substantially bind to an Fc receptor and/or induce effector function. In a particular aspect the Fc receptor is an Fcγ receptor. In one aspect, the Fc receptor is a human Fc receptor. In one aspect, the Fc receptor is an activating Fc receptor. In a specific aspect, the Fc receptor is an activating human Fcγ receptor, more specifically human FcγRIIIa, FcγRI or FcγRIIa, most specifically human FcγRIIIa. In one aspect, the Fc receptor is an inhibitory Fc receptor. In a specific aspect, the Fc receptor is an inhibitory human Fcγ receptor, more specifically human Fc□RIIB. In one aspect the effector function is one or more of CDC, ADCC, ADCP, and cytokine secretion. In a particular aspect, the effector function is ADCC. In one aspect, the Fc domain exhibits substantially similar binding affinity to neonatal Fc receptor (FcRn), as compared to a native IgG1 Fc domain. Substantially similar binding to FcRn is achieved when the Fc domain (or the bispecific antigen binding molecule of the invention comprising said Fc domain) exhibits greater than about 70%, particularly greater than about 80%, more particularly greater than about 90% of the binding affinity of a native IgG1 Fc domain (or the bispecific antigen binding molecule of the invention comprising a native IgG1 Fc domain) to FcRn.

In a particular aspect, the Fc domain is engineered to have reduced binding affinity to an Fc receptor and/or reduced effector function, as compared to a non-engineered Fc domain. In a particular aspect, the Fc domain of the bispecific antigen binding molecule of the invention comprises one or more amino acid mutation that reduces the binding affinity of the Fc domain to an Fc receptor and/or effector function. Typically, the same one or more amino acid mutation is present in each of the two subunits of the Fc domain. In one aspect, the amino acid mutation reduces the binding affinity of the Fc domain to an Fc receptor. In another aspect, the amino acid mutation reduces the binding affinity of the Fc domain to an Fc receptor by at least 2-fold, at least 5-fold, or at least 10-fold. In one aspect, the bispecific antigen binding molecule of the invention comprising an engineered Fc domain exhibits less than 20%, particularly less than 10%, more particularly less than 5% of the binding affinity to an Fc receptor as compared to bispecific antibodies of the invention comprising a non-engineered Fc domain. In a particular aspect, the Fc receptor is an Fcγ receptor. In other aspects, the Fc receptor is a human Fc receptor. In one aspect, the Fc receptor is an inhibitory Fc receptor. In a specific aspect, the Fc receptor is an inhibitory human Fcγ receptor, more specifically human Fc□RIIB. In some aspects the Fc receptor is an activating Fc receptor. In a specific aspect, the Fc receptor is an activating human Fcγ receptor, more specifically human FcγRIIIa, FcγRI or FcγRIIa, most specifically human FcγRIIIa. Preferably, binding to each of these receptors is reduced. In some aspects, binding affinity to a complement component, specifically binding affinity to C1q, is also reduced. In one aspect, binding affinity to neonatal Fc receptor (FcRn) is not reduced. Substantially similar binding to FcRn, i.e. preservation of the binding affinity of the Fc domain to said receptor, is achieved when the Fc domain (or the bispecific antigen binding molecule of the invention comprising said Fc domain) exhibits greater than about 70% of the binding affinity of a non-engineered form of the Fc domain (or the bispecific antigen binding molecule of the invention comprising said non-engineered form of the Fc domain) to FcRn. The Fc domain, or the bispecific antigen binding molecule of the invention comprising said Fc domain, may exhibit greater than about 80% and even greater than about 90% of such affinity. In certain embodiments the Fc domain of the bispecific antigen binding molecule of the invention is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). Certain antibody variants with improved or diminished binding to FcRs are described. (e.g. U.S. Pat. No. 6,737,056; WO 2004/056312, and Shields, R. L. et al., J. Biol. Chem. 276 (2001) 6591-6604).

In one aspect of the invention, the Fc domain comprises an amino acid substitution at a position of E233, L234, L235, N297, P331 and P329. In some aspects, the Fc domain comprises the amino acid substitutions L234A and L235A (“LALA”). In one such embodiment, the Fc domain is an IgG1 Fc domain, particularly a human IgG1 Fc domain. In one aspect, the Fc domain comprises an amino acid substitution at position P329. In a more specific aspect, the amino acid substitution is P329A or P329G, particularly P329G. In one embodiment the Fc domain comprises an amino acid substitution at position P329 and a further amino acid substitution selected from the group consisting of E233P, L234A, L235A, L235E, N297A, N297D or P331S. In more particular embodiments the Fc domain comprises the amino acid mutations L234A, L235A and P329G (“P329G LALA”). The “P329G LALA” combination of amino acid substitutions almost completely abolishes Fcγ receptor binding of a human IgG1 Fc domain, as described in PCT Patent Application No. WO 2012/130831 A1. Said document also describes methods of preparing such mutant Fc domains and methods for determining its properties such as Fc receptor binding or effector functions. Such antibody is an IgG1 with mutations L234A and L235A or with mutations L234A, L235A and P329G (numbering according to EU index of Kabat et al, Kabat et al., Sequences of Proteins of Immunological Interest, 5th Ed. Public Health Service, National Institutes of Health, Bethesda, MD, 1991).

In one aspect, the Fc domain is an IgG4 Fc domain. In a more specific embodiment, the Fc domain is an IgG4 Fc domain comprising an amino acid substitution at position S228 (Kabat numbering), particularly the amino acid substitution S228P. In a more specific embodiment, the Fc domain is an IgG4 Fc domain comprising amino acid substitutions L235E and S228P and P329G. This amino acid substitution reduces in vivo Fab arm exchange of IgG4 antibodies (see Stubenrauch et al., Drug Metabolism and Disposition 38, 84-91 (2010)).

Antibodies with increased half-lives and improved binding to the neonatal Fc receptor (FcRn), which is responsible for the transfer of maternal IgGs to the fetus (Guyer, R. L. et al., J. Immunol. 117 (1976) 587-593, and Kim, J. K. et al., J. Immunol. 24 (1994) 2429-2434), are described in US 2005/0014934. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan, A. R. and Winter, G., Nature 322 (1988) 738-740; U.S. Pat. Nos. 5,648,260; 5,624,821; and WO 94/29351 concerning other examples of Fc region variants.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing FcγIIIa receptor. Effector function of an Fc domain, or bispecific antigen binding molecules of the invention comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, CA); and CytoTox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g. in an animal model such as that disclosed in Clynes et al., Proc Natl Acad Sci USA 95, 652-656 (1998).

The following section describes preferred aspects of the bispecific antigen binding molecules of the invention comprising Fc domain modifications reducing Fc receptor binding and/or effector function. In one aspect, the invention relates to the bispecific antigen binding molecule (a) at least one antigen binding domain capable of specific binding to CD40, (b) at least one antigen binding domain capable of specific binding to FAP, and (c) a Fc domain composed of a first and a second subunit capable of stable association, wherein the Fc domain comprises one or more amino acid substitution that reduces the binding affinity of the antibody to an Fc receptor, in particular towards Fc receptor. In another aspect, the invention relates to the bispecific antigen binding molecule comprising (a) at least one antigen binding domain capable of specific binding to CD40, (b) at least one antigen binding domain capable of specific binding to a target cell antigen, and (c) a Fc domain composed of a first and a second subunit capable of stable association, wherein the Fc domain comprises one or more amino acid substitution that reduces effector function. In particular aspect, the Fc domain is of human IgG1 subclass with the amino acid mutations L234A, L235A and P329G (numbering according to Kabat EU index).

### Fc Domain Modifications Promoting Heterodimerization

The bispecific antigen binding molecules of the invention comprise different antigen-binding sites, fused to one or the other of the two subunits of the Fc domain, thus the two subunits of the Fc domain may be comprised in two non-identical polypeptide chains. Recombinant co-expression of these polypeptides and subsequent dimerization leads to several possible combinations of the two polypeptides. To improve the yield and purity of the bispecific antigen binding molecules of the invention in recombinant production, it will thus be advantageous to introduce in the Fc domain of the bispecific antigen binding molecules of the invention a modification promoting the association of the desired polypeptides.

Accordingly, in particular aspects the invention relates to the bispecific antigen binding molecule comprising (a) at least one antigen binding domain capable of specific binding to CD40, (b) at least one antigen binding domain capable of specific binding to FAP, and (c) a Fc domain composed of a first and a second subunit capable of stable association, wherein the Fc domain comprises a modification promoting the association of the first and second subunit of the Fc domain. The site of most extensive protein-protein interaction between the two subunits of a human IgG Fc domain is in the CH3 domain of the Fc domain. Thus, in one aspect said modification is in the CH3 domain of the Fc domain.

In a specific aspect said modification is a so-called “knob-into-hole” modification, comprising a “knob” modification in one of the two subunits of the Fc domain and a “hole” modification in the other one of the two subunits of the Fc domain. Thus, the invention relates to the bispecific antigen binding molecule comprising (a) at least one antigen binding domain capable of specific binding to CD40, (b) at least one antigen binding domain capable of specific binding to a target cell antigen, and (c) a Fc domain composed of a first and a second subunit capable of stable association, wherein the first subunit of the Fc domain comprises knobs and the second subunit of the Fc domain comprises holes according to the knobs into holes method. In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in one aspect, in the CH3 domain of the first subunit of the Fc domain of the bispecific antigen binding molecules of the invention an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable. The protuberance and cavity can be made by altering the nucleic acid encoding the polypeptides, e.g. by site-specific mutagenesis, or by peptide synthesis. In a specific aspect, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one aspect, in the second subunit of the Fc domain additionally the threonine residue at position 366 is replaced with a serine residue (T366S) and the leucine residue at position 368 is replaced with an alanine residue (L368A).

In yet a further aspect, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues results in formation of a disulfide bridge between the two subunits of the Fc domain, further stabilizing the dimer (Carter (2001), J Immunol Methods 248, 7-15). In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

The C-terminus of the heavy chain of the bispecific antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, a bispecific antibody comprising a heavy chain including a C-terminal CH3 domain as specified herein, comprises the C-terminal glycine-lysine dipeptide (G446 and K447, numbering according to Kabat EU index). In one embodiment of all aspects as reported herein, a bispecific antibody comprising a heavy chain including a C-terminal CH3 domain, as specified herein, comprises a C-terminal glycine residue (G446, numbering according to Kabat EU index).

### Modifications in the Fab Domains

In one aspect, the invention relates to a bispecific antigen binding molecule comprising (a) a first Fab fragment capable of specific binding to CD40, (b) a second Fab fragment capable of specific binding to FAP, and (c) a Fc domain composed of a first and a second subunit capable of stable association, wherein in one of the Fab fragments either the variable domains VH and VL or the constant domains CH1 and CL are exchanged. The bispecific antibodies are prepared according to the Crossmab technology.

Multispecific antibodies with a domain replacement/exchange in one binding arm (CrossMabVH-VL or CrossMabCH-CL) are described in detail in WO2009/080252 and Schaefer, W. et al, PNAS, 108 (2011) 11187-1191. They clearly reduce the byproducts caused by the mismatch of a light chain against a first antigen with the wrong heavy chain against the second antigen (compared to approaches without such domain exchange).

In one aspect, the invention relates to a bispecific antigen binding molecule comprising (a) a first Fab fragment capable of specific binding to CD40, (b) a second Fab fragment capable of specific binding to FAP, and (c) a Fc domain composed of a first and a second subunit capable of stable association, wherein in one of the Fab fragments the constant domains CL and CH1 are replaced by each other so that the CH1 domain is part of the light chain and the CL domain is part of the heavy chain. More particularly, in the second Fab fragment capable of specific binding to a target cell antigen the constant domains CL and CH1 are replaced by each other so that the CH1 domain is part of the light chain and the CL domain is part of the heavy chain.

In a particular aspect, the invention relates a bispecific antigen binding molecule comprising (a) a first Fab fragment capable of specific binding to CD40, (b) a second Fab fragment capable of specific binding to FAP, wherein the constant domains CL and CH1 are replaced by each other so that the CH1 domain is part of the light chain and the CL domain is part of the heavy chain. Such a molecule binds monovalently to both CD40 and FAP.

In another aspect, the invention relates to a bispecific antigen binding molecule, comprising (a) two light chains and two heavy chains of an antibody comprising two Fab fragments capable of specific binding to CD40 and the Fc domain, and (b) two additional Fab fragments capable of specific binding to FAP, wherein said additional Fab fragments are each connected via a peptide linker to the C-terminus of the heavy chains of (a). In a particular aspect, the additional Fab fragments are Fab fragments, wherein the variable domains VL and VH are replaced by each other so that the VH domain is part of the light chain and the VL domain is part of the heavy chain.

Thus, in a particular aspect, the invention comprises a bispecific antigen binding molecule, comprising (a) two light chains and two heavy chains of an antibody comprising two Fab fragments capable of specific binding to CD40 and the Fc domain, and (b) two additional Fab fragments capable of specific binding to FAP, wherein said two additional Fab fragments capable of specific binding to a target cell antigen are crossover Fab fragments wherein the variable domains VL and VH are replaced by each other and the VL-CH chains are each connected via a peptide linker to the C-terminus of the heavy chains of (a).

In another aspect, and to further improve correct pairing, the bispecific antigen binding molecule comprising (a) a first Fab fragment capable of specific binding to CD40, (b) a second Fab fragment capable of specific binding to FAP, and (c) a Fc domain composed of a first and a second subunit capable of stable association, can contain different charged amino acid substitutions (so-called “charged residues”). These modifications are introduced in the crossed or non-crossed CH1 and CL domains. In a particular aspect, the invention relates to a bispecific antigen binding molecule, wherein in one of CL domains the amino acid at position 123 (EU numbering) has been replaced by arginine (R) and/or wherein the amino acid at position 124 (EU numbering) has been substituted by lysine (K) and wherein in one of the CH1 domains the amino acids at position 147 (EU numbering) and/or at position 213 (EU numbering) have been substituted by glutamic acid (E).

### Exemplary Antibodies of the Invention

In one aspect, the invention provides new antibodies and antibody fragments that specifically bind to FAP. These antibodies bind to a different epitope than the known FAP antibodies 4B) or 28H1 that make them especially suitable for the incorporation into bispecific antigen binding molecules that can be used in combination with other FAP-targeted molecules. The new antibodies are further characterized in that they are producable in high amounts and with high titers, that they show high thermal stability (as measured by the aggregation temperature Tagg), that they are supposed to possess excellent PK properties and that they bind with high affinity to human FAP as measured by Biacore assay.

In one aspect, provided is an antibody that specifically binds to FAP (clone 212), wherein said antibody comprises a heavy chain variable region (V_{H}FAP) comprising (i) CDR-H1 comprising the amino acid sequence of SEQ ID NO:3, (ii) CDR-H2 comprising the amino acid sequence selected from the group consisting of SEQ ID NO:4, SEQ ID NO: 11 and SEQ ID NO:12, and (iii) CDR-H3 comprising the amino acid sequence of SEQ ID NO: 5, and a light chain variable region (V_{L}FAP) comprising (iv) CDR-L1 comprising the amino acid sequence selected from the group consisting of SEQ ID NO:6, SEQ ID NO: 13 and SEQ ID NO:14, (v) CDR-L2 comprising the amino acid sequence of SEQ ID NO: 7, and (vi) CDR-L3 comprising the amino acid sequence of SEQ ID NO:8.

In one aspect, provided is a humanized antibody that specifically binds to FAP, wherein said antibody comprises a heavy chain variable region (V_{H}FAP) comprising (i) CDR-H1 comprising the amino acid sequence of SEQ ID NO:3, (ii) CDR-H2 comprising the amino acid sequence selected from the group consisting of SEQ ID NO:4, SEQ ID NO: 11 and SEQ ID NO:12, and (iii) CDR-H3 comprising the amino acid sequence of SEQ ID NO: 5, and a light chain variable region (V_{L}FAP) comprising (iv) CDR-L1 comprising the amino acid sequence selected from the group consisting of SEQ ID NO:6, SEQ ID NO: 13 and SEQ ID NO:14, (v) CDR-L2 comprising the amino acid sequence of SEQ ID NO: 7, and (vi) CDR-L3 comprising the amino acid sequence of SEQ ID NO:8.

In another aspect, provided is an antibody that competes for binding with an antibody that specifically binds to FAP, wherein said antibody comprises any of the heavy chain variable regions (V_{H}FAP) comprising an amino acid sequence selected from the group consisting of SEQ ID NO:15, SEQ ID NO:16, SEQ ID NO:17, SEQ ID NO:18. SEQ ID NO: 19 and SEQ ID NO:20, and any of the light chain variable regions (V_{L}FAP) comprising an amino acid sequence selected from the group consisting of SEQ ID NO:21, SEQ ID NO:22, SEQ ID NO:23, SEQ ID NO:24, SEQ ID NO:25 and SEQ ID NO:26.

In one aspect, provided is an antibody that competes for binding with an antibody that specifically binds to FAP, wherein said antibody comprises a heavy chain variable region VH comprising an amino acid sequence of SEQ ID NO:15 and a light chain variable region VL comprising an amino acid sequence of SEQ ID NO:21.

In a further aspect, provided is an antibody that specifically binds to FAP, wherein said antibody comprises
(a) a heavy chain variable region (V_{H}FAP) comprising the amino acid sequence of SEQ ID NO: 15 and a light chain variable region (V_{L}FAP) comprising the amino acid sequence of SEQ ID NO:21,
(b) a heavy chain variable region (V_{H}FAP) comprising the amino acid sequence of SEQ ID NO: 16 and a light chain variable region (V_{L}FAP) comprising the amino acid sequence of SEQ ID NO:21,
(c) a heavy chain variable region (V_{H}FAP) comprising the amino acid sequence of SEQ ID NO: 16 and a light chain variable region (V_{L}FAP) comprising the amino acid sequence of SEQ ID NO:22, or
(d) a heavy chain variable region (V_{H}FAP) comprising the amino acid sequence of SEQ ID NO: 19 and a light chain variable region (V_{L}FAP) comprising the amino acid sequence of SEQ ID NO:25.

In a further aspect, provided is an antibody that specifically binds to FAP comprising a heavy chain variable region (V_{H}FAP) comprising the amino acid sequence of SEQ ID NO: 15 and a light chain variable region (V_{L}FAP) comprising the amino acid sequence of SEQ ID NO:21.

### Polynucleotides

The invention further provides isolated nucleic acid encoding a bispecific antigen binding molecule as described herein or a fragment thereof or isolated nucleic acid encoding an antibody as described herein.

The isolated polynucleotides encoding bispecific antigen binding molecules of the invention may be expressed as a single polynucleotide that encodes the entire antigen binding molecule or as multiple (e.g., two or more) polynucleotides that are co-expressed. Polypeptides encoded by polynucleotides that are co-expressed may associate through, e.g., disulfide bonds or other means to form a functional antigen binding molecule. For example, the light chain portion of an immunoglobulin may be encoded by a separate polynucleotide from the heavy chain portion of the immunoglobulin. When co-expressed, the heavy chain polypeptides will associate with the light chain polypeptides to form the immunoglobulin.

In some aspects, the isolated polynucleotide encodes a polypeptide comprised in the bispecific molecule according to the invention as described herein.

In one aspect, the present invention is directed to an isolated polynucleotide encoding a bispecific antigen binding molecule, comprising (a) at least one antigen binding domain capable of specific binding to CD40, (b) at least one antigen binding domain capable of specific binding to a FAP comprising a heavy chain variable region (V_{H}FAP) comprising (i) CDR-H1 comprising the amino acid sequence of SEQ ID NO:3, (ii) CDR-H2 comprising the amino acid sequence selected from the group consisting of SEQ ID NO:4, SEQ ID NO:11 and SEQ ID NO:12, and (iii) CDR-H3 comprising the amino acid sequence of SEQ ID NO:5, and a light chain variable region (V_{L}FAP) comprising (iv) CDR-L1 comprising the amino acid sequence selected from the group consisting of SEQ ID NO: 6, SEQ ID NO:13 and SEQ ID NO:14, (v) CDR-L2 comprising the amino acid sequence of SEQ ID NO:7, and (vi) CDR-L3 comprising the amino acid sequence of SEQ ID NO: 8, and (c) a Fc domain composed of a first and a second subunit capable of stable association.

In certain embodiments the polynucleotide or nucleic acid is DNA. In other embodiments, a polynucleotide of the present invention is RNA, for example, in the form of messenger RNA (mRNA). RNA of the present invention may be single stranded or double stranded.

### Recombinant Methods

Bispecific antigen binding molecules of the invention may be obtained, for example, by recombinant production. For recombinant production one or more polynucleotide encoding the bispecific antigen binding molecule or polypeptide fragments thereof are provided. The one or more polynucleotide encoding the bispecific antigen binding molecule are isolated and inserted into one or more vectors for further cloning and/or expression in a host cell. Such polynucleotide may be readily isolated and sequenced using conventional procedures. In one aspect of the invention, a vector, preferably an expression vector, comprising one or more of the polynucleotides of the invention is provided. Methods which are well known to those skilled in the art can be used to construct expression vectors containing the coding sequence of the bispecific antigen binding molecule (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989). The expression vector can be part of a plasmid, virus, or may be a nucleic acid fragment. The expression vector includes an expression cassette into which the polynucleotide encoding the bispecific antigen binding molecule or polypeptide fragments thereof (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the bispecific antigen binding molecule of the invention or polypeptide fragments thereof, or variants or derivatives thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription.

Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit â-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the bispecific antigen binding molecule or polypeptide fragments thereof is desired, DNA encoding a signal sequence may be placed upstream of the nucleic acid encoding the bispecific antigen binding molecule of the invention or polypeptide fragments thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the fusion protein may be included within or at the ends of the polynucleotide encoding a bispecific antigen binding molecule of the invention or polypeptide fragments thereof.

In a further aspect of the invention, a host cell comprising one or more polynucleotides of the invention is provided. In certain aspects, a host cell comprising one or more vectors of the invention is provided. The polynucleotides and vectors may incorporate any of the features, singly or in combination, described herein in relation to polynucleotides and vectors, respectively. In one aspect, a host cell comprises (e.g. has been transformed or transfected with) a vector comprising a polynucleotide that encodes (part of) a bispecific antigen binding molecule of the invention of the invention. As used herein, the term “host cell” refers to any kind of cellular system which can be engineered to generate the fusion proteins of the invention or fragments thereof. Host cells suitable for replicating and for supporting expression of antigen binding molecules are well known in the art. Such cells may be transfected or transduced as appropriate with the particular expression vector and large quantities of vector containing cells can be grown for seeding large scale fermenters to obtain sufficient quantities of the antigen binding molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006).

Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr-CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B.K.C. Lo, ed., Humana Press, Totowa, NJ), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., Y0, NS0, Sp20 cell). Standard technologies are known in the art to express foreign genes in these systems. Cells expressing a polypeptide comprising either the heavy or the light chain of an immunoglobulin, may be engineered so as to also express the other of the immunoglobulin chains such that the expressed product is an immunoglobulin that has both a heavy and a light chain.

In one aspect, a method of producing a bispecific antigen binding molecule of the invention or polypeptide fragments thereof is provided, wherein the method comprises culturing a host cell comprising polynucleotides encoding the bispecific antigen binding molecule of the invention or polypeptide fragments thereof, as provided herein, under conditions suitable for expression of the bispecific antigen binding molecule of the invention or polypeptide fragments thereof, and recovering the bispecific antigen binding molecule of the invention or polypeptide fragments thereof from the host cell (or host cell culture medium).

Bispecific molecules of the invention prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the bispecific antigen binding molecule binds. For example, for affinity chromatography purification of fusion proteins of the invention, a matrix with protein A or protein G may be used. Sequential Protein A or G affinity chromatography and size exclusion chromatography can be used to isolate an antigen binding molecule essentially as described in the examples. The purity of the bispecific antigen binding molecule or fragments thereof can be determined by any of a variety of well-known analytical methods including gel electrophoresis, high pressure liquid chromatography, and the like. For example, the bispecific antigen binding molecules expressed as described in the Examples were shown to be intact and properly assembled as demonstrated by reducing and non-reducing SDS-PAGE.

### Assays

The antigen binding molecules provided herein may be characterized for their binding properties and/or biological activity by various assays known in the art. In particular, they are characterized by the assays described in more detail in the examples.

### 1. Binding Assay

Binding of the bispecific antigen binding molecule provided herein to the corresponding target expressing cells may be evaluated for example by using a murine fibroblast cell line expressing human Fibroblast Activation Protein (FAP) and flow cytometry (FACS) analysis. Binding of the bispecific antigen binding molecules provided herein to CD40 may be determined by using primary B cells as described in Example 4.2.

### 2. Activity Assays

Bispecific antigen binding molecules of the invention are tested for biological activity. Biological activity may include efficacy and specificity of the bispecific antigen binding molecules. Efficacy and specificity are demonstrated by assays showing agonistic signaling through the CD40 receptor upon binding of the target antigen. Furthermore, in vitro T cell priming assays are conducted using dendritic cells (DCs) that have been incubated with the bispecific antigen binding molecules.

### Pharmaceutical Compositions, Formulations and Routes of Administration

In a further aspect, the invention provides pharmaceutical compositions comprising any of the bispecific antigen binding molecules provided herein, e.g., for use in any of the below therapeutic methods. In one embodiment, a pharmaceutical composition comprises any of the bispecific antigen binding molecules provided herein and at least one pharmaceutically acceptable excipient. In another embodiment, a pharmaceutical composition comprises any of the bispecific antigen binding molecules provided herein and at least one additional therapeutic agent, e.g., as described below.

Pharmaceutical compositions of the present invention comprise a therapeutically effective amount of one or more bispecific antigen binding molecules dissolved or dispersed in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one bispecific antigen binding molecule according to the invention and optionally an additional active ingredient will be known to those of skill in the art in light of the present disclosure, as exemplified by Remington's Pharmaceutical Sciences, 18th Ed. Mack Printing Company, 1990, incorporated herein by reference. In particular, the compositions are lyophilized formulations or aqueous solutions. As used herein, “pharmaceutically acceptable excipient” includes any and all solvents, buffers, dispersion media, coatings, surfactants, antioxidants, preservatives (e.g. antibacterial agents, antifungal agents), isotonic agents, salts, stabilizers and combinations thereof, as would be known to one of ordinary skill in the art.

Parenteral compositions include those designed for administration by injection, e.g. subcutaneous, intradermal, intra-lesional, intravenous, intra-arterial, intramuscular, intrathecal or intraperitoneal injection. For injection, the bispecific antigen binding molecules of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the bispecific antigen binding molecules may be in powder form for constitution with a suitable vehicle, e.g., sterile pyrogen-free water, before use. Sterile injectable solutions are prepared by incorporating the antigen binding molecules of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein. Suitable pharmaceutically acceptable excipients include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

Exemplary pharmaceutically acceptable excipients herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

In addition to the compositions described previously, the antigen binding molecules may also be formulated as a depot preparation. Such long acting formulations may be administered by implantation (for example subcutaneously or intramuscularly) or by intramuscular injection. Thus, for example, the fusion proteins may be formulated with suitable polymeric or hydrophobic materials (for example as emulsion in an acceptable oil) or ion exchange resins, or as sparingly soluble derivatives, for example, as a sparingly soluble salt.

Pharmaceutical compositions comprising the bispecific antigen binding molecules of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The bispecific antigen binding molecules may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The composition herein may also contain more than one active ingredients as necessary for the particular indication being treated, preferably those with complementary activities that do not adversely affect each other. Such active ingredients are suitably present in combination in amounts that are effective for the purpose intended.

The formulations to be used for in vivo administration are generally sterile. Sterility may be readily accomplished, e.g., by filtration through sterile filtration membranes.

### Therapeutic Methods and Compositions

Any of the bispecific antigen binding molecules provided herein may be used in therapeutic methods. For use in therapeutic methods, bispecific antigen binding molecules of the invention can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, bispecific antigen binding molecules of the invention for use as a medicament are provided.

In further aspects, bispecific antigen binding molecules of the invention for use (i) in inducing immune stimulation by CD40+ antigen-presenting cells (APCs), (ii) in stimulating tumor-specific T cell response, (iii) in causing apoptosis of tumor cells, (iv) in the treatment of cancer, (v) in delaying progression of cancer, (vi) in prolonging the survival of a patient suffering from cancer, (vii) in the treatment of infections are provided. In a particular aspect, bispecific antigen binding molecules of the invention for use in treating a disease, in particular for use in the treatment of cancer, are provided.

In certain aspects, bispecific antigen binding molecules of the invention for use in a method of treatment are provided. In one aspect, the invention provides a bispecific antigen binding molecule as described herein for use in the treatment of a disease in an individual in need thereof. In certain aspects, the invention provides a bispecific antigen binding molecule for use in a method of treating an individual having a disease comprising administering to the individual a therapeutically effective amount of the bispecific antigen binding molecule. In certain aspects the disease to be treated is cancer. The subject, patient, or “individual” in need of treatment is typically a mammal, more specifically a human.

In one aspect, provided is a method for i) inducing immune stimulation by CD40+ antigen-presenting cells (APCs), (ii) stimulating tumor-specific T cell response, (iii) causing apoptosis of tumor cells, (iv) treating of cancer, (v) delaying progression of cancer, (vi) prolonging the survival of a patient suffering from cancer, or (vii) treating of infections, wherein the method comprises administering a therapeutically effective amount of the bispecific antigen binding molecule of the invention to an individual in need thereof.

In a further aspect, the invention provides for the use of the bispecific antigen binding molecule of the invention in the manufacture or preparation of a medicament for the treatment of a disease in an individual in need thereof. In one aspect, the medicament is for use in a method of treating a disease comprising administering to an individual having the disease a therapeutically effective amount of the medicament. In certain aspects, the disease to be treated is a proliferative disorder, particularly cancer. Examples of cancers include, but are not limited to, bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other examples of cancer include carcinoma, lymphoma (e.g., Hodgkin's and non-Hodgkin's lymphoma), blastoma, sarcoma, and leukemia. Other cell proliferation disorders that can be treated using the bispecific antigen binding molecule or antibody of the invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. A skilled artisan readily recognizes that in many cases the bispecific antigen binding molecule or antibody of the invention may not provide a cure but may provide a benefit. In some aspects, a physiological change having some benefit is also considered therapeutically beneficial. Thus, in some aspects, an amount of the bispecific antigen binding molecule or antibody of the invention that provides a physiological change is considered an “effective amount” or a “therapeutically effective amount”.

For the prevention or treatment of disease, the appropriate dosage of a bispecific antigen binding molecule of the invention (when used alone or in combination with one or more other additional therapeutic agents) will depend on the type of disease to be treated, the route of administration, the body weight of the patient, the specific molecule, the severity and course of the disease, whether the bispecific antigen binding molecule of the invention is administered for preventive or therapeutic purposes, previous or concurrent therapeutic interventions, the patient's clinical history and response to the bispecific antigen binding molecule, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The bispecific antigen binding molecule of the invention is suitably administered to the patient at one time or over a series of treatments. Depending on the type and severity of the disease, about 1 μg/kg to 15 mg/kg (e.g. 0.1 mg/kg-10 mg/kg) of the bispecific antigen binding molecule can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the bispecific antigen binding molecule of the invention would be in the range from about 0.005 mg/kg to about 10 mg/kg. In other examples, a dose may also comprise from about 1 μg/kg body weight, about 5 μg/kg body weight, about 10 μg/kg body weight, about 50 μg/kg body weight, about 100 μg/kg body weight, about 200 μg/kg body weight, about 350 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, to about 1000 mg/kg body weight or more per administration, and any range derivable therein. In examples of a derivable range from the numbers listed herein, a range of about 0.1 mg/kg body weight to about 20 mg/kg body weight, about 5 μg/kg body weight to about 1 mg/kg body weight etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the fusion protein). In a particular aspect, the bispecific antigen binding molecule will be administered every three weeks. An initial higher loading dose, followed by one or more lower doses may be administered. However, other dosage regimens may be useful. The progress of this therapy is easily monitored by conventional techniques and assays.

The bispecific antigen binding molecule of the invention will generally be used in an amount effective to achieve the intended purpose. For use to treat or prevent a disease condition, the bispecific antigen binding molecule of the invention, or pharmaceutical compositions thereof, are administered or applied in a therapeutically effective amount. Determination of a therapeutically effective amount is well within the capabilities of those skilled in the art, especially in light of the detailed disclosure provided herein. For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the bispecific antigen binding molecule of the invention which are sufficient to maintain therapeutic effect. Usual patient dosages for administration by injection range from about 0.1 to 50 mg/kg/day, typically from about 0.1 to 1 mg/kg/day. Therapeutically effective plasma levels may be achieved by administering multiple doses each day. Levels in plasma may be measured, for example, by HPLC. In cases of local administration or selective uptake, the effective local concentration of the bispecific antigen binding molecule or antibody of the invention may not be related to plasma concentration. One skilled in the art will be able to optimize therapeutically effective local dosages without undue experimentation.

A therapeutically effective dose of the bispecific antigen binding molecule of the invention described herein will generally provide therapeutic benefit without causing substantial toxicity. Toxicity and therapeutic efficacy of a fusion protein can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Bispecific antigen binding molecules that exhibit large therapeutic indices are preferred. In one aspect, the bispecific antigen binding molecule or antibody of the invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety).

The attending physician for patients treated with fusion proteins of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

### Other Agents and Treatments

The bispecific antigen binding molecule of the invention may be administered in combination with one or more other agents in therapy. For instance, the bispecific antigen binding molecule or antibody of the invention of the invention may be co-administered with at least one additional therapeutic agent. The term “therapeutic agent” encompasses any agent that can be administered for treating a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is another anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent. In certain aspects, an additional therapeutic agent is an immunomodulatory agent, a cytostatic agent, an inhibitor of cell adhesion, a cytotoxic or cytostatic agent, an activator of cell apoptosis, or an agent that increases the sensitivity of cells to apoptotic inducers.

Thus, provided are bispecific antigen binding molecules of the invention or pharmaceutical compositions comprising them for use in the treatment of cancer, wherein the bispecific antigen binding molecule is administered in combination with a chemotherapeutic agent, radiation and/or other agents for use in cancer immunotherapy.

Such other agents are suitably present in combination in amounts that are effective for the purpose intended. The effective amount of such other agents depends on the amount of fusion protein used, the type of disorder or treatment, and other factors discussed above. The bispecific antigen binding molecule or antibody of the invention are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the bispecific antigen binding molecule or antibody of the invention can occur prior to, simultaneously, and/or following, administration of the additional therapeutic agent and/or adjuvant.

In a further aspect, provided is the bispecific antigen binding molecule as described herein before for use in the treatment of cancer, wherein the bispecific antigen binding molecule is administered in combination with another immunomodulator.

The term “immunomodulator” refers to any substance including a monoclonal antibody that effects the immune system. The molecules of the inventions can be considered immunomodulators. Immunomodulators can be used as anti-neoplastic agents for the treatment of cancer. In one aspect, immunomodulators include, but are not limited to anti-CTLA4 antibodies (e.g. ipilimumab), anti-PD1 antibodies (e.g. nivolumab or pembrolizumab), PD-L1 antibodies (e.g. atezolizumab, avelumab or durvalumab), OX-40 antibodies, 4-1BB antibodies and GITR antibodies. In a further aspect, provided is the bispecific antigen binding molecule as described herein before for use in the treatment of cancer, wherein the bispecific antigen binding molecule is administered in combination with an agent blocking PD-L1/PD-1 interaction. In one aspect, the agent blocking PD-L1/PD-1 interaction is an anti-PD-L1 antibody or an anti-PD1 antibody. More particularly, the agent blocking PD-L1/PD-1 interaction is an anti-PD-L1 antibody, in particular an anti-PD-L1 antibody selected from the group consisting of atezolizumab, durvalumab, pembrolizumab and nivolumab. In one specific aspect, the agent blocking PD-L1/PD-1 interaction is atezolizumab (MPDL3280A, RG7446). In another aspect, the agent blocking PD-L1/PD-1 interaction is an anti-PD-L1 antibody comprising a heavy chain variable domain VH (PDL-1) of SEQ ID NO:107 and a light chain variable domain VL (PDL-1) of SEQ ID NO:108. In another aspect, the agent blocking PD-L1/PD-1 interaction is an anti-PD-L1 antibody comprising a heavy chain variable domain VH (PDL-1) of SEQ ID NO: 109 and a light chain variable domain VL (PDL-1) of SEQ ID NO:110. In another aspect, the agent blocking PD-L1/PD-1 interaction is an anti-PD1 antibody, in particular an anti-PD1 antibody selected from pembrolizumab or nivolumab. Such other agents are suitably present in combination in amounts that are effective for the purpose intended. The effective amount of such other agents depends on the amount of bispecific antigen binding molecule used, the type of disorder or treatment, and other factors discussed above. The bispecific antigen binding molecules as described herein before are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the bispecific antigen binding molecule can occur prior to, simultaneously, and/or following, administration of the additional therapeutic agent and/or adjuvant.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a bispecific antigen binding molecule of the invention.

The label or package insert indicates that the composition is used for treating the condition of choice. Moreover, the article of manufacture may comprise (a) a first container with a composition contained therein, wherein the composition comprises a bispecific antigen binding molecule of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

TABLE B
(Sequences):

| SEQ | | |
|---|---|---|
| ID NO: | Name | Sequence |
| 1 | hu CD40 | UniProt no. P25942, version 200 |
| | | MVRLPLQCVL WGCLLTAVHP |
| | | EPPTACREKQ YLINSQCCSL |
| | | CQPGQKLVSD CTEFTETECL PCGESEFLDT |
| | | WNRETHCHQH KYCDPNLGLR |
| | | VQQKGTSETD TICTCEEGWH |
| | | CTSEACESCV LHRSCSPGFG |
| | | VKQIATGVSD TICEPCPVGF FSNVSSAFEK |
| | | CHPWTSCETK DLVVQQAGTN |
| | | KTDVVCGPQD RLRALVVIPI IFGILFAILL |
| | | VLVFIKKVAK KPTNKAPHPK |
| | | QEPQEINFPD DLPGSNTAAP |
| | | VQETLHGCQP VTQEDGKESR ISVQERQ |
| 2 | hu FAP | UniProt no. Q12884, version 168 |
| | | MKTWVKIVFG VATSAVLALL |
| | | VMCIVLRPSR VHNSEENTMR |
| | | ALTLKDILNG TFSYKTFFPN WISGQEYLHQ |
| | | SADNNIVLYN IETGQSYTIL SNRTMKSVNA |
| | | SNYGLSPDRQ FVYLESDYSK |
| | | LWRYSYTATY YIYDLSNGEF |
| | | VRGNELPRPI QYLCWSPVGS |
| | | KLAYVYQNNI YLKQRPGDPP |
| | | FQITFNGREN KIFNGIPDWV |
| | | YEEEMLATKY ALWWSPNGKF |
| | | LAYAEFNDTD IPVIAYSYYG DEQYPRTINI |
| | | PYPKAGAKNP VVRIFIIDTT YPAYVGPQEV |
| | | PVPAMIASSD YYFSWLTWVT |
| | | DERVCLQWLK RVQNVSVLSI |
| | | CDFREDWQTW DCPKTQEHIE |
| | | ESRTGWAGGF FVSTPVFSYD AISYYKIFSD |
| | | KDGYKHIHYI KDTVENAIQI TSGKWEAINI |
| | | FRVTQDSLFY SSNEFEEYPG RRNIYRISIG |
| | | SYPPSKKCVT CHLRKERCQY |
| | | YTASFSDYAK YYALVCYGPG |
| | | IPISTLHDGR TDQEIKILEE NKELENALKN |
| | | IQLPKEEIKK LEVDEITLWY KMILPPQFDR |
| | | SKKYPLLIQV YGGPCSQSVR |
| | | SVFAVNWISY LASKEGMVIA |
| | | LVDGRGTAFQ GDKLLYAVYR |
| | | KLGVYEVEDQ ITAVRKFIEM GFIDEKRIAI |
| | | WGWSYGGYVS SLALASGTGL |
| | | FKCGIAVAPV SSWEYYASVY |
| | | TERFMGLPTK DDNLEHYKNS |
| | | TVMARAEYFR NVDYLLIFIGT |
| | | ADDNVHFQNS AQIAKALVNA |
| | | QVDFQAMWYS DQNHGLSGLS |
| | | TNHLYTHMTH FLKQCFSLSD |
| 3 | FAP (212) CDR-H1 | DYNMD |
| 4 | FAP (212) CDR-H2 | DIYPNTGGTIYNQKFKG |
| 5 | FAP (212) CDR-H3 | FRGIHYAMDY |
| 6 | FAP (212) CDR-L1 | RASESVDNYGLSFIN |
| 7 | FAP (212) CDR-L2 | GTSNRGS |
| 8 | FAP (212) CDR-L3 | QQSNEVPYT |
| 9 | FAP (212) VH | EVLLQQSGPELVKPGASVKIACKASGYTLT |
| | | DYNMDWVRQSHGKSLEWIGDIYPNTGGTI |
| | | YNQKFKGKATLTIDKSSSTAYMDLRSLTSE |
| | | DTAVYYCTRFRGIHYAMDYWGQGTSVTV |
| | | SS |
| 10 | FAP (212) VL | DIVLTQSPVSLAVSLGQRATISCRASESVDN |
| | | YGLSFINWFQQKPGQPPKLLIYGTSNRGSG |
| | | VPARFSGSGSGTDFSLNIHPMEEDDTAMYF |
| | | CQQSNEVPYTFGGGTNLEIK |
| 11 | FAP (VH1G3a) CDR-H2 | DIYPNTGGTIYAQKFQG |
| 12 | FAP (VH2G3a) CDR-H2 | DIYPNTGGTIYADSVKG |
| 13 | FAP (VL1G3a) CDR-L1 | RASESVDNYGLSFLA |
| 14 | FAP (VL2G3a) CDR-L1 | RASESIDNYGLSFLN |
| 15 | FAP (VH1G1a) | See Table 10 |
| 16 | FAP (VH1G2a) | See Table 10 |
| 17 | FAP (VH1G3a) | See Table 10 |
| 18 | FAP (VH2G1a) | See Table 10 |
| 19 | FAP (VH2G2a) | See Table 10 |
| 20 | FAP (VH2G3a) | See Table 10 |
| 21 | FAP (VL1G1a) | See Table 10 |
| 22 | FAP (VL1G2a) | See Table 10 |
| 23 | FAP (VL1G3a) | See Table 10 |
| 24 | FAP (VL2G1a) | See Table 10 |
| 25 | FAP (VL2G2a) | See Table 10 |
| 26 | FAP (VL2G3a) | See Table 10 |
| 27 | hu CD40 CDR-H1 | GYYIH |
| 28 | hu CD40 CDR-H2 | RVIPNAGGTSYNQKFKG |
| 29 | hu CD40 CDR-H3 | EGIYW |
| 30 | hu CD40 CDR-L1 | RSSQSLVHSNGNTFLH |
| 31 | hu CD40 CDR-L2 | TVSNRFS |
| 32 | hu CD40 CDR-L3 | SQTTHVPWT |
| 33 | hu CD40 VH | EVQLVESGGGLVQPGGSLRLSCAASGYSFT |
| | | GYYIEWVRQAPGKGLEWVARVIPNAGGTS |
| | | YNQKFKGRFTLSVDNSKNTAYLQMNSLRA |
| | | EDTAVYYCAREGIYWWGQGTLVTVSS |
| 34 | hu CD40 VL | DIQMTQSPSSLSASVGDRVTITCRSSQSLVH |
| | | SNGNTFLHWYQQKPGKAPKLLIYTVSNRFS |
| | | GVPSRFSGSGSGTDFTLTISSLQPEDFATYF |
| | | CSQTTHVPWTFGQGTKVEIK |
| 35 | CD40 (S2C6) VH | EVQLQQSGPD LVKPGASVKI |
| | | SCKASGYSFT GYYIHWVKQS |
| | | HGKSLEWIGR VIPNNGGTSY |
| | | NQKFKGKAIL TVDKSSSTAY |
| | | MELRSLTSED SAVYYCAREG |
| | | IYWWGHGTTL TVSS |
| 36 | CD40 (S2C6) VL | DVVVTQTPLS LPVSLGAQAS ISCRSSQSLV |
| | | HSNGNTFLHW YLQKPGQSPK |
| | | LLIYTVSNRF SGVPDRFSGS GSGTDFTLKI |
| | | SRVEAEDLGV YFCSQTTHVP |
| | | WTFGGGTKLE IQ |
| 37 | VH1a (CD40) | see Table 17 |
| 38 | VH1b (CD40) | see Table 17 |
| 39 | VH1c (CD40) | see Table 17 |
| 40 | VH1d (CD40) | see Table 17 |
| 41 | VL1a (CD40) | see Table 17 |
| 42 | VL1b (CD40) | see Table 17 |
| 43 | VL1c (CD40) | see Table 17 |
| 44 | VL1d (CD40) | see Table 17 |
| 45 | VH2a (CD40) | see Table 18 |
| 46 | VH2b (CD40) | see Table 18 |
| 47 | VH2c (CD40) | see Table 18 |
| 48 | VH2d (CD40) | see Table 18 |
| 49 | VH2ab (CD40) | see Table 18 |
| 50 | VH2ac (CD40) | see Table 18 |
| 51 | VL2a (CD40) | see Table 18 |
| 52 | VL2b (CD40) | see Table 18 |
| 53 | VL2ab (CD40) | see Table 18 |
| 54 | VL2ac (CD40) | see Table 18 |
| 55 | P1AE0400 heavy chain | see Table 20 |
| 56 | P1AE0400 light chain | see Table 20 |
| 57 | P1AE0403 heavy chain | see Table 20 |
| 58 | P1AE0403 light chain | see Table 20 |
| 59 | P1AE0817 heavy chain | see Table 20 |
| 60 | P1AE0817 light chain | see Table 20 |
| 61 | (P1AE1689) light chain | see Table 24 |
| | cross VH-Ckappa | |
| 62 | VL1a (CD40) light chain | see Table 24 |
| | (charged) | |
| 63 | VH1a (CD40) (VHCH1 | see Table 24 |
| | charged) Fc | |
| | knob_PGLALA_(P1AE1689) | |
| | (VL-CH1) | |
| 64 | VH1a (CD40) (VHCH1 | see Table 24 |
| | charged) Fc hole_PGLALA | |
| 65 | (P1AE1689) light chain | see Table 24 |
| | cross VL-CH1 | |
| 66 | VL1a (CD40) light chain | see Table 24 |
| 67 | VH1a (CD40) (VHCH1) Fc | see Table 24 |
| | knob_PGLALA_(P1AE1689) | |
| | (VH-Ckappa) | |
| 68 | VH1a (CD40) (VHCH1) Fc | see Table 24 |
| | hole_PGLALA | |
| 69 | VH1a (CD40) (VHCH1 | see Table 24 |
| | charged_VH1a (CD40) | |
| | (VHCH1 charged)-Fc | |
| | knob_PGLALA_(P1AE1689) | |
| | (VL-CH1) | |
| 70 | VH1a (CD40) (VHCH1 | see Table 24 |
| | charged)_VH1a (CD40) | |
| | (VHCH1 charged)-Fc | |
| | hole_PGLALA | |
| 71 | 4B9 light chain cross | see Table 24 |
| | VL-CH1 | |
| 72 | VH1a (CD40) (VHCH1) Fc | see Table 24 |
| | knob_PGLALA_4B9 (VH- | |
| | Ckappa) | |
| 73 | VH1a (CD40) (VHCH1)_ | see Table 24 |
| | VH1a (CD40) (VHCH1)- | |
| | Fc knob_PGLALA_(4B9) | |
| | (VH-Ckappa) | |
| 74 | VH1a (CD40) | see Table 24 |
| | (VHCH1)_VH1a (CD40) | |
| | (VHCH1)-Fc | |
| | hole_PGLALA | |
| 75 | 28H1 light chain cross | see Table 24 |
| | VH-Ckappa | |
| 76 | VH1a (CD40) (VHCH1 | see Table 24 |
| | charged) Fc | |
| | knob_PGLALA_28H1 | |
| | (VL-CH1) | |
| 77 | VH1a (CD40) (VHCH1 | see Table 24 |
| | charged)_VH1a (CD40) | |
| | (VHCH1 charged) Fc | |
| | knob_PGLALA_28H1 | |
| | (VL-CH1) | |
| 78 | hu FAP ectodomain+poly- | RPSRVHNSEENTMRALTLKDILNGTFSYKT |
| | lys-tag+hs₆-tag | FFPNWISGQEYLHQSADNNIVLYNIETGQS |
| | | YTILSNRTMKSVNASNYGLSPDRQFVYLES |
| | | DYSKLWRYSYTATYYIYDLSNGEFVRGNE |
| | | LPRPIQYLCWSPVGSKLAYVYQNNIYLKQR |
| | | PGDPPFQITFNGRENKIFNGIPDWVYEEEML |
| | | ATKYALWWSPNGKFLAYAEFNDTDIPVIA |
| | | YSYYGDEQYPRTINIPYPKAGAKNPVVRIFI |
| | | IDTTYPAYVGPQEVPVPAMIASSDYYFSWL |
| | | TWVTDERVCLQWLKRVQNVSVLSICDFRE |
| | | DWQTWDCPKTQEHIEESRTGWAGGFFVST |
| | | PVFSYDAISYYKIFSDKDGYKHIHYIKDTVE |
| | | NAIQITSGKWEAINIFRVTQDSLFYSSNEFEE |
| | | YPGRRNIYRISIGSYPPSKKCVTCHLRKERC |
| | | QYYTASFSDYAKYYALVCYGPGIPISTLHD |
| | | GRTDQEIKILEENKELENALKNIQLPKEEIK |
| | | KLEVDEITLWYKMILPPQFDRSKKYPLLIQ |
| | | VYGGPCSQSVRSVFAVNWISYLASKEGMV |
| | | IALVDGRGTAFQGDKLLYAVYRKLGVYEV |
| | | EDQITAVRKFIEMGFIDEKRIAIWGWSYGG |
| | | YVSSLALASGTGLFKCGIAVAPVSSWEYYA |
| | | SVYTERFMGLPTKDDNLEHYKNSTVMARA |
| | | EYFRNVDYLLIHGTADDNVHFQNSAQIAK |
| | | ALVNAQVDFQAMWYSDQNHGLSGLSTNH |
| | | LYTHMTHFLKQCFSLSDGKKKKKKGHHH |
| | | HHH |
| 79 | mouse FAP | UniProt no. P97321 |
| 80 | Murine FAP | RPSRVYKPEGNTKRALTLKDILNGTFSYKT |
| | ectodomain+poly-lys- | YFPNWISEQEYLHQSEDDNIVFYNIETRESY |
| | tag+his₆-tag | IILSNSTMKSVNATDYGLSPDRQFVYLESD |
| | | YSKLWRYSYTATYYIYDLQNGEFVRGYEL |
| | | PRPIQYLCWSPVGSKLAYVYQNNIYLKQRP |
| | | GDPPFQITYTGRENRIFNGIPDWVYEEEML |
| | | ATKYALWWSPDGKFLAYVEFNDSDIPIIAY |
| | | SYYGDGQYPRTINIPYPKAGAKNPVVRVFI |
| | | VDTTYPHHVGPMEVPVPEMIASSDYYFSW |
| | | LTWVSSERVCLQWLKRVQNVSVLSICDFR |
| | | EDWHAWECPKNQEHVEESRTGWAGGFFV |
| | | STPAFSQDATSYYKIFSDKDGYKHIHYIKDT |
| | | VENAIQITSGKWEAIYIFRVTQDSLFYSSNE |
| | | FEGYPGRRNIYRISIGNSPPSKKCVTCHLRK |
| | | ERCQYYTASFSYKAKYYALVCYGPGLPIST |
| | | LHDGRTDQEIQVLEENKELENSLRNIQLPK |
| | | VEIKKLKDGGLTFWYKMILPPQFDRSKKYP |
| | | LLIQVYGGPCSQSVKSVFAVNWITYLASKE |
| | | GIVIALVDGRGTAFQGDKFLHAVYRKLGV |
| | | YEVEDQLTAVRKFIEMGFIDEERIAIWGWS |
| | | YGGYVSSLALASGTGLFKCGIAVAPVSSWE |
| | | YYASIYSERFMGLPTKDDNLEHYKNSTVM |
| | | ARAEYFRNVDYLLIHGTADDNVHFQNSAQ |
| | | IAKALVNAQVDFQAMWYSDQNHGILSGRS |
| | | QNHLYTHMTHFLKQCFSLSDGKKKKKKG |
| | | HHHHHH |
| 81 | Cynomolgus FAP | RPPRVHNSEENTMRALTLKDILNGTFSYKT |
| | ectodomain+poly-lys- | FFPNWISGQEYLHQSADNNIVLYNIETGQS |
| | tag+his₆-tag | YTILSNRTMKSVNASNYGLSPDRQFVYLES |
| | | DYSKLWRYSYTATYYIYDLSNGEFVRGNE |
| | | LPRPIQYLCWSPVGSKLAYVYQNNIYLKQR |
| | | PGDPPFQITFNGRENKIFNGIPDWVYEEEML |
| | | ATKYALWWSPNGKFLAYAEFNDTDIPVIA |
| | | YSYYGDEQYPRTINIPYPKAGAKNPFVRIFII |
| | | DTTYPAYVGPQEVPVPAMIASSDYYFSWL |
| | | TWVTDERVCLQWLKRVQNVSVLSICDFRE |
| | | DWQTWDCPKTQEHIEESRTGWAGGFFVST |
| | | PVFSYDAISYYKIFSDKDGYKHIHYIKDTVE |
| | | NAIQITSGKWEAINIFRVTQDSLFYSSNEFE |
| | | DYPGRRNIYRISIGSYPPSKKCVTCHLRKER |
| | | CQYYTASFSDYAKYYALVCYGPGIPISTLH |
| | | DGRTDQEIKILEENKELENALKNIQLPKEEI |
| | | KKLEVDEITLWYKMILPPQFDRSKKYPLLI |
| | | QVYGGPCSQSVRSVFAVNWISYLASKEGM |
| | | VIALVDGRGTAFQGDKLLYAVYRKLGVYE |
| | | VEDQITAVRKFIEMGFIDEKRIAIWGWSYG |
| | | GYVSSLALASGTGLFKCGIAVAPVSSWEYY |
| | | ASVYTERFMGLPTKDDNLEHYKNSTVMAR |
| | | AEYFRNVDYLLIHGTADDNVHFQNSAQIA |
| | | KALVNAQVDFQAMWYSDQNHGLSGLSTN |
| | | HLYTHMTHFLKQCFSLSDGKKKKKKGHH |
| | | HHHH |
| 82 | Peptide linker (G4S) | GGGGS |
| 83 | Peptide linker (G4S)2 | GGGGSGGGGS |
| 84 | Peptide linker (SG4)2 | SGGGGSGGGG |
| 85 | Peptide linker G4(SG4)2 | GGGGSGGGGSGGGG |
| 86 | peptide linker | GSPGSSSSGS |
| 87 | (G4S)₃ peptide linker | GGGGSGGGGSGGGGS₃ |
| 88 | (G4S)₄ peptide linker | GGGGSGGGGSGGGGSGGGGS |
| 89 | peptide linker | GSGSGSGS |
| 90 | peptide linker | GSGSGNGS |
| 91 | peptide linker | GGSGSGSG |
| 92 | peptide linker | GGSGSG |
| 93 | peptide linker | GGSG |
| 94 | peptide linker | GGSGNGSG |
| 95 | peptide linker | GGNGSGSG |
| 96 | peptide linker | GGNGSG |
| 97 | Fc knob chain | DKTHTCPPCPAPELLGGPSVFLFPPKPKDTL |
| | | MISRTPEVTCVVVDVSHEDPEVKFNWYVD |
| | | GVEVHNAKTKPREEQYNSTYRVVSVLTVL |
| | | HQDWLNGKEYKCKVSNKALPAPIEKTISK |
| | | AKGQPREPQVYTLPPCRDELTKNQVSLWC |
| | | LVKGFYPSDIAVEWESNGQPENNYKTTPPV |
| | | LDSDGSFFLYSKLTVDKSRWQQGNVFSCS |
| | | VMHEALHNHYTQKSLSLSPGK |
| 98 | Fc hole chain | DKTHTCPPCPAPELLGGPSVFLFPPKPKDTL |
| | | MISRTPEVTCVVVDVSHEDPEVKFNWYVD |
| | | GVEVHNAKTKPREEQYNSTYRVVSVLTVL |
| | | HQDWLNGKEYKCKVSNKALPAPIEKTISK |
| | | AKGQPREPQVCTLPPSRDELTKNQVSLSCA |
| | | VKGFYPSDIAVEWESNGQPENNYKTTPPVL |
| | | DSDGSFFLVSKLTVDKSRWQQGNVFSCSV |
| | | MHEALHNHYTQKSLSLSPGK |
| 99 | Acceptor framework | YYYYYGMDVWGQGTTVTVSS |
| | IGHJ6*01/02 (page 106) | |
| 100 | Acceptor framework | LTFGGGTKVEIK |
| | IGKJ4*01/02 (page 106) | |
| 101 | Acceptor framework 1 | YYYYYGMDVWGQGTTVTVSS |
| | IGHJ6*01/02 (page 110) | |
| 102 | Acceptor framework 1 | LTFGGGTKVEIK |
| | IGKJ4*01/02 (page 110) | |
| 103 | Acceptor framework 2 | YYYYYGMDVWGQGTTVTVSS |
| | IGHJ6*01/02 (page 111) | |
| 104 | Acceptor framework 2 | LTFGGGTKVEIK |
| | IGKJ4*01/02 (page 111) | |
| 105 | huCD40 light chain | See Table 24 |
| | (charged) | |
| 106 | huCD40 (VHCH1 charged) | See Table 24 |
| | Fc PGLALA FAP | |
| | (VL-CH1) | |
| 107 | VH (PD-L1) | EVQLVESGGGLVQPGGSLRLSCAASGFTFS |
| | | DSWIHWVRQAPGKGLEWVAWISPYGGST |
| | | YYADSVKGRFTISADTSKNTAYLQMNSLR |
| | | AEDTAVYYCARRHWPGGFDYWGQGTLVT |
| | | VSS |
| 108 | VL (PD-L1) | DIQMTQSPSSLSASVGDRVTITCRASQDVST |
| | | AVAWYQQKPGKAPKLLIYSASFLYSGVPS |
| | | RFSGSGSGTDFTLTISSLQPEDFATYYCQQY |
| | | LYHPATFGQGTKVEIK |
| 109 | VH (PD-L1) | EVQLVESGGGLVQPGGSLRLSCAASGFTFS |
| | | RYWMSWVRQAPGKGLEWVANIKQDGSEK |
| | | YYVDSVKGRFTISRDNAKNSLYLQMNSLR |
| | | AEDTAVYYCAREGGWFGELAFDYWGQGT |
| | | LVTVSS |
| 110 | VL (PD-L1) | EIVLTQSPGTLSLSPGERATLSCRASQRVSS |
| | | SYLAWYQQKPGQAPRLLIYDASSRATGIPD |
| | | RFSGSGSGTDFTLTISRLEPEDFAVYYCQQY |
| | | GSLPWTFGQGTKVEIK |

The following numbered paragraphs (paras) describe aspects of the present invention:
1. A bispecific antigen binding molecule, comprising
   (a) at least one antigen binding domain capable of specific binding to CD40, and
   (b) at least one antigen binding domain capable of specific binding to Fibroblast Activation Protein (FAP) comprising a heavy chain variable region (V_{H}FAP) comprising
   (i) CDR-H1 comprising the amino acid sequence of SEQ ID NO:3, (ii) CDR-H2 comprising the amino acid sequence selected from the group consisting of SEQ ID NO:4, SEQ ID NO: 11 and SEQ ID NO: 12, and (iii) CDR-H3 comprising the amino acid sequence of SEQ ID NO:5, and a light chain variable region (V_{L}FAP) comprising (iv) CDR-L1 comprising the amino acid sequence selected from the group consisting of SEQ ID NO: 6, SEQ ID NO:13 and SEQ ID NO:14, (v) CDR-L2 comprising the amino acid sequence of SEQ ID NO:7, and (vi) CDR-L3 comprising the amino acid sequence of SEQ ID NO: 8.
2. The bispecific antigen binding molecule of para 1, additionally comprising
   (c) a Fc region composed of a first and a second subunit capable of stable association.
3. The bispecific antigen binding molecule of paras 1 or 2, wherein the antigen binding domain capable of specific binding to FAP comprises a heavy chain variable region (V_{H}FAP) comprising an amino acid sequence that is at least about 90% identical to the amino acid sequence of SEQ ID NO:9, and a light chain variable region (V_{L}FAP) comprising an amino acid sequence that is at least about 90% identical to the amino acid sequence of SEQ ID NO:10.
4. The bispecific antigen binding molecule of any one of paras 1 to 3, wherein the antigen binding domain capable of specific binding to FAP comprises
a heavy chain variable region (V_{H}FAP) comprising an amino acid sequence selected from the group consisting of SEQ ID NO:15, SEQ ID NO:16, SEQ ID NO:17, SEQ ID NO: 18. SEQ ID NO:19 and SEQ ID NO:20, and
a light chain variable region (V_{L}FAP) comprising an amino acid sequence selected from the group consisting of SEQ ID NO:21, SEQ ID NO:22, SEQ ID NO:23, SEQ ID NO: 24, SEQ ID NO:25 and SEQ ID NO:26.
5. The bispecific antigen binding molecule of any one of paras 1 to 4, wherein the antigen binding domain capable of specific binding to FAP comprises
(a) a heavy chain variable region (V_{H}FAP) comprising the amino acid sequence of SEQ ID NO:15 and a light chain variable region (V_{L}FAP) comprising the amino acid sequence of SEQ ID NO:21,
(b) a heavy chain variable region (V_{H}FAP) comprising the amino acid sequence of SEQ ID NO:16 and a light chain variable region (V_{L}FAP) comprising the amino acid sequence of SEQ ID NO:21,
(c) a heavy chain variable region (V_{H}FAP) comprising the amino acid sequence of SEQ ID NO:16 and a light chain variable region (V_{L}FAP) comprising the amino acid sequence of SEQ ID NO:22, or
(d) a heavy chain variable region (V_{H}FAP) comprising the amino acid sequence of SEQ ID NO:19 and a light chain variable region (V_{L}FAP) comprising the amino acid sequence of SEQ ID NO:25.
6. The bispecific antigen binding molecule of any one of paras 1 to 5, wherein the antigen binding domain capable of specific binding to CD40 comprises a heavy chain variable region (V_{H}CD40) comprising (i) CDR-H1 comprising the amino acid sequence of SEQ ID NO:27, (ii) CDR-H2 comprising the amino acid sequence of SEQ ID NO:28, and (iii) CDR-H3 comprising the amino acid sequence of SEQ ID NO:29, and a light chain variable region (V_{L}CD40) comprising (iv) CDR-L1 comprising the amino acid sequence of SEQ ID NO:30, (v) CDR-L2 comprising the amino acid sequence of SEQ ID NO:31, and (vi) CDR-L3 comprising the amino acid sequence of SEQ ID NO:32.
7. The bispecific antigen binding molecule of any one of paras 1 to 6, wherein the antigen binding domain capable of specific binding to CD40 comprises
(i) a heavy chain variable region (V_{H}CD40) comprising an amino acid sequence selected from the group consisting of SEQ ID NO:37, SEQ ID NO:38, SEQ ID NO:39 and SEQ ID NO:40, and
(ii) a light chain variable region (V_{L}CD40) comprising the amino acid sequence selected from the group consisting of SEQ ID NO:41, SEQ ID NO:42, SEQ ID NO:43, and SEQ ID NO:44.
8. The bispecific antigen binding molecule of any one of paras 1 to 6, wherein the antigen binding domain capable of specific binding to CD40 comprises
(i) a heavy chain variable region (V_{H}CD40) comprising an amino acid sequence selected from the group consisting of SEQ ID NO:45, SEQ ID NO:46, SEQ ID NO:47, SEQ ID NO:48, SEQ ID NO:49 and SEQ ID NO:50, and
(ii) a light chain variable region (V_{L}CD40) comprising the amino acid sequence selected from the group consisting of SEQ ID NO:51, SEQ ID NO:52, SEQ ID NO:53, and SEQ ID NO:54.
9. The bispecific antigen binding molecule of any one of paras 1 to 5 or 7, wherein the antigen binding domain capable of specific binding to CD40 comprises
(a) a VH comprising the amino acid sequence of SEQ ID NO:37 and a VL comprising the amino acid sequence of SEQ ID NO:41, or
(b) a VH comprising the amino acid sequence of SEQ ID NO:37 and a VL comprising the amino acid sequence of SEQ ID NO:42, or
(c) a VH comprising the amino acid sequence of SEQ ID NO:37 and a VL comprising the amino acid sequence of SEQ ID NO:43, or
(d) a VH comprising the amino acid sequence of SEQ ID NO:37 and a VL comprising the amino acid sequence of SEQ ID NO:44, or
(e) a VH comprising the amino acid sequence of SEQ ID NO:38 and a VL comprising the amino acid sequence of SEQ ID NO:41, or
(f) a VH comprising the amino acid sequence of SEQ ID NO:38 and a VL comprising the amino acid sequence of SEQ ID NO:42, or
(g) a VH comprising the amino acid sequence of SEQ ID NO:38 and a VL comprising the amino acid sequence of SEQ ID NO:43, or
(h) a VH comprising the amino acid sequence of SEQ ID NO:38 and a VL comprising the amino acid sequence of SEQ ID NO:44, or
(i) a VH comprising the amino acid sequence of SEQ ID NO:39 and a VL comprising the amino acid sequence of SEQ ID NO:41, or
(j) a VH comprising the amino acid sequence of SEQ ID NO:39 and a VL comprising the amino acid sequence of SEQ ID NO:42, or
(k) a VH comprising the amino acid sequence of SEQ ID NO:39 and a VL comprising the amino acid sequence of SEQ ID NO:43, or
(l) a VH comprising the amino acid sequence of SEQ ID NO:39 and a VL comprising the amino acid sequence of SEQ ID NO:44, or
(m) a VH comprising the amino acid sequence of SEQ ID NO:40 and a VL comprising the amino acid sequence of SEQ ID NO:41, or
(n) a VH comprising the amino acid sequence of SEQ ID NO:40 and a VL comprising the amino acid sequence of SEQ ID NO:42, or
(o) a VH comprising the amino acid sequence of SEQ ID NO:40 and a VL comprising the amino acid sequence of SEQ ID NO:43, or
(p) a VH comprising the amino acid sequence of SEQ ID NO:40 and a VL comprising the amino acid sequence of SEQ ID NO:44.
10. The bispecific antigen binding molecule of any one of paras 1 to 5 or 7 or 9, wherein the antigen binding domain capable of specific binding to CD40 comprises a VH comprising the amino acid sequence of SEQ ID NO:37 and a VL comprising the amino acid sequence of SEQ ID NO:41.
11. The bispecific antigen binding molecule of any one of paras 1 to 5 or 8, wherein the antigen binding domain capable of specific binding to CD40 comprises
(a) a VH comprising the amino acid sequence of SEQ ID NO:45 and a VL comprising the amino acid sequence of SEQ ID NO:51, or
(b) a VH comprising the amino acid sequence of SEQ ID NO:46 and a VL comprising the amino acid sequence of SEQ ID NO:51, or
(c) a VH comprising the amino acid sequence of SEQ ID NO:47 and a VL comprising the amino acid sequence of SEQ ID NO:51, or
(d) a VH comprising the amino acid sequence of SEQ ID NO:48 and a VL comprising the amino acid sequence of SEQ ID NO:51, or
(e) a VH comprising the amino acid sequence of SEQ ID NO:45 and a VL comprising the amino acid sequence of SEQ ID NO:52, or
(f) a VH comprising the amino acid sequence of SEQ ID NO:46 and a VL comprising the amino acid sequence of SEQ ID NO:52, or
(g) a VH comprising the amino acid sequence of SEQ ID NO:47 and a VL comprising the amino acid sequence of SEQ ID NO:52, or
(h) a VH comprising the amino acid sequence of SEQ ID NO:48 and a VL comprising the amino acid sequence of SEQ ID NO:52, or
(i) a VH comprising the amino acid sequence of SEQ ID NO:49 and a VL comprising the amino acid sequence of SEQ ID NO:53, or
(j) a VH comprising the amino acid sequence of SEQ ID NO:50 and a VL comprising the amino acid sequence of SEQ ID NO:53, or
(k) a VH comprising the amino acid sequence of SEQ ID NO:49 and a VL comprising the amino acid sequence of SEQ ID NO:54, or
(l) a VH comprising the amino acid sequence of SEQ ID NO:50 and a VL comprising the amino acid sequence of SEQ ID NO:54.
12. The bispecific antigen binding molecule of any one of paras 1 to 5 or 8 or 11, wherein the antigen binding domain capable of specific binding to CD40 comprises a VH comprising the amino acid sequence of SEQ ID NO:45 and a VL comprising the amino acid sequence of SEQ ID NO:51 or wherein the antigen binding domain capable of specific binding to CD40 comprises a VH comprising the amino acid sequence of SEQ ID NO:48 and a VL comprising the amino acid sequence of SEQ ID NO:51.
13. The bispecific antigen binding molecule of any one of paras 1 to 7, comprising
(i) at least one antigen binding domain capable of specific binding to CD40, comprising a heavy chain variable region (V_{H}CD40) comprising the amino acid sequence of SEQ ID NO:37 and a light chain variable region (V_{L}CD40) comprising the amino acid sequence of SEQ ID NO:41, and
(ii) at least one antigen binding domain capable of specific binding to FAP, comprising a heavy chain variable region (V_{H}FAP) comprising an amino acid sequence of SEQ ID NO:15 and a light chain variable region (V_{L}FAP) comprising an amino acid sequence of SEQ ID NO:21.
14. The bispecific antigen binding molecule of any one of paras 2 to 13, wherein the Fc region is an IgG, particularly an IgG1 Fc region or an IgG4 Fc region and wherein the Fc region comprises one or more amino acid substitution that reduces the binding affinity of the antibody to an Fc receptor and/or effector function.
15. The bispecific antigen binding molecule of any one of paras 2 to 14, wherein the Fc region is of human IgG1 subclass with the amino acid mutations L234A, L235A and P329G (numbering according to Kabat EU index).
16. The bispecific antigen binding molecule of any one of paras 1 to 15, wherein the bispecific antigen binding molecule comprises
(a) at least two Fab fragments capable of specific binding to CD40 connected to a Fc region, and
(b) one antigen binding domain capable of specific binding to FAP connected to the C-terminus of the Fc region.
17. The bispecific antigen binding molecule of any one of paras 1 to 16, wherein the bispecific antigen binding molecule comprises
(a) at least two Fab fragments capable of specific binding to CD40 fused to a Fc region, and
(b) a cross-fab fragment capable of specific binding to FAP fused to the C-terminus of the Fc region.
18. The bispecific antigen binding molecule of para 17, wherein the VH-Ckappa chain of the cross-fab fragment capable of specific binding to FAP is fused to the C-terminus of the Fc region.
19. The bispecific antigen binding molecule of any one of paras 1 to 18, wherein the bispecific antigen binding molecule comprises four Fab fragments capable of specific binding to CD40.
20. An antibody that specifically binds to FAP, wherein said antibody comprises a heavy chain variable region (V_{H}FAP) comprising (i) CDR-H1 comprising the amino acid sequence of SEQ ID NO:3, (ii) CDR-H2 comprising the amino acid sequence selected from the group consisting of SEQ ID NO:4, SEQ ID NO:11 and SEQ ID NO:12, and (iii) CDR-H3 comprising the amino acid sequence of SEQ ID NO:5, and a light chain variable region (V_{L}FAP) comprising (iv) CDR-L1 comprising the amino acid sequence selected from the group consisting of SEQ ID NO:6, SEQ ID NO: 13 and SEQ ID NO:14, (v) CDR-L2 comprising the amino acid sequence of SEQ ID NO:7, and (vi) CDR-L3 comprising the amino acid sequence of SEQ ID NO:8.
21. The antibody of para 20, wherein said antibody comprises
(a) a heavy chain variable region (V_{H}FAP) comprising the amino acid sequence of SEQ ID NO:15 and a light chain variable region (V_{L}FAP) comprising the amino acid sequence of SEQ ID NO:21,
(b) a heavy chain variable region (V_{H}FAP) comprising the amino acid sequence of SEQ ID NO:16 and a light chain variable region (V_{L}FAP) comprising the amino acid sequence of SEQ ID NO:21,
(c) a heavy chain variable region (V_{H}FAP) comprising the amino acid sequence of SEQ ID NO:16 and a light chain variable region (V_{L}FAP) comprising the amino acid sequence of SEQ ID NO:22, or
(d) a heavy chain variable region (V_{H}FAP) comprising the amino acid sequence of SEQ ID NO:19 and a light chain variable region (V_{L}FAP) comprising the amino acid sequence of SEQ ID NO:25.
22. Isolated nucleic acid encoding the bispecific antigen binding molecule of any one of paras 1 to 19 or the antibody of paras 20 or 21.
23. An expression vector comprising the isolated nucleic acid of para 22.
24. A host cell comprising isolated nucleic acid of para 22 or the expression vector of para 23.
25. A method of producing a bispecific antigen binding molecule, comprising culturing the host cell of para 24 under conditions suitable for the expression of the bispecific antigen binding molecule, and isolating the bispecific antigen binding molecule.
26. A pharmaceutical composition comprising the bispecific antigen binding molecule of any one of paras 1 to 19 or the antibody of paras 20 or 21 and a pharmaceutically acceptable carrier.
25. The pharmaceutical composition of para 24, further comprising an additional therapeutic agent.
26. The bispecific antigen binding molecule of any one of paras 1 to 19, or the pharmaceutical composition of para 24, for use as a medicament.
27. The bispecific antigen binding molecule of any one of paras 1 to 19, or the pharmaceutical composition of para 24, for use
(i) in inducing immune stimulation by CD40 expressing antigen-presenting cells (APCs),
(ii) in stimulating tumor-specific T cell response,
(iii) in causing apoptosis of tumor cells,
(iv) in the treatment of cancer,
(v) in delaying progression of cancer,
(vi) in prolonging the survival of a patient suffering from cancer,
(vii) in the treatment of infections.
28. The bispecific antigen binding molecule of any one of paras 1 to 19, or the pharmaceutical composition of para 24, for use in the treatment of cancer.
29. Use of the bispecific antigen binding molecule of any one of paras 1 to 19, or the pharmaceutical composition of para 24, in the manufacture of a medicament for the treatment of cancer.
30. A method of treating an individual having cancer comprising administering to the individual an effective amount of the bispecific antigen binding molecule of any one of paras 1 to 19, or the pharmaceutical composition of para 24.
31. The bispecific antigen binding molecule according to any one of paras 1 to 19 or the pharmaceutical composition according to para 24 for use in the treatment of cancer, wherein the bispecific antigen binding molecule is administered in combination with a chemotherapeutic agent, radiation and/or other agents for use in cancer immunotherapy.

### EXAMPLES

The following are examples of methods and compositions of the invention. It is understood that various other embodiments may be practiced, given the general description provided above.

### Recombinant DNA Techniques

Standard methods were used to manipulate DNA as described in Sambrook et al., Molecular cloning: A laboratory manual; Cold Spring Harbor Laboratory Press, Cold Spring Harbor, New York, 1989. The molecular biological reagents were used according to the manufacturer's instructions. General information regarding the nucleotide sequences of human immunoglobulin light and heavy chains is given in: Kabat, E. A. et al., (1991) Sequences of Proteins of Immunological Interest, Fifth Ed., NIH Publication No 91-3242.

### DNA Sequencing

DNA sequences were determined by double strand sequencing.

### Gene Synthesis

Desired gene segments were either generated by PCR using appropriate templates or were synthesized by Geneart AG (Regensburg, Germany) from synthetic oligonucleotides and PCR products by automated gene synthesis. In cases where no exact gene sequence was available, oligonucleotide primers were designed based on sequences from closest homologues and the genes were isolated by RT-PCR from RNA originating from the appropriate tissue. The gene segments flanked by singular restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

### Protein Purification

Proteins were purified from filtered cell culture supernatants referring to standard protocols. In brief, antibodies were applied to a Protein A Sepharose column (GE healthcare) and washed with PBS. Elution of antibodies was achieved at pH 2.8 followed by immediate neutralization of the sample. Aggregated protein was separated from monomeric antibodies by size exclusion chromatography (Superdex 200, GE Healthcare) in PBS or in 20 mM Histidine, 150 mM NaCl pH 6.0. Monomeric antibody fractions were pooled, concentrated (if required) using e.g., a MILLIPORE Amicon Ultra (30 MWCO) centrifugal concentrator, frozen and stored at −20° C. or −80° C. Part of the samples were provided for subsequent protein analytics and analytical characterization e.g. by SDS-PAGE, size exclusion chromatography (SEC) or mass spectrometry.

### SDS-PAGE

The NuPAGE® Pre-Cast gel system (Invitrogen) was used according to the manufacturer's instruction. In particular, 10% or 4-12% NuPAGE® Novex® Bis-TRIS Pre-Cast gels (pH 6.4) and a NuPAGE® MES (reduced gels, with NuPAGE® Antioxidant running buffer additive) or MOPS (non-reduced gels) running buffer was used.

### CE-SDS

Purity, antibody integrity and molecular weight of bispecific and control antibodies were analyzed by CE-SDS using microfluidic Labchip technology (Caliper Life Science, USA). 5 μl of protein solution was prepared for CE-SDS analysis using the HT Protein Express Reagent Kit according manufacturer's instructions and analysed on LabChip GXII system using a HT Protein Express Chip. Data were analyzed using LabChip GX Software version 3.0.618.0.

### Analytical Size Exclusion Chromatography

Size exclusion chromatography (SEC) for the determination of the aggregation and oligomeric state of antibodies was performed by HPLC chromatography. Briefly, Protein A purified antibodies were applied to a Tosoh TSKgel G3000SW column in 300 mM NaCl, 50 mM KH₂PO₄/K₂HPO₄, pH 7.5 on an Agilent HPLC 1100 system or to a Superdex 200 column (GE Healthcare) in 2×PBS on a Dionex HPLC-System. The eluted protein was quantified by UV absorbance and integration of peak areas. BioRad Gel Filtration Standard 151-1901 served as a standard.

### Mass Spectrometry

This section describes the characterization of the multispecific antibodies with VH/VL or CH/CL exchange (CrossMabs) with emphasis on their correct assembly. The expected primary structures were analyzed by electrospray ionization mass spectrometry (ESI-MS) of the deglycosylated intact CrossMabs and deglycosylated/FabALACTICA or alternatively deglycosylated/GingisKHAN digested CrossMabs.

The CrossMabs were deglycosylated with N-Glycosidase F in a phosphate or Tris buffer at 37° C. for up to 17 h at a protein concentration of 1 mg/ml. The FabALACTICA or GingisKHAN (Genovis AB; Sweden) digestions were performed in the buffers supplied by the vendor with 100 μg deglycosylated CrossMabs. Prior to mass spectrometry the samples were desalted via HPLC on a Sephadex G25 column (GE Healthcare). The total mass was determined via ESI-MS on a maXis 4G UHR-QTOF MS system (Bruker Daltonik) equipped with a TriVersa NanoMate source (Advion).

### Example 1

### Generation of New Antibodies Against Fibroblast Activation Protein (FAP)

### 1.1 Immunization of Mice

Balb/c and NMRI mice were used for immunization. The animals were housed according to the Appendix A “Guidelines for accommodation and care of animals” in an AAALACi accredited animal facility. All animal immunization protocols and experiments were approved by the Government of Upper Bavaria (permit number 55.2-1-54-2531-19-10) and performed according to the German Animal Welfare Act and the Directive 2010/63 of the European Parliament and Council. Balb/c and NMRI mice (n=5), 6-8 week old, received four rounds of immunization with recombinant produced extracellular domain of human fibroblast activation protein alpha (amino acid 27-759; accession number NP_004451) covalently attached to a His tag (SEQ ID NO:78). Before each immunization, mice were anesthetized with a gas mixture of oxygen and isoflurane. For the first immunization, 30 μg protein dissolved in PBS, pH 7.4, were mixed with an equal volume of CFA (BD Difco, #263810) and administered intraperitoneal (i.p.) Another 10 μg of protein emulsified in Abisco adjuvant was administered subcutaneously (s.c.) at week 6. A third dose of 5 μg protein without adjuvant was administered i.p. at week 10. Finally, three days prior to the preparation of splenocytes for antibody development using hybridoma technology, the mice were subjected to intravenous (i.v.) booster immunizations with 50 μg of protein. Serum was tested for antigen-specific total IgG antibody production by ELISA. Three days after the final immunization, mice were euthanized and the spleen was isolated aseptically and prepared for hybridoma generation. The mouse lymphocytes were isolated and fused with a mouse myeloma cell line using PEG based standard protocols to generate hybridomas. The resulting hybridoma cells were plated at approximately 10⁴ in flat bottom 96 well micro titer plate, followed by about two weeks of incubation in selective medium and then screened for the production of antigen-specific antibodies. Once extensive hybridoma growth occurs, the antibody secreting hybridomas are replated. Hybridoma supernatants were screened for specific binding to recombinant human fibroblast activation protein alpha (huFAP) by ELISA, followed by evaluation of kinetic binding parameters to recombinant huFAP using Biacore measurement.

Culture of hybridomas: Generated muMAb hybridomas were cultured in RPMI 1640 (PAN—Catalogue No. (Cat. No.) PO4-17500) supplemented with 2 mM L-glutamine (GIBCO—Cat. No. 35050-038), 1 mM Na-Pyruvat (GIBCO—Cat. No. 11360-039), 1×NEAA (GIBCO—Cat. No. 11140-035), 10% FCS (PAA—Cat. No. A15-649), 1× Pen Strep (Roche—Cat. No. 1074440), 1× Nutridoma CS (Roche—Cat. No. 1363743), 50 μM Mercaptoethanol (GIBCO—Cat. No. 31350-010) and 50 U/ml IL 6 mouse (Roche—Cat. No. 1 444 581) at 37° C. and 5% CO₂.

### 1.2 Competitive Cellular Binding of Anti-huFAP Antibodies to FAP Clone 4B9 and 28H1

The resulting clones were tested for their binding behavior in comparison to FAP clone 4B9. The generation and preparation of FAP clones 4B9 and 28H1 is described in WO 2012/020006 A2, which is incorporated herein by reference. To determine whether the murine FAP clones recognize different epitopes as clones 4B9 and 28H1 a competition binding to human FAP expressed on transfected HEK cells was performed.

Briefly, the target cells were harvested with Cell Dissociation buffer, washed with FACS Buffer (PBS+2% FCS+5 mM EDTA+0.25% sodium acide) and seeded into 96-U bottom plates (1×105 cells/well). Unlabeled primary anti-human FAP antibodies (mu IgG1) were added to the cells (final concentrations 60 μg/ml to 0.2 μg/ml; 1:3 dilutions) and incubated for 20 min at 4° C. before addition of AlexaFluor647-labeled anti FAP antibody 4B9 or 28H1 (end concentration 20 μg/ml). After 30 min incubation at 4° C., cells were washed, fixed and the fluorescent signal intensities of the AF647-labeled clones 4B9 and 28H1 were measured using a Miltenyi MACSQuant.

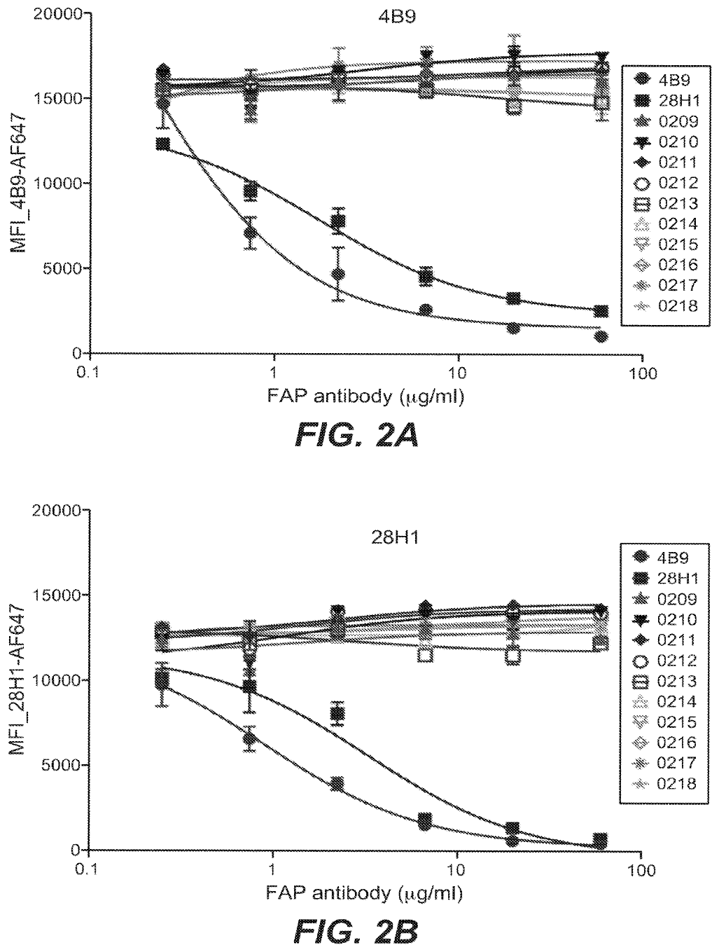

As can be seen in FIG. 2A and FIG. 2B, 10 hybridoma-derived murine antibodies were identified (named clones 209, 210, 211, 212, 213, 214, 215, 216, 217 and 218) that did not compete for binding with anti FAP antibodies 4B9 or 28H1.

### 1.3 Target Binding Specificity of Anti-huFAP Murine Antibodies

Fibroblast activation protein (FAP, FAP-α, seprase) is a type II transmembrane serine protease, belonging to the prolyl oligopeptidase family. This family comprises serine proteases that cleave peptides preferentially after proline residues. Other important members of this family that are expressed in the human proteome are prolyl oligopeptidase (PREP) and the dipeptidyl peptidases (DPPs). DPP-IV is the closest homolog of FAP. In contrast to FAP, DPP-IV is ubiquitously expressed and plays a role in various biological processes such as T cell co-stimulation, chemokine biology, glucose metabolism, and tumorigenesis and therefore the desired anti-human FAP antibodies should not bind to human DPP-IV.

Binding to human FAP and human DPP-IV was determined by flow cytometry using human FAP or human DPPIV-transfected HEK cells. Briefly, the target cells were harvested with Cell Dissociation buffer, washed with FACS Buffer (PBS+2% FCS+5 mM EDTA+0.25% sodium acide) and seeded into 96-U bottom plates (1×10⁵ cells/well). Unlabeled primary antibodies were added to the cells (final concentration 10 μg/ml) and incubated for 30 min at 4° C. After washing, cells were incubated with a goat anti-mouse IgG-PE F(ab′)₂ (Serotec) for 30 min at 4° C. in the dark. Afterwards, cells were washed, fixed and measured using a BD FACS Canto™ II. No unspecific binding to human DPP-IV was detected for any of the 10 hybridoma derived anti-human FAP antibodies.

1.4 Generation of Anti-huFAP Antibodies in huIgG1_LALA_PG Format

The DNA sequences of the new anti-huFAP antibodies were determined with standard sequencing methods. Based on the VH and VL domains new anti-FAP antibodies were expressed as huIgG1 antibodies with an effector silent Fc (P329G; L234, L235A) to receptors according to the method described in WO 2012/130831 abrogate binding to Fc A1. In detail, antibodies were expressed by transient transfection of HEK293-F cells grown in suspension with expression vectors encoding the different peptide chains. Transfection into HEK293-F cells (Invitrogen, USA) was performed according to the cell supplier's instructions using Maxiprep (Qiagen, Germany) preparations of the antibody vectors, F17 based medium (Invitrogen, USA), PEIpro (Polyscience Europe GmbH) and an initial cell density of 1-2 million viable cells/ml in serum free FreeStyle 293 expression medium (Invitrogen). Cell culture supernatants were harvested after 7 days of cultivation in shake flasks or stirred fermenters by centrifugation at 14000 g for 30 minutes and filtered through a 0.22 μm filter.

The antibodies were purified from cell culture supernatants by affinity chromatography using MabSelectSure-Sepharose™ (GE Healthcare, Sweden) chromatography. Briefly, sterile filtered cell culture supernatants were captured on a MabSelect SuRe resin equilibrated with PBS buffer (10 mM Na₂HPO₄, 1 mM KH₂PO₄, 137 mM NaCl and 2.7 mM KCl, pH 7.4), washed with equilibration buffer and eluted with 25 mM citrate, pH 3.0. After neutralization with 1 M Tris pH 9.0, aggregated protein was separated from monomeric antibody species by size exclusion chromatography (Superdex 200, GE Healthcare) in 20 mM histidine, 140 mM NaCl, pH 6.0. Monomeric protein fractions were pooled, concentrated if required using e.g. a MILLIPORE Amicon Ultra (30KD MWCO) centrifugal concentrator and stored at −80° C. Sample aliquots were used for subsequent analytical characterization e.g. by CE-SDS, size exclusion chromatography, mass spectrometry and endotoxin determination.

### 1.5 Cellular Binding of Anti-huFAP Antibodies

The binding of anti-FAP antibodies with a human IgG1 P329G LALA Fc to human FAP was determined by flow cytometry using human FAP-transfected HEK cells. Briefly, the target cells were harvested with Cell Dissociation buffer, washed with FACS Buffer (PBS+2% FCS+5 mM EDTA+0.25% sodium acide) and seeded into 96-U bottom plates (1×105 cells/well). Unlabeled primary antibodies were added to the cells (final concentrations 10 μg/ml to 0.64 ng/ml; 1:5 dilutions) and incubated for 30 min at 4° C. After washing, cells were incubated with a PE-conjugated AffiPure F(ab)₂ Fragment Goat anti-human IgG, Fc□ specific (Jackson Immunoresearch) for 30 min at 4° C. in the dark. Afterwards, cells were washed, fixed and measured using a BD FACS LSR Fortessa™.

All anti-FAP antibodies showed similar binding to human FAP as seen before. The EC₅₀ values of selected binders are shown in Table 1 below.

TABLE 1
Cellular binding of anti-FAP antibodies to huFAP expressing cells

| | | EC₅₀ [μg/ml] |
|---|---|---|
| | | cellular binding to |
| | | FAP-transfected |
| Sample ID | clone | HEK cells |
| | 4B9 | 0.089 |
| P1AD9427 | 209 | 0.145 |
| P1AD9436 | 210 | 0.125 |
| P1AD9437 | 211 | 0.198 |
| P1AD9438 | 212 | 0.118 |
| P1AD9440 | 214 | 0.086 |

### 1.6 Cellular Internalization of Anti-huFAP Antibodies

Internalization of FAP binders was determined using human FAP-transfected HEK cells as targets. Briefly, the target cells were harvested with Cell Dissociation buffer, washed with cold FACS Buffer (PBS+2% FCS+5 mM EDTA+0.25% sodium acide) and resuspended at 1.5×10⁶ cells/ml in cold FACS Buffer. Cells were distributed in 15 ml tubes (each tube containing 3×10⁶ cells in 2 ml). 2 ml of anti-human FAP antibody solutions were added to the cells (final concentration 20 μg/ml) and incubated for 45 min at 4° C. Afterwards, cells were washed, resuspended in cold FACS Buffer and cells for time point “0” were seeded immediately into 96-U bottom plates (1.5×10⁵ cells/well) and kept at 4° C. whereas all other cells were centrifuged, resuspended in warm RPMI1640 medium containing 10% FCS and 1% Glutamax (1.5×10⁶ cells/ml) and shifted to 37° C. in a humidified incubator (5% CO₂). After each indicated time point, 100 μl/tube of cell suspension was transferred to plates, immediately cooled down with cold FACS Buffer and stored in the fridge until all time points have been collected. After collection of all time points, cells were washed with cold FACS Buffer and incubated with PE-labeled secondary antibody for 30 min at 4° C. Afterwards, cells were washed, fixed and measured using a BD FACS Canto™ II.

The signals caused by the labeled secondary antibody stayed nearly constant over time, which means that no loss of antibody was observed over time, none of the tested anti-hu FAP antibodies was internalized.

### 1.7 Binding Kinetics of Anti-huFAP Antibodies

To evaluate human FAP binding kinetics, biotinylated human FAP was immobilized on a Series S Biacore CAPture Chip (GE Healthcare 28-9202-34) according to the manufacturer's instructions, resulting in a surface density of approximately 20 resonance units (RU). As running and dilution buffer, HBS-P+ (10 mM HEPES, 150 mM NaCl pH 7.4, 0.05% Surfactant P20) was used. A dilution series of anti-huFAP Fabs (3.7-300 nM, 1:3 dilution) was successively injected for 120 s each, dissociation was monitored for 1800 s at a flow rate of 30 μl/min (single cycle kinetics). The surface was regenerated by injecting 6 M guanidine-HCl, 0.25 M NaOH for 120 s. Bulk refractive index differences were corrected by subtracting blank injections and by subtracting the response obtained from the control flow cell without captured human FAP. Curve fitting was performed using the 1:1 Langmuir binding model within the Biacore evaluation software. The affinity data are shown in Table 2 below.

TABLE 2
Affinity of anti-FAP Fabs to human FAP as measured by Biacore

| Sample ID | clone | ka (1/Ms) | kd (1/s) | KD |
|---|---|---|---|---|
| | 4B9_Fab | 1.82E+06 | 7.80E−04 | 430 pM |
| P1AD9427_Fab | 209 | 3.50E+06 | 1.77E−03 | 510 pM |
| P1AD9436_Fab | 210 | 1.87E+06 | <E−06 | <10 pM |
| P1AD9437_Fab | 211 | 8.13E+05 | 4.61E−05 | 60 pM |
| P1AD9438_Fab | 212 | 1.06E+06 | <E−06 | <10 pM |
| P1AD9440_Fab | 214 | 1.99E+06 | <E−06 | <10 pM |

### 1.8 Format-Depending Binding of Anti-huFAP Clones

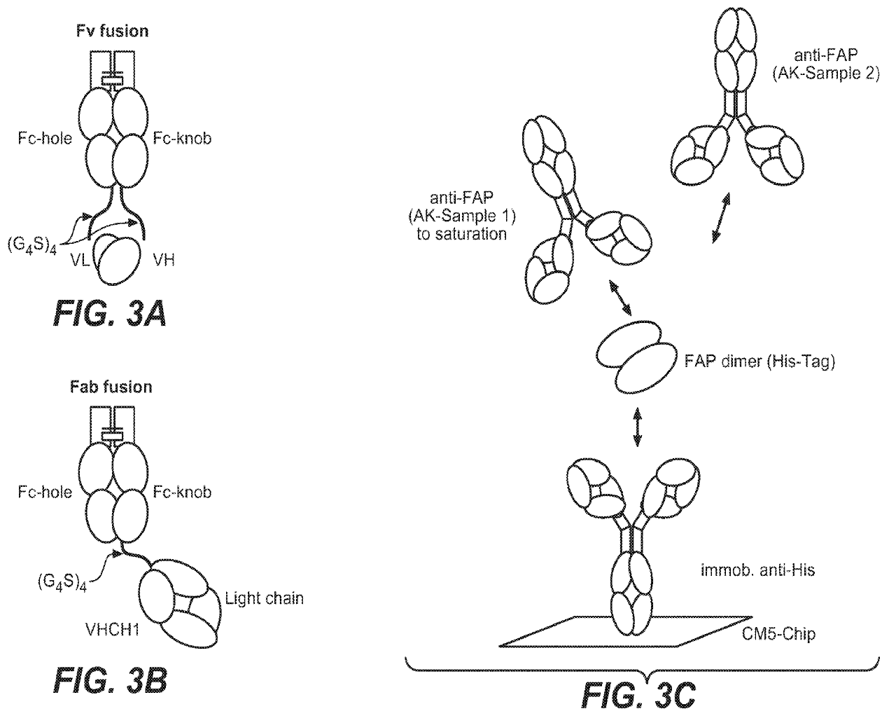

In order to determine if the binding properties of the anti-FAP clones are not lost when they are C-terminally fused to an Fc domain, constructs comprising a Fc knob chain and a Fc hole chain wherein the VH domain is fused to the C-terminus of the Fc knob chain and the VL domain is fused to the C-terminus of the Fc hole chain (FIG. 3A, C-term VH/VL fusion) and constructs comprising a Fc knob chain and a Fc hole chain wherein the whole Fab is fused with its VH domain to the C-terminus of the Fc knob chain (FIG. 3B, C-term Fab fusion). The Fc knob chain has the amino acid sequence of SEQ ID NO:97 and the Fc hole chain has the amino acid sequences of SEQ ID NO:98.

The affinity of the constructs towards biotinylated recombinant human FAP and biotinylated recombinant cynomolgus FAP as compared to the antibodies is shown in Table 3 below.

TABLE 3
Affinity to human FAP and cynomolgus
FAP as measured by Biacore

| | Affinity to human | | | Affinity to cynomolgus | | |
|---|---|---|---|---|---|---|
| | FAP KD [nM] | | | FAP KD [nM] | | |
| | | C-term | C-term | | C-term | C-term |
| | free | Fab | VH/VL | | Fab | VH/VL |
| clone | Fab | fusion | fusion | IgG | fusion | fusion |
| 209 | 0.31 | 1.52 | 42.40 | 0.33 | 1.60 | 50.00 |
| 210 | 0.07 | 0.17 | 3.95 | 0.12 | 0.20 | 3.44 |
| 211 | 0.28 | 1.20 | 10.90 | 0.32 | 1.30 | 11.40 |
| 212 | 0.12 | 0.62 | 5.72 | 0.14 | 0.64 | 6.19 |
| 214 | 0.06 | 0.19 | 2.49 | 0.09 | 0.21 | 2.77 |

Cellular binding of the constructs to FAP-transfected HEK cells has also been determined as described herein before. The EC₅₀ values are shown in Table 4. The C-terminal fusion constructs of all anti-FAP antibodies were able to bind to human and cynomolgus FAP, however the constructs wherein the whole Fab is fused with its VH domain to the C-terminus of the Fc knob chain were superior to those wherein the VH domain is fused to the C-terminus of the Fc knob chain and the VL domain is fused to the C-terminus of the Fc hole chain.

TABLE 4
Cellular binding to huFAP expressing cells

| | Cellular binding to human | | | Cellular binding to cynomolgus | | |
|---|---|---|---|---|---|---|
| | FAP EC₅₀ [μg/ml] | | | FAP EC₅₀ [μg/ml] | | |
| | | C-term | C-term | | C-term | C-term |
| | | Fab | VH/VL | | Fab | VH/VL |
| clone | IgG | fusion | fusion | IgG | fusion | fusion |
| 209 | 0.15 | 1.2 | 5.7 | 0.4 | 1.1 | 7.9 |
| 210 | 0.13 | 1.8 | 9.0 | 0.4 | 1.3 | 7.1 |
| 211 | 0.20 | 3.7 | 9.3 | 0.3 | 2.9 | 6.7 |
| 212 | 0.12 | 2.8 | 8.8 | 0.3 | 2.3 | 11.1 |
| 214 | 0.09 | 1.7 | 9.4 | 0.3 | 1.3 | 3.6 |

### 1.9 Competitive Binding of Anti-Human FAP Clones as Determined by Biacore

Epitope binning was performed using a surface plasmon resonance (SPR) based assay on a Biacore T200 instrument. FAP antigen was captured by an immobilized anti-His antibody. In a first step the FAP-binder was injected until saturation. A second FAP-binder was injected subsequently. The assay design is schematically shown in FIG. 3C. An increase in binding signal after addition of the second antibody indicates its binding to a different epitope from the first antibody. No additional binding indicated that the first and the second antibody recognize the same epitope region.

An anti-His antibody (GE Healthcare Kit 28-9950-56) with a concentration of 20 μg/ml was immobilized by amine coupling (GE Healthcare Kit BR-1000-50) to the surface of a CM5 sensor chip (GE Healthcare BR-1005-30). Injection time was 600 seconds at a flow rate of 10 μl/min to yield 12000 response units (RU) on two flow cells, one used as reference and one used as active flow cell. Running buffer was HBS-N (GE Healthcare BR-1006-70). For the measurement PBS-P+ (GE Healthcare 28-9950-84) was used as running and dilution buffer. Flow cell temperature was set to 25° C., sample compartment to 12° C. The flow rate was set to 10 μl/min for the whole run.

His-tagged FAP antigen was captured with a concentration of 20 μg/ml for 180 seconds on the active flow cell. The first and second antibody (FAP-binder) were injected successively, each for 120 seconds at a concentration of 10 μg/ml over both flow cells. After each cycle the surface was regenerated with 10 mM glycine pH1.5 for 60 seconds (GE Healthcare BR-1003-54).

The results are shown in Table 5 below:

TABLE 5
Competitive Binding of anti-FAP antibodies to 4B9

| | 4B9 | 209 | 210 | 211 | 212 | 214 |
|---|---|---|---|---|---|---|
| 4B9 | Competitive | Simultaneous | Simultaneous | Simultaneous | Simultaneous | Simultaneous |
| | Binding | Binding | Binding | Binding | Binding | Binding |
| 209 | Simultaneous | Competitive | Simultaneous | Simultaneous | Simultaneous | Simultaneous |
| | Binding | Binding | Binding | Binding | Binding | Binding |
| 210 | Simultaneous | Simultaneous | Competitive | Competitive | Competitive | Competitive |
| | Binding | Binding | Binding | Binding | Binding | Binding |
| 211 | Simultaneous | Simultaneous | Competitive | Competitive | Competitive | Competitive |
| | Binding | Binding | Binding | Binding | Binding | Binding |
| 212 | Simultaneous | Simultaneous | Competitive | Competitive | Competitive | Competitive |
| | Binding | Binding | Binding | Binding | Binding | Binding |
| 214 | Simultaneous | Simultaneous | Competitive | Competitive | Competitive | Competitive |
| | Binding | Binding | Binding | Binding | Binding | Binding |

Thus, three epitope bins were identified. As requested, none of the anti-FAP antibodies did compete for binding with antibody 4B9 (Epitope bin 1). Antibodies 210, 211, 212 and 214 competed with each other for binding and thus form one group (Epitope bin 3), whereas antibody 209 did not compete for binding with any other of the antibodies (Epitope bin 2).

### 1.9 Thermal Stability Evaluation of Anti-FAP Antibodies

Samples are prepared at a concentration of 1 mg/mL in 20 mM Histidine/Histidine chloride, 140 mM NaCl, pH 6.0, transferred into an optical 384-well plate by centrifugation through a 0.4 μm filter plate and covered with paraffine oil. The hydrodynamic radius is measured repeatedly by dynamic light scattering on a DynaPro Plate Reader (Wyatt) while the samples are heated with a rate of 0.05° C./min from 25° C. to 80° C. Alternatively, samples were transferred into a 10 μL micro-cuvette array and static light scattering data as well as fluorescence data upon excitation with a 266 nm laser were recorded with an Optim1000 instrument (Avacta Inc.), while they were heated at a rate of 0.1° C./min from 25° C. to 90° C. The aggregation onset temperature (T_{agg}) is defined as the temperature at which the hydrodynamic radius (DLS) or the scattered light intensity (Optim1000) starts to increase. The melting temperature is defined as the inflection point in a graph showing fluorescence intensity vs. wavelength. The aggregation onset temperatures of selected anti-FAP antibodies is shown in Table 6.

TABLE 6
Aggregation onset temperatures of anti-FAP antibodies

| | 4B9 | 209 | 210 | 212 | 214 |
|---|---|---|---|---|---|
| T_{agg} (° C.) | 60 | 66 | 61 | 67 | 61 |

The anti-FAP clone 212 was chosen for humanization as it binds with a comparable high affinity to human FAP as antibody 4B9 and showed favorable properties for the development. In silico analysis of its sequences indicated only one predicted degradation hotspot (Trp at position 401). The sequences of murine clone 212 are shown in Table 7.

TABLE 7
Amino acid sequences of the variable domains
of murine anti-FAP clone 212

| | | Seq ID |
|---|---|---|
| Description | Sequence | No |
| FAP(212) VH | EVLLQQSGPELVKPGASVKIACKASGYTLT | 9 |
| | DYNMDWVRQSHGKSLEWIGDIYPNTGGTIY | |
| | NQKFKGKATLTIDKSSSTAYMDLRSLTSED | |
| | TAVYYCTRFRGIHYAMDYWGQGTSVTVSS | |
| FAP(212) VL | DIVLTQSPVSLAVSLGQRATISCRASESVD | 10 |
| | NYGLSFINWFQQKPGQPPKLLIYGTSNRGS | |
| | GVPARFSGSGSGTDFSLNIHPMEEDDTAMY | |
| | FCQQSNEVPYTFGGGTNLEIK | |

### 1.10 Humanization of Anti-FAP Clone 212

### 1.10.1 Methodology

Suitable human acceptor frameworks were identified by querying a BLASTp database of human V- and J-region sequences for the murine input sequences (cropped to the variable part). Selective criteria for the choice of human acceptor framework were sequence homology, same or similar CDR lengths, and the estimated frequency of the human germline, but also the conservation of certain amino acids at the VH-VL domain interface. Following the germline identification step, the CDRs of the murine input sequences were grafted onto the human acceptor framework regions. Each amino acid difference between these initial CDR grafts and the parental antibodies was rated for possible impact on the structural integrity of the respective variable region, and “back mutations” towards the parental sequence were introduced whenever deemed appropriate. The structural assessment was based on Fv region homology models of both the parental antibody and the humanization variants, created with an in-house antibody structure homology modeling protocol implemented using the Biovia Discovery Studio Environment, version 17R2. In some humanization variants, “forward mutations” were included, i.e., amino acid exchanges that change the original amino acid occurring at a given CDR position of the parental binder to the amino acid found at the equivalent position of the human acceptor germline. The aim is to increase the overall human character of the humanization variants (beyond the framework regions) to further reduce the immunogenicity risk.

An in silico tool developed in-house was used to predict the VH-VL domain orientation of the paired VH and VL humanization variants (see WO 2016/062734). The results were compared to the predicted VH-VL domain orientation of the parental binders to select for framework combinations which are close in geometry to the original antibodies. The rationale is to detect possible amino acid exchanges in the VH-VL interface region that might lead to disruptive changes in the pairing of the two domains that in turn might have detrimental effects on the binding properties.

### 1.10.2 Choice of Acceptor Framework and Adaptations Thereof

The following acceptor frameworks were chosen:

TABLE 8
Acceptor framework

| | | | Choice of | Identity to human |
|---|---|---|---|---|
| | Murine | | human acceptor | V-region germline |
| | V-region | Graft | V-region | after grafting |
| | germline | variant | germline | (BLASTp): |
| FAP | IGHV1-18*01 | VH1 | IGHV1-46*01 | 87.8% |
| (212) | | VH2 | IGHV3-23*03 | 82.7% |
| VH | | | | |
| FAP | IGKV3-2*01 | VL1 | IGKV3-11*01 | 85.1% |
| (212) | | VL2 | IGKV1-39*01 | 82.8% |
| VL | | | | |

Post-CDR3 framework regions were adapted from human IGHJ germline IGHJ6*01/02 (YYYYYGMDVWGQGTTVTVSS) and human IGKJ germline IGKJ4*01/02 (LTFGGGTKVEIK). The part relevant for the acceptor framework is indicated in bold script.

Based on structural considerations, back mutations from the human acceptor framework to the amino acid in the parental binder were introduced at positions H43 (Q>K), H44 (G>S), H48 (M>I), H71 (R>I), H73 (T>K), H93 (A>T) [VH1], H49 (S>G), H71 (R>I), H73 (N>K), H78 (L>A), H93 (A>T), H94 (K>R) [VH2], L36 (Y>F), L43 (A>P), L87 (Y>F) [VL1] and L36 (Y>F), L42 (K>Q), L43 (A>P), L85 (T>M), L87 (Y>F) [VL2].

Furthermore, the positions H60 (N>A), H64 (K>Q) [VH1], H60 (N>A), H61 (Q>D), H62 (K>S), H63 (F>V) [VH2], L33 (I>L), L34 (N>A) [VL1] and L27b (V>I), L33 (I>L) [VL2] were identified as promising candidates for forward mutations. All positions are given in the Kabat EU numbering scheme.

TABLE 9
list of variants

| | | Identity to human |
|---|---|---|
| Variant | | V-region germline |
| name | Back/forward mutations | (BLASTp) |
| VH1G1a | bM48I, bR71I, bA93T | 84.7% |
| VH1G2a | bQ43K, bG44S, bM48I, bR71I, | 81.6% |
| | bT73K, bA93T | |
| VH1G3a | bM48I, fN60A, fK64Q, bR71I, | 86.7% |
| | bA93T | |
| VH2G1a | bS49G, bA93T, bK94R | 79.6% |
| VH2G2a | bS49G, bR71I, bN73K, bL78A, | 76.5% |
| | bA93T, bK94R | |
| VH2G3a | bS49G, fN60A, fQ61D, fK62S, | 83.7% |
| | fF63V, bA93T, bK94R | |
| VL1G1a | bY36F, bY87F | 83% |
| VL1G2a | bY36F, bA43P, bY87F | 81.9% |
| VL1G3a | fI33L, fN34A, bY36F, bY87F | 85.1% |
| VL2G1a | bY36F, bY87F | 80.8% |
| VL2G2a | bY36F, bK42Q, bA43P, bT85M, | 77.8% |
| | bY87F | |
| VL2G3a | fV27bI, fI33L, bY36F, bY87F | 82.8% |

Note:
Back mutations are prefixed with b, forward mutations with f, e.g., bM48I refers to a back mutation (human germline amino acid to parental antibody amino acid) from methionine to isoleucine at position 48 (Kabat).

The resulting VH and VL domains of humanized FAP antibodies based on the acceptor framework can be found in Table 10 below.

TABLE 10
Amino acid sequences of the VH and VL
domains of humanized FAP antibodies

| | | Seq ID |
|---|---|---|
| Description | Sequence | No |
| VH1G1a | QVQLVQSGAEVKKPGASVKVSCKASGYTLTDYNM | 15 |
| | DWVRQAPGQGLEWIGDIYPNTGGTIYNQKFKGRVT | |
| | MTIDTSTSTVYMELSSLRSEDTAVYYCTRFRGIHYA | |
| | MDYWGQGTTVTVSS | |
| VH1G2a | QVQLVQSGAEVKKPGASVKVSCKASGYTLTDYNM | 16 |
| | DWVRQAPGKSLEWIGDIYPNTGGTIYNQKFKGRVT | |
| | MTIDKSTSTVYMELSSLRSEDTAVYYCTRFRGIHYA | |
| | MDYWGQGTTVTVSS | |
| VH1G3a | QVQLVQSGAEVKKPGASVKVSCKASGYTLTDYNM | 17 |
| | DWVRQAPGQGLEWIGDIYPNTGGTIYAQKFQGRVT | |
| | MTIDTSTSTVYMELSSLRSEDTAVYYCTRFRGIHYA | |
| | MDYWGQGTTVTVSS | |
| VH2G1a | EVQLLESGGGLVQPGGSLRLSCAASGYTLTDYNMD | 18 |
| | WVRQAPGKGLEWVGDIYPNTGGTIYNQKFKGRFTIS | |
| | RDNSKNTLYLQMNSLRAEDTAVYYCTRFRGIHYAM | |
| | DYWGQGTTVTVSS | |
| VH2G2a | EVQLLESGGGLVQPGGSLRLSCAASGYTLTDYNMD | 19 |
| | WVRQAPGKGLEWVGDIYPNTGGTIYNQKFKGRFTIS | |
| | IDKSKNTAYLQMNSLRAEDTAVYYCTRFRGIHYAM | |
| | DYWGQGTTVTVSS | |
| VH2G3a | EVQLLESGGGLVQPGGSLRLSCAASGYTLTDYNMD | 20 |
| | WVRQAPGKGLEWVGDIYPNTGGTIYADSVKGRFTIS | |
| | RDNSKNTLYLQMNSLRAEDTAVYYCTRFRGIHYAM | |
| | DYWGQGTTVTVSS | |
| VL1G1a | EIVLTQSPATLSLSPGERATLSCRASESVDNYGLSFIN | 21 |
| | WFQQKPGQAPRLLIYGTSNRGSGIPARFSGSGSGTDF | |
| | TLTISSLEPEDFAVYFCQQSNEVPYTFGGGTKVEIK | |
| VL1G2a | EIVLTQSPATLSLSPGERATLSCRASESVDNYGLSFIN | 22 |
| | WFQQKPGQPPRLLIYGTSNRGSGIPARFSGSGSGTDF | |
| | TLTISSLEPEDFAVYFCQQSNEVPYTFGGGTKVEIK | |
| VL1G3a | EIVLTQSPATLSLSPGERATLSCRASESVDNYGLSFLA | 23 |
| | WFQQKPGQAPRLLIYGTSNRGSGIPARFSGSGSGTDF | |
| | TLTISSLEPEDFAVYFCQQSNEVPYTFGGGTKVEIK | |
| VL2G1a | DIQMTQSPSSLSASVGDRVTITCRASESVDNYGLSFI | 24 |
| | NWFQQKPGKAPKLLIYGTSNRGSGVPSRFSGSGSGT | |
| | DFTLTISSLQPEDFATYFCQQSNEVPYTFGGGTKVEI | |
| | K | |
| VL2G2a | DIQMTQSPSSLSASVGDRVTITCRASESVDNYGLSFI | 25 |
| | NWFQQKPGQPPKLLIYGTSNRGSGVPSRFSGSGSGT | |
| | DFTLTISSLQPEDFAMYFCQQSNEVPYTFGGGTKVEI | |
| | K | |
| VL2G3a | DIQMTQSPSSLSASVGDRVTITCRASESIDNYGLSFLN | 26 |
| | WFQQKPGKAPKLLIYGTSNRGSGVPSRFSGSGSGTD | |
| | FTLTISSLQPEDFATYFCQQSNEVPYTFGGGTKVEIK | |

### 1.10.3 New Humanized Anti-FAP Fabs

Based on the new humanization variants of VH and VL new anti-FAP Fabs were expressed.

TABLE 11
Nomenclature for VH/VL combinations expressed as Fabs

| | VL1G1a | VL1G2a | VL1G3a | VL2G1a | VL2G2a | VL2G3a |
|---|---|---|---|---|---|---|
| VH1G1a | P1AE1689 | | | | P1AE1702 | |
| VH1G2a | P1AE1690 | P1AE1693 | | | | |
| VH1G3a | | | | | | |
| VH2G1a | | | | | | |
| VH2G2a | | | | | | |
| VH2G3a | | | | | | |

The affinity of the new humanized anti-FAP variants based on clone 212 was analyzed in comparison with anti-FAP antibody 4B9. Furthermore, the humanness of the humanized variants was calculated and its aggregation onset temperature was measured.

TABLE 12
Affinity of humanization variants
of clone 212 as measured by Biacore

| | | | | | Identity | |
|---|---|---|---|---|---|---|
| | ka | kd | KD | T ½ | to hu V | T_{agg} |
| Sample ID | (1/Ms) | (1/s) | (pM) | (min) | germline | [° C.] |
| P1AE1689_ | 4.43E+ | 4.21E− | 95 | 274 | 83/84.7 | 72.7 |
| Fab | 05 | 05 | | | | |
| P1AE1690_ | 5.51E+ | 6.30E− | 114 | 183 | 83/81.7 | 75.4 |
| Fab | 05 | 05 | | | | |
| P1AE1693_ | 5.30E+ | 7.18E− | 135 | 161 | 81.9/81.7 | 75.4 |
| Fab | 05 | 05 | | | | |
| P1AE1702_ | 5.02E+ | 1.07E− | 213 | 108 | 77.8/76.5 | 71.6 |
| Fab | 05 | 04 | | | | |
| 4B9_Fab | 7.47E+ | 2.08E− | 279 | 55 | | 60 |
| | 05 | 04 | | | | |

### 1.11 FcRn/Heparin Binding and in Silico Charge Distribution

The charge distribution of antibodies 4B9 and P1AE1689 in PBS, pH 7.4, was calculated in an in-silico model. According to the model, 4B9 has a large positive patch which is sometimes correlated with increased heparin binding. P1AE1689, on the other hand, shows a large negative charge patch which might be indicative for weak heparin interaction.

These predictions were confirmed by chromatography of both antibodies using a FcRn affinity column and pH gradient as well as a heparin affinity column and pH gradient. WO 2015/140126 discloses a method for the prediction of the in vivo half-life of an antibody based on the retention time determined on an FcRn affinity chromatography column, whereas heparin binding correlates with non-specific interactions with cell surface structures.

### Example 2

### Generation and Production of Humanized Variants of Anti-CD40 Antibody S2C6

### 2.1 Generation of Humanized Variants of Anti-CD40 Antibody S2C6

### 2.2.1 Methodology

For the identification of a suitable human acceptor framework during the humanization of the anti-CD40 binder S2C6 a combination of two methodologies was used. On the one hand, a classical approach was taken by searching for an acceptor framework with high sequence homology, grafting of the CDRs on this framework, and evaluating which back-mutations can be envisaged. More explicitly, each amino acid difference of the identified frameworks to the parental antibody was judged for impact on the structural integrity of the binder, and back mutations towards the parental sequence were introduced whenever appropriate. The structural assessment was based on Fv region homology models of both the parental antibody and its humanized versions created with an in-house antibody structure homology modeling tool implemented using the Biovia Discovery Studio Environment, version 4.5.

On the other hand, an in-house developed in silico tool was used to predict the orientation of the VH and VL domains of the humanized versions towards each other (see WO 2016062734 incorporated herein by reference). The results were compared to the predicted VH-VL domain orientation of the parental binder to select for framework combinations which are close in geometry to the starting antibody. The rational is to detect possible amino acid exchange in the VH-VL interface region that might lead to disruptive changes in the pairing of the two domains.

### 2.2.2 Choice of Acceptor Framework and Adaptations Thereof

Two different acceptor frameworks were chosen as described in Table 16 and Table 18 below.

TABLE 13
Acceptor framework 1: “IGHV1-IGKV2D”

| | | Choice of human | Identity to human V- |
|---|---|---|---|
| | Murine V-region | acceptor V-region | region germline after |
| | germline | germline | grafting (BLASTp): |
| S2C6 VH | IGHV1-26*01 | IGHV1-2*05 | 91.8% |
| S2C6 VL | IGKV1-110*01 | IGKV2D-29*02 | 88.0% |

Post-CDR3 framework regions were adapted from human IGHJ germline IGHJ6*01/02 (YYYYYGMDVWGQGTTVTVSS) and human IGKJ germline IGKJ4*01/02 (LTFGGGTKVEIK). The part relevant for the acceptor framework is indicated in bold script.

Based on structural considerations, back mutations from the human acceptor framework to the amino acid in the parental binder were introduced at positions H43 (Q>K), H44 (G>S), H69 (M>L), H71 (R>V), H73 (T>K), H88 (V>A) and H105 (Q>H) of the VH region and at positions L2 (I>V), L4 (M>V), L87 (Y>F) and L104 (V>L) of the VL region. In one variant, mutation T70S (VH) was included to study the effect of a slightly more hydrophilic residue at this position.

All variants include the N54A mutation (VH) to address a putative developability hotspot (asparagine deamidation). All positions are given in the Kabat EU numbering scheme.

In the following Table 14 the Humanization variant VH-VL pairing matrix is shown:

| | | | | VL1c | VL1d |
|---|---|---|---|---|---|
| | | | VL1b | bI2V, | bI2V, |
| | | VL1a | bM4V, | bM4V, | bM4V, |
| | | bY87F | bY87F | bY83F | bY783F, |
| VH1a | bG44S, bM69L, | x | x | x | x |
| | bR71V, bT73K, | | | | |
| | bV88A | | | | |
| VH1b | bQ43K, bG44S, | x | x | x | x |
| | bM69L, bR71V, | | | | |
| | bT73K, bV88A | | | | |
| VH1c | bG44S, bM69L, | x | x | x | x |
| | bR71V, bT73K, | | | | |
| | bV88A, bQ105H | | | | |
| VH1d | bG44S, bM69L, | x | x | x | x |
| | bR71V, bT73K, | | | | |
| | bV88A, xT70S | | | | |

Mutation N54A applies to all VH variants and is not explicitly mentioned. Back mutations prefixed with b, forward mutations prefixed with f, and other mutations prefixed with x

TABLE 15
Acceptor framework 2: “IGHV3-IGKV1”

| | | Choice of human | Identity to human V- |
|---|---|---|---|
| | Murine V-region | acceptor V-region | region germline after |
| | germline | germline | grafting (BLASTp): |
| S2C6 VH | IGHV1-26*01 | IGHV3-23*02 | 79.6% |
| S2C6 VL | IGKV1-110*01 | IGKV1-39*01 | 79.0% |

Post-CDR3 framework regions were adapted from human IGHJ germline IGHJ6*01/02 (YYYYYGMDVWGQGTTVTVSS) and human IGKJ germline IGKJ4*01/02 (LTFGGGTKVEIK). The part relevant for the acceptor framework is indicated in bold script.

Based on structural considerations, back mutations from the human acceptor framework to the amino acid in the parental binder were introduced at positions H44 (G>S), H49 (S>G), H71 (R>V), H78 (L>A), H94 (K>R) and H105 (Q>H) of the VH region and at positions L42 (K>Q), L43 (A>S) and L87 (Y>F) of the VL region. Furthermore, four positions in CDR-H2 were identified as promising candidates for forward mutations, i.e., amino acid exchanges from parental binder to human acceptor germline in order to increase overall human character, namely H60 (N>G), H61 (Q>D), H62 (K>S) and H63 (F>V).

All variants include the N54A mutation (VH) to address a putative developability hotspot (asparagine deamidation). All positions are given in the Kabat EU numbering scheme.

In the following Table 16 the Humanization variant VH-VL pairing matrix is shown:

| | | | VL2b |
|---|---|---|---|
| | | VL2a | bK42Q, |
| | | bY87F | bA43S, |
| VH2a | bS49G, bR71V, bL78A, bK94R | x | x |
| VH2b | bG44S, bS49G, bR71V, bL78A, bK94R | x | x |
| VH2c | bS49G, bR71V, bL78A, bK94R, bQ105H | x | x |
| VH2d | bS49G, fN60G, fQ61D, fK62S, fF63V, | x | x |
| | bR71V, bL78A, bK94R | | |

Back mutations prefixed with b, forward mutations prefixed with f.

### 2.2.3 VH and VL Domains of the Resulting Humanized CD40 Antibodies

The resulting VH and VL domains of humanized CD40 antibodies based on acceptor framework 1 can be found in Table 17 below and the resulting VH and VL domains of humanized CD40 antibodies based on acceptor framework 2 are listed in Table 18 below.

TABLE 17
Amino acid sequences of the VH and VL domains of
humanized CD40 antibodies based on acceptor framework 1

| | | Seq ID |
|---|---|---|
| Description | Sequence | No |
| VH1a | QVQLVQSGAEVKKPGASVKVSCKASGYSFTGYYIHW | 37 |
| | VRQAPGQSLEWMGRVIPNAGGTSYNQKFKGRVTLTV | |
| | DKSISTAYMELSRLRSDDTAVYYCAREGIYWWGQGT | |
| | TVTVSS | |
| VH1b | QVQLVQSGAEVKKPGASVKVSCKASGYSFTGYYIHW | 38 |
| | VRQAPGKSLEWMGRVIPNAGGTSYNQKFKGRVTLTV | |
| | DKSISTAYMELSRLRSDDTAVYYCAREGIYWWGQGT | |
| | TVTVSS | |
| VH1c | QVQLVQSGAEVKKPGASVKVSCKASGYSFTGYYIHW | 39 |
| | VRQAPGQSLEWMGRVIPNAGGTSYNQKFKGRVTLTV | |
| | DKSISTAYMELSRLRSDDTAVYYCAREGIYWWGHGT | |
| | TVTVSS | |
| VH1d | QVQLVQSGAEVKKPGASVKVSCKASGYSFTGYYIHW | 40 |
| | VRQAPGQSLEWMGRVIPNAGGTSYNQKFKGRVTLSV | |
| | DKSISTAYMELSRLRSDDTAVYYCAREGIYWWGQGT | |
| | TVTVSS | |
| VL1a | DIVMTQTPLSLSVTPGQPASISCRSSQSLVHSNGNTFL | 41 |
| | HWYLQKPGQSPQLLIYTVSNRFSGVPDRFSGSGSGTD | |
| | FTLKISRVEAEDVGVYFCSQTTHVPWTFGGGTKVEIK | |
| VL1b | DIVVTQTPLSLSVTPGQPASISCRSSQSLVHSNGNTFLH | 42 |
| | WYLQKPGQSPQLLIYTVSNRFSGVPDRFSGSGSGTDF | |
| | TLKISRVEAEDVGVYFCSQTTHVPWTFGGGTKVEIK | |
| VL1c | DVVVTQTPLSLSVTPGQPASISCRSSQSLVHSNGNTFL | 43 |
| | HWYLQKPGQSPQLLIYTVSNRFSGVPDRFSGSGSGTD | |
| | FTLKISRVEAEDVGVYFCSQTTHVPWTFGGGTKVEIK | |
| VL1d | DVVVTQTPLSLSVTPGQPASISCRSSQSLVHSNGNTFL | 44 |
| | HWYLQKPGQSPQLLIYTVSNRFSGVPDRFSGSGSGTD | |
| | FTLKISRVEAEDVGVYFCSQTTHVPWTFGGGTKLEIK | |

TABLE 18
Amino acid sequences of the VH and VL domains of
humanized CD40 antibodies based on acceptor framework 2

| | | Seq ID |
|---|---|---|
| Description | Sequence | No |
| VH2a | EVQLLESGGGLVQPGGSLRLSCAASGYSFTGYYIHWV | 45 |
| | RQAPGKGLEWVGRVIPNAGGTSYNQKFKGRFTISVD | |
| | NSKNTAYLQMNSLRAEDTAVYYCAREGIYWWGQGT | |
| | TVTVSS | |
| VH2b | EVQLLESGGGLVQPGGSLRLSCAASGYSFTGYYIHWV | 46 |
| | RQAPGKSLEWVGRVIPNAGGTSYNQKFKGRFTISVDN | |
| | SKNTAYLQMNSLRAEDTAVYYCAREGIYWWGQGTT | |
| | VTVSS | |
| VH2c | EVQLLESGGGLVQPGGSLRLSCAASGYSFTGYYIHWV | 47 |
| | RQAPGKGLEWVGRVIPNAGGTSYNQKFKGRFTISVD | |
| | NSKNTAYLQMNSLRAEDTAVYYCAREGIYWWGHGT | |
| | TVTVSS | |
| VH2d | EVQLLESGGGLVQPGGSLRLSCAASGYSFTGYYIHWV | 48 |
| | RQAPGKGLEWVGRVIPNAGGTSYGDSVKGRFTISVD | |
| | NSKNTAYLQMNSLRAEDTAVYYCAREGIYWWGQGT | |
| | TVTVSS | |
| VH2ab | EVQLLESGGGLVQPGGSLRLSCAASGYSFTGYYMHW | 49 |
| | VRQAPGKGLEWVGRVIPNAGGTSYNQKFKGRFTISV | |
| | DNSKNTAYLQMNSLRAEDTAVYYCAREGIYWWGQG | |
| | TTVTVSS | |
| VH2ac | EVQLLESGGGLVQPGGSLRLSCAASGYSFTGYYIHWV | 50 |
| | RQAPGKGLEWVGRVIPNAGGTSYNQKVKGRFTISVD | |
| | NSKNTAYLQMNSLRAEDTAVYYCAREGIYWWGQGT | |
| | TVTVSS | |
| VL2a | DIQMTQSPSSLSASVGDRVTITCRSSQSLVHSNGNTFL | 51 |
| | HWYQQKPGKAPKLLIYTVSNRFSGVPSRFSGSGSGTD | |
| | FTLTISSLQPEDFATYFCSQTTHVPWTFGGGTKVEIK | |
| VL2b | DIQMTQSPSSLSASVGDRVTITCRSSQSLVHSNGNTFL | 52 |
| | HWYQQKPGQSPKLLIYTVSNRFSGVPSRFSGSGSGTD | |
| | FTLTISSLQPEDFATYFCSQTTHVPWTFGGGTKVEIK | |
| VL2ab | DIQMTQSPSSLSASVGDRVTITCRASQSLVHSNGNTFL | 53 |
| | HWYQQKPGKAPKLLIYTVSNRFSGVPSRFSGSGSGTD | |
| | FTLTISSLQPEDFATYFCSQTTHVPWTFGGGTKVEIK | |
| VL2ac | DIQMTQSPSSLSASVGDRVTITCRSSQSIVHSNGNTFL | 54 |
| | HWYQQKPGKAPKLLIYTVSNRFSGVPSRFSGSGSGTD | |
| | FTLTISSLQPEDFATYFCSQTTHVPWTFGGGTKVEIK | |

2.2.4 New Humanized CD40 Antibodies in huIgG1_LALA_PG Format

Based on the new humanization variants of VH and VL new CD40 antibodies were expressed as huIgG1 antibodies with an effector silent Fc (P329G; L234, L235A) to abrogate binding to Fc□ receptors according to the method described in WO 2012/130831 A1.

TABLE 19
Nomenclature for VH/VL combinations expressed as huIgG1_LALA_PG antibodies

| | VL1a | VL1b | VL1c | VL1d | VL2a | VL2b | VL2ab | VL2ac |
|---|---|---|---|---|---|---|---|---|
| VH1a | P1AE0817 | P1AE1001 | P1AE0993 | P1AE0996 | | | | |
| VH1b | P1AE1002 | P1AE1003 | P1AE1004 | P1AE1005 | | | | |
| VH1c | P1AE0997 | P1AE1006 | P1AE0818 | P1AE0998 | | | | |
| VH1d | P1AE0999 | P1AE1007 | P1AE1000 | P1AE0819 | | | | |
| VH2a | | | | | P1AE0400 | P1AE0404 | | |
| VH2b | | | | | P1AE0401 | P1AE0405 | | |
| VH2c | | | | | P1AE0402 | P1AE0406 | | |
| VH2d | | | | | P1AE0403 | P1AE0407 | | |
| VH2ab | | | | | | | P1AE1125 | P1AE1126 |
| VH2ac | | | | | | | P1AE1134 | P1AE1135 |

Exemplary full-length sequences of humanized CD40 antibodies as human IgG1_LALAPG antibodies can be found in Table 20.

TABLE 20
Amino acid sequences of the humanized CD40
IgG1_LALAPG antibodies

| | | Seq ID |
|---|---|---|
| Antibody | Sequence | No |
| P1AE0400 | EVQLLESGGGLVQPGGSLRLSCAASGYSFTGYYIHW | 55 |
| heavy chain | VRQAPGKGLEWVGRVIPNAGGTSYNQKFKGRFTISV | |
| | DNSKNTAYLQMNSLRAEDTAVYYCAREGIYWWGQ | |
| | GTTVTVSSASTKGPSVFPLAPSSKSTSGGTAALGCLV | |
| | KDYFPEPVTVSWNSGALTSGVHTFPAVLQSSGLYSL | |
| | SSVVTVPSSSLGTQTYICNVNHKPSNTKVDKKVEPK | |
| | SCDKTHTCPPCPAPEAAGGPSVFLFPPKPKDTLMISR | |
| | TPEVTCVVVDVSHEDPEVKFNWYVDGVEVHNAKT | |
| | KPREEQYNSTYRVVSVLTVLHQDWLNGKEYKCKVS | |
| | NKALGAPIEKTISKAKGQPREPQVYTLPPSRDELTKN | |
| | QVSLTCLVKGFYPSDIAVEWESNGQPENNYKTTPPV | |
| | LDSDGSFFLYSKLTVDKSRWQQGNVFSCSVMHEAL | |
| | HNHYTQKSLSLSPG | |
| P1AE0400 | DIQMTQSPSSLSASVGDRVTITCRSSQSLVHSNGNTF | 56 |
| light chain | LHWYQQKPGKAPKLLIYTVSNRFSGVPSRFSGSGSG | |
| | TDFTLTISSLQPEDFATYFCSQTTHVPWTFGGGTKVE | |
| | IKRTVAAPSVFIFPPSDEQLKSGTASVVCLLNNFYPRE | |
| | AKVQWKVDNALQSGNSQESVTEQDSKDSTYSLSST | |
| | LTLSKADYEKHKVYACEVTHQGLSSPVTKSFNRGEC | |
| P1AE0403 | EVQLLESGGGLVQPGGSLRLSCAASGYSFTGYYIHW | 60 |
| heavy chain | VRQAPGKGLEWVGRVIPNAGGTSYGDSVKGRFTISV | |
| | DNSKNTAYLQMNSLRAEDTAVYYCAREGIYWWGQ | |
| | GTTVTVSSASTKGPSVFPLAPSSKSTSGGTAALGCLV | |
| | KDYFPEPVTVSWNSGALTSGVHTFPAVLQSSGLYSL | |
| | SSVVTVPSSSLGTQTYICNVNHKPSNTKVDKKVEPK | |
| | SCDKTHTCPPCPAPEAAGGPSVFLFPPKPKDTLMISR | |
| | TPEVTCVVVDVSHEDPEVKFNWYVDGVEVHNAKT | |
| | KPREEQYNSTYRVVSVLTVLHQDWLNGKEYKCKVS | |
| | NKALGAPIEKTISKAKGQPREPQVYTLPPSRDELTKN | |
| | QVSLTCLVKGFYPSDIAVEWESNGQPENNYKTTPPV | |
| | LDSDGSFFLYSKLTVDKSRWQQGNVFSCSVMHEAL | |
| | HNHYTQKSLSLSPG | |
| P1AE0403 | DIQMTQSPSSLSASVGDRVTITCRSSQSLVHSNGNTF | 61 |
| light chain | LHWYQQKPGKAPKLLIYTVSNRFSGVPSRFSGSGSG | |
| | TDFTLTISSLQPEDFATYFCSQTTHVPWTFGGGTKVE | |
| | IKRTVAAPSVFIFPPSDEQLKSGTASVVCLLNNFYPRE | |
| | AKVQWKVDNALQSGNSQESVTEQDSKDSTYSLSST | |
| | LTLSKADYEKHKVYACEVTHQGLSSPVTKSFNRGEC | |
| P1AE0817 | QVQLVQSGAEVKKPGASVKVSCKASGYSFTGYYIH | 62 |
| heavy chain | WVRQAPGQSLEWMGRVIPNAGGTSYNQKFKGRVT | |
| | LTVDKSISTAYMELSRLRSDDTAVYYCAREGIYWW | |
| | GQGTTVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVEP | |
| | KSCDKTHTCPPCPAPEAAGGPSVFLFPPKPKDTLMIS | |
| | RTPEVTCVVVDVSHEDPEVKFNWYVDGVEVHNAK | |
| | TKPREEQYNSTYRVVSVLTVLHQDWLNGKEYKCKV | |
| | SNKALGAPIEKTISKAKGQPREPQVYTLPPSRDELTK | |
| | NQVSLTCLVKGFYPSDIAVEWESNGQPENNYKTTPP | |
| | VLDSDGSFFLYSKLTVDKSRWQQGNVFSCSVMHEA | |
| | LHNHYTQKSLSLSPG | |
| P1AE0817 | DIVMTQTPLSLSVTPGQPASISCRSSQSLVHSNGNTFL | 63 |
| light chain | HWYLQKPGQSPQLLIYTVSNRFSGVPDRFSGSGSGT | |
| | DFTLKISRVEAEDVGVYFCSQTTHVPWTFGGGTKVE | |
| | IKRTVAAPSVFIFPPSDEQLKSGTASVVCLLNNFYPRE | |
| | AKVQWKVDNALQSGNSQESVTEQDSKDSTYSLSST | |
| | LTLSKADYEKHKVYACEVTHQGLSSPVTKSFNRGEC | |

2.2.5 Production of the New Humanized CD40 Antibodies in huIgG1_LALA_PG Format

The antibodies were expressed by transient transfection of HEK293-F cells grown in suspension with expression vectors encoding the different peptide chains. Transfection into HEK293-F cells (Invitrogen, USA) was performed according to the cell supplier's instructions using Maxiprep (Qiagen, Germany) preparations of the antibody vectors, F17 based medium (Invitrogen, USA), PEIpro (Polyscience Europe GmbH) and an initial cell density of 1-2 million viable cells/ml in serum free FreeStyle 293 expression medium (Invitrogen). Cell culture supernatants were harvested after 7 days of cultivation in shake flasks or stirred fermenters by centrifugation at 14000 g for 30 minutes and filtered through a 0.22 μm filter.

The antibodies were purified from cell culture supernatants by affinity chromatography using MabSelectSure-Sepharose™ (GE Healthcare, Sweden) chromatography. Briefly, sterile filtered cell culture supernatants were captured on a MabSelect SuRe resin equilibrated with PBS buffer (10 mM Na₂HPO₄, 1 mM KH₂PO₄, 137 mM NaCl and 2.7 mM KCl, pH 7.4), washed with equilibration buffer and eluted with 25 mM citrate, pH 3.0, followed by neutralization with 1 M Tris pH 9.0. Dependent on product quality received after ProteinA purification, a hydrophobic interaction chromatography (HIC) purification step was included using Butyl-Sepharose 4FF (GE Healthcare, Sweden) resin. Prior HIC purification, the protein was dialysed against HIC equilibration buffer. HIC purification was performed using 40 mM acetate, 1.5 M ammonium sulfate, pH 5.5 as equilibration/washing buffer and 40 mM acetate pH 5.5 as elution buffer and a linear gradient was applied for purification. Subsequently, aggregated protein was separated from monomeric antibody species by size exclusion chromatography (Superdex 200, GE Healthcare) in 20 mM histidine, 140 mM NaCl, pH 6.0. Monomeric protein fractions were pooled, concentrated if required using e.g. a MILLIPORE Amicon Ultra (30KD MWCO) centrifugal concentrator and stored at −80° C. Sample aliquots were used for subsequent analytical characterization e.g. by CE-SDS, size exclusion chromatography, mass spectrometry and endotoxin determination.

The production yield for the different humanized CD40 antibodies is shown in Table 21 as titer values calculated from the yield after preparative affinity chromatography using MabSelectSure-Sepharose™ chromatography.

Purity and molecular weight of the molecule after the final purification step were analyzed by CE-SDS analyses in the presence and absence of a reducing agent. The Caliper LabChip GXII system (Caliper Lifescience) was used according to the manufacturer's instruction.

The aggregate content of the molecule was analyzed using a TSKgel G3000 SW XL analytical size-exclusion column (Tosoh) in 25 mM potassium phosphate, 125 mM sodium chloride, 200 mM L-arginine monohydrocloride, 0.02% (w/v) NaN₃, pH 6.7 running buffer at 25° C.

For direct comparison of all antibodies the thermal stability was monitored by Static Light Scattering (SLS) and by measuring the intrinsic protein fluorescence in response to applied temperature stress. 30 □g of filtered protein sample with a protein concentration of 1 mg/ml was applied in duplicate to an Optim 2 instrument (Avacta Analytical Ltd). The temperature was ramped from 25 to 85° C. at 0.1° C./min, with the radius and total scattering intensity being collected. For determination of intrinsic protein fluorescence the sample was excited at 266 nm and emission was collected between 275 nm and 460 nm. For all antibodies the aggregation temperature (Tagg) was between 64° C. and 69° C. and is provided in Table 21 or Table 22 below.

The production yield for the humanized CD40 antibodies with the different frameworks is shown in Table 21 or Table 22 below.

TABLE 21
Production titer, humanness and aggregation temperature of
humanized CD40 antibodies based on acceptor framework 2

| | | Titer | humanness | |
|---|---|---|---|---|
| Antibody | VH/VL | [□g/ml] | (VH/VL in %) | T_{agg} |
| P1AD4470 | control | 140 | 77.6/78 | 68 |
| P1AE0400 | VL2a/VH2a | 219 | 77.6/78 | 69 |
| P1AE0401 | VL2a/VH2b | 162 | 76.5/78 | 69 |
| P1AE0402 | VL2a/VH2c | 196 | 77.6/78 | 69 |
| P1AE0403 | VL2a/VH2d | 137 | 80.6/78 | 67 |
| P1AE0404 | VL2b/VH2a | 165 | 77.6/76 | 69 |
| P1AE0405 | VL2b/VH2b | 128 | 76.5/76 | 69 |
| P1AE0406 | VL2b/VH2c | 154 | 77.6/76 | 69 |
| P1AE0407 | VL2b/VH2d | 102 | 80.6/76 | 67 |

TABLE 22
Production titer, humanness and aggregation temperature of
humanized CD40 antibodies based on acceptor framework 1

| | | Titer | humanness | |
|---|---|---|---|---|
| Antibody | VH/VL | [□g/ml] | (VH/VL in %) | T_{agg} |
| P1AE0816 | contro1 | 8.5 | 84.7/84 | 64 |
| P1AE0817 | VH1a/VL1a | 62 | 86.7/87 | 67 |
| P1AE0818 | VH1c/VL1c | 47 | 86.7/85 | 66 |
| P1AE0819 | VH1d/VL1d | 90 | 85.7/85 | 67 |
| P1AE0993 | VH1a/VL1c | 34 | 86.7/85 | 67 |
| P1AE0996 | VH1a/VL1d | 16 | 86.7/85 | 67 |
| P1AE0997 | VH1c/VL1a | 44 | 86.7/87 | 66 |
| P1AE0998 | VH1c/VL1d | 24 | 86.7/85 | 66 |
| P1AE0999 | VH1d/VL1a | 34 | 85.7/87 | 67 |
| P1AE1000 | VH1d/VL1c | 16 | 85.7/85 | 66 |
| P1AE1001 | VH1a/VL1b | 34 | 86.7/86 | 65 |
| P1AE1002 | VH1b/VL1a | 46 | 85.7/87 | 67 |
| P1AE1003 | VH1b/VL1b | 49 | 85.7/86 | 66 |
| P1AE1004 | VH1b/VL1c | 60 | 85.7/85 | 67 |
| P1AE1005 | VH1b/VL1d | 7 | 85.7/85 | 65 |
| P1AE1006 | VH1c/VL1b | 24 | 86.7/86 | 65 |
| P1AE1007 | VH1d/VL1b | 34 | 85.7/86 | 67 |

### 2.2.6 Generation of Recombinant Human and Cynomolgus Monkey CD40 Extracellular Domain Protein

Following constructs were cloned and expressed by transient expression in HEK293 cells:
1) Human CD40 extracellular domain (amino acids 21-193 of SEQ ID NO:1, NCBI accession number NP_001241) with C-terminal His-AviTag™ tag (SEQ ID NO:266)
2) Cynomolgus monkey (Macaca fascicularis) CD40 extracellular domain (amino acids 21-193, cynomolgus CD40 extracellular domain sequence was taken from Roche cynomolgus cDNA database, unpublished data) with C-terminal His-AviTag™ tag (SEQ ID NO: 267)

CD40 extracellular domain antigens for binding analysis were generated by gene synthesis (Eurofins Genomics GmbH service, Germany), cloned via unique restriction sites into Roche's in house expression vector using standard cloning procedures. Cloning of all constructs was verified by sequencing. All antigens were expressed under the control of the CMV-promoter. For transient expression of the CD40 extracellular domain constructs, suspension-adapted HEK293-F cells (Life Technologies, USA) were transfected with the respective plasmids: In general, 1 L of HEK293-F cells at about 2×10⁶ cells/ml were transfected with a total of 500 μg plasmid DNA complexed by the PEIpro Transfection Reagent (Polysciences Europe GmbH, Germany) according to manufacturer's instructions. After transfection, HEK293-F cells were incubated for 6 days. The cells were subsequently harvested by centrifugation and the protein-containing supernatant was filtered using a 0.22 μm vacuum filtration system (Millipore). The His-AviTag™ tagged proteins were purified by IMAC affinity chromatography using complete-His-Tag resin (Roche Diagnostics). After washing with 50 mM Na₂PO₄, 300 mM NaCl, pH 8.0, His-AviTag™ fusion proteins were eluted using washing buffer supplemented with 500 mM Imidazol at pH 7.0. Aggregated protein was separated from monomeric fusion proteins by size exclusion chromatography (Superdex 75, GE Healthcare) in 20 mM Tris, 150 mM NaCl, pH 7.4. Monomeric protein fractions were pooled, concentrated if required using e.g. a MILLIPORE Amicon Ultra (10KD MWCO) centrifugal concentrator and stored at −80° C. Sample aliquots were used for subsequent analytical characterization e.g. by CE-SDS, size exclusion chromatography and mass spectrometry.

### Biotinylation of CD40 Extracellular Domain:

Enzymatic site specific biotinylation of human or cynomolgus CD40 extracellular domain constructs containing a C-terminal AviTag™ was performed by using the BirA biotin-protein ligase kit (Avidity LLC, USA) according to manufactures instruction. Briefly, 1/10 volume of BiomixA (10× concentration: 0.5M bicine buffer, pH 8.3) and BiomixB (10× concentration: 100 mM ATP, 100 mM MgOAc, 500 μM d-biotin) was added to AviTag™ containing protein followed by addition of 2.5 μg BirA ligase per 10 nmol protein. The reaction mixture was incubated at 30° C. for 1 h and purified by size exclusion chromatography on a Superdex75 prep grade prepacked HiLoad column (GE Healthcare, Sweden).

### 2.2.7 Human/Cynomolgus CD40 Binding Surface Plasmon Resonance Spectroscopy Assay

Around 12000 resonance units (RU) of the capturing system (10 μg/ml goat anti human F(ab)′₂; Order Code: 28958325; GE Healthcare Bio-Sciences AB, Sweden) were coupled on a CM5 chip (GE Healthcare BR-1005-30) at pH 5.0 by using an amine coupling kit supplied by the GE Healthcare. The sample and system buffer was PBS-T (10 mM phosphate buffered saline including 0.05% Tween20) pH 7.4. The flow cell was set to 25° C.—and the sample block set to 12° C.—and primed with running buffer twice. The antibody was captured by injecting a 50 nM solution for 30 sec at a flow of 5 μl/min. Association was measured by injection of human CD40 extra cellular domain or cynomolgus monkey CD40 extracellular domain in various concentrations in solution for 300 sec at a flow of 30 μl/min starting with 300 nM in 1:3 dilutions. The dissociation phase was monitored for up to 1200 sec and triggered by switching from the sample solution to running buffer. The surface was regenerated by 60 sec washing with a Glycine pH 2.1 solution at a flow rate of 30 μl/min. Bulk refractive index differences were corrected by subtracting the response obtained from a goat anti human F(ab′)₂ surface. Blank injections are also subtracted (=double referencing). For calculation of apparent K_{D} and other kinetic parameters the Langmuir 1:1 model was used. The apparent Kd was calculated using the Biacore™ B4000 evaluation software (version 1.1).

### 2.2.8 Cellular Binding Assay for Characterisation of CD40-Specific Humanized Antibodies

CD40 positive cells (Raji cells) were detatched from the culture bottle using Trypsin and were counted using a Casy cell counter. After pelleting at 4° C., the cells were resuspended in FACS Buffer (2.5% FCS in PBS), adjusted to 2.0E+06 cells/mL, and dispensed to 96-well PP V-bottom-plates (25 μL/well=5.0E+04Zellen/well).

The CD40 specific antibodies were adjusted to 20 μg/mL in FACS buffer, resulting in a final concentration of 10 μg/mL. 20 μl were added to 25 μl cell suspension and incubated for 1 h at 4° C. The cells were then washed twice in FACS buffer. After washing, the cells were resuspended in 50 μL FACS-buffer containing secondary antibody (<huIgG>-Alexa488, c=10 μg/mL) and incubated 1 h bei 4° C. The cells were then washed twice in FACS buffer and resuspended in 70 μl/well FACS buffer for measurement using a FACS Canto (BD, Pharmingen).

In Table 23 the affinity of the humanized CD40 antibodies (measured by Biacore) and the cellular binding to CD40 expressing cells (Raji cells) is shown.

TABLE 23
Affinity and cellular binding of humanized
CD40 antibodies to CD40 expressing cells

| | | | | | EC₅₀ |
|---|---|---|---|---|---|
| | | | | | [μg/ml] |
| | | | | | cellular |
| | | Affinity | | | binding |
| ID | VH/VL | [nM] | Ka (1/Ms) | Kd (1/s) | (Raji) |
| P1AD4470 | contro1 | 4.6 | 1.69E+06 | 7.81E−03 | 0.09 |
| P1AE0400 | VL2a/VH2a | 4.2 | 1.68E+06 | 6.99E−03 | 0.12 |
| P1AE0401 | VL2a/VH2b | 4.6 | 1.69E+06 | 7.87E−03 | 0.13 |
| P1AE0402 | VL2a/VH2c | 4.2 | 1.67E+06 | 7.09E−03 | 0.13 |
| P1AE0403 | VL2a/VH2d | 29 | 1.40E+06 | 4.07E−02 | 0.12 |
| P1AE0404 | VL2b/VH2a | 4.2 | 1.63E+06 | 6.93E−03 | 0.11 |
| P1AE0405 | VL2b/VH2b | 5.1 | 1.61E+06 | 8.14E−03 | 0.09 |
| P1AE0406 | VL2b/VH2c | 4.2 | 1.67E+06 | 7.09E−03 | 0.09 |
| P1AE0407 | VL2b/VH2d | 30 | 1.19E+06 | 3.55E−02 | 0.12 |
| P1AE0816 | contro1 | 8.7 | 2.53E+06 | 2.19E−02 | 0.09 |
| P1AE0817 | VH1a/VL1a | 2.5 | 2.40E+06 | 5.93E−03 | 0.09 |
| P1AE0818 | VH1c/VL1c | 3.2 | 2.63E+06 | 8.47E−03 | 0.14 |
| P1AE0819 | VH1d/VL1d | 3.4 | 2.59E+06 | 8.77E−03 | 0.11 |
| P1AE0993 | VH1a/VL1c | 3.4 | 2.68E+06 | 8.98E−03 | 0.13 |
| P1AE0996 | VH1a/VL1d | 3.5 | 2.59E+06 | 9.08E−03 | 0.12 |
| P1AE0997 | VH1c/VL1a | 2.3 | 2.59E+06 | 6.03E−03 | 0.12 |
| P1AE0998 | VH1c/VL1d | 3.3 | 2.70E+06 | 8.96E−03 | 0.12 |
| P1AE0999 | VH1d/VL1a | 2.4 | 2.45E+06 | 5.92E−03 | 0.15 |
| P1AE1000 | VH1d/VL1c | 3.2 | 2.68E+06 | 8.62E−03 | 0.14 |
| P1AE1001 | VH1a/VL1b | 2.7 | 2.56E+06 | 6.81E−03 | 0.08 |
| P1AE1002 | VH1b/VL1a | 2.2 | 2.54E+06 | 5.57E−03 | 0.13 |
| P1AE1003 | VH1b/VL1b | 2.5 | 2.46E+06 | 6.06E−03 | 0.13 |
| P1AE1004 | VH1b/VL1c | 3 | 2.63E+06 | 7.95E−03 | 0.14 |
| P1AE1005 | VH1b/VL1d | 3.2 | 2.58E+06 | 8.16E−03 | 0.11 |
| P1AE1006 | VH1c/VL1b | 2.6 | 2.53E+06 | 6.51E−03 | 0.14 |
| P1AE1007 | VH1d/VL1b | 2.7 | 2.50E+06 | 6.62E−03 | 0.12 |

### 2.2.9 Antibody Characterisation by UHR-ESI-QTOF Mass Spectrometry

The samples were desalted by HPLC on a Sephadex G25 5×250 mm column (Amersham Biosciences, Freiburg, Germany) using 40% acetonitrile with 2% formic acid (v/v). The total mass was determined by UHR-ESI-QTOF MS on a maXis 4G UHR-QTOF MS system (Bruker Daltonik, Bremen, Germany) equipped with a TriVersa NanoMate source (Advion, Ithaca, NY). Data acquisition was done at 900-4000 m/z (ISCID: 0.0 eV). The raw mass spectra were evaluated and transformed into individual relative molar masses using an in-house developed software tool.

### 2.2.10 Thermal Stability Evaluation of Antibodies

Samples are prepared at a concentration of 1 mg/mL in 20 mM Histidine/Histidine chloride, 140 mM NaCl, pH 6.0, transferred into an optical 384-well plate by centrifugation through a 0.4 μm filter plate and covered with paraffine oil. The hydrodynamic radius is measured repeatedly by dynamic light scattering on a DynaPro Plate Reader (Wyatt) while the samples are heated with a rate of 0.05° C./min from 25° C. to 80° C. Alternatively, samples were transferred into a 10 μL micro-cuvette array and static light scattering data as well as fluorescence data upon excitation with a 266 nm laser were recorded with an Optim1000 instrument (Avacta Inc.), while they were heated at a rate of 0.1° C./min from 25° C. to 90° C. The aggregation onset temperature is defined as the temperature at which the hydrodynamic radius (DLS) or the scattered light intensity (Optim1000) starts to increase. The melting temperature is defined as the inflection point in a graph showing fluorescence intensity vs. wavelength.

### Example 3

Generation and Production of Bispecific Constructs with New Anti-FAP Clone 212 and its Humanized Variants

### 3.1 Generation of Bispecific Antigen Binding Molecules Targeting Fibroblast Activation Protein (FAP) and CD40

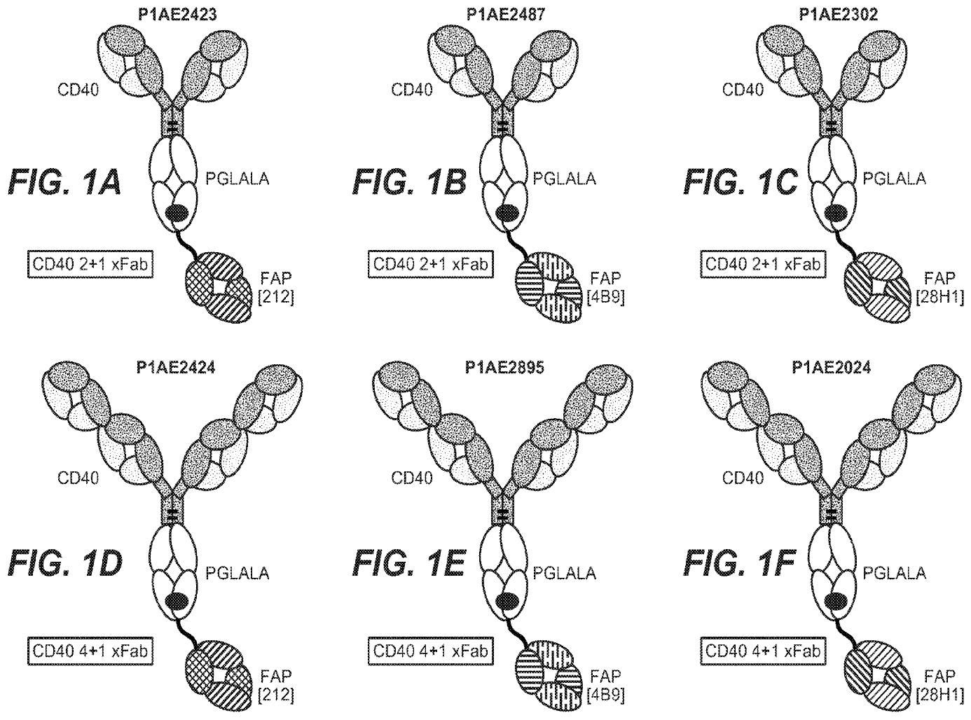

Bispecific CD40-FAP antibodies were prepared in 2+1 format consisting of two CD40 binding moieties combined with one FAP binding moiety at the C-terminus of an Fc (FIG. 1A to FIG. 1C) or in 4+1 format consisting of four CD40 binding moieties combined with one FAP binding moiety at the C-terminus of an Fc (FIG. 1D to FIG. 1F). The bispecific CD40-FAP antibodies included new anti-FAP clone 212 (FIG. 1A and FIG. 1D), however for comparison corresponding molecules with FAP clones 4B9 (FIG. 1B and FIG. 1E) and 28H1 (FIG. 1C and FIG. 1F) were prepared. The generation and preparation of FAP binders 28H1 and 4B9 has been described in WO 2012/020006 A2, which is incorporated herein by reference. To generate the 4+1 and the 2+1 molecules the knob-into-hole technology was used to achieve heterodimerization. The S354C/T366W mutations were introduced in the first heavy chain HCl (Fc knob heavy chain) and the Y349C/T366S/L368A/Y407V mutations were introduced in the second heavy chain HC2 (Fc hole heavy chain). Independent of the bispecific format, in all cases an effector silent Fc (P329G; L234, 234A) was used to abrogate binding to Fc receptors according to the method described in WO 2012/130831 A1. Sequences of the bispecific molecules are shown in Table 24.

All genes are transiently expressed under control of a chimeric MPSV promoter consisting of the MPSV core promoter combined with the CMV promoter enhancer fragment. The expression cassette also contains a synthetic polyA signal at the 3′ end of the cDNAs. The expression vector also contains the oriP region for episomal replication in EBNA (Epstein Barr Virus Nuclear Antigen) containing host cells.

TABLE 24
Amino acid sequences of the bispecific antigen binding molecules

| | | |
|---|---|---|
| | | Seq ID |
| Construct | Sequence | No |

P1AE2423
CD40 (P1AE0817) × FAP (P1AE1689) (2 + 1) C-terminal crossfab fusion

| | | |
|---|---|---|
| (P1AE1689) | QVQLVQSGAEVKKPGASVKVSCKASGYTLTDYNM | 61 |
| light chain cross | DWVRQAPGQGLEWIGDIYPNTGGTIYNQKFKGRV | |
| VH-Ckappa | TMTIDTSTSTVYMELSSLRSEDTAVYYCTRFRGIHY | |
| | AMDYWGQGTTVTVSSASVAAPSVFIFPPSDEQLKS | |
| | GTASVVCLLNNFYPREAKVQWKVDNALQSGNSQE | |
| | SVTEQDSKDSTYSLSSTLTLSKADYEKHKVYACEV | |
| | THQGLSSPVTKSFNRGEC | |
| VL1a (CD40) | DIVMTQTPLSLSVTPGQPASISCRSSQSLVHSNGNTF | 62 |
| light chain | LHWYLQKPGQSPQLLIYTVSNRFSGVPDRFSGSGS | |
| (charged) | GTDFTLKISRVEAEDVGVYFCSQTTHVPWTFGGGT | |
| | KVEIKRTVAAPSVFIFPPSDRKLKSGTASVVCLLNN | |
| | FYPREAKVQWKVDNALQSGNSQESVTEQDSKDST | |
| | YSLSSTLTLSKADYEKHKVYACEVTHQGLSSPVTK | |
| | SFNRGEC | |
| VH1a (CD40) | QVQLVQSGAEVKKPGASVKVSCKASGYSFTGYYI | 63 |
| (VHCH1 | HWVRQAPGQSLEWMGRVIPNAGGTSYNQKFKGR | |
| charged) Fc | VTLTVDKSISTAYMELSRLRSDDTAVYYCAREGIY | |
| knob_PGLALA_(P1AE1689) | WWGQGTTVTVSSASTKGPSVFPLAPSSKSTSGGTA | |
| (VL-CH1) | ALGCLVEDYFPEPVTVSWNSGALTSGVHTFPAVLQ | |
| | SSGLYSLSSVVTVPSSSLGTQTYICNVNHKPSNTKV | |
| | DEKVEPKSCDKTHTCPPCPAPEAAGGPSVFLFPPKP | |
| | KDTLMISRTPEVTCVVVDVSHEDPEVKFNWYVDG | |
| | VEVHNAKTKPREEQYNSTYRVVSVLTVLHQDWLN | |
| | GKEYKCKVSNKALGAPIEKTISKAKGQPREPQVYT | |
| | LPPCRDELTKNQVSLWCLVKGFYPSDIAVEWESNG | |
| | QPENNYKTTPPVLDSDGSFFLYSKLTVDKSRWQQG | |
| | NVFSCSVMHEALHNHYTQKSLSLSPGGGGGSGGG | |
| | GSGGGGSGGGGSEIVLTQSPATLSLSPGERATLSCR | |
| | ASESVDNYGLSFINWFQQKPGQAPRLLIYGTSNRGS | |
| | GIPARFSGSGSGTDFTLTISSLEPEDFAVYFCQQSNE | |
| | VPYTFGGGTKVEIKSSASTKGPSVFPLAPSSKSTSG | |
| | GTAALGCLVKDYFPEPVTVSWNSGALTSGVHTFPA | |
| | VLQSSGLYSLSSVVTVPSSSLGTQTYICNVNHKPSN | |
| | TKVDKKVEPKSC | |
| VH1a (CD40) | QVQLVQSGAEVKKPGASVKVSCKASGYSFTGYYI | 64 |
| (VHCH1 | HWVRQAPGQSLEWMGRVIPNAGGTSYNQKFKGR | |
| charged) Fc | VTLTVDKSISTAYMELSRLRSDDTAVYYCAREGIY | |
| hole_PGLALA | WWGQGTTVTVSSASTKGPSVFPLAPSSKSTSGGTA | |
| | ALGCLVEDYFPEPVTVSWNSGALTSGVHTFPAVLQ | |
| | SSGLYSLSSVVTVPSSSLGTQTYICNVNHKPSNTKV | |
| | DEKVEPKSCDKTHTCPPCPAPEAAGGPSVFLFPPKP | |
| | KDTLMISRTPEVTCVVVDVSHEDPEVKFNWYVDG | |
| | VEVHNAKTKPREEQYNSTYRVVSVLTVLHQDWLN | |
| | GKEYKCKVSNKALGAPIEKTISKAKGQPREPQVCT | |
| | LPPSRDELTKNQVSLSCAVKGFYPSDIAVEWESNG | |
| | QPENNYKTTPPVLDSDGSFFLVSKLTVDKSRWQQG | |
| | NVFSCSVMHEALHNHYTQKSLSLSPG | |

P1AE2734
CD40 (P1AE0817) × FAP (P1AE1689) (2 + 1) with different
C-terminal crossfab fusion

| | | |
|---|---|---|
| (P1AE1689) | EIVLTQSPATLSLSPGERATLSCRASESVDNYGLSFI | 65 |
| light chain cross | NWFQQKPGQAPRLLIYGTSNRGSGIPARFSGSGSGT | |
| VL-CH1 | DFTLTISSLEPEDFAVYFCQQSNEVPYTFGGGTKVEI | |
| | KSSASTKGPSVFPLAPSSKSTSGGTAALGCLVKDYF | |
| | PEPVTVSWNSGALTSGVHTFPAVLQSSGLYSLSSV | |
| | VTVPSSSLGTQTYICNVNHKPSNTKVDKKVEPKSC | |
| VL1a (CD40) | DIVMTQTPLSLSVTPGQPASISCRSSQSLVHSNGNTF | 66 |
| light chain | LHWYLQKPGQSPQLLIYTVSNRFSGVPDRFSGSGS | |
| | GTDFTLKISRVEAEDVGVYFCSQTTHVPWTFGGGT | |
| | KVEIKRTVAAPSVFIFPPSDEQLKSGTASVVCLLNN | |
| | FYPREAKVQWKVDNALQSGNSQESVTEQDSKDST | |
| | YSLSSTLTLSKADYEKHKVYACEVTHQGLSSPVTK | |
| | SFNRGEC | |
| VH1a (CD40) | QVQLVQSGAEVKKPGASVKVSCKASGYSFTGYYI | 67 |
| (VHCH1) Fc | HWVRQAPGQSLEWMGRVIPNAGGTSYNQKFKGR | |
| knob_PGLALA_(P1AE1689) | VTLTVDKSISTAYMELSRLRSDDTAVYYCAREGIY | |
| (VH-Ckappa) | WWGQGTTVTVSSASTKGPSVFPLAPSSKSTSGGTA | |
| | ALGCLVKDYFPEPVTVSWNSGALTSGVHTFPAVLQ | |
| | SSGLYSLSSVVTVPSSSLGTQTYICNVNHKPSNTKV | |
| | DKKVEPKSCDKTHTCPPCPAPEAAGGPSVFLFPPKP | |
| | KDTLMISRTPEVTCVVVDVSHEDPEVKFNWYVDG | |
| | VEVHNAKTKPREEQYNSTYRVVSVLTVLHQDWLN | |
| | GKEYKCKVSNKALGAPIEKTISKAKGQPREPQVYT | |
| | LPPCRDELTKNQVSLWCLVKGFYPSDIAVEWESNG | |
| | QPENNYKTTPPVLDSDGSFFLYSKLTVDKSRWQQG | |
| | NVFSCSVMHEALHNHYTQKSLSLSPGGGGGSGGG | |
| | GSGGGGSGGGGSQVQLVQSGAEVKKPGASVKVSC | |
| | KASGYTLTDYNMDWVRQAPGQGLEWIGDIYPNTG | |
| | GTIYNQKFKGRVTMTIDTSTSTVYMELSSLRSEDTA | |
| | VYYCTRFRGIHYAMDYWGQGTTVTVSSASVAAPS | |
| | VFIFPPSDEQLKSGTASVVCLLNNFYPREAKVQWK | |
| | VDNALQSGNSQESVTEQDSKDSTYSLSSTLTLSKA | |
| | DYEKHKVYACEVTHQGLSSPVTKSFNRGEC | |
| VH1a (CD40) | QVQLVQSGAEVKKPGASVKVSCKASGYSFTGYYI | 68 |
| (VHCH1) Fc | HWVRQAPGQSLEWMGRVIPNAGGTSYNQKFKGR | |
| hole_PGLALA | VTLTVDKSISTAYMELSRLRSDDTAVYYCAREGIY | |
| | WWGQGTTVTVSSASTKGPSVFPLAPSSKSTSGGTA | |
| | ALGCLVKDYFPEPVTVSWNSGALTSGVHTFPAVLQ | |
| | SSGLYSLSSVVTVPSSSLGTQTYICNVNHKPSNTKV | |
| | DKKVEPKSCDKTHTCPPCPAPEAAGGPSVFLFPPKP | |
| | KDTLMISRTPEVTCVVVDVSHEDPEVKFNWYVDG | |
| | VEVHNAKTKPREEQYNSTYRVVSVLTVLHQDWLN | |
| | GKEYKCKVSNKALGAPIEKTISKAKGQPREPQVCT | |
| | LPPSRDELTKNQVSLSCAVKGFYPSDIAVEWESNG | |
| | QPENNYKTTPPVLDSDGSFFLVSKLTVDKSRWQQG | |
| | NVFSCSVMHEALHNHYTQKSLSLSPG | |

P1AE2424
CD40 (P1AE0817) × FAP (P1AE1689) (4 + 1) with
C-terminal crossfab fusion

| | | |
|---|---|---|
| (P1AE1689) | QVQLVQSGAEVKKPGASVKVSCKASGYTLTDYNM | 61 |
| light chain cross | DWVRQAPGQGLEWIGDIYPNTGGTIYNQKFKGRV | |
| VH-Ckappa | TMTIDTSTSTVYMELSSLRSEDTAVYYCTRFRGIHY | |
| | AMDYWGQGTTVTVSSASVAAPSVFIFPPSDEQLKS | |
| | GTASVVCLLNNFYPREAKVQWKVDNALQSGNSQE | |
| | SVTEQDSKDSTYSLSSTLTLSKADYEKHKVYACEV | |
| | THQGLSSPVTKSFNRGEC | |
| VL1a (CD40) | DIVMTQTPLSLSVTPGQPASISCRSSQSLVHSNGNTF | 62 |
| light chain | LHWYLQKPGQSPQLLIYTVSNRFSGVPDRFSGSGS | |
| (charged) | GTDFTLKISRVEAEDVGVYFCSQTTHVPWTFGGGT | |
| | KVEIKRTVAAPSVFIFPPSDRKLKSGTASVVCLLNN | |
| | FYPREAKVQWKVDNALQSGNSQESVTEQDSKDST | |
| | YSLSSTLTLSKADYEKHKVYACEVTHQGLSSPVTK | |
| | SFNRGEC | |
| VH1a (CD40) | QVQLVQSGAEVKKPGASVKVSCKASGYSFTGYYI | 69 |
| (VHCH1 | HWVRQAPGQSLEWMGRVIPNAGGTSYNQKFKGR | |
| charged_VH1a | VTLTVDKSISTAYMELSRLRSDDTAVYYCAREGIY | |
| (CD40) | WWGQGTTVTVSSASTKGPSVFPLAPSSKSTSGGTA | |
| (VHCH1 | ALGCLVEDYFPEPVTVSWNSGALTSGVHTFPAVLQ | |
| charged)-Fc | SSGLYSLSSVVTVPSSSLGTQTYICNVNHKPSNTKV | |
| knob_PGLALA_(P1AE1689) | DEKVEPKSCDGGGGSGGGGSQVQLVQSGAEVKKP | |
| (VL-CH1) | GASVKVSCKASGYSFTGYYIHWVRQAPGQSLEWM | |
| | GRVIPNAGGTSYNQKFKGRVTLTVDKSISTAYMEL | |
| | SRLRSDDTAVYYCAREGIYWWGQGTTVTVSSAST | |
| | KGPSVFPLAPSSKSTSGGTAALGCLVEDYFPEPVTV | |
| | SWNSGALTSGVHTFPAVLQSSGLYSLSSVVTVPSSS | |
| | LGTQTYICNVNHKPSNTKVDEKVEPKSCDKTHTCP | |
| | PCPAPEAAGGPSVFLFPPKPKDTLMISRTPEVTCVV | |
| | VDVSHEDPEVKFNWYVDGVEVHNAKTKPREEQY | |
| | NSTYRVVSVLTVLHQDWLNGKEYKCKVSNKALG | |
| | APIEKTISKAKGQPREPQVYTLPPCRDELTKNQVSL | |
| | WCLVKGFYPSDIAVEWESNGQPENNYKTTPPVLDS | |
| | DGSFFLYSKLTVDKSRWQQGNVFSCSVMHEALHN | |
| | HYTQKSLSLSPGGGGGSGGGGSGGGGSGGGGSEIV | |
| | LTQSPATLSLSPGERATLSCRASESVDNYGLSFINW | |
| | FQQKPGQAPRLLIYGTSNRGSGIPARFSGSGSGTDF | |
| | TLTISSLEPEDFAVYFCQQSNEVPYTFGGGTKVEIKS | |
| | SASTKGPSVFPLAPSSKSTSGGTAALGCLVKDYFPE | |
| | PVTVSWNSGALTSGVHTFPAVLQSSGLYSLSSVVT | |
| | VPSSSLGTQTYICNVNHKPSNTKVDKKVEPKSC | |
| VH1a (CD40) | QVQLVQSGAEVKKPGASVKVSCKASGYSFTGYYI | 70 |
| (VHCH1 | HWVRQAPGQSLEWMGRVIPNAGGTSYNQKFKGR | |
| charged)_VH1a | VTLTVDKSISTAYMELSRLRSDDTAVYYCAREGIY | |
| (CD40) | WWGQGTTVTVSSASTKGPSVFPLAPSSKSTSGGTA | |
| (VHCH1 | ALGCLVEDYFPEPVTVSWNSGALTSGVHTFPAVLQ | |
| charged)-Fc | SSGLYSLSSVVTVPSSSLGTQTYICNVNHKPSNTKV | |
| hole_PGLALA | DEKVEPKSCDGGGGSGGGGSQVQLVQSGAEVKKP | |
| | GASVKVSCKASGYSFTGYYIHWVRQAPGQSLEWM | |
| | GRVIPNAGGTSYNQKFKGRVTLTVDKSISTAYMEL | |
| | SRLRSDDTAVYYCAREGIYWWGQGTTVTVSSAST | |
| | KGPSVFPLAPSSKSTSGGTAALGCLVEDYFPEPVTV | |
| | SWNSGALTSGVHTFPAVLQSSGLYSLSSVVTVPSSS | |
| | LGTQTYICNVNHKPSNTKVDEKVEPKSCDKTHTCP | |
| | PCPAPEAAGGPSVFLFPPKPKDTLMISRTPEVTCVV | |
| | VDVSHEDPEVKFNWYVDGVEVHNAKTKPREEQY | |
| | NSTYRVVSVLTVLHQDWLNGKEYKCKVSNKALG | |
| | APIEKTISKAKGQPREPQVCTLPPSRDELTKNQVSLS | |
| | CAVKGFYPSDIAVEWESNGQPENNYKTTPPVLDSD | |
| | GSFFLVSKLTVDKSRWQQGNVFSCSVMHEALHNH | |
| | YTQKSLSLSPG | |

P1AE2487
CD40 (P1AE0817) × FAP (4B9) (2 + 1) C-terminal crossfab fusion

| | | |
|---|---|---|
| 4B9 light chain | EIVLTQSPGTLSLSPGERATLSCRASQSVTSSYLAW | 71 |
| cross VL-CH1 | YQQKPGQAPRLLINVGSRRATGIPDRFSGSGSGTDF | |
| | TLTISRLEPEDFAVYYCQQGIMLPPTFGQGTKVEIK | |
| | SSASTKGPSVFPLAPSSKSTSGGTAALGCLVKDYFP | |
| | EPVTVSWNSGALTSGVHTFPAVLQSSGLYSLSSVV | |
| | TVPSSSLGTQTYICNVNHKPSNTKVDKKVEPKSC | |
| VL1a (CD40) | DIVMTQTPLSLSVTPGQPASISCRSSQSLVHSNGNTF | 66 |
| light chain | LHWYLQKPGQSPQLLIYTVSNRFSGVPDRFSGSGS | |
| | GTDFTLKISRVEAEDVGVYFCSQTTHVPWTFGGGT | |
| | KVEIKRTVAAPSVFIFPPSDEQLKSGTASVVCLLNN | |
| | FYPREAKVQWKVDNALQSGNSQESVTEQDSKDST | |
| | YSLSSTLTLSKADYEKHKVYACEVTHQGLSSPVTK | |
| | SFNRGEC | |
| VH1a (CD40) | QVQLVQSGAEVKKPGASVKVSCKASGYSFTGYYI | 72 |
| (VHCH1) Fc | HWVRQAPGQSLEWMGRVIPNAGGTSYNQKFKGR | |
| knob_PGLALA_4B9 | VTLTVDKSISTAYMELSRLRSDDTAVYYCAREGIY | |
| (VH-Ckappa) | WWGQGTTVTVSSASTKGPSVFPLAPSSKSTSGGTA | |
| | ALGCLVKDYFPEPVTVSWNSGALTSGVHTFPAVLQ | |
| | SSGLYSLSSVVTVPSSSLGTQTYICNVNHKPSNTKV | |
| | DKKVEPKSCDKTHTCPPCPAPEAAGGPSVFLFPPKP | |
| | KDTLMISRTPEVTCVVVDVSHEDPEVKFNWYVDG | |
| | VEVHNAKTKPREEQYNSTYRVVSVLTVLHQDWLN | |
| | GKEYKCKVSNKALGAPIEKTISKAKGQPREPQVYT | |
| | LPPCRDELTKNQVSLWCLVKGFYPSDIAVEWESNG | |
| | QPENNYKTTPPVLDSDGSFFLYSKLTVDKSRWQQG | |
| | NVFSCSVMHEALHNHYTQKSLSLSPGGGGGSGGG | |
| | GSGGGGSGGGGSEVQLLESGGGLVQPGGSLRLSCA | |
| | ASGFTFSSYAMSWVRQAPGKGLEWVSAIIGSGAST | |
| | YYADSVKGRFTISRDNSKNTLYLQMNSLRAEDTAV | |
| | YYCAKGWFGGFNYWGQGTLVTVSSASVAAPSVFI | |
| | FPPSDEQLKSGTASVVCLLNNFYPREAKVQWKVD | |
| | NALQSGNSQESVTEQDSKDSTYSLSSTLTLSKADYE | |
| | KHKVYACEVTHQGLSSPVTKSFNRGEC | |
| VH1a (CD40) | QVQLVQSGAEVKKPGASVKVSCKASGYSFTGYYI | 68 |
| (VHCH1) Fc | HWVRQAPGQSLEWMGRVIPNAGGTSYNQKFKGR | |
| hole_PGLALA | VTLTVDKSISTAYMELSRLRSDDTAVYYCAREGIY | |
| | WWGQGTTVTVSSASTKGPSVFPLAPSSKSTSGGTA | |
| | ALGCLVKDYFPEPVTVSWNSGALTSGVHTFPAVLQ | |
| | SSGLYSLSSVVTVPSSSLGTQTYICNVNHKPSNTKV | |
| | DKKVEPKSCDKTHTCPPCPAPEAAGGPSVFLFPPKP | |
| | KDTLMISRTPEVTCVVVDVSHEDPEVKFNWYVDG | |
| | VEVHNAKTKPREEQYNSTYRVVSVLTVLHQDWLN | |
| | GKEYKCKVSNKALGAPIEKTISKAKGQPREPQVCT | |
| | LPPSRDELTKNQVSLSCAVKGFYPSDIAVEWESNG | |
| | QPENNYKTTPPVLDSDGSFFLVSKLTVDKSRWQQG | |
| | NVFSCSVMHEALHNHYTQKSLSLSPG | |

P1AE2895
CD40 (P1AE0817) × FAP (4B9) (4 + 1) C-terminal crossfab fusion

| | | |
|---|---|---|
| 4B9 light chain | EIVLTQSPGTLSLSPGERATLSCRASQSVTSSYLAW | 71 |
| cross VL-CH1 | YQQKPGQAPRLLINVGSRRATGIPDRFSGSGSGTDF | |
| | TLTISRLEPEDFAVYYCQQGIMLPPTFGQGTKVEIK | |
| | SSASTKGPSVFPLAPSSKSTSGGTAALGCLVKDYFP | |
| | EPVTVSWNSGALTSGVHTFPAVLQSSGLYSLSSVV | |
| | TVPSSSLGTQTYICNVNHKPSNTKVDKKVEPKSC | |
| VL1a (CD40) | DIVMTQTPLSLSVTPGQPASISCRSSQSLVHSNGNTF | 66 |
| light chain | LHWYLQKPGQSPQLLIYTVSNRFSGVPDRFSGSGS | |
| | GTDFTLKISRVEAEDVGVYFCSQTTHVPWTFGGGT | |
| | KVEIKRTVAAPSVFIFPPSDEQLKSGTASVVCLLNN | |
| | FYPREAKVQWKVDNALQSGNSQESVTEQDSKDST | |
| | YSLSSTLTLSKADYEKHKVYACEVTHQGLSSPVTK | |
| | SFNRGEC | |
| VH1a (CD40) | QVQLVQSGAEVKKPGASVKVSCKASGYSFTGYYI | 73 |
| (VHCH1)_VH1a | HWVRQAPGQSLEWMGRVIPNAGGTSYNQKFKGR | |
| (CD40) | VTLTVDKSISTAYMELSRLRSDDTAVYYCAREGIY | |
| (VHCH1)-Fc | WWGQGTTVTVSSASTKGPSVFPLAPSSKSTSGGTA | |
| knob_PGLALA_(4B9) | ALGCLVKDYFPEPVTVSWNSGALTSGVHTFPAVLQ | |
| (VH-Ckappa) | SSGLYSLSSVVTVPSSSLGTQTYICNVNHKPSNTKV | |
| | DKKVEPKSCDGGGGSGGGGSQVQLVQSGAEVKKP | |
| | GASVKVSCKASGYSFTGYYIHWVRQAPGQSLEWM | |
| | GRVIPNAGGTSYNQKFKGRVTLTVDKSISTAYMEL | |
| | SRLRSDDTAVYYCAREGIYWWGQGTTVTVSSAST | |
| | KGPSVFPLAPSSKSTSGGTAALGCLVKDYFPEPVTV | |
| | SWNSGALTSGVHTFPAVLQSSGLYSLSSVVTVPSSS | |
| | LGTQTYICNVNHKPSNTKVDKKVEPKSCDKTHTCP | |
| | PCPAPEAAGGPSVFLFPPKPKDTLMISRTPEVTCVV | |
| | VDVSHEDPEVKFNWYVDGVEVHNAKTKPREEQY | |
| | NSTYRVVSVLTVLHQDWLNGKEYKCKVSNKALG | |
| | APIEKTISKAKGQPREPQVYTLPPCRDELTKNQVSL | |
| | WCLVKGFYPSDIAVEWESNGQPENNYKTTPPVLDS | |
| | DGSFFLYSKLTVDKSRWQQGNVFSCSVMHEALHN | |
| | HYTQKSLSLSPGGGGGSGGGGSGGGGSGGGGSEV | |
| | QLLESGGGLVQPGGSLRLSCAASGFTFSSYAMSWV | |
| | RQAPGKGLEWVSAIIGSGASTYYADSVKGRFTISRD | |
| | NSKNTLYLQMNSLRAEDTAVYYCAKGWFGGFNY | |
| | WGQGTLVTVSSASVAAPSVFIFPPSDEQLKSGTASV | |
| | VCLLNNFYPREAKVQWKVDNALQSGNSQESVTEQ | |
| | DSKDSTYSLSSTLTLSKADYEKHKVYACEVTHQGL | |
| | SSPVTKSFNRGEC | |
| VH1a (CD40) | QVQLVQSGAEVKKPGASVKVSCKASGYSFTGYYI | 74 |
| (VHCH1)_VH1a | HWVRQAPGQSLEWMGRVIPNAGGTSYNQKFKGR | |
| (CD40) | VTLTVDKSISTAYMELSRLRSDDTAVYYCAREGIY | |
| (VHCH1)-Fc | WWGQGTTVTVSSASTKGPSVFPLAPSSKSTSGGTA | |
| hole_PGLALA | ALGCLVKDYFPEPVTVSWNSGALTSGVHTFPAVLQ | |
| | SSGLYSLSSVVTVPSSSLGTQTYICNVNHKPSNTKV | |
| | DKKVEPKSCDGGGGSGGGGSQVQLVQSGAEVKKP | |
| | GASVKVSCKASGYSFTGYYIHWVRQAPGQSLEWM | |
| | GRVIPNAGGTSYNQKFKGRVTLTVDKSISTAYMEL | |
| | SRLRSDDTAVYYCAREGIYWWGQGTTVTVSSAST | |
| | KGPSVFPLAPSSKSTSGGTAALGCLVKDYFPEPVTV | |
| | SWNSGALTSGVHTFPAVLQSSGLYSLSSVVTVPSSS | |
| | LGTQTYICNVNHKPSNTKVDKKVEPKSCDKTHTCP | |
| | PCPAPEAAGGPSVFLFPPKPKDTLMISRTPEVTCVV | |
| | VDVSHEDPEVKFNWYVDGVEVHNAKTKPREEQY | |
| | NSTYRVVSVLTVLHQDWLNGKEYKCKVSNKALG | |
| | APIEKTISKAKGQPREPQVCTLPPSRDELTKNQVSLS | |
| | CAVKGFYPSDIAVEWESNGQPENNYKTTPPVLDSD | |
| | GSFFLVSKLTVDKSRWQQGNVFSCSVMHEALHNH | |
| | YTQKSLSLSPG | |

P1AE2302
CD40 (P1AE0817) × FAP (28H1) (2 + 1) C-terminal crossfab fusion

| | | |
|---|---|---|
| 28H1 light chain | EVQLLESGGGLVQPGGSLRLSCAASGFTFSSHAMS | 75 |
| cross VH- | WVRQAPGKGLEWVSAIWASGEQYYADSVKGRFTI | |
| Ckappa | SRDNSKNTLYLQMNSLRAEDTAVYYCAKGWLGN | |
| | FDYWGQGTLVTVSSASVAAPSVFIFPPSDEQLKSGT | |
| | ASVVCLLNNFYPREAKVQWKVDNALQSGNSQESV | |
| | TEQDSKDSTYSLSSTLTLSKADYEKHKVYACEVTH | |
| | QGLSSPVTKSFNRGEC | |
| VL1a (CD40) | DIVMTQTPLSLSVTPGQPASISCRSSQSLVHSNGNTF | 62 |
| light chain | LHWYLQKPGQSPQLLIYTVSNRFSGVPDRFSGSGS | |
| (charged) | GTDFTLKISRVEAEDVGVYFCSQTTHVPWTFGGGT | |
| | KVEIKRTVAAPSVFIFPPSDRKLKSGTASVVCLLNN | |
| | FYPREAKVQWKVDNALQSGNSQESVTEQDSKDST | |
| | YSLSSTLTLSKADYEKHKVYACEVTHQGLSSPVTK | |
| | SFNRGEC | |
| VH1a (CD40) | QVQLVQSGAEVKKPGASVKVSCKASGYSFTGYYI | 76 |
| (VHCH1 | HWVRQAPGQSLEWMGRVIPNAGGTSYNQKFKGR | |
| charged) Fc | VTLTVDKSISTAYMELSRLRSDDTAVYYCAREGIY | |
| knob_PGLALA_28H1 | WWGQGTTVTVSSASTKGPSVFPLAPSSKSTSGGTA | |
| (VL-CH1) | ALGCLVEDYFPEPVTVSWNSGALTSGVHTFPAVLQ | |
| | SSGLYSLSSVVTVPSSSLGTQTYICNVNHKPSNTKV | |
| | DEKVEPKSCDKTHTCPPCPAPEAAGGPSVFLFPPKP | |
| | KDTLMISRTPEVTCVVVDVSHEDPEVKFNWYVDG | |
| | VEVHNAKTKPREEQYNSTYRVVSVLTVLHQDWLN | |
| | GKEYKCKVSNKALGAPIEKTISKAKGQPREPQVYT | |
| | LPPCRDELTKNQVSLWCLVKGFYPSDIAVEWESNG | |
| | QPENNYKTTPPVLDSDGSFFLYSKLTVDKSRWQQG | |
| | NVFSCSVMHEALHNHYTQKSLSLSPGGGGGSGGG | |
| | GSGGGGSGGGGSEIVLTQSPGTLSLSPGERATLSCR | |
| | ASQSVSRSYLAWYQQKPGQAPRLLIIGASTRATGIP | |
| | DRFSGSGSGTDFTLTISRLEPEDFAVYYCQQGQVIP | |
| | PTFGQGTKVEIKSSASTKGPSVFPLAPSSKSTSGGTA | |
| | ALGCLVKDYFPEPVTVSWNSGALTSGVHTFPAVLQ | |
| | SSGLYSLSSVVTVPSSSLGTQTYICNVNHKPSNTKV | |
| | DKKVEPKSC | |
| VH1a (CD40) | QVQLVQSGAEVKKPGASVKVSCKASGYSFTGYYI | 64 |
| (VHCH1 | HWVRQAPGQSLEWMGRVIPNAGGTSYNQKFKGR | |
| charged) Fc | VTLTVDKSISTAYMELSRLRSDDTAVYYCAREGIY | |
| hole_PGLALA | WWGQGTTVTVSSASTKGPSVFPLAPSSKSTSGGTA | |
| | ALGCLVEDYFPEPVTVSWNSGALTSGVHTFPAVLQ | |
| | SSGLYSLSSVVTVPSSSLGTQTYICNVNHKPSNTKV | |
| | DEKVEPKSCDKTHTCPPCPAPEAAGGPSVFLFPPKP | |
| | KDTLMISRTPEVTCVVVDVSHEDPEVKFNWYVDG | |
| | VEVHNAKTKPREEQYNSTYRVVSVLTVLHQDWLN | |
| | GKEYKCKVSNKALGAPIEKTISKAKGQPREPQVCT | |
| | LPPSRDELTKNQVSLSCAVKGFYPSDIAVEWESNG | |
| | QPENNYKTTPPVLDSDGSFFLVSKLTVDKSRWQQG | |
| | NVFSCSVMHEALHNHYTQKSLSLSPG | |

P1AE2024
CD40 (P1AE0817) × FAP (28H1) (4 + 1) C-terminal crossfab fusion

| | | |
|---|---|---|
| 28H1 light chain | EVQLLESGGGLVQPGGSLRLSCAASGFTFSSHAMS | 75 |
| cross VH-Ckappa | WVRQAPGKGLEWVSAIWASGEQYYADSVKGRFTI | |
| | SRDNSKNTLYLQMNSLRAEDTAVYYCAKGWLGN | |
| | FDYWGQGTLVTVSSASVAAPSVFIFPPSDEQLKSGT | |
| | ASVVCLLNNFYPREAKVQWKVDNALQSGNSQESV | |
| | TEQDSKDSTYSLSSTLTLSKADYEKHKVYACEVTH | |
| | QGLSSPVTKSFNRGEC | |
| VL1a (CD40) | DIVMTQTPLSLSVTPGQPASISCRSSQSLVHSNGNTF | 62 |
| light chain | LHWYLQKPGQSPQLLIYTVSNRFSGVPDRFSGSGS | |
| (charged) | GTDFTLKISRVEAEDVGVYFCSQTTHVPWTFGGGT | |
| | KVEIKRTVAAPSVFIFPPSDRKLKSGTASVVCLLNN | |
| | FYPREAKVQWKVDNALQSGNSQESVTEQDSKDST | |
| | YSLSSTLTLSKADYEKHKVYACEVTHQGLSSPVTK | |
| | SFNRGEC | |
| VH1a (CD40) | QVQLVQSGAEVKKPGASVKVSCKASGYSFTGYYI | 77 |
| (VHCH1 | HWVRQAPGQSLEWMGRVIPNAGGTSYNQKFKGR | |
| charged)_VH1a | VTLTVDKSISTAYMELSRLRSDDTAVYYCAREGIY | |
| (CD40) | WWGQGTTVTVSSASTKGPSVFPLAPSSKSTSGGTA | |
| (VHCH1 | ALGCLVEDYFPEPVTVSWNSGALTSGVHTFPAVLQ | |
| charged) Fc | SSGLYSLSSVVTVPSSSLGTQTYICNVNHKPSNTKV | |
| knob_PGLALA_28H1 | DEKVEPKSCDGGGGSGGGGSQVQLVQSGAEVKKP | |
| (VL-CH1) | GASVKVSCKASGYSFTGYYIHWVRQAPGQSLEWM | |
| | GRVIPNAGGTSYNQKFKGRVTLTVDKSISTAYMEL | |
| | SRLRSDDTAVYYCAREGIYWWGQGTTVTVSSAST | |
| | KGPSVFPLAPSSKSTSGGTAALGCLVEDYFPEPVTV | |
| | SWNSGALTSGVHTFPAVLQSSGLYSLSSVVTVPSSS | |
| | LGTQTYICNVNHKPSNTKVDEKVEPKSCDKTHTCP | |
| | PCPAPEAAGGPSVFLFPPKPKDTLMISRTPEVTCVV | |
| | VDVSHEDPEVKFNWYVDGVEVHNAKTKPREEQY | |
| | NSTYRVVSVLTVLHQDWLNGKEYKCKVSNKALG | |
| | APIEKTISKAKGQPREPQVYTLPPCRDELTKNQVSL | |
| | WCLVKGFYPSDIAVEWESNGQPENNYKTTPPVLDS | |
| | DGSFFLYSKLTVDKSRWQQGNVFSCSVMHEALHN | |
| | HYTQKSLSLSPGGGGGSGGGGSGGGGSGGGGSEIV | |
| | LTQSPGTLSLSPGERATLSCRASQSVSRSYLAWYQ | |
| | QKPGQAPRLLIIGASTRATGIPDRFSGSGSGTDFTLT | |
| | ISRLEPEDFAVYYCQQGQVIPPTFGQGTKVEIKSSAS | |
| | TKGPSVFPLAPSSKSTSGGTAALGCLVKDYFPEPVT | |
| | VSWNSGALTSGVHTFPAVLQSSGLYSLSSVVTVPSS | |
| | SLGTQTYICNVNHKPSNTKVDKKVEPKSC | |
| VH1a (CD40) | QVQLVQSGAEVKKPGASVKVSCKASGYSFTGYYI | 70 |
| (VHCH1 | HWVRQAPGQSLEWMGRVIPNAGGTSYNQKFKGR | |
| charged)_VH1a | VTLTVDKSISTAYMELSRLRSDDTAVYYCAREGIY | |
| (CD40) | WWGQGTTVTVSSASTKGPSVFPLAPSSKSTSGGTA | |
| (VHCH1 | ALGCLVEDYFPEPVTVSWNSGALTSGVHTFPAVLQ | |
| charged)-Fc | SSGLYSLSSVVTVPSSSLGTQTYICNVNHKPSNTKV | |
| hole_PGLALA | DEKVEPKSCDGGGGSGGGGSQVQLVQSGAEVKKP | |
| | GASVKVSCKASGYSFTGYYIHWVRQAPGQSLEWM | |
| | GRVIPNAGGTSYNQKFKGRVTLTVDKSISTAYMEL | |
| | SRLRSDDTAVYYCAREGIYWWGQGTTVTVSSAST | |
| | KGPSVFPLAPSSKSTSGGTAALGCLVEDYFPEPVTV | |
| | SWNSGALTSGVHTFPAVLQSSGLYSLSSVVTVPSSS | |
| | LGTQTYICNVNHKPSNTKVDEKVEPKSCDKTHTCP | |
| | PCPAPEAAGGPSVFLFPPKPKDTLMISRTPEVTCVV | |
| | VDVSHEDPEVKFNWYVDGVEVHNAKTKPREEQY | |
| | NSTYRVVSVLTVLHQDWLNGKEYKCKVSNKALG | |
| | APIEKTISKAKGQPREPQVCTLPPSRDELTKNQVSLS | |
| | CAVKGFYPSDIAVEWESNGQPENNYKTTPPVLDSD | |
| | GSFFLVSKLTVDKSRWQQGNVFSCSVMHEALHNH | |
| | YTQKSLSLSPG | |

P1AF0873
CD40 (P1AE0817) × FAP (P1AE1689) (1 + 1) C-terminal crossfab fusion

| | | |
|---|---|---|
| (P1AE1689) | QVQLVQSGAEVKKPGASVKVSCKASGYTLTDYNM | 61 |
| light chain cross | DWVRQAPGQGLEWIGDIYPNTGGTIYNQKFKGRV | |
| VH-Ckappa | TMTIDTSTSTVYMELSSLRSEDTAVYYCTRFRGIHY | |
| | AMDYWGQGTTVTVSSASVAAPSVFIFPPSDEQLKS | |
| | GTASVVCLLNNFYPREAKVQWKVDNALQSGNSQE | |
| | SVTEQDSKDSTYSLSSTLTLSKADYEKHKVYACEV | |
| | THQGLSSPVTKSFNRGEC | |
| VL1a (CD40) | DIVMTQTPLSLSVTPGQPASISCRSSQSLVHSNGNTF | 62 |
| light chain | LHWYLQKPGQSPQLLIYTVSNRFSGVPDRFSGSGS | |
| (charged) | GTDFTLKISRVEAEDVGVYFCSQTTHVPWTFGGGT | |
| | KVEIKRTVAAPSVFIFPPSDRKLKSGTASVVCLLNN | |
| | FYPREAKVQWKVDNALQSGNSQESVTEQDSKDST | |
| | YSLSSTLTLSKADYEKHKVYACEVTHQGLSSPVTK | |
| | SFNRGEC | |
| VH1a (CD40) | QVQLVQSGAEVKKPGASVKVSCKASGYSFTGYYI | 63 |
| (VHCH1 | HWVRQAPGQSLEWMGRVIPNAGGTSYNQKFKGR | |
| charged) Fc | VTLTVDKSISTAYMELSRLRSDDTAVYYCAREGIY | |
| knob_PGLALA | WWGQGTTVTVSSASTKGPSVFPLAPSSKSTSGGTA | |
| (P1AE1689) | ALGCLVEDYFPEPVTVSWNSGALTSGVHTFPAVLQ | |
| (VL-CH1) | SSGLYSLSSVVTVPSSSLGTQTYICNVNHKPSNTKV | |
| | DEKVEPKSCDKTHTCPPCPAPEAAGGPSVFLFPPKP | |
| | KDTLMISRTPEVTCVVVDVSHEDPEVKFNWYVDG | |
| | VEVHNAKTKPREEQYNSTYRVVSVLTVLHQDWLN | |
| | GKEYKCKVSNKALGAPIEKTISKAKGQPREPQVYT | |
| | LPPCRDELTKNQVSLWCLVKGFYPSDIAVEWESNG | |
| | QPENNYKTTPPVLDSDGSFFLYSKLTVDKSRWQQG | |
| | NVFSCSVMHEALHNHYTQKSLSLSPGGGGGSGGG | |
| | GSGGGGSGGGGSEIVLTQSPATLSLSPGERATLSCR | |
| | ASESVDNYGLSFINWFQQKPGQAPRLLIYGTSNRGS | |
| | GIPARFSGSGSGTDFTLTISSLEPEDFAVYFCQQSNE | |
| | VPYTFGGGTKVEIKSSASTKGPSVFPLAPSSKSTSG | |
| | GTAALGCLVKDYFPEPVTVSWNSGALTSGVHTFPA | |
| | VLQSSGLYSLSSVVTVPSSSLGTQTYICNVNHKPSN | |
| | TKVDKKVEPKSC | |
| Fc hole | DKTHTCPPCPAPEAAGGPSVFLFPPKPKDTLMISRT | 98 |
| | PEVTCVVVDVSHEDPEVKFNWYVDGVEVHNAKT | |
| | KPREEQYNSTYRVVSVLTVLHQDWLNGKEYKCKV | |
| | SNKALGAPIEKTISKAKGQPREPQVCTLPPSRDELT | |
| | KNQVSLSCAVKGFYPSDIAVEWESNGQPENNYKTT | |
| | PPVLDSDGSFFLVSKLTVDKSRWQQGNWSCSVMH | |
| | EALHNHYTQKSLSLSPG | |

P1AA9663
CD40 (huCD40) × FAP (28H1) (2 + 2) C-terminal crossfab fusion

| | | |
|---|---|---|
| 28H1 light chain | EVQLLESGGGLVQPGGSLRLSCAASGFTFSSHAMS | 75 |
| cross VH-Ckappa | WVRQAPGKGLEWVSAIWASGEQYYADSVKGRFTI | |
| | SRDNSKNTLYLQMNSLRAEDTAVYYCAKGWLGN | |
| | FDYWGQGTLVTVSSASVAAPSVFIFPPSDEQLKSGT | |
| | ASVVCLLNNFYPREAKVQWKVDNALQSGNSQESV | |
| | TEQDSKDSTYSLSSTLTLSKADYEKHKVYACEVTH | |
| | QGLSSPVTKSFNRGEC | |
| CD40 light | DIQMTQSPSSLSASVGDRVTITCRSSQSLVHSNGNT | 105 |
| chain (charged) | FLHWYQQKPGKAPKLLIYTVSNRFSGVPSRFSGSGS | |
| | GTDFTLTISSLQPEDFATYFCSQTTHVPWTFGQGTK | |
| | VEIKRTVAAPSVFIFPPSDRKLKSGTASVVCLLNNF | |
| | YPREAKVQWKVDNALQSGNSQESVTEQDSKDSTY | |
| | SLSSTLTLSKADYEKHKVYACEVTHQGLSSPVTKS | |
| | FNRGEC | |
| CD40 (VHCH1 | EVQLVESGGGLVQPGGSLRLSCAASGYSFTGYYIH | 106 |
| charged) Fc | WVRQAPGKGLEWVARVIPNAGGTSYNQKFKGRFT | |
| PGLALA FAP | LSVDNSKNTAYLQMNSLRAEDTAVYYCAREGIYW | |
| (VL-CH1) | WGQGTLVTVSSASTKGPSVFPLAPSSKSTSGGTAA | |
| | LGCLVEDYFPEPVTVSWNSGALTSGVHTFPAVLQS | |
| | SGLYSLSSVVTVPSSSLGTQTYICNVNHKPSNTKVD | |
| | EKVEPKSCDKTHTCPPCPAPEAAGGPSVFLFPPKPK | |
| | DTLMISRTPEVTCVVVDVSHEDPEVKFNWYVDGV | |
| | EVHNAKTKPREEQYNSTYRVVSVLTVLHQDWLNG | |
| | KEYKCKVSNKALGAPIEKTISKAKGQPREPQVYTL | |
| | PPSRDELTKNQVSLTCLVKGFYPSDIAVEWESNGQ | |
| | PENNYKTTPPVLDSDGSFFLYSKLTVDKSRWQQGN | |
| | VFSCSVMHEALHNHYTQKSLSLSPGGGGGSGGGG | |
| | SGGGGSGGGGSEIVLTQSPGTLSLSPGERATLSCRA | |
| | SQSVSRSYLAWYQQKPGQAPRLLIIGASTRATGIPD | |
| | RFSGSGSGTDFTLTISRLEPEDFAVYYCQQGQVIPPT | |
| | FGQGTKVEIKSSASTKGPSVFPLAPSSKSTSGGTAA | |
| | LGCLVKDYFPEPVTVSWNSGALTSGVHTFPAVLQS | |
| | SGLYSLSSVVTVPSSSLGTQTYICNVNHKPSNTKVD | |
| | KKVEPKSC | |

### 3.2 Production of Bispecific Antigen Binding Molecules Targeting FAP and CD40

The bispecific antigen binding molecules targeting fibroblast activation protein (FAP) and CD40 were expressed by transient transfection of HEK cells grown in suspension with expression vectors encoding the 4 different peptide chains. Transfection into HEK293-F cells (Invitrogen) was performed according to the cell supplier's instructions using Maxiprep (Qiagen) preparations of the antibody vectors, F17 medium (Invitrogen, USA), Peipro (Polyscience Europe GmbH) and an initial cell density of 1-2 million viable cells/ml in serum free FreeStyle 293 expression medium (Invitrogen). Cell culture supernatants were harvested after 7 days of cultivation in shake flasks or stirred fermenters by centrifugation at 14000 g for 30 minutes and filtered through a 0.22 μm filter.

The bispecific antibodies were purified from cell culture supernatants by Protein A affinity chromatography using MabSelectSure-Sepharose™ (GE Healthcare, Sweden) chromatography. Briefly, sterile filtered cell culture supernatants were captured on a MabSelect SuRe resin equilibrated with PBS buffer (10 mM Na₂HPO₄, 1 mM KH₂PO₄, 137 mM NaCl and 2.7 mM KCl, pH 7.4), washed with equilibration buffer and eluted with 25 mM cirate, pH 3.0. After neutralization with 1 M Tris pH 9.0, aggregated protein was separated from monomeric antibody species by size exclusion chromatography (Superdex 200, GE Healthcare) in 20 mM histidine, 140 mM NaCl, pH 6.0. Monomeric protein fractions were pooled, concentrated if required using e.g. a MILLIPORE Amicon Ultra (30KD MWCO) centrifugal concentrator and stored at −80° C. Sample aliquots were used for subsequent analytical characterization e.g. by CE-SDS, size exclusion chromatography, mass spectrometry and endotoxin determination.

TABLE 25
Production yield of bispecific CD40-FAP antigen binding molecules

| | Yield | | % | | % | % |
|---|---|---|---|---|---|---|
| | after ProtA | % | product | Yield | monomer | product peak |
| | from 0.9 L | monomer | peak by | after prep. | by SEC | by CE-SDS |
| | transient HEK | by SEC | CE-SDS | HIC and | after prep. | after prep. |
| | expression | after | after | prep. SEC | HIC and | HIC and |
| Sample | in mg | ProtA | ProtA | [mg/L] | prep. SEC | prep. SEC |
| P1AE2423 | 16.5 | 92 | >95 | 11.25 | >95 | >95 |
| P1AE2424 | 10.4 | 93 | >95 | 6.4 | >95 | >95 |
| P1AE2734 | 25 | 89 | 18 | | | |
| P1AE2487 | 14 | 93 | 82 | 4.8 | >95 | >95 |
| P1AE2895 | 14.4 | 94 | 91 | 4.1 | >95 | >95 |
| P1AE2302 | | | | 12 | | |
| P1AE2024 | | | | 6.6 | | |

P1AE2734 is a bispecific antibody wherein the VH-Ckappa chain of the crossfab is fused to the C-terminus of one of the Fc domain heavy chains. Thîs bispecific antibody was much more difficult to produce and to purify than the bispecific antibody P1AE2423, wherein the VL-CH1 chain of the crossfab is fused to the C-terminus of one of the Fc domain heavy chains. Further purification steps had to be applied, i.e. an ion exchange chromatography as well as an additional CE-SDS led to 1 mg of substance.

### 3.3 Binding Kinetics of Bispecific Anti-huFAP Antibodies

To evaluate human FAP binding kinetics of bispecific FAP-targeted antibodies, the binding kinetics were measured as described in Example 1.7, however with using a HIS-tagged human FAP ectodomain as ligand. The affinity data of the bispecific 2+1 CD40×FAP antibodies with different fusion of the crossfab are shown in Table 25A below.

TABLE 25A
Affinity of 2 + 1 CD40 × FAP bispecific
antibodies to human FAP as measured by SPR

| Sample ID | ka (1/Ms) | kd (1/s) | KD [nM] |
|---|---|---|---|
| P1AE2423 | 3.4E+05 | 3.2E−05 | 9.4E−11 |
| VL-CH1 fused to Fc | | | |
| P1AE2734 | 2.5E+05 | 5.2E−05 | 2.1E−10 |
| VH-Ckappa fused to Fc | | | |

FAP binding affinities of bispecific antibodies as C-terminal Fab-fusion are in a similar range between the two crossing variants (measured by SPR).

### Example 4

### Characterization of Bispecific Constructs Targeting CD40 and FAP

### 4.1 Binding to Human FAP-Expressing Murine Fibroblast Cells

The binding to cell surface FAP was tested using the human fibroblast activating protein (huFAP) expressing NIH/3T3-hFAP clone 19. The NIH/3T3-huFAP clone 19 was generated by the transfection of the mouse embryonic fibroblast NIH/3T3 cell line (ATCC CRL-1658) with the expression vector pETR4921 to express under 1.5 μg/mL Puromycin selection huFAP.

NIH/3T3-huFAP cells were cultured with 1× Dulbecco's Modified Eagle's Medium (DMEM) (gibco, Cat. No. 42430-025) supplemented with 10% Fetal Bovine Serum (FBS) (life technologies, Cat. No. 16140, Lot No. 1797306A). 1.5 g/mL Puromycin (gibco, Cat. No. A11138-03) was added to the medium for selection of FAP-expressing cells. NIH/3T3-huFAP cells were removed from culture flasks by using enzyme-free Cell Dissociation Buffer (gibco, Cat. No. 13151014). 0.3×10⁵ NIH/3T3-hFAP clone 19 cells were added in 200 μl of 1×DMEM with 10% FBS to each well of a round-bottom 96-well plate (greiner bio-one, cellstar, Cat. No. 650185). Plates were centrifuged 5 minutes at 1700 rpm and supernatants were flicked off. Cells were washed once with 200 μL of 4° C. cold FACS buffer (eBioscience, Cat. No. 00-4222-26). All samples were resuspended in 50 μL/well of 4° C. cold FACS buffer containing the bispecific antigen binding molecules (primary antibody) or the isotype control antibody DP47 at the indicated range of concentrations (in duplicates) and incubated for 120 minutes at 4° C. Afterwards the cells were washed three times with 200 μL 4° C. cold FACS buffer. Cells were further stained with 25 μL/well of 4° C. cold secondary antibody solution (1:50 dilution of secondary antibody) containing the R-Phycoerythrin (PE) conjugated AffiniPure F(ab′)₂ Fragment Goat Anti-Human IgG, Fcγ Fragment Specific (Jackson ImmunoResearch, Cat. No. 109-116-098) secondary antibody and incubated for 60 minutes at 4° C. in the dark. Cells were washed with 200 μl FACS buffer and resuspended in 85 μL/well FACS buffer containing 0.2 μg/mL DAPI (Roche, Cat. No. 10236276001) and acquired the same day using a 5-laser LSR-Fortessa (BD Bioscience with DIVA software). Data analysis was performed using the FlowJo version 10 software (FlowJo LLC).

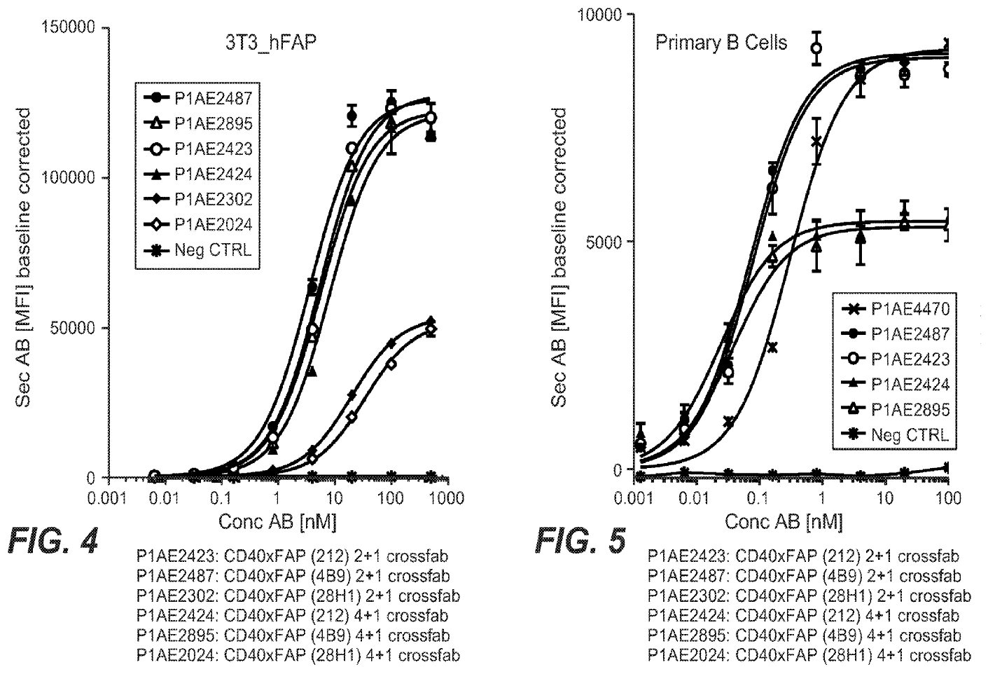

As shown in FIG. 4, the bispecific antibodies monovalent for FAP bind to human FAP-expressing target cells. Therefore, the FAP-targeted anti-CD40 antigen binding molecules show direct tumor-targeting properties. The FAP constructs with a C-terminal FAP (212 (P1AE1689)) or FAP (4B9) binding domain bind stronger than the FAP constructs with a C-terminal FAP (28H1) binding domain explained by a higher binding affinity of the FAP (212) and FAP (4B9) binders towards human FAP compared to the FAP (28H1) binder. The strongest FAP binding was observed for the 2+1 construct with a FAP (4B9) binding moiety (P1AE2487). No binding of the isotype control antibody DP47 (human germline control) to the NIH/3T3-hFAP cells was detected. The EC₅₀ values as measured for different bispecific antibodies are shown in Table 26 below.

TABLE 26
Human FAP binding characterization of 212 (P1AE1689),
4B9 and 28H1 in different bispecific antibody formats

| Molecule | | EC₅₀ [nM] |
|---|---|---|
| P1AE2423 | CD40 × FAP (212) 2 + 1 crossfab | 5.19 |
| P1AE2487 | CD40 × FAP (4B9) 2 + 1 crossfab | 3.70 |
| P1AE2302 | CD40 × FAP (28H1) 2 + 1 crossfab | 20.01 |
| P1AE2424 | CD40 × FAP (212) 4 + 1 crossfab | 7.52 |
| P1AE2895 | CD40 × FAP (4B9) 4 + 1 crossfab | 5.27 |
| P1AE2024 | CD40 × FAP (28H1) 4 + 1 crossfab | 38.67 |

### 4.2 Binding to Human CD40-Expressing Primary B Cells

The binding to cell surface CD40 was tested using human primary B cells isolated from peripheral blood mononuclear cells (PBMCs). In order to isolate PBMCs a buffy coat was obtained from the Stiftung Zürcher Blutspendedienst SRK. 50 mL of buffy coat were diluted in the same volume of PBS (gibco, Cat. No. 10010023). 50 mL polypropylene centrifuge tubes (TPP, Cat. No. 91050) were supplied with 15 mL of Lymphoprep™ (STEMCELL Technologies, Cat. No. 07851) and 25 mL of the buffy coat/PBS solution per tube were carefully layered above the Lymphoprep™. The tubes were centrifuged at 2000 rpm for 24 minutes at room temperature with low acceleration and without break. Afterwards the PBMCs were collected from the interface, washed three times with PBS, resuspended in 10 mL of PBS and cells were analyzed for cell type and number with a Beckman Coulter cell counter Ac·T™ 5diff OV (Beckman Coulter, Cat. No. 6605580). Prior to the B cell isolation from the PBMCs, the CD14-positive fraction was removed by magnetic labeling of the CD14-positive cells with CD14 microbeads (Miltenyi, Cat. No. 130-050-201) and subsequent isolation with an autoMACS® Pro Separator (Miltenyi, Cat. No. 130-092-545). The CD14-negative fraction was used for subsequent B cell isolation with the Miltenyi B cell isolation kit II (Cat. No. 130-091-151) and autoMACS® separation. 0.3×10⁵ B cells were added in 200 μl of R10 medium consisting of Roswell Park Memorial Institute medium (RPMI) 1640 (gibco, Cat. No. 31870-025) supplied with % (v/v) FBS, 1% (v/v) Penicillin Streptomycin (gibco, Cat. No. 15070-063), 1% (v/v) L-Glutamine (gibco, Cat. No. 25030-024), 1% (v/v) Sodium-Pyruvate (gibco, Cat. No. 11360-039), 1% (v/v) MEM non-essential amino acids (gibco, Cat. No. 11140-035) and 50 μM β-Mercaptoethanol (gibco, Cat. No. 31350-010) to each well of a round-bottom 96-well plate (greiner bio-one, cellstar, Cat. No. 650185). Plates were centrifuged 5 minutes at 1700 rpm and supernatants were flicked off. Cells were washed once with 200 μL of 4° C. cold FACS buffer (eBioscience, Cat. No. 00-4222-26). All samples were resuspended in 50 μL/well of 4° C. cold FACS buffer containing the bispecific antigen binding molecules (primary antibody) or the isotype control antibody DP47 at the indicated range of concentrations (in duplicates) and incubated for 120 minutes at 4° C. Afterwards the cells were washed three times with 200 μL 4° C. cold FACS buffer. Cells were further stained with 25 μL/well of 4° C. cold secondary antibody solution (1:50 dilution of secondary antibody) containing the R-Phycoerythrin (PE) conjugated AffiniPure F(ab′)₂ Fragment Goat Anti-Human IgG, Fcγ Fragment Specific (Jackson ImmunoResearch, Cat. No. 109-116-098) secondary antibody and incubated for 60 minutes at 4° C. in the dark. Cells were washed with 200 μl FACS buffer and resuspended in 85 μL/well FACS-buffer containing 0.2 μg/mL DAPI (Roche, Cat. No. 10236276001) and acquired the same day using a 5-laser LSR-Fortessa (BD Bioscience with DIVA software). Data analysis was performed using the FlowJo version 10 software (FlowJo LLC).

As shown in FIG. 5, all depicted clones bind to CD40 but vary in their binding strength (EC₅₀ values as well as signal strength) to CD40-positive B cells. Irrespective of their FAP binding moiety, bivalent anti-CD40 antibodies show higher EC₅₀ levels and reach higher binding plateaus compared to tetravalent anti-CD40 antibodies explained by more occupied CD40 binding sites per antibody and a gain of avidity of the tetravalent relative to the bivalent CD40 formats. No binding of the negative control antibody to B cells was detected. The EC₅₀ values as measured for different bispecific antibodies are shown in Table 27 below.

TABLE 27
Human CD40 binding characterization of CD40 antibodies
in different bispecific antibody formats

| Molecule | | EC₅₀ [nM] |
|---|---|---|
| P1AD4470 | CD40 IgG1 | 0.333 |
| P1AE2423 | CD40 × FAP (212) 2 + 1 crossfab | 0.095 |
| P1AE2487 | CD40 × FAP (4B9) 2 + 1 crossfab | 0.086 |
| P1AE2424 | CD40 × FAP (212) 4 + 1 crossfab | 0.036 |
| P1AE2895 | CD40 × FAP (4B9) 4 + 1 crossfab | 0.049 |

### Example 5

### Functional Properties of FAP-Targeted Anti-Human CD40 Binding Molecules

### 5.1 CD40-Mediated Activation of Human B Cells by FAP-Targeted Anti-Human CD40 Binding Molecules

Ligation of CD40 induces B cell and dendritic cell (DC) maturation as well as activation and promotes survival of these cell types. Upon CD40 signaling cytokine production and costimulatory molecule expression on the surface of B cells and DCs is increased (S. Quezada et al., Annu RevImmunol. 2004, 22, 307-328; S. Danese et al., Gut. 2004, 53, 1035-1043; G. Bishop et al., Adv Exp Med Biol. 2007, 597, 131-151).

In order to test the agonistic properties and the FAP specificity of the different FAP-dependent anti-CD40 antibodies, Daudi cells or primary B cells obtained from human buffy coats were incubated with the FAP-dependent agonistic anti-human CD40 antibodies in the presence of FAP-coated beads and the B cell activation was measured by FACS.

### 5.1.1. Activation of Human Daudi Cells by FAP-Targeted Anti-Human CD40 Binding Molecules Using FAP-Coated Dynabeads® as Source of Antigen

1×10⁵ Daudi cells, a human B lymphoblast cell line with high expression levels of human CD40 (ATCC CCL-213), were added in 100 μl of 1× Dulbecco's Modified Eagle's Medium (DMEM) (gibco, Cat. No. 42430-025) supplemented with 10% Fetal Bovine Serum (FBS) (life technologies, Cat. No. 16140, Lot No. 1797306A) per well of a 96-well flat-bottom plate. Streptavidin Dynabeads® (ThermoFisher Scientific, Cat. No.: 11205D) were coated with biotinylated human FAP (produced in-house) (binding capacity of 6.5×10⁴ beads: 0.01 μg of protein) according to the manufacturer's instructions and added to the Daudi cells in a beads to cell ratio of 2:1 in 50 μl of 1×DMEM with 10% FBS. As control, non-coated beads were added to the Daudi cells. FAP-targeted anti-human CD40 antibodies were added in 50 μl of 1×DMEM with 10% FBS medium to the Daudi cells at concentrations ranging from 6.7 nM to 0.003 nM (3× dilution series). As positive control, the FAP-independent agonistic anti-human CD40 antibody SGN-40 (IgG1, INN: Dacetuzumab) was used. The antibody is bivalent for CD40. Since it is described in the literature that the SGN-40 antibody requires Fc receptor cross-linking for biological activity (C. Law et al., Cancer Res 2005, 65, 8331-8338), the antibody was incubated with a cross-linking goat anti-human IgG Fcγ fragment specific F(ab′)₂ fragment (Jackson ImmunoResearch, Cat. No. 109-006-008) for 30 minutes before the antibody was added to the Daudi cells. After 48 hours, cells were transferred into a 96-well round-bottom plate, washed once with PBS and incubated with 50 μl of 3 μg/mL of Fc receptor blocking Mouse IgG Isotype Control (ThermoFisher Scientific, Cat. No. 10400C) in PBS. After 15 minutes of incubation at 4° C., cells were washed with PBS and 50 μl of a mixture of fluorescently labelled antibodies in PBS were added to the cells. The following fluorescently labelled antibodies were used: anti-human CD83 BV421 (Biolegend, clone HB15e, Cat. No. 305324), anti-human CD80 BV605 (BD Biosciences, clone L307.4, Cat. No. 563315), anti-HLA-ABC FITC (BD Biosciences, clone G46-2.6, Cat. No. 555552), anti-human CD14 PerCP-Cy5.5 (Biolegend, clone HCD14, Cat. No. 325622), anti-human CD3 PerCP-Cy5.5 (Biolegend, clone UCHT1, Cat. No. 300430), anti-human CD70 PE (Biolegend, clone 113-16, Cat. No. 355104), anti-human CD86 PE-CF594 (BD Biosciences, clone FUN-1, Cat. No. 562390), anti-HLA-DR APC (BD Biosciences, clone G46-6, Cat. No. 559866) and anti-human CD19 APC-H7 (BD Biosciences, clone SJ25C1, Cat. No. 560177). In order to distinguish between live and dead cells, the viability dye Zombie Aqua™ (Biolegend, Cat. No. 423102) was added to the antibody mixture. After 30 minutes of incubation at 4° C., cells were washed twice with PBS and resuspended in 200 μl of PBS. Cells were analyzed the same day using a 5-laser LSR-Fortessa (BD Bioscience with DIVA software). Data analysis was performed using the FlowJo version 10 software (FlowJo LLC). Live (aqua negative) cells, negative for CD14 and CD3 and positive for CD19 were analyzed for CD70, CD80, CD83 and CD86 expression.

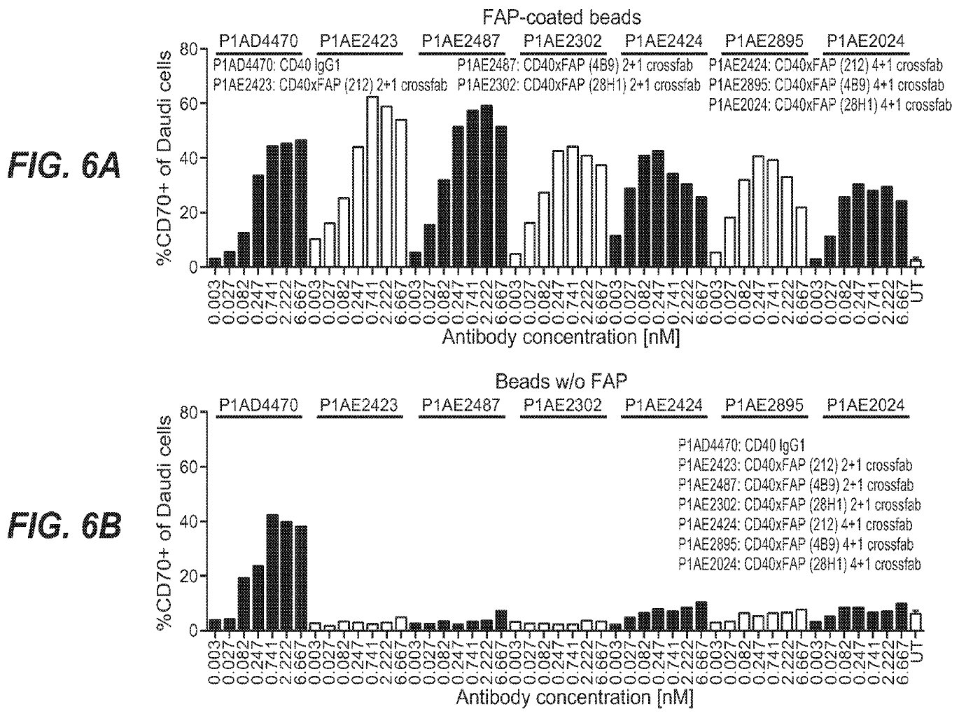

Daudi cells analyzed after 2 days of incubation with agonistic anti-CD40 antibodies showed an increase in CD70 expression for all depicted antibodies (see FIG. 6A and FIG. 6B). The upregulation of this activation marker was dependent on FAP in case of the different FAP-targeted antibodies. At lower antibody concentrations the increase of expression induced by these FAP-dependent antibodies was higher compared to the increase induced by the cross-linked CD40 antibody (P1AD4470). In addition, the CD70 upregulation by bispecific FAP-CD40 antibodies in a 2+1 format with a FAP (212) or FAP (4B9) binding domain was higher compared to the upregulation induced by the bispecific FAP-CD40 antibody in a 2+1 format with a FAP (28H1) binding domain, the bispecific FAP-CD40 antibodies in a 4+1 format with a FAP (212), FAP (4B9) or FAP (28H1) binding domain or the FAP-independent positive control antibody. In the absence of FAP (uncoated beads) no increase of CD70 was observed with the depicted bispecific antibodies bivalent for CD40, while tetravalent CD40 binding molecules induced an upregulation of CD70, but to a lesser extent than in the presence of FAP indicating a low but detectable FAP-independent CD40 activation of tetravalent CD40 binders in Daudi cells.

### 5.1.2 Activation of Human B Cells by FAP-Targeted Anti-Human CD40 Binding Molecules Using FAP-Coated Dynabeads® as Source of Antigen

B cells were isolated from buffy coats as described in Example 4.2 and 1×10⁵ B cells in 100 μl of R10 medium were added per well of a 96-well flat-bottom plate. Streptavidin Dynabeads® (ThermoFisher Scientific, Cat. No.: 11205D) were coated with biotinylated human FAP (produced in-house) (binding capacity of 6.5×10⁴ beads: 0.01 μg of protein) according to the manufacturer's instructions and added to the B cells in a beads to cell ratio of 2:1 in 50 μl of R10 medium. As control non-coated beads were added to the B cells. The FAP-targeted anti-human CD40 antibodies or positive control antibodies (described in Example 5.1.1) were added in 50 μl of R10 medium to the B cells. After 2 days B cells were analyzed by FACS following the staining and analysis procedures specified in Example 5.1.1.

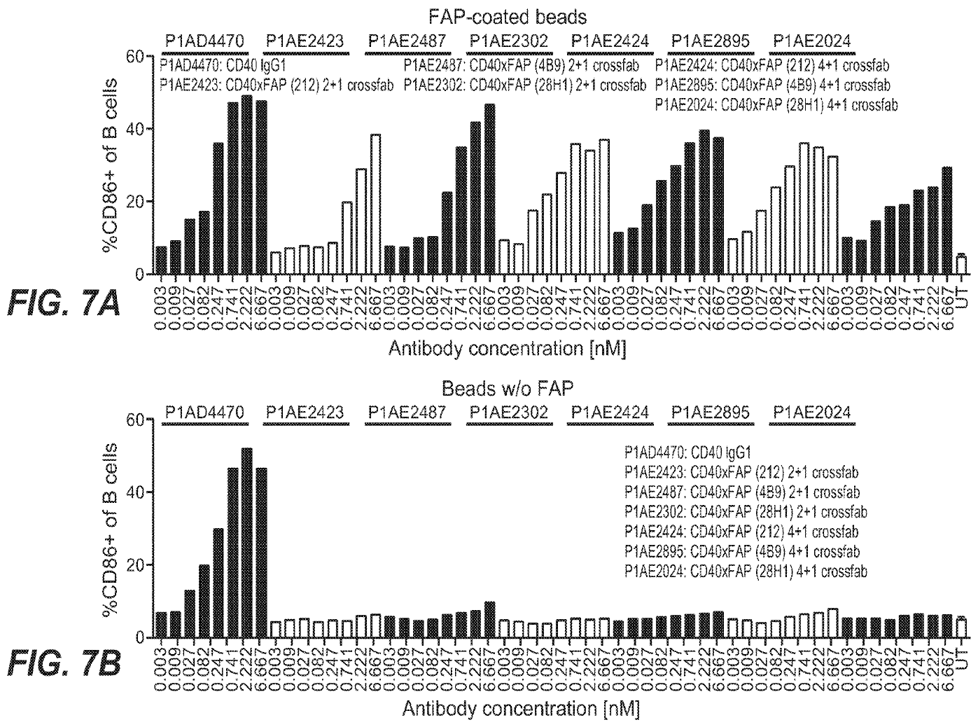

B cells analyzed after 2 days of incubation with agonistic anti-CD40 antibodies showed an increase in CD86 expression for all depicted antibodies (see FIG. 7A and FIG. 7B). The upregulation of CD86 was dependent on FAP in case of the different FAP-targeted antibodies and the increase of expression induced by these FAP-dependent antibodies was comparable or slightly lower to the increase induced by the cross-linked anti-CD40 antibody P1AD4470. The bispecific antibodies monovalent for FAP with one FAP (212) binding domain induced a similar increase of activation marker expression as the molecules with one FAP (4B9) binding moiety. At lower antibody concentrations the 4+1 formats, irrespective of their FAP binding moiety, induced a higher B cell activation compared to the 2+1 formats with a FAP (212) or FAP (4B9) binding moiety.

### 5.2 CD40-Mediated Activation of DCs by FAP-Targeted Anti-CD40 Binding Molecules and Subsequent Priming of T Cells

In order to demonstrate the ability of DCs activated by the FAP-dependent anti-human CD40 antibodies to efficiently prime T cells, in vitro T cell priming assays were established. For these assays DCs from the spleens of transgenic mice expressing the human CD40 receptor (huCD40tg mice; mice with similar human and murine CD40 receptor expression pattern; C57BL/6 background; generated by Taconic) were isolated, pulsed with either SIINFEKL peptide or with ovalbumin (OVA; DEC-205 receptor-mediated antigen uptake) and incubated with different agonistic anti-human CD40 antibodies. FAP was provided via FAP-coated Dynabeads® in order to show FAP-dependency of the bispecific antigen binding molecules. 24 hours later, CD8-positive T cells were isolated from spleens of OT1 mice (CD8-positive T cells of these mice all possess a transgenic TCR recognizing SIINFEKL in the context of H2-Kb; C57BL/6-Tg(TcraTcrb)1100Mjb/Crl, Charles River), carboxyfluorescein succinimidyl ester (CFSE) labelled and added to the pulsed DCs. On day four of the experiment the T cell proliferation was analyzed by FACS.

### 5.2.1 T Cell Priming Via OVA-Pulsed DCs Activated by FAP-Targeted Anti-CD40 Binding Molecules

DCs were isolated from the spleens of huCD40tg mice. In order to isolate splenic DCs, the spleen from a huCD40tg mouse was put into one well of a 6-well plate containing 2.25 mL Hank's Balanced Salt Solution (HBSS) with Calcium²⁺ (gibco, Cat. No. 14025-05), 250 μl of a 10 mg/mL solution of collagenase D (end concentration 1 mg/mL) (Sigma-Aldrich, Cat. No. 11088866001) and 12.5 μl of a 10 mg/mL DNase solution (end concentration 0.05 mg/mL) (Sigma-Aldrich, D5025-150KU, Lot. No. SLBR0535V). The spleen was ballooned using a 3 mL syringe (BD, Cat. No. 309658) with a 21G needle (Braun, Cat. No. 4657527) and subsequently, with the help of scissors, torn into small pieces. After a 25 minutes incubation at 37° C., 50 μL of 0.5 M ethylenediaminetetraacetic acid (EDTA) (Applichem, Cat. No. A4892.1000) were added, followed by a second incubation step at 37° C. for five minutes. The solution containing splenocytes and small pieces of splenic tissue was filtered through a 40 μm filter (Corning, Cat. No. 352340) into a 50 mL polypropylene centrifuge tube. Remaining splenic tissue pieces were smashed through the filter with the end of a 3 mL syringe plug. In the next step the 50 mL tube was centrifuged at 1500 rpm for 5 minutes at room temperature, the supernatant was discarded and 1 mL of 1× cell lysis buffer (diluted 1:10 with distilled water) (BD, Cat. No. 555899) was added to the splenocytes in order to lyse the red blood cells. After four minutes of incubation at room temperature, 20 mL of R10 were added followed by a centrifugation step at 1500 rpm for 5 minutes at room temperature. The supernatant was removed, the splenocytes were resuspended in 30 mL of R10 and cell numbers as well as viability were determined with the automated EVE cell counter (VWR, Cat. No. 734-2675). The mouse CD11c UltraPure microbeads (Miltenyi, Cat. No. 130-108-338) were used according to the manufacturer's instruction to isolate DCs by autoMACS® separation. Subsequently 0.25×105 DCs were seeded in 50 μl of R10 per well of a 96-well flat-bottom plate.

The DCs were then either pulsed with 1 ng/mL SIINFEKL (Ovalbumin residues 257-264, Eurogentec, Cat. No. AS-60193-5, Lot. No. 1360618), which requires no uptake and processing by the DCs, as positive control or loaded with OVA protein as antigen. In order to promote the OVA uptake in a Toll-like receptor (TLR) stimulus independent way (additional TLR stimuli might lead to a high overall activation of DCs, making the detection of different activation states due to stimulation with agonistic anti-CD40 antibodies impossible) the OVA Antigen Delivery Reagent (Miltenyi, Cat. No. 130-094-663) in combination with a biotinylated anti-mouse DEC205 antibody (Miltenyi, clone NLDC-145, Cat. No. 130-101-854) was used according to the manufacturer's protocol. In brief, DCs were incubated with a biotinylated antibody that binds to the DEC205 receptor, which is highly expressed on CD8-positive cross-presenting DCs (M. Lahoud et al., Int Immunol. 2000, 12 (5), 731-735). Afterwards, the OVA delivery reagent, an anti-biotin antibody coupled to FITC and OVA, was added to the cells leading to DEC205 receptor-mediated uptake of OVA. In order to provide a negative control, DCs were only labelled with the anti-DEC205 antibody without the addition of OVA. In addition, human FAP-coated or non-coated Dynabeads® were added in 50 μL of R10 to the DCs at a 2:1 beads to cell ratio as described in Example 5.1.1. In the next step different agonistic anti-CD40 antibodies were added in 50 μL of R10 at concentrations ranging from 6.7 nM to 0.01 nM (10× dilution series). In this experimental setup, the bispecific 2+1 and 4+1 anti-human CD40 antibodies containing one FAP (212) or FAP (4B9) FAP binding site were compared to the cross-linked SGN-40 antibody.

On the next day, splenic CD8-positive cells from OT1 mice were isolated. In order to do so, the spleen of an OT1 mouse was smashed through a 40 μm filter with the end of a 3 mL syringe plug into a 50 mL tube. The filter was washed with R10 and the splenocytes were centrifuged at 1500 rpm for 5 minutes at room temperature. 1 mL of 1× cell lysis buffer (diluted 1:10 with distilled water) was added to the cells and after four minutes of incubation at room temperature, 20 mL of R10 were added. The tube was centrifuged at 1500 rpm for 5 minutes at room temperature and the supernatant was discarded. The splenocytes were resuspended in 30 mL of R10 and cell counts as well as viability were determined with the automated EVE cell counter. CD8-positive cells were isolated in a negative selection process using the mouse CD8a⁺ T Cell Isolation Kit (Miltenyi, Cat. No. 130-104-075) and autoMACS® separation according to the manufacturer's instructions. CD8-positive cells that were found in the negative fraction after the separation were then washed with pre-warmed PBS, counted with the EVE cell counter and the cell number was adjusted to 2×10⁷ cells/mL in pre-warmed PBS. 10 mM CFSE solution (CellTrace™ CFSE Cell Proliferation Kit, ThermoFisher, Cat. No. C34554) was 5000-fold diluted in pre-warmed PBS and added to the cells resuspended in PBS in a 1:1 ratio (CFSE end concentration 1 μM). After a short vortex, cells were incubated for five minutes at room temperature. The labelling reaction was stopped by adding 40 mL of pre-warmed R10 medium to the cells. After two washing steps with PBS, CD8-positive cells were resuspended in R10 and 0.5×10⁵ cells were added in 100 μl R10 to the pulsed DCs. On day four of the experiment, the T cell proliferation was analyzed by flow cytometry. Therefore, the cells were transferred from the 96-well flat-bottom plates into 96-well round-bottom plates, washed once with PBS and incubated with 50 μl of 3 μg/mL of Fc receptor blocking Mouse IgG Isotype Control in PBS. After 15 minutes of incubation at 4° C., cells were washed with PBS and 50 μl of a mixture of fluorescently labelled antibodies in PBS were added to the cells. The following antibodies were used: anti-mouse CD4 BV421 (Biolegend, clone GK1.5, Cat. No. 100438), anti-mouse CD86 BV785 (Biolegend, clone GL-1, Cat. No. 105043), anti-I-A/I-E PerCp-Cy5.5 (Biolegend, clone M5/114.15.2, Cat. No. 107626), anti-mouse CD70 PE (eBioscience, clone FR70, Cat. No. 12-0701-82), anti-mouse CD3 PE-CF594 (BD Biosciences, clone 145-2C11, Cat. No. 562286), anti-mouse CD25 PE-Cy7 (eBioscience, clone PC61.5, Cat. No. 25-0251-82), anti-mouse CD11c APC (BD Biosciences, clone HL3, Cat. No. 561119), anti-mouse CD44 Alexa Fluor 700 (BD Biosciences, clone IM7, Cat. No. 560567) and anti-mouse CD8 APC-Cy7 (Biolegend, clone 53-6.7, Cat. No. 100714). In order to distinguish between live and dead cells, the viability dye Zombie Aqua™ was added to the antibody mixture. Cells were incubated for 30 minutes at 4° C. with 50 μl of the staining antibody mix. Afterwards, cells were washed two times with PBS, resuspended in 200 μl of PBS and analyzed using a 5-laser LSR-Fortessa. Data analysis was performed using the FlowJo version 10 software. Viable CD3- and CD8-positive cells were analyzed for CFSE signal as well as CD25 and CD44 expression.

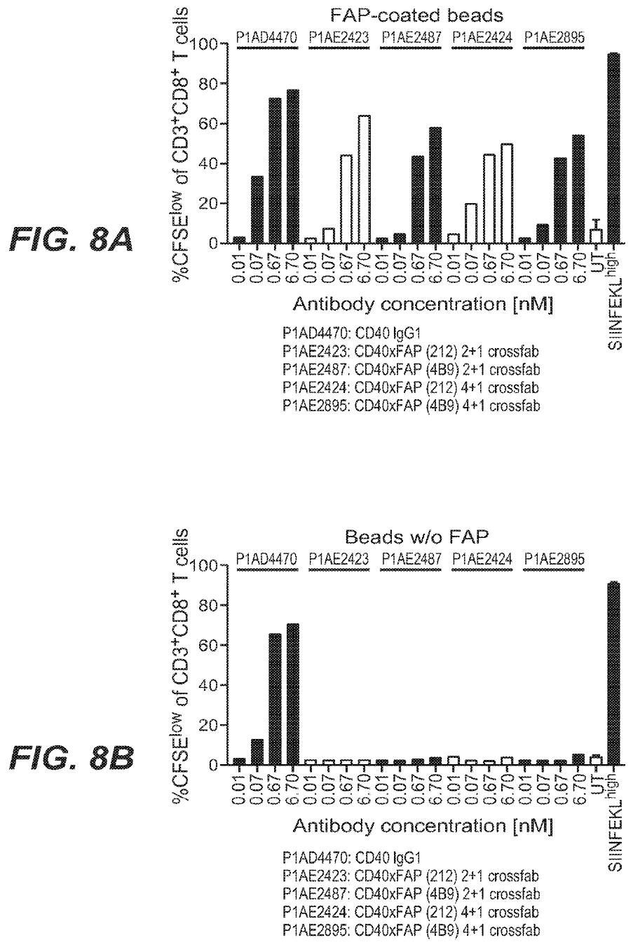

FIG. 8A and FIG. 8B show that DCs incubated with the OVA delivery reagent and stimulated with the bispecific antigen binding molecules targeting human CD40 and FAP highly enhance CD8-positive OT1 T cell proliferation. These effects were FAP-dependent. The increase of T cell proliferation induced by the depicted FAP-dependent antibodies was slightly lower compared to the increase induced by the cross-linked CD40 antibody (P1AD4470). The levels of proliferation induced by DCs stimulated with the 2+1 or 4+1 bispecific anti-CD40 antibodies with one FAP (212) or FAP (4B9) binding moiety were comparable.

### 5.3 Two Cell Line Bridging Assay for FAP Binding and CD40 Signal Transduction

The mode of action of the bispecific antibodies is reflected in the two cell line bridging assay: the antibodies bind to FAP on the FAP expressing cell line and to CD40 receptor on the reporter gene cell line. In case of clustering of the CD40 receptor the signal transduction results in the induction of NF□B-dependent production of secreted embryonic alkaline phosphatase

In brief: The reporter cell line HEK-Blue™ CD40L (Invivogen, transfected with plasmids encoding human CD40 receptor and a NFκB-inducible secreted embryonic alkaline phosphatase (SEAP)) was detached by Accutase (Life Technologies) and 6.8×10⁶ cells were transferred into a sterile 50 mL centrifuge tube (Greiner) and placed on a roller mixer (Heidolph) until use.

The native target cells (WM-266-4, ATCC) expressing FAP on the surface were used as ready-to-use frozen cells. Each vial contained 2×10⁷ cells/2 mL DMEM, 10% FBS and 5% DMSO. The maintenance culture of the cells should be done in DMEM with high glucose and HEPES (Life Technologies). The target cells were thawed for 2 minutes in a 37° C. pre-warmed water bath. The 2 mL cell suspension was transferred immediately into a 50 mL centrifuge tube with 30 mL DMEM, 10% FBS (Life Technologies). An aliquot of 13.6×10⁶ cells was transferred into a 50 mL centrifuge tube and placed on a roller mixer until use. The thawing of the cells was done after preparation of the antibody dilution series.

The HEK-Blue™ Detection Medium (Invivogen) was used for preparation of cell suspensions and antibody dilutions. Three pouches were solved in 150 mL destilled water (Life Technologies) and filtered through a 0.22 μm membrane (Millipore) into a sterile glass bottle (Schott). 150 μl Gentamicin (50 mg/mL, Life Technologies) was added.

The antibody dilutions were made 2-fold concentrated in HEK-Blue™ Detection Medium. Eight concentrations in a serial dilution series (1:4) resulted in the final assay concentrations from 518 pM to 0.0316 pM. 100 μL of each antibody concentration was transferred into clear 96-well micro titer tissue culture plates (Greiner). Duplicates on 3 plates per molecule were evaluated.

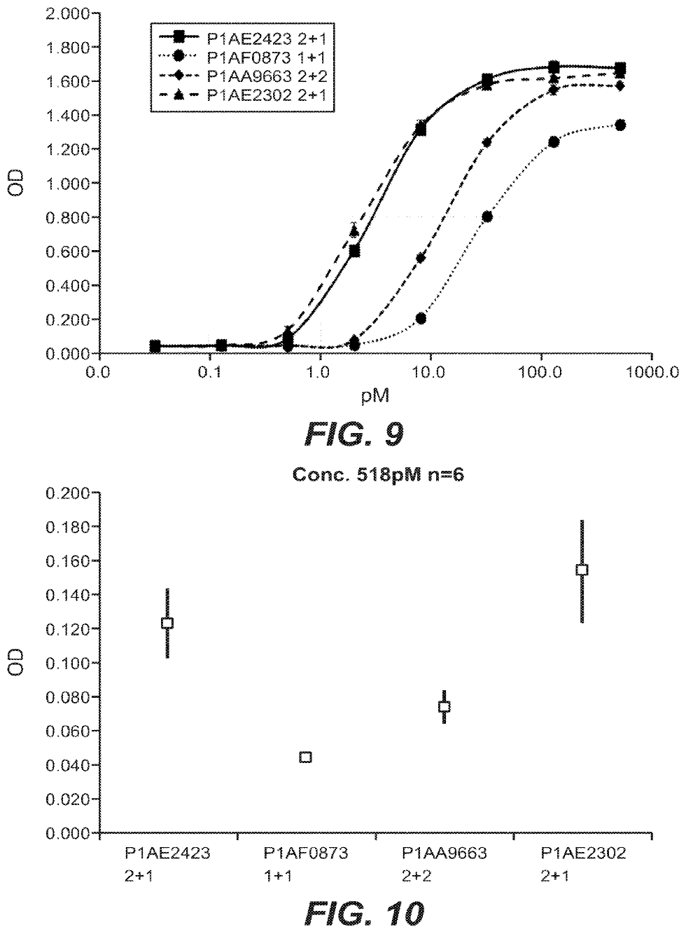

Both cell lines (reporter and target cell line) were centrifuged 4 minutes at 180×g at room temperature. The supernatants were removed and each cell line was resuspended in 17 mL HEK-Blue™ Detection Medium. By combining both cell lines, finally the mix contained 2×10⁵ HEK-Blue™ CD40L cells per mL and 4×10⁵ WM-266 4 cells per mL. 100 μL of the cell mixture was added into each well of the assay plates. Finally, 20.000 HEK-Blue™ CD40L cells and 40.000 WM-266-4 cells were present in each well. The plates were pre-incubated for 15 minutes at room temperature to allow cell distribution and settlement and then incubated for 20 hours at 37° C. and 5% CO₂ (cell incubator). Activity of SEAP was measured with HEK-Blue™ Detection Medium at 650 nm (Victor X4, Perkin Elmer). The optical density (OD) was plotted against the concentration of the antibodies (see FIG. 9). The EC₅₀ values are shown in Table 28 below.

TABLE 28
EC₅₀ values as measured in the Two cell line bridging assay

| Molecule | | EC₅₀ [pM] |
|---|---|---|
| P1AE2423 | CD40 x FAP (212) 2 + 1 crossfab | 3.2 |
| P1AF0873 | CD40 x FAP (212) 1 + 1 crossfab | 26.5 |
| P1AA9663 | CD40 x FAP (28H1) 2 + 2 crossfab | 13.2 |
| P1AE2302 | CD40 x FAP (28H1) 2 + 1 crossfab | 2.5 |

The EC₅₀ values are very similar for the 2+1 molecules P1AE2423 and P1AE2302 comprising two CD40-binding Fab fragment and FAP-binding crossfab fragment. The EC₅₀ is significantly higher for the 1+1 crossfab construct P1AF0873 and the 2+2 construct P1AA9663. In addition, the 1+1 construct P1AF0873 shows a significant less pronounced higher asymptote meaning less effectivity.

### 5.4 FAP-Independent CD40 Specific Assay

The potential activation of the CD40 pathway without the clustering via FAP was evaluated. In case of activation of the CD40 receptor the signal transduction results in the induction of NF□B-dependent production of secreted embryonic alkaline phosphatase.

In brief, the reporter cell line HEK-Blue™ CD40L (Invivogen, transfected with plasmids encoding human CD40 receptor and a NF□B-inducible secreted embryonic alkaline phosphatase (SEAP)) was detached by Accutase (Life Technologies) and 3.4×10⁶ cells were transferred into a sterile 50 mL centrifuge tube (Greiner) and placed on a roller mixer (Heidolph) until use. The HEK-Blue™ Detection Medium (Invivogen) was used from the “two cell line bridging assay for FAP binding and CD40 signal transduction”. The antibody dilution was made 2-fold concentrated in HEK-Blue™ Detection Medium. The final assay concentration was 518 pM. Of the three independent dilution series of the “two cell line bridging assay for FAP binding and CD40 signal transduction” 100 μL of the highest antibody concentration was transferred in duplicates into a clear 96-well micro titer tissue culture plate (Greiner). This resulted in 6 values per molecule being evaluated on 1 plate.

The reporter cell line was centrifuged 4 minutes at 180×g at room temperature. The supernatant was removed and the cells were resuspended in 17 mL HEK-Blue™ Detection Medium. 100 μL comprising 20.000 HEK-Blue™ CD40L cells were present in the wells. The plates were pre-incubated for 15 minutes at room temperature to allow cell distribution and settlement and then incubated for 20 hours at 37° C. and 5% CO₂ (cell incubator). Activity of SEAP was measured with HEK-Blue™ Detection Medium at 650 nm (Victor X4, Perkin Elmer). The mean of the optical density (OD) of n=6 and the 3×STDEV was plotted against the molecules (see FIG. 10).

The highest concentration used in the FAP dependent assay was used to incubate the CD40 specific reporter cells with the molecules. Without clustering via the FAP binding the activation of CD40 receptor and subsequent signal transduction was very low. It was even lower for P1AF0873 (1+1 crossmab) and P1AA9663 (2+2 crossmab) than for the 2+1 molecules P1AE2423 and P1AE2302 (overlapping error bars).

### Example 6

Assessment of the Anti-Tumor Efficacy of FAP-Targeted Anti-Human CD40 Binding Molecules in Combination with PD-L1

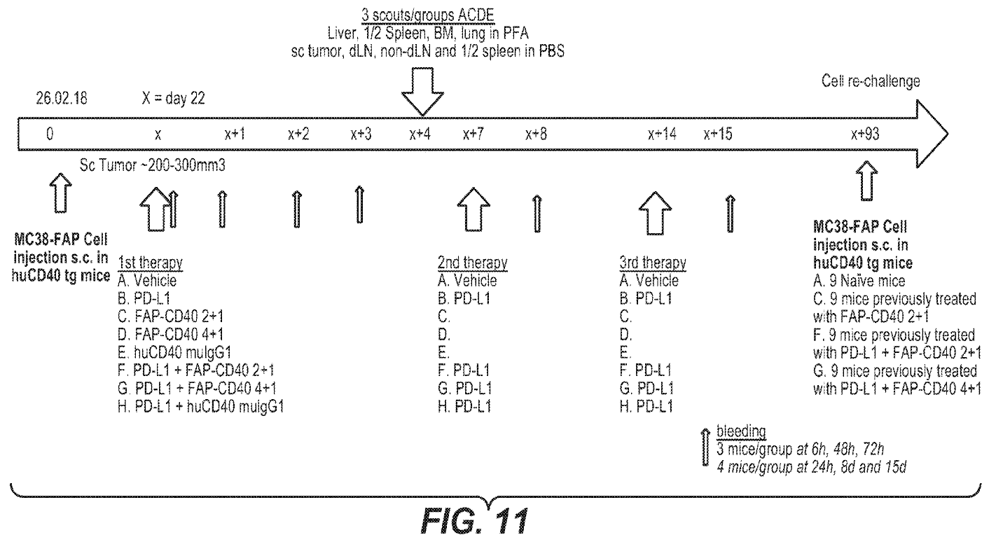

An in vivo study was designed (FIG. 11) in order to determine the effect of agonistic anti-FAP×CD40 antibodies on FAP-expressing tumor cells in immunocompetent mice. In particular, the difference between a bispecific FAP-CD40 antibody in a 4+1 format with a 28H1 FAP binding domain binding to murine FAP, a bispecific FAP-CD40 antibody in a 2+1 format with a 28H1 FAP binding domain and a FAP-independent CD40 control antibody with or without aPD-L1 treatment was evaluated.

A total of 97+9 (needed for the re-challenge experiments) huCD40 tg HO female mice, age 6-9 weeks at the start of experiment (purchased from Charles Rivers, France) and originally obtained from Taconic, were maintained under specific-pathogen-free condition with daily cycles of 12 h light/12 h darkness according to committed guidelines (GV-Solas; Felasa; TierschG). The experimental study protocol was reviewed and approved by the local government (ZH225-17). After arrival, animals were maintained for one week to get accustomed to the new environment and for observation. The mice were afterwards implanted with a transponder subcutaneously on the right side of the back for identification and maintained one more week for recovery. Continuous health monitoring was carried out on regular basis. Animals were controlled daily for clinical symptoms and detection of adverse effects.

MC38-muFAP invipa cells (CRC) were obtained from an in-vivo-passage performed at Roche Glycart AG and after expansion deposited in the Glycart internal cell bank. MC38-muFAP invipa cells were cultured in DMEM containing 10% FCS (PAA Laboratories, Austria), 1 mM Pyruvate, 1×NEAA and 6 □g/ml Puromycine. Cells were cultured at 37° C. in a water-saturated atmosphere at 5% CO₂. Cells were injected at an in vitro passage of 10 and a viability of 94.5%.

For tumor cell inoculation mice were anesthetized and injected subcutaneously, on the left flank, with a 29G needle (BD) with 100 μl of a solution containing 2×10⁶ MC38-muFAP single cell suspension in 50% RPMI+50% Matrigel medium. For the re-challenge with the same tumor cell line 45 mice were injected on the right flank following the same procedure 93 days upon the 1st therapy.

At day 22, when the tumor mean reached 200 mm³, the mice were randomized into 8 different groups of 9 mice each with a similar mean of tumor size (Table 29). After randomization the mice were injected intra-peritoneal with a 29G needle with the different compounds (Table 30) according to the study groups (Table 31). 7 days and 14 days upon the 1st therapy, groups B, F, G and H were injected with a second and third dose of anti-PD-L1 intraperitoneal.

After randomization and therapy injection, the tumor size was followed 3 times a week by caliper measurement. Daily monitoring was performed to ensure the compliance with the welfare of the animals. Among the license criteria (ZH225-17), the mice were sacrificed in case of ulcerated tumors, tumors reaching a tumor volume above 3000 mm3 or mice which suffered a weight loss higher than 20% of their original body weight (at day 0). When termination was needed, the mice were anesthetized with isoflurane and a mechanical cervical dislocation was performed.

TABLE 29
Randomization of animals according to the tumor volume at day 22

| | Tumor Volume (mm³) | | | | | | | |
|---|---|---|---|---|---|---|---|---|
| | | | | | IQR | IQR | N | N |
| | Mean_ | Median_ | StDev_ | SEM_ | (25%) | (75%) | (Animals) | (Measurements) |
| TG1 | 205.87 | 183.09 | 117.82 | 39.27 | 83.77 | 118.89 | 9 | 9 |
| TG2 | 212.06 | 166.15 | 129.07 | 43.02 | 61.73 | 134.20 | 9 | 9 |
| TG3 | 203.06 | 195.36 | 109.90 | 36.63 | 86.46 | 71.22 | 9 | 9 |
| TG4 | 207.98 | 197.16 | 120.68 | 40.23 | 80.87 | 98.34 | 9 | 9 |
| TG5 | 222.03 | 193.11 | 160.66 | 53.55 | 84.25 | 69.29 | 9 | 9 |
| TG6 | 206.73 | 181.89 | 119.04 | 39.68 | 69.52 | 117.74 | 9 | 9 |
| TG7 | 207.83 | 195.20 | 132.17 | 44.06 | 84.94 | 62.45 | 9 | 9 |
| TG8 | 201.83 | 190.58 | 114.43 | 38.14 | 73.22 | 76.46 | 9 | 9 |
| excluded | | | | | | | 25 | |

TABLE 30
Antigen binding molecules used

| | | Substance |
|---|---|---|
| Species | Molecule name | Identifier |
| mouse | PD-L1 6E11 mIgG2a. PG LALA | P1AE0099 |
| human | FAP-CD40 4 + 1: 4 + 1_huIgG1_PGLALA_C- | P1AE2024 |
| | term_xFab_P1AE0817_28H1 | |
| human | FAP-CD40 2 + 1: 2 + 1_huIgG1_PGLALA_C- | P1AE2302 |
| | term_xFab_P1AE0817_28H1 | |
| human | CD40: muIgG1_wt_SGN40 | P1AE2301 |

TABLE 31
Study Groups for treatment

| | 1^{st} therapy | 2^{nd} therapy | 3^{rd} therapy |
|---|---|---|---|
| Group | Day 22 | Day 29 | Day 36 |
| A | Vehicle | Vehicle | Vehicle |
| B | PD-L1 | PD-L1 | PD-L1 |
| C | FAP-CD40 2 + 1 | | |
| D | FAP-CD40 4 + 1 | | |
| E | huCD40 muIgG1 | | |
| F | PD-L1 + FAP-CD40 2 + 1 | PD-L1 | PD-L1 |
| G | PD-L1 + FAP-CD40 4 + 1 | PD-L1 | PD-L1 |
| H | PD-L1 + huCD40 muIgG1 | PD-L1 | PD-L1 |

From the excluded animals, 12 animals were selected and treated with the therapies from group A, C, D and E (3 animals per therapy) in order to be sacrificed as scouts to analyze the immunopharmacodynamic effects at day 4 upon the 1st therapy injection.

### 6.1 FAP-Targeted Anti-CD40 Binding Molecules Induce a Complete Tumor Remission and an Efficient Anti-Tumor Immune Memory Response

The tumor size was monitored 3 times a week by caliper measurement. The JMP12 software was used for the statistical analysis of the tumor growth data. To test for significant differences in group means for multiple comparisons, the standard analysis of variance (ANOVA) using the Tukey-Kramer method was applied. Tukey-Kramer gives a test for all pairwise differences among the means; it is an exact alpha-level test if the samples sizes are the same.

93 days upon the 1st therapy, mice that showed a complete tumor regression were injected subcutaneously on the opposite flank with MC38-FAP tumor cells in order to determine the formation of an immune cell memory against this tumor cell line. As a control, 9 naïve C57bl/6-huCD40 tg mice were injected with the same MC38-FAP tumor cells and the tumor growth was followed by caliper measurements 2 to 3 times a week, according to the vet license.

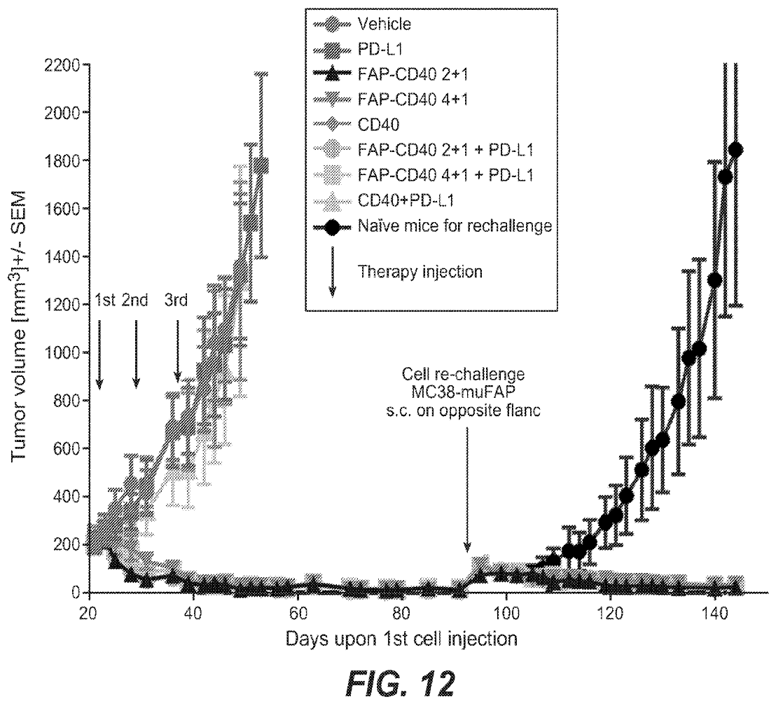

As shown in FIG. 12, both FAP-CD40 bispecific molecules (2+1 and 4+1) induced a complete tumor remission with and without PD-L1 treatment. In mice treated with non-targeted CD40, PD-L1 or both in combination tumor growth was not delayed compared to the vehicle group. In addition, upon re-challenge, no MC38-FAP tumor grew in the groups previously treated with FAP-CD40 antibody whereas in naïve mice 100% of the tumors grew. These results indicate a strong MC38-FAP anti-tumor efficacy and tumor growth regression of the targeted-CD40 therapy in contrast to the non-targeted CD40 therapy. Since all mice treated with an anti-FAP-CD40 antibody showed a 100% tumor remission there was no apparent anti-PD-L1 add-on effect in these mice. In addition, there was no demonstrated difference between the 2+1 and 4-1 format in terms of tumor growth inhibition. The re-challenge data indicate the formation of an efficient immune memory response against MC38-FAP tumor cells in all mice treated with a FAP-CD40 antibody.

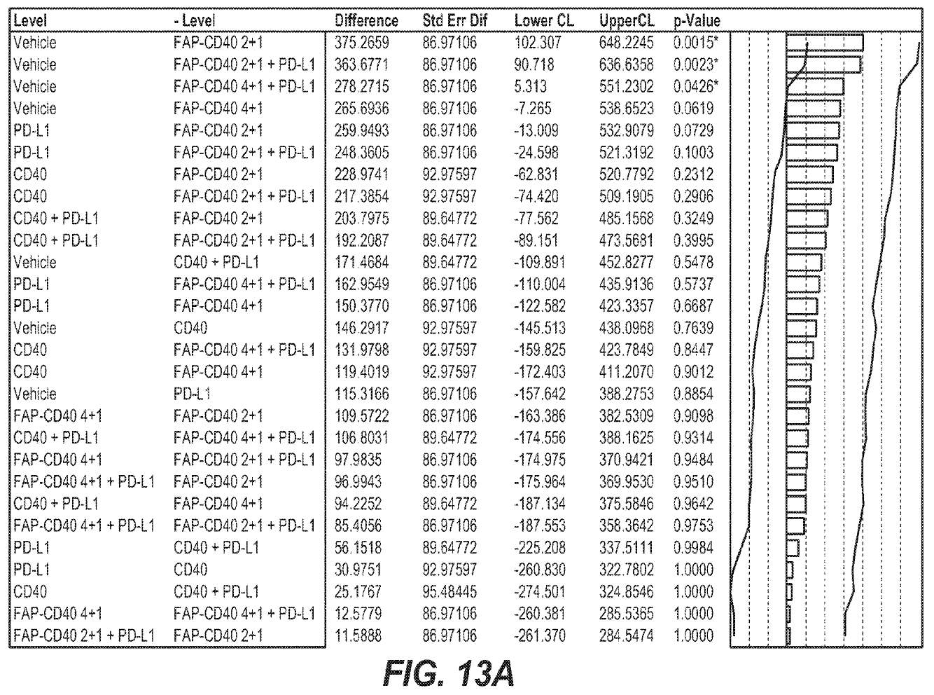

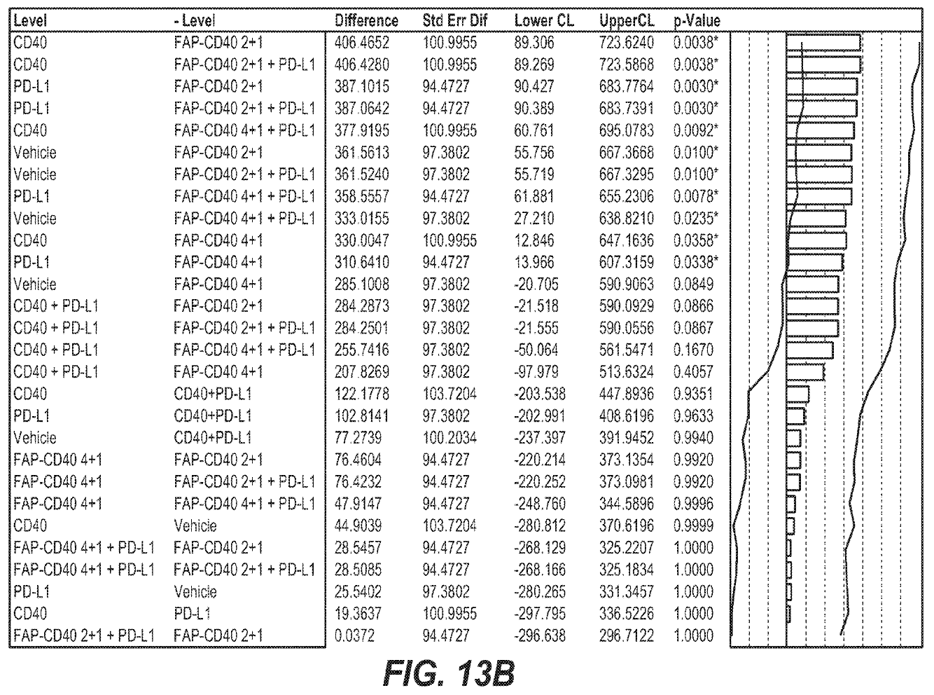

The statistical analysis indicated that the data start to be significantly different as compared to the vehicle from day 28 on (6 days upon therapy) for the 2+1 FAP-CD40 antibody-treated group (FIG. 13A, Statistical analysis comparing all groups at day 28) and from day 31 on (9 days upon therapy) for the 4+1 FAP-CD40 antibody-treated group (FIG. 13B, Statistical analysis comparing all groups at day 31).

### 6.2 the 4+1 FAP-Targeted Anti-CD40 Binding Molecule has a Higher Serum Clearance Compared to the 2+1 FAP-Targeted Anti-CD40 Binding Molecule

Blood from 3 to 4 mice per group were collected in order to follow the pharmacokinetic profile of the drugs upon intraperitoneal injection. Serum samples were collected 6 h, 24 h, 48 h, 72 h, 96 h, 8 days and 15 days following the first antibody therapy to analyze the blood anti-CD40 levels. A maximum of 100 μl of blood per time point was collected. The mice body temperature was slightly raised by a 39° C.-warmed box to ensure a good dilatation of the blood vessels. A puncture on the tail vein was performed with a 22G needle. The blood was collected in 1.1 ml Z-Gel micro tubes (SARSTEDT) and the serum was extracted by collecting the supernatant after centrifugation. Samples were stored at −20° C. and later analyzed for anti-CD40 levels using the respective antibody for calibration.

Anti-CD40 concentrations in the serum were tested using a CD40/Fc chimera (R&D, 1493-CD-050) ELISA for quantification of anti-CD40 huIgG, according to the protocol (Roche-Glycart, Switzerland). The reagents capture protein: human IgG-Fc biotin (Abcam, ab98561) and CD40/Fc chimera (R&D, 1493-CD-050), detection antibody: anti-mouse IgG (HRP) (Abcam, ab97040) or anti-huCH1-DIG (Roche-Penzberg, Germany) and anti-DIG-POD (Roche, 11633716001) were used. Serum samples were analyzed in serial dilutions depending on the injected dosage (mg/kg) and time of collection after anti-CD40 injection (6 h, 24 h, 48 h, 96 h). The absorption was measured using a measuring wavelength of 405 nm and a reference wavelength of 490 nm (SpectraMax i3 microplate reader, Molecular Devices).

For the PK analysis, the Phoenix 64, WinNonlin 6.4 software was used to calculate the area under the curve as well as the Cmax. Since the pharmacokinetics was only monitored up to 96 hours the PK parameters such as clearance and half-life are not depicted.

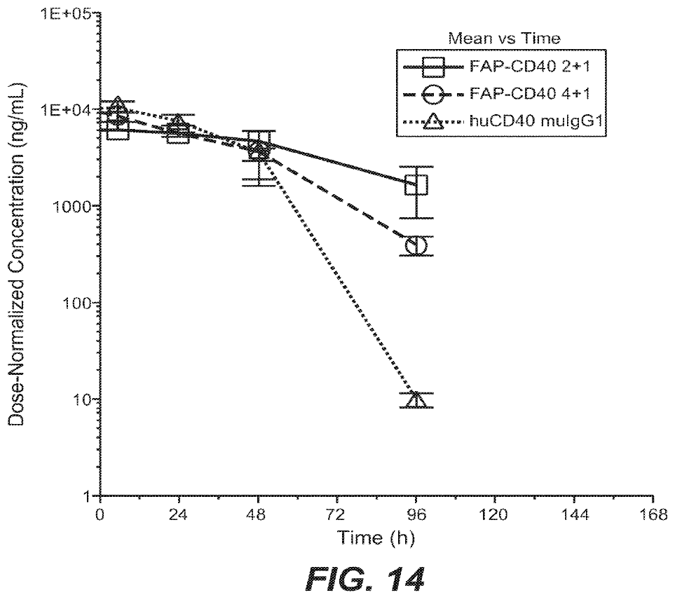

As shown in FIG. 14 and Table 32, a higher clearance was observed for the 4+1 format compared to the 2+1 format. This higher clearance can be explained by the CD40-specific Target-Mediated-Drug-Disposition (TMDD) that is more prominent for a tetravalent CD40 binder (4+1 format) than for a bivalent CD40 binder (2+1 format).

Also, the non-targeted CD40 showed a higher clearance as compared to both FAP-CD40 molecules. This clearance can't be explained by the TMDD hypothesis but could be due to Anti-Drug Antibodies (ADAs) formation upon CD40 therapy.

TABLE 32
PK parameters

| | Dose | Cmax | AUClast |
|---|---|---|---|
| | (mg/kg) | (ng/mL) | (day*ng/mL) |
| FAP-CD40 2 + 1 | 13 | 78914 | 229808 |
| FAP-CD40 4 + 1 | 20 | 168667 | 334143 |
| huCD40 muIgG1 | 10 | 109762 | 193079 |

6.3 CD40-Mediated Activation of DCs, B Cells and T Cells by FAP-Targeted Anti-CD40 Binding Molecules 96 Hours after Treatment

96 h after the 1st therapy, 3 mice per group were sacrificed and the following organs were collected in PBS for flow cytometry analysis: tumor, spleen, inguinal draining lymph nodes and inguinal, axial and brachial non-draining lymph nodes.

For flow cytometer analysis, single cell suspensions of all collected organs were prepared as described in Example 5.2.1 and stained with fluorescently labelled antibodies. For this purpose, the prepared single cell suspensions were transferred into 96-well flat-bottom plates, washed with PBS and incubated with 50 μl of 3 μg/ml of Fc receptor blocking Mouse IgG Isotype Control (ThermoFisher Scientific, Cat. No. 10400C) in PBS. After 15 minutes of incubation at 4° C., cells were washed with PBS and 50 μl of a mixture of fluorescently labelled antibodies in PBS was added to the cells. The following antibodies were used: anti-mouse CD3 Pacific Blue™ (BD Bioscience, clone 500A2, Cat. No. 558214), anti-mouse CD86 BV605 (Biolegend, clone GL-1, Cat. No. 105037), CD45 Alexa Fluor 700 (eBioscience, clone 30-F11, Cat. No. 56-0451-82), anti-mouse CD19 BUV395 (BD Biosciences, clone 1D3, Cat. No. 563557), anti-mouse CD11c BV785 (Biolegend, clone N418, Cat. No. 117336), anti-mouse B220 APC-Cy7 (Biolegend, clone RA3-6B2, Cat. No. 103224), anti-mouse CD69 BUV737 (BD Biosciences, clone H1.2F3, Cat. No. 612793), anti-I-A/I-E PerCp-Cy5.5 (Biolegend, clone M5/114.15.2, Cat. No. 107626). In order to distinguish between live and dead cells, the viability dye Zombie Aqua™ or Fixable blue (both Life technologies) were added to the antibody mixture. Cells were incubated for 30 minutes at 4° C. with the extracellular staining antibody solution. Afterwards, cells were washed two times with PBS, permeabilized and intracellularly stained for Ki-67 using anti-mouse Ki-67 PE-Cy7 (eBioscience, clone SolA15, Cat. No. 25-5698-82) with the Foxp3/Transcription Factor Staining Buffer Set (eBioscience, Cat. No. 00-5523-00) according to the manufacturer's protocol. Cells were resuspended in 200 μl of PBS and analyzed the same day using a 5-laser LSR-Fortessa. Data analysis was performed using the FlowJo version 10 software. Viable CD45+, CD3+ and CD8+ T cells were analyzed for Ki-67 expression. Viable dendritic cells (CD45+, MHCII+, CD11c+) were analyzed for expression of the DC activation marker CD86 and viable B cells (CD45+, CD19+, B220+) were analyzed for expression of the B cell activation marker CD69.

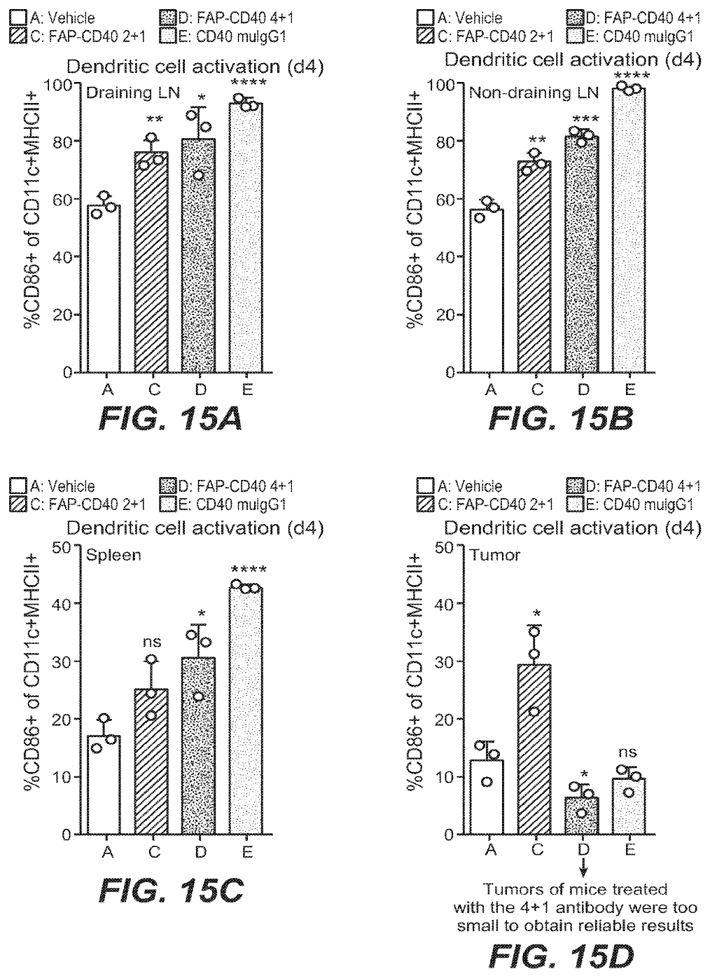

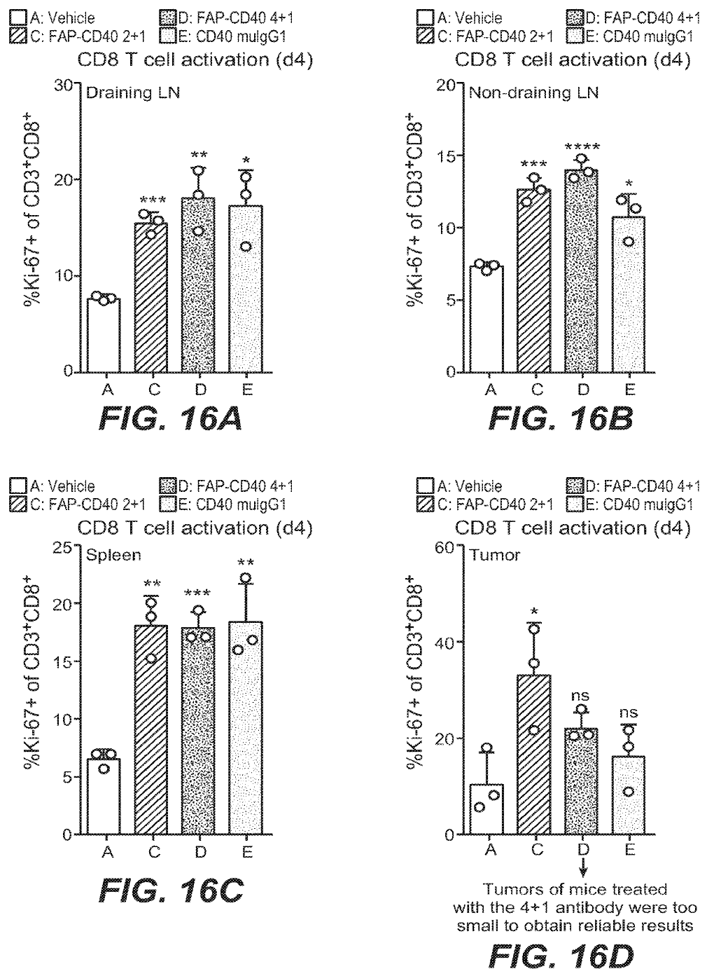

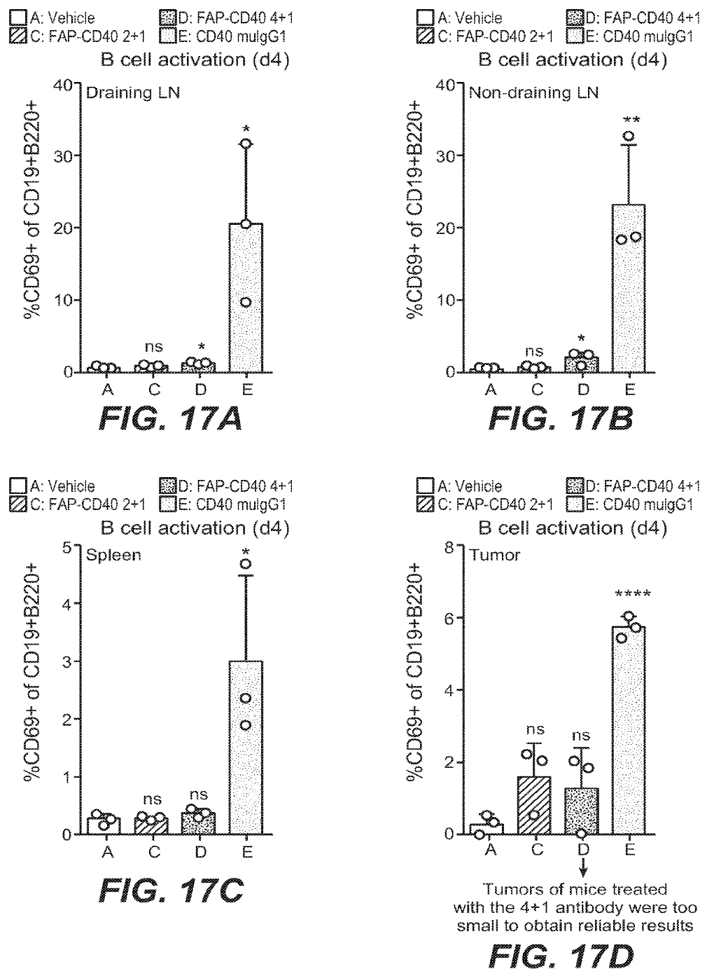

As shown in FIG. 15A to FIG. 15D, both FAP-targeted anti-CD40 antibodies formats (2+1 and 4+1) induced a significant increase in DC activation (CD86 expression) in tumor-draining lymph nodes, non-draining lymph nodes and the spleen four days after treatment although to a lesser extent than the non-targeted anti-CD40 antibody. In contrast, only the FAP-targeted anti-CD40 antibody in a 2+1 format induced a significant DC activation in tumors compared to the vehicle group. As shown in FIG. 16A to FIG. 16D, the same pattern was observed for the activation of T cells (Ki-67 expression). A significant B cell activation (CD69 expression) in all analyzed tissue samples compared to the vehicle group was only observed in mice treated with the non-targeted anti-CD40 antibody (FIG. 17A to FIG. 17D).

### 6.4 FAP-Targeted Anti-CD40 Binding Molecules Induce Less Side Effects Compared to Non-Targeted CD40 Antibody in Mice

6.4.1 Mice Treated with FAP-Targeted Anti-CD40 Antibody Show No Weight Reduction in Contrast to Mice Treated with Non-Targeted Anti-CD40 Antibody

After therapy injection, the weight of each mouse was measured daily for 15 days. Mice with a weight loss higher than 20% of their original body weight (at day 0) were sacrificed (here, no mice needed to be sacrificed).

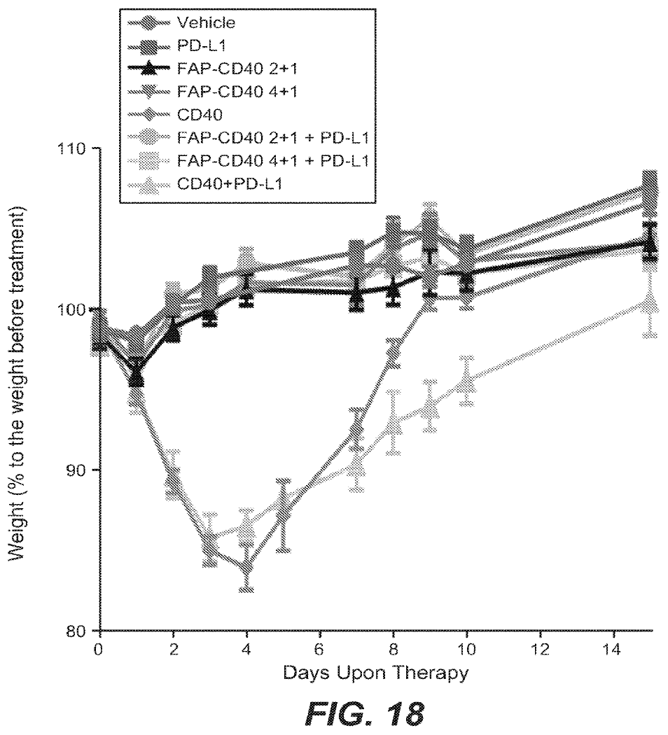

As FIG. 18 illustrates mice treated with non-targeted anti-CD40 antibody showed a clear weight loss upon antibody injection and this weight loss lasted longer in the case of a concomitant injection of anti-CD40 with anti-PD-L1 antibody. No weight loss was observed in the groups injected with 4+1 or 2+1 FAP-CD40 single agents, PD-L1 single agent and in the combination groups FAP-CD40 (4+1 or 2+1)+PD-L1. This result indicates a safe profile of the FAP-targeted anti-CD40 binding molecules as compared to the non-targeted anti-CD40 antibody.

6.4.1 Mice Treated with FAP-Targeted Anti-CD40 Antibody Show Less Anti-PD-L1-Mediated Adverse Events Compared to Mice Treated with Non-Targeted Anti-CD40 Antibody

Some adverse events were observed upon the 3rd injection of the anti-PD-L1 antibody. No mice needed to be sacrificed but the symptoms were described as decreased activity of the mouse within 5 minutes after therapy injection, arched back and scruffy fur. Table 33 indicates the percent of mice showing those symptoms upon the 3rd therapy.

TABLE 33
Percentage of animals showing adverse
events upon 3rd anti-PD-L1 therapy.

| | % of mice showing | Recovery |
|---|---|---|
| | adverse events upon | Time |
| Group | 3rd injection of PD-L1 | (minutes) |
| PD-L1 | 33% | 30-40 |
| FAP-CD40 2 + 1 + PD-L1 | 44% | 10-20 |
| FAP-CD40 4 + 1 + PD-L1 | 55% | 10-20 |
| CD40 + PD-L1 | 100% | 30-40 |

The results show a clear increase of adverse events in the combination group PD-L1+CD40 whereas the percent of adverse events observed in the anti-PD-L1+FAP-CD40 groups (4+1 and 2+1) were only slightly increased compared to the anti-PD-L1 single agent-treated group.

The observed adverse events could be the result of a strong immune reaction (cytokine storm) due to a ADAs formation against anti-PD-L1, which is further increased in the presents of a non-targeted anti-CD40 antibody. This observation indicates a better safety profile of the targeted FAP-CD40 molecule compared to the non-targeted anti-CD40 antibody when both molecules are combined with an anti-PD-L1 antibody therapy.

In summary, the FAP-targeted anti-CD40 molecules with FAP-dependent activation of CD40 in a 4+1 and 2+1 format induce a potent anti-tumor immune response in tumor-bearing mice with reduced systemic toxicity compared to the untargeted anti-CD40 parental antibody.

1.-34. (canceled)

35. A bispecific antigen-binding molecule that specifically binds CD40 and Fibroblast Activation Protein (FAP), wherein said bispecific antigen-binding molecule comprises:
a. a first light chain comprising the amino acid sequence of SEQ ID NO: 61; a second light chain comprising the amino acid sequence of SEQ ID NO: 62; a first heavy chain comprising the amino acid sequence of SEQ ID NO: 63; and a second heavy chain comprising the amino acid sequence of SEQ ID NO: 64; or
b. a first light chain comprising the amino acid sequence of SEQ ID NO: 65; a second light chain comprising the amino acid sequence of SEQ ID NO: 66; a first heavy chain comprising the amino acid sequence of SEQ ID NO: 67; and a second heavy chain comprising the amino acid sequence of SEQ ID NO: 68; or
c. a first light chain comprising the amino acid sequence of SEQ ID NO: 61; a second light chain comprising the amino acid sequence of SEQ ID NO: 62; a first heavy chain comprising the amino acid sequence of SEQ ID NO: 69; and a second heavy chain comprising the amino acid sequence of SEQ ID NO: 70; or
d. a first light chain comprising the amino acid sequence of SEQ ID NO: 71; a second light chain comprising the amino acid sequence of SEQ ID NO: 66; a first heavy chain comprising the amino acid sequence of SEQ ID NO: 72; and a second heavy chain comprising the amino acid sequence of SEQ ID NO: 68; or
e. a first light chain comprising the amino acid sequence of SEQ ID NO: 71; a second light chain comprising the amino acid sequence of SEQ ID NO: 66; a first heavy chain comprising the amino acid sequence of SEQ ID NO: 73; and a second heavy chain comprising the amino acid sequence of SEQ ID NO: 74; or
f. a first light chain comprising the amino acid sequence of SEQ ID NO: 75; a second light chain comprising the amino acid sequence of SEQ ID NO: 62; a first heavy chain comprising the amino acid sequence of SEQ ID NO: 76; and a second heavy chain comprising the amino acid sequence of SEQ ID NO: 64; or
g. a first light chain comprising the amino acid sequence of SEQ ID NO: 75; a second light chain comprising the amino acid sequence of SEQ ID NO: 62; a first heavy chain comprising the amino acid sequence of SEQ ID NO: 77; and a second heavy chain comprising the amino acid sequence of SEQ ID NO: 70; or
h. a first light chain comprising the amino acid sequence of SEQ ID NO: 61; a second light chain comprising the amino acid sequence of SEQ ID NO: 62; a first heavy chain comprising the amino acid sequence of SEQ ID NO: 63; and a second heavy chain comprising the amino acid sequence of SEQ ID NO: 98; or
i. a first light chain comprising the amino acid sequence of SEQ ID NO: 75; a second light chain comprising the amino acid sequence of SEQ ID NO: 105; a first heavy chain comprising the amino acid sequence of SEQ ID NO: 106; and a second heavy chain comprising the amino acid sequence of SEQ ID NO: 106.

36. A pharmaceutical composition comprising the bispecific antigen-binding molecule of claim 35, and a pharmaceutically-acceptable carrier.

37. The pharmaceutical composition of claim 36, further comprising an additional therapeutic agent.

38. A nucleic acid or set of nucleic acids encoding the bispecific antigen-binding molecule of claim 35.

39. A vector or set of vectors comprising the nucleic acid or set of nucleic acids of claim 38.

40. A host cell comprising the nucleic acid or set of nucleic acids of claim 38.

41. A host cell comprising the vector or set of vectors of claim 38.

42. A method of producing a bispecific antigen-binding molecule comprising culturing the host cell of claim 41 under conditions suitable for the expression of the bispecific antigen binding molecule, and isolating the bispecific antigen binding molecule.

43. A method of treating an individual having a Fibroblast Activation Protein (FAP)-expressing cancer comprising administering to the individual an effective amount of a bispecific antigen binding molecule that comprises:
a. a light chain comprising the amino acid sequence of SEQ ID NO: 61; a light chain comprising the amino acid sequence of SEQ ID NO: 62; a heavy chain comprising the amino acid sequence of SEQ ID NO: 63; and a heavy chain comprising the amino acid sequence of SEQ ID NO: 64; or
b. a light chain comprising the amino acid sequence of SEQ ID NO: 65; a light chain comprising the amino acid sequence of SEQ ID NO: 66; a heavy chain comprising the amino acid sequence of SEQ ID NO: 67; and a heavy chain comprising the amino acid sequence of SEQ ID NO: 68; or
c. a light chain comprising the amino acid sequence of SEQ ID NO: 61; a light chain comprising the amino acid sequence of SEQ ID NO: 62; a heavy chain comprising the amino acid sequence of SEQ ID NO: 69; and a heavy chain comprising the amino acid sequence of SEQ ID NO: 70; or
d. a light chain comprising the amino acid sequence of SEQ ID NO: 71; a light chain comprising the amino acid sequence of SEQ ID NO: 66; a heavy chain comprising the amino acid sequence of SEQ ID NO: 72; and a heavy chain comprising the amino acid sequence of SEQ ID NO: 68; or
e. a light chain comprising the amino acid sequence of SEQ ID NO: 71; a light chain comprising the amino acid sequence of SEQ ID NO: 66; a heavy chain comprising the amino acid sequence of SEQ ID NO: 73; and a heavy chain comprising the amino acid sequence of SEQ ID NO: 74; or
f. a light chain comprising the amino acid sequence of SEQ ID NO: 75; a light chain comprising the amino acid sequence of SEQ ID NO: 62; a heavy chain comprising the amino acid sequence of SEQ ID NO: 76; and a heavy chain comprising the amino acid sequence of SEQ ID NO: 64; or
g. a light chain comprising the amino acid sequence of SEQ ID NO: 75; a light chain comprising the amino acid sequence of SEQ ID NO: 62; a heavy chain comprising the amino acid sequence of SEQ ID NO: 77; and a heavy chain comprising the amino acid sequence of SEQ ID NO: 70; or
h. a light chain comprising the amino acid sequence of SEQ ID NO: 61; a light chain comprising the amino acid sequence of SEQ ID NO: 62; a heavy chain comprising the amino acid sequence of SEQ ID NO: 63; and a heavy chain comprising the amino acid sequence of SEQ ID NO: 98; or
i. a light chain comprising the amino acid sequence of SEQ ID NO: 75; a light chain comprising the amino acid sequence of SEQ ID NO: 105; a heavy chain comprising the amino acid sequence of SEQ ID NO: 106; and a heavy chain comprising the amino acid sequence of SEQ ID NO: 106.

44. The method of claim 43, wherein said FAP-expressing cancer is colorectal carcinoma, gastric cancer, skin cancer, breast cancer, lung cancer, bladder cancer, or ovarian cancer.
